(19) United States
(12) Patent Application Publication
(10) Pub. No.: US 2025/0313546 A1
(43) Pub. Date: Oct. 9, 2025

(54) NUCLEIC ACID BINDERS

(71) Applicant: UNIVERSITY OF STRATHCLYDE, Glasgow (GB)

(72) Inventors: Colin James SUCKLING, Glasgow (GB); Abedawn Ibrahim KHALAF, Glasgow (GB); Fraser SCOTT, Glasgow (GB)

(21) Appl. No.: 18/864,977

(22) PCT Filed: May 11, 2023
(86) PCT No.: PCT/GB2023/051235

§ 371 Date: Nov. 12, 2024

(30) Foreign Application Priority Data
May 12, 2022 (GB) 2206931.4

(51) Int. Cl. C07D 401/14; A61K 31/4025; A61K 31/4245; A61K 31/428; A61K 31/454; A61K 31/455; A61K 31/4709; A61K 31/496; A61K 31/506; A61K 31/5377; A61K 31/541; A61K 31/55; A61P 31/04; A61P 31/10; A61P 31/14; A61P 31/16; A61P 33/02; C07D 207/34; C07D 403/14; C07D 405/14; C07D 413/14; C07D 417/14

(52) U.S. Cl. CPC C07D 401/14; A61K 31/4025; A61K 31/4245; A61K 31/428; A61K 31/454; A61K 31/455; A61K 31/4709; A61K 31/496; A61K 31/506; A61K 31/5377; A61K 31/541; A61K 31/55; A61P 31/04; A61P 31/10; A61P 31/14; A61P 31/16; A61P 33/02; C07D 207/34; C07D 403/14; C07D 405/14; C07D 413/14; C07D 417/14

(57) ABSTRACT

The present invention concerns compounds suitable as RNA or DNA binders. RNA or DNA binders are useful in the treatment of various conditions, including those caused by microbial infection and cancer. The present invention concerns specific compounds, and such compounds for use in methods of treatment, such as the treatment of antimicrobial infection and/or cancer.

### FIELD OF THE INVENTION

The present invention concerns compounds suitable as RNA or DNA binders. RNA or DNA binders are useful in the treatment of various conditions, including those caused by microbial infection and cancer. The present invention concerns specific compounds, and such compounds for use in methods of treatment, such as the treatment of antimicrobial infection and/or cancer.

### BACKGROUND OF THE INVENTION

Many available antimicrobials have been, and will be, rendered ineffective due to the acquisition of resistance by pathogenic organisms. In certain applications, such as emergency prophylaxis, either in the clinic or on the battlefield, broad-spectrum anti-infective agents are desirable. These may be broad-spectrum within a particular type of pathogen, such as broad-spectrum anti-fungals, or broad-spectrum across different pathogen types, such as compounds that are simultaneously antibacterial, antifungal, antiparasitic and antiviral. In certain applications, when the specific pathogen causing an infection is known, narrow-spectrum agents are desirable, as this can prevent side effects, such as when the gut microbiome is disturbed during treatment with an antibiotic, or can reduce the rate of development of antimicrobial resistance.

DNA is an important target for drug action, such as antimicrobial drug action. DNA binders are typically classified by their mode of binding—either as intercalators or as groove binders. DNA intercalators are typically planar, aromatic compounds and are able to fit in between the base pairs of DNA, whilst groove binders are typically aromatic compounds and are able to bind to either or both of the two channels on the outer surface of double-helical DNA (typically in B-form), namely the major and minor grooves. The major groove contains approximately twice the number of potential hydrogen-bonding contacts than the minor groove. In view of this, the major groove is the preferred recognition site for cellular proteins such as control proteins, promoters and repressors. In contrast, the minor groove is relatively unoccupied. The vulnerability of the minor groove makes it a particularly useful target for compounds that bind to DNA and it is the binding site for some naturally occurring antibiotics (such as netropsin and distamycin).

RNA is the genetic material of many pathogenic viruses, and so is also an important target for drug action. RNA exists in either single- or double-stranded forms, and is commonly single-stranded. RNA fluctuates in structure but most often exists in a helical conformation known as the A-form. Double-stranded RNA also typically adopts an A-form helical conformation. The minor and major grooves of A-form RNA duplexes differ significantly from those of B-form DNA: the minor and major grooves are different in shape (the major groove is narrower and deeper and the minor groove is wider and shallower in A-form RNA with respect to B-form DNA) and in chemical environment the 2′-OH of RNA is situated in the minor groove.

Compounds having an affinity for DNA (particularly MGBs) are described in WO 2008/038018 (University of Strathclyde). These compounds are described as having anti-infective effects through binding to the minor groove of DNA and interfering with DNA-centric processes within pathogens. More recently, compounds with related structures have been identified that can also bind to RNA, and these are known as Nucleic Acid Binders (NABs) to account for this additional mechanism of action.

Despite having potent activities, many NABs are unsuitable as drugs owing to an unfavourable selectivity index between pathogen and host. There is a need in the art for alternative NABs, preferably with improved selectivity, and the present invention addresses this need.

### SUMMARY OF THE INVENTION

The present invention is based on the unexpected finding that modification of NAB structure, via increasing or decreasing chain lengths within the structure, leads to the production of NABs with selective toxicity, combined with effective DNA and RNA binding and potent anti-infective activity. Accordingly, the present invention provides alternative NABs with improved selectivity, useful in the treatment of antimicrobial infection and/or cancer.

Viewed from a first aspect, the invention provides a compound of any one of formulae I, II, III and IV:

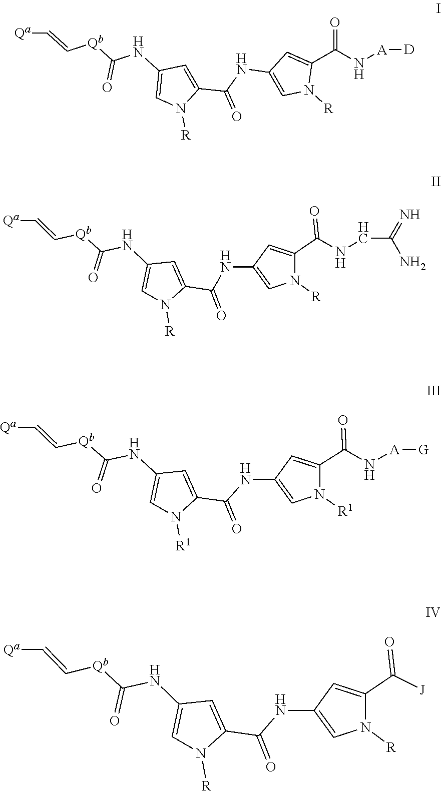

wherein:
Q^{a} is optionally substituted aryl or optionally substituted heteroaryl and Q^{b} is optionally substituted arylene or optionally substituted heteroarylene, wherein the aryl, heteroaryl, arylene and heteroarylene are optionally substituted with any one or a combination selected from the group consisting of N(C₁₋₆alkyl)₂, haloC₁₋₆alkyl, cyano, C₁₋₆alkyl, C₁₋₆alkoxy, NH(C₁₋₆alkyl), NH₂, halo and OH;
each R is independently selected from the group consisting of C₁₋₆alkyl, H and haloC₁₋₆alkyl;
each R¹ is independently selected from the group consisting of C₁₋₆alkyl, H and haloC₁₋₆alkyl, wherein at least one R¹ is H;
A is C₁₋₆alkylene or haloC₁₋₆alkylene;
E is C₄₋₆alkylene or haloC₄₋₆alkylene;
A-D is selected from the group consisting of formulae Ia to Id:

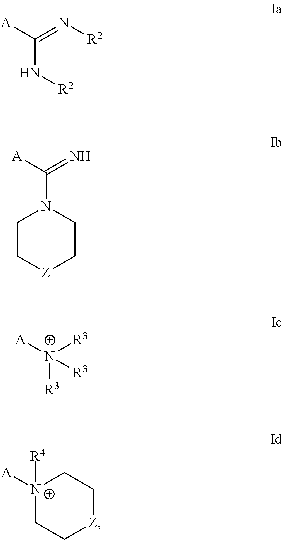

wherein:
Z is selected from the group consisting of O, CH₂, N(C₁₋₆alkyl) and S; and
each R² is independently selected from the group consisting of H, C₁₋₂₀alkyl, C₁₋₂₀alkoxy, C₂₋₂₀alkenyl, C₄₋₂₀dialkenyl and C₈₋₂₀trialkenyl, wherein at least one R² is not H, and wherein the C₁₋₂₀alkyl, C₁₋₂₀alkoxy, C₂₋₂₀alkenyl, C₄₋₂₀dialkenyl and C₆₋₂₀trialkenyl are optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo;
each R³ is independently selected from the group consisting of C₁₋₂₀alkyl, C₂₋₂₀alkenyl, C₄₋₂₀dialkenyl and C₆₋₂₀trialkenyl, optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O, and OH, wherein the aryl and heterocyclyl are optionally substituted with any one or more independently selected from the group consisting of C₁₋₄alkyl, haloC₁₋₄alkyl, C₁₋₄alkoxy and halo;
R⁴ is selected from the group consisting of C₂₋₂₀alkyl, C₂₋₂₀alkenyl, C₄₋₂₀dialkenyl and C₆₋₂₀trialkenyl, optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo;
A-G is selected from the group consisting of formulae IIIa and IIIb:

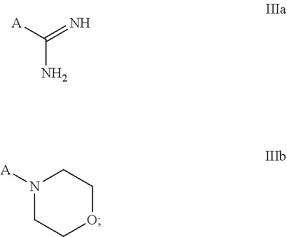

C(O)-J is any one selected from the group consisting of formulae IVa and IVb:

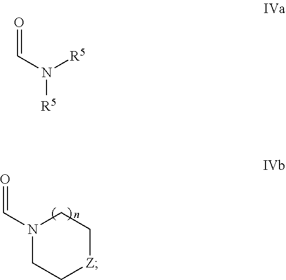

wherein:
each R⁵ is independently selected from the group consisting of H, C₁₋₂₀alkyl, C₂₋₂₀alkenyl, C₄₋₂₀dialkenyl and C₈₋₂₀trialkenyl, wherein the C₂₋₂₀alkenyl, C₄₋₂₀dialkenyl and C₆₋₂₀trialkenyl are optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, piperidinyl, piperazinyl, morpholinyl, thiomorpholinyl, aryl, halo, cyano, C₁₋₆alkoxy, ═O and OH, and the C₁₋₂₀alkyl is optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl, halo, cyano, C₁₋₆alkoxy, ═O and OH, wherein the cycloC₃₋₈alkyl, pyrrolidinyl, piperidinyl, piperazinyl, morpholinyl, thiomorpholinyl, 1-(3-propyl)piperidine, 1-(3-propyl)piperazine, 4-(3-propyl)morpholine, 4-(3-propyl)thiomorpholine and aryl are optionally substituted with any one or more independently selected from the group consisting of C₁₋₄alkyl, haloC₁₋₄alkyl, C₁₋₄alkoxy and halo; and
n is 1 to 3.

The inventors have found that modification of the length of groups R, A, D, E, R¹, G and J leads to the production of compounds with unexpectedly improved selectivity, measured as an increase in selectivity index. The selectivity index has been calculated by the inventors as the minimum inhibitory concentration (MIC) required to inhibit 50% mammalian cell growth divided by the minimum inhibitory concentration (MIC) required to inhibit a specific percentage (50%, 80% or 99%) of microbial cell growth. In other words, a greater selectivity index corresponds to a greater inhibition of pathogen cell over host cell, thus more selective compounds are advantageous as drugs.

Viewed from a second aspect, the invention provides a composition comprising one or more compounds of the first aspect and a pharmaceutically acceptable excipient.

As described above, the compounds of the invention exhibit improved selectivity, and are useful in the treatment of antimicrobial infection and/or cancer. Therefore, viewed from a third aspect, the invention provides a compound of the first aspect or a composition of the second aspect for use as a medicament.

Viewed from a fourth aspect, the invention provides a compound of the first aspect or a composition of the second aspect for use in the treatment of any one or more selected from the group consisting of viral infection, bacterial infection, fungal infection, parasitic infection and cancer.

Viewed from a fifth aspect, the invention provides a method of treatment of any one or more selected from the group consisting of a viral infection, bacterial infection, fungal infection, parasitic infection and cancer, said method comprising administering an effective amount of a compound of the first aspect or a composition of the second aspect to a patient in need thereof.

As described above, the inventors have found that the compounds of the invention are able to bind to DNA and RNA, and are thus known as Nucleic Acid Binders (NABs). Viewed from a sixth aspect, therefore, the invention provides the use of a compound of the first aspect or a composition of the second aspect in binding RNA or DNA, wherein said binding is ex vivo or in vitro.

### DETAILED DESCRIPTION OF THE INVENTION

As described above, the inventors have found that modification of NAB structure, via increasing or decreasing chain lengths within the structure, leads to the production of compounds with improved selectivity, useful in the treatment of antimicrobial infection and/or cancer.

In the discussion that follows, reference is made to a number of terms, which are to be understood to have the meanings provided below, unless a context indicates to the contrary. The nomenclature used herein for defining compounds, in particular the compounds described herein, is intended to be in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 68, 2287-2311 (1996)). For the avoidance of doubt, if an IUPAC rule is contrary to a definition provided herein, the definition herein is to prevail.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof will be understood to imply the inclusion of a stated element, integer or step, or group of elements, integers or steps, and the exclusion of any other element, integer or step or group of elements, integers or steps.

The term “about” herein, when qualifying a number or value, is used to refer to values that lie within ±5% of the value specified. For example, if a suitable daily dose is indicated to be about 0.1 to about 100 mg/kg, doses of 0.095 to 105 mg/kg are included.

The term “alkyl” is well known in the art and defines univalent groups derived from alkanes by removal of a hydrogen atom from any carbon atom, wherein the term “alkane” is intended to define acyclic branched or unbranched hydrocarbons having the general formula CₙH₂ₙ₊₂, wherein n is an integer 1. Examples of alkyl groups include methyl, ethyl, n-propyl, iso-propyl, n-butyl, sec-butyl, iso-butyl and tert-butyl.

The term “haloalkyl” refers to alkyl groups in which at least one hydrogen atom has been replaced with a halo atom, such as fluoro, chloro or bromo, often fluoro. Trifluoromethyl is an example of a haloalkyl.

The term “alkylene” is used synonymously with the term “alkanediyl” and defines bivalent groups derived from alkanes by removal of two hydrogen atoms from any carbon atoms (including the removal of two hydrogen atoms from the same carbon atom). C₂-C₄alkylene refers to any one selected from the group consisting of ethylene, n-propylene, iso-propylene, n-butylene, sec-butylene, iso-butylene and tert-butylene.

The term “haloalkylene” refers to alkylene groups in which at least one hydrogen atom has been replaced with a halo atom, such as fluoro, chloro or bromo, often fluoro. Tetrafluoroethylene is an example of a haloalkylene.

The term “cycloalkane” defines saturated monocyclic unbranched hydrocarbons, having the general formula CₙH₂ₙ, wherein n is an integer≥3. C₃₋₆cycloalkyl refers to any one selected from the group consisting of cyclopropyl, cyclobutyl, cyclopentyl and cyclohexyl.

The term “cycloalkyl” defines all monovalent groups derived from cycloalkanes by removal of one hydrogen atom from a ring carbon atom.

The term “alkenyl” defines univalent groups derived from alkenes by removal of a hydrogen atom from any carbon atom, wherein the term “alkene” is intended to define acyclic branched or unbranched hydrocarbons having the general formula CₙH₂ₙ, wherein n is an integer≥2. Examples of alkenyl groups include ethylenyl, n-propylenyl, iso-propylenyl, n-butylenyl, sec-butylenyl, iso-butylenyl and tert-butylenyl.

The term “dialkenyl” means univalent groups derived from dialkenes by removal of a hydrogen atom from any carbon atom, wherein the term “dialkene” defines acyclic branched or unbranched hydrocarbons having the general formula CₙH₂ₙ₊₂. Similarly, the term “trialkenyl” means univalent groups derived from trialkenes by removal of a hydrogen atom from any carbon atom, wherein the term “trialkene” defines acyclic branched or unbranched hydrocarbons having the general formula CₙH₂ₙ₋₄.

The term “alkoxy” defines monovalent groups derived from alcohols by removal of the hydrogen atom bonded to the hydroxyl group. The term “alcohols” defines groups derived from alkanes, in which one hydrogen atom has been replaced with a hydroxyl group. Methoxy is an example of a C₁alkoxy group.

The term “aryl” defines all univalent groups formed on removing a hydrogen atom from an arene ring carbon and the term “arylene” defines all bivalent groups formed on removing two hydrogen atoms from an arene ring carbon. The term “arene” defines monocyclic or polycyclic aromatic hydrocarbons, where “aromatic” defines a cyclically conjugated molecular entity with a stability (due to delocalisation) significantly greater than that of a hypothetical localised structure. The Hückel rule is often used in the art to assess aromatic character; monocyclic planar (or almost planar) systems of trigonally (or sometimes digonally) hybridised atoms that contain (4n+2) π-electrons (where n is a non-negative integer) will exhibit aromatic character. The rule is generally limited to n=0 to 5.

The term “naphthylene” refers to univalent groups derived from naphthalene by removal of a hydrogen atom from a carbon atom.

The term “heteroarene” defines compounds formally derived from arenes by replacement of one or more methine (—C═) and/or vinylene (—CH═CH—) groups by trivalent or divalent heteroatoms, respectively, in such a way as to maintain the continuous π-electron system characteristic of aromatic systems. The term “heteroaryl” defines all univalent groups formed on removing a hydrogen atom from a heteroarene ring atom such as carbon or nitrogen and the term “heteroarylene” defines all bivalent groups formed on removing two hydrogen atoms from a heteroarene ring atom such as carbon or nitrogen. Typically, the heteroaryl or heteroarylene groups herein comprise any one or a combination of heteroatoms selected from the group consisting of nitrogen, oxygen and sulfur atoms.

The term “heterocyclyl” defines all univalent groups formed on removing a hydrogen atom from a heterocycle ring atom such as carbon or nitrogen. The term “heterocycle” defines cyclic compounds having as ring members atoms of at least two different elements, typically carbon and one or more heteroatoms, such as nitrogen, oxygen and/or sulfur. This term encompasses aromatic (heteroarenes) and non-aromatic compounds.

For the avoidance of doubt, by methoxy(ethoxy)₁₋₃, is meant —(O—CH₂CH₂)₁₋₃—O—CH₃ and by hydroxy(ethoxy)₁₋₃ is meant —(O—CH₂CH₂)₁₋₃—OH. Also, by di(C₁₋₄alkyl)amino is meant N(C₁₋₄alkyl)₂, C₁₋₄alkylamino is meant NH(C₁₋₄alkyl) and amino is meant NH₂.

The term “treatment” defines the therapeutic treatment of a subject that may be a human or non-human animal (e.g. a domesticated animal such as a farmed animal), in order to impede or reduce or halt the rate of progress of a condition, or to ameliorate or cure the condition. Prophylaxis of the condition as a result of treatment is also included. References to prophylaxis are intended herein not to require complete prevention of a condition: its development may instead be hindered through treatment in accordance with the invention. In certain instances, the term “treatment” is used to define the therapeutic treatment of a subject in order to impede or reduce or halt the rate of progress of a condition, or to ameliorate or cure the condition. In those instances, the subject has the condition.

By an “effective amount” herein defines an amount of any one or a combination of the compounds or compositions described herein that is sufficient to impede a condition and thus produces the desired therapeutic or inhibitory effect.

The term “ex vivo” is used herein to refer to processes conducted in or on tissue from an organism in an external environment.

The term “in vitro” is used herein to refer to processes performed with microorganisms, cells, or biological molecules outside their normal biological context.

The term “stereoisomer” is used herein to refer to isomers that possess identical molecular formulae and sequence of bonded atoms, but which differ in the arrangement of their atoms in space.

The term “enantiomer” defines one of a pair of molecular entities that are mirror images of each other and non-superimposable, i.e. cannot be brought into coincidence by translation and rigid rotation transformations. Enantiomers are chiral molecules, i.e. are distinguishable from their mirror image.

The term “racemic” is used herein to pertain to a racemate. As used herein, a racemate defines a substantially equimolar mixture (about 50% of one enantiomer and about 50% of the other enantiomer) of a pair of enantiomers.

The term “diastereoisomers” (also known as diastereomers) defines stereoisomers that are not related as mirror images.

The term “solvate” is used herein to refer to a complex comprising a solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

The term “isotope” is used herein to define a variant of a particular chemical element, in which the nucleus necessarily has the same atomic number but has a different mass number owing to it possessing a different number of neutrons.

The term “prodrug” is used herein to refer to a compound which acts as a drug precursor and which, upon administration to a subject, undergoes conversion by metabolic or other chemical processes to yield a compound disclosed herein.

The term “pharmaceutically acceptable excipient” defines substances other than a pharmacologically active drug or prodrug, which are included in a pharmaceutical product.

The term “enteral” is used to refer to administration of a compound through the gastrointestinal tract. Enteral administration may be oral administration, i.e. administration through the mouth.

The term “parenteral” is used to refer to administration of a compound into the body via means other than the gastrointestinal tract. Parenteral administration includes intravenous administration (directly into a vein), intramuscular administration (into the muscle), intradermal administration (into the skin) or subcutaneous administration (beneath the skin, e.g. into fat). Parenteral administration may be carried out via a bolus injection, in which a discrete amount of compound is administered in one injection.

Unless otherwise specified, the term “RNA” refers to either single or double stranded RNA and the term “RNA sequence” includes any part of (or the whole of) an RNA oligomer or polymer spanning three or more bases. “DNA” refers to either single or double-stranded DNA and the term “DNA sequence” includes any part of (or the whole of) a DNA oligomer or polymer spanning three or more base pairs.

The term “virus comprising RNA” is used interchangeably herein with the term “RNA virus” and refers to a virus comprising RNA as its genetic material. RNA viruses are those belonging to Group III, Group IV, Group V or Group VI of the Baltimore classification, i.e. those comprising double stranded RNA, positive sense single stranded RNA (including those with DNA intermediates in their life cycle, such as retroviruses), or antisense (or negative sense) single stranded RNA.

As described above, in a first aspect, the present invention provides a compound of any one of formulae I, II, III and IV:

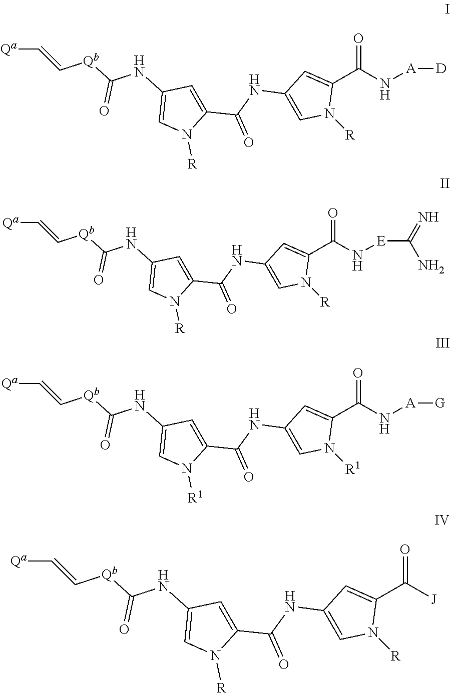

wherein:
Q^{a} is optionally substituted aryl or optionally substituted heteroaryl and Q^{b} is optionally substituted arylene or optionally substituted heteroarylene, wherein the aryl, heteroaryl, arylene and heteroarylene are optionally substituted with any one or a combination selected from the group consisting of N(C₁₋₆alkyl)₂, haloC₁₋₆alkyl, cyano, C₁₋₆alkyl, C₁₋₆alkoxy, NH(C₁₋₆alkyl), NH₂, halo and OH;
each R is independently selected from the group consisting of C₁₋₆alkyl, H and haloC₁₋₆alkyl;
each R¹ is independently selected from the group consisting of C₁₋₆alkyl, H and haloC₁₋₆alkyl, wherein at least one R¹ is H;
A is C₁₋₆alkylene or haloC₁₋₆alkylene;
E is C₄₋₆alkylene or haloC₄₋₆alkylene;
A-D is selected from the group consisting of formulae Ia to Id:

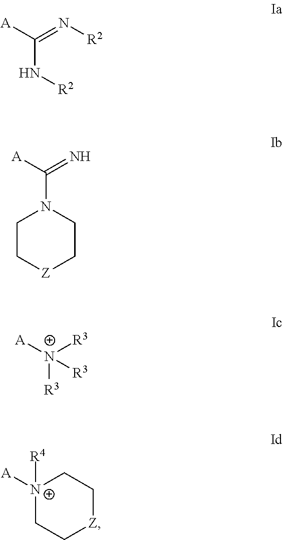

wherein:
Z is selected from the group consisting of O, CH₂, N(C₁₋₆alkyl) and S; and
each R² is independently selected from the group consisting of H, C₁₋₂₀alkyl, C₁₋₂₀alkoxy, C₂₋₂₀alkenyl, C₄₋₂₀dialkenyl and C₈₋₂₀trialkenyl, wherein at least one R² is not H, and wherein the C₁₋₂₀alkyl, C₁₋₂₀alkoxy, C₂₋₂₀alkenyl, C₄₋₂₀dialkenyl and C₆₋₂₀trialkenyl are optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo;
each R³ is independently selected from the group consisting of C₁₋₂₀alkyl, C₂₋₂₀alkenyl, C₄₋₂₀dialkenyl and C₆₋₂₀trialkenyl, optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O, and OH, wherein the aryl and heterocyclyl are optionally substituted with any one or more independently selected from the group consisting of C₁₋₄alkyl, haloC₁₋₄alkyl, C₁₋₄alkoxy and halo;
R⁴ is selected from the group consisting of C₂₋₂₀alkyl, C₂₋₂₀alkenyl, C₄₋₂₀dialkenyl and C₆₋₂₀trialkenyl, optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo;
A-G is selected from the group consisting of formulae IIIa and IIIb:

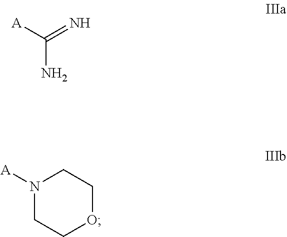

C(O)-J is any one selected from the group consisting of formulae IVa and IVb:

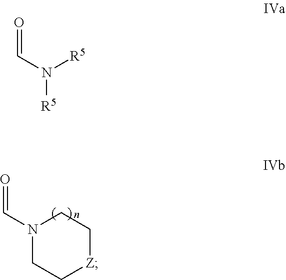

wherein:
each R⁵ is independently selected from the group consisting of H, C₁₋₂₀alkyl, C₂₋₂₀alkenyl, C₄₋₂₀dialkenyl and C₆₋₂₀trialkenyl, wherein the C₂₋₂₀alkenyl, C₄₋₂₀dialkenyl and C₆₋₂₀trialkenyl are optionally substituted with any one or more independently selected from the group consisting of methoxy(ethoxy)₁₋₃, hydroxy(ethoxy)₁₋₃, di(C₁₋₄alkyl)amino, (C₁₋₄alkylamino, amino, cycloC₃₋₈alkyl, pyrrolidinyl, piperidinyl, piperazinyl, morpholinyl, thiomorpholinyl, aryl, halo, cyano, C₁₋₆alkoxy, ═O and OH, and the C₁₋₂₀alkyl is optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl, halo, cyano, C₁₋₆alkoxy, ═O and OH, wherein the cycloC₃₋₈alkyl, pyrrolidinyl, piperidinyl, piperazinyl, morpholinyl, thiomorpholinyl, 1-(3-propyl)piperidine, 1-(3-propyl)piperazine, 4-(3-propyl)morpholine, 4-(3-propyl)thiomorpholine and aryl are optionally substituted with any one or more independently selected from the group consisting of C₁₋₄alkyl, haloC₁₋₄alkyl, C₁₋₄alkoxy and halo; and
n is 1 to 3.

As described above, Q^{a} (of formulae I, II, III and IV) is optionally substituted aryl or optionally substituted heteroaryl and Q^{b} (of formulae I, II, III and IV) is optionally substituted arylene or optionally substituted heteroarylene. For the avoidance of doubt, Q^{b} is a biradical connected to —CH═ and —C(O)—.

The aryl and heteroaryl of Q^{a} and the arylene and heteroarylene of Q^{b} are optionally substituted with any one or a combination selected from the group consisting of N(C₁₋₆alkyl)₂, haloC₁₋₆alkyl, cyano, C₁₋₆alkyl, C₁₋₆alkoxy, NH(C₁₋₆alkyl), NH₂, halo and OH. Often, the haloC₁₋₆alkyl is fluoroC₁₋₆alkyl and the halo is fluoro. Sometimes, the C₁₋₆alkyl of each relevant substituent is C₁₋₄alkyl, such as methyl, i.e. the N(C₁₋₆alkyl)₂ is dimethylamino, the haloC₁₋₆alkyl is halomethyl, the C₁₋₆alkyl is methyl, the C₁₋₆alkoxy is methoxy, and the NH(C₁₋₆alkyl) is methylamino.

In some embodiments, Q^{a} and Q^{b} are optionally substituted with any one or a combination selected from the group consisting of N(C₁₋₆alkyl)₂ (such as N(C₁₋₆alkyl)₂, e.g. dimethylamino), haloC₁₋₆alkyl (such as haloC₁₋₆alkyl, e.g. trifluoromethyl or trifluoroethyl), cyano, C₁₋₆alkyl (such as C₁₋₄alkyl, e.g. methyl or ethyl) and C₁₋₆alkoxy (such as C₁₋₄alkoxy, e.g. methoxy).

In some embodiments, Q^{a} and Q^{b} are optionally substituted with any one or a combination selected from the group consisting of N(C₁₋₄alkyl)₂ (such as dimethylamino), haloC₁₋₄alkyl (such as trifluoromethyl or trifluoroethyl), cyano, C₁₋₄alkyl (such as methyl or ethyl), C₁₋₄alkoxy (such as methoxy) and halo (such as fluoro).

In some embodiments, Q^{a} and Q^{b} are optionally substituted with any one or a combination selected from the group consisting of N(C₁₋₄alkyl)₂ (such as dimethylamino), haloC₁₋₄alkyl (such as trifluoromethyl or trifluoroethyl), cyano, C₁₋₄alkyl (such as methyl or ethyl) and C₁₋₄alkoxy (such as methoxy).

In some embodiments, Q^{a} and Q^{b} are optionally substituted with any one or a combination selected from the group consisting of N(CH₃)₂, halomethyl, cyano, methyl, methoxy and fluoro.

In some embodiments, Q^{a} and Q^{b} are optionally substituted with any one or a combination selected from the group consisting of N(CH₃)₂, halomethyl, cyano, methyl and methoxy.

Often, when Q^{a} is a heteroaryl, it is unsubstituted.

In some embodiments, Q^{a} is selected from the group consisting of optionally substituted phenyl, quinolinyl (such as quinolin-3-yl), benzoxadiazolyl (such as benzoxidiazol-5-yl), naphthalenyl (such as naphthalen-2-yl), benzothiazolyl (such as benzothiazol-2-yl), isoquinolinyl, thiazolyl, pyridinyl, pyrimidinyl, thiophenyl, pyridazinyl, phthalazinyl, imidazolyl and pyrollyl. Often, the quinolinyl (such as quinolin-3-yl), benzoxadiazolyl (such as benzoxidiazol-5-yl), benzothiazolyl (such as benzothiazol-2-yl), isoquinolinyl, thiazolyl, pyridinyl, pyrimidinyl, thiophenyl, pyridazinyl, phthalazinyl, imidazolyl and pyrollyl are unsubstituted.

In some embodiments, Q^{a} is selected from the group consisting of optionally substituted phenyl, quinolinyl (such as quinolin-3-yl), benzoxadiazolyl (such as benzoxadiazol-5-yl), naphthalenyl (such as naphthalen-2-yl) and benzothiazolyl (such as benzothiazol-2-yl), such as optionally substituted phenyl, unsubstituted quinolinyl, unsubstituted benzoxadiazolyl, optionally substituted naphthalenyl and unsubstituted benzothiazolyl. Sometimes, Q^{a} is selected from the group consisting of optionally substituted phenyl, unsubstituted quinolin-3-yl, unsubstituted benzoxadiazol-5-yl, optionally substituted naphthalene-2-yl and unsubstituted benzothiazol-2-yl.

In some embodiments, Q^{b} is monocyclic. Sometimes, Q^{b} is selected from the group consisting of optionally substituted phendiyl (such as benzene-1,4-diyl), pyridindiyl (such as pyridine-2,5-diyl), thiazoldiyl, pyrimidindiyl, thiophendiyl, pyridazindiyl, imidazoldiyl and pyrolldiyl, such as optionally substituted phendiyl and pyridindiyl. Often, Q^{b} is optionally substituted benzene-1,4-diyl or pyridine-2,5-diyl).

In some embodiments, e.g. when monocyclic, Q^{b} is unsubstituted such as unsubstituted benzene-1,4-diyl or pyridine-2,5-diyl.

As described above, each R (of formulae I, II and IV) is independently selected from the group consisting of C₁₋₆alkyl, H and haloC₁₋₆alkyl, such as C₁₋₄alkyl, H and haloC₁₋₄alkyl (e.g. fluoroC₁₋₄alkyl). Often, each R is independently selected from the group consisting of methyl, H and fluoromethyl (such as trifluoromethyl).

In some embodiments, each R is independently selected from the group consisting of C₁₋₄alkyl and H, such as methyl and H. Often, each R is methyl.

As described above, A (of formulae I and Ill) is C₁₋₆alkylene or haloC₁₋₆alkylene (such as fluoroC₁₋₆alkylene). In some embodiments, A is C₁₋₆alkylene, such as C₂₋₅alkylene. Typically, A is unbranched such as any one selected from the group consisting of n-ethylene, n-propylene, n-butylene and n-pentylene.

Z (of formulae Id and IVb) is selected from the group consisting of O, CH₂, N(C₁₋₆alkyl) (such as N(C₁₋₄alkyl)) and S. In some embodiments, Z is selected from the group consisting of O, CH₂, N(CH₃) and S. In some embodiments, Z of formula IVb is CH₂ or S.

Sometimes, the compound is of formula I. In such cases, A-D is often selected from the group consisting of formulae Ia, Ib and Ic, such as Ia and Ib (e.g. Ia).

As described above, each R² (of formula Ia) is independently selected from the group consisting of H, C₁₋₂₀alkyl, C₁₋₂₀alkoxy, C₂₋₂₀alkenyl, C₄₋₂₀dialkenyl and C₆₋₂₀trialkenyl, wherein at least one R² is not H, and wherein the C₁₋₂₀alkyl, C₁₋₂₀alkoxy, C₂₋₂₀alkenyl, C₄₋₂₀dialkenyl and C₆₋₂₀trialkenyl are optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄ alkyl, C₁₋₄alkoxy and halo. Typically, the optionally substituted C₁₋₂₀alkyl, C₁₋₂₀alkoxy, C₂₋₂₀alkenyl, C₄₋₂₀dialkenyl and C₆₋₂₀trialkenyl of R² are optionally substituted C₁₋₆alkyl, C₁₋₉alkoxy, C₂₋₉alkenyl, C₄₋₉dialkenyl and C₆₋₉trialkenyl.

Often, each R² is independently selected from group consisting of H, optionally substituted C₁₋₆alkyl and optionally substituted C₁₋₆alkoxy, wherein at least one R² is not H. Sometimes, the optionally substituted C₁₋₉alkoxy is methoxy, i.e. each R² is independently selected from group consisting of H, optionally substituted C₁₋₆alkyl and methoxy, wherein at least one R² is not H.

As described above, the optional substituents of the alkyl, alkoxy, alkenyl, dialkenyl and trialkenyl groups of R² consist of any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo.

Often, the optionally substituted aryl and heterocyclyl of R² are monocyclic. Sometimes, the optionally substituted aryl of R² is optionally substituted phenyl (such as unsubstituted phenyl). Sometimes, the optionally substituted heterocyclyl of R² is optionally substituted piperazinyl (such as 1-(C₁₋₄alkyl)piperazinyl), optionally substituted tetrahydropyranyl (such as unsubstituted tetrahydropyranyl), or optionally substituted thiomorphinyl (such as unsubstituted thiomorphinyl). Sometimes, the optionally substituted heterocyclyl of R² is optionally substituted piperazinyl (such as 1-(C₁₋₆alkyl)piperazinyl) or optionally substituted tetrahydropyranyl (such as unsubstituted tetrahydropyranyl).

Often, the optional substituents of the alkyl, alkoxy, alkenyl, dialkenyl and trialkenyl groups of R² consist of any one or more independently selected from the group consisting of phenyl, 1-(C₁₋₆alkyl)piperazinyl, tetrahydropyranyl, N(C₁₋₆alkyl)₂ (such as N(C₁₋₃alkyl)₂), methoxy(ethoxy)₁₋₃ (such as methoxy(ethoxy)₁₋₂), hydroxy(ethoxy)₁₋₃ (such as hydroxy(ethoxy)₁₋₂), thiomorphinyl, C₁₋₃alkoxy (such as methoxy) and halo (such as fluoro).

Often, the optional substituents of the alkyl, alkoxy, alkenyl, dialkenyl and trialkenyl groups of R² consist of any one or more independently selected from the group consisting of phenyl, 1-(C₁₋₆alkyl)piperazinyl, tetrahydropyranyl, N(C₁₋₆alkyl)₂ (such as N(C₁₋₃alkyl)₂), methoxy(ethoxy)₁₋₃ (such as methoxy(ethoxy)₁₋₂), and hydroxy(ethoxy)₁₋₃ (such as hydroxy(ethoxy)₁₋₂).

In some embodiments, each R² is independently selected from the group consisting of H, C₁₋₆alkyl, C₁₋₆alkoxy, C₂₋₉alkenyl, C₄₋₉dialkenyl and C₆₋₉trialkenyl, wherein at least one R² is not H, and wherein the C₁₋₉alkyl, C₁₋₉alkoxy, C₂₋₉alkenyl, C₄₋₉dialkenyl and C₆₋₉trialkenyl are optionally substituted with any one or more independently selected from the group consisting of phenyl, 1-(C₁₋₆alkyl)piperazinyl, tetrahydropyranyl, N(C₁₋₆alkyl)₂, methoxy(ethoxy)₁₋₃, and hydroxy(ethoxy)₁₋₃.

In some embodiments, each R² is independently selected from the group consisting of H, C₁₋₉alkyl and C₁₋₉alkoxy, wherein at least one R² is not H, and wherein the C₁₋₉alkyl and C₁₋₉alkoxy are optionally substituted with any one or more independently selected from the group consisting of phenyl, 1-(methyl)piperazinyl, tetrahydropyranyl, N(C₁₋₃alkyl)₂, methoxy(ethoxy)₁₋₂, hydroxy(ethoxy)₁₋₂, thiomorphinyl, methoxy and fluoro.

In more specific embodiments, each R² is independently selected from the group consisting of H, C₁₋₉alkyl and C₁₋₉alkoxy, wherein at least one R² is not H, and wherein the C₁₋₉alkyl and C₁₋₉alkoxy are optionally substituted with any one or more independently selected from the group consisting of phenyl, 1-(methyl)piperazinyl, tetrahydropyranyl, N(C₁₋₃alkyl)₂, methoxy(ethoxy)₁₋₂, and hydroxy(ethoxy)₁₋₂.

Sometimes, when one R² is ethyl, the other is not ethyl. Sometimes, when R² is iso-propyl, Q^{a} is not methoxyphenyl. Sometimes, R² is not iso-propyl.

As described above, each R³ (of formula Ic) is independently selected from the group consisting of C₁₋₂₀alkyl, C₂₋₂₀alkenyl, C₄₋₂₀dialkenyl and C₆₋₂₀trialkenyl, optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₉alkoxy, NH(C₁₋₆alkyl), NH₂, ═O, and OH, wherein the aryl and heterocyclyl are optionally substituted with any one or more independently selected from the group consisting of C₁₋₄alkyl, haloC₁₋₄alkyl, C₁₋₄alkoxy and halo. Typically, each R³ is optionally substituted C₁₋₂₀alkyl.

Often, the optionally substituted aryl and heterocyclyl of R³ are monocyclic. Sometimes, the optionally substituted aryl of R³ is optionally substituted phenyl (such as unsubstituted phenyl). Sometimes, the optionally substituted heterocyclyl of R³ is optionally substituted piperazinyl (such as 1-(C₁₋₆alkyl)piperazinyl, e.g. 1-methylpiperazinyl) or optionally substituted tetrahydropyranyl (such as unsubstituted tetrahydropyranyl).

Often, the optional substituents of the alkyl, alkenyl, dialkenyl and trialkenyl groups of R³ consist of any one or more independently selected from the group consisting of phenyl, 1-(C₁₋₆alkyl)piperazinyl (such as 1-(methyl)piperazinyl), tetrahydropyranyl, N(C₁₋₆alkyl)₂ (such as N(C₁₋₃alkyl)₂), methoxy(ethoxy)₁₋₃ (such as methoxy(ethoxy)₁₋₂), and hydroxy(ethoxy)₁₋₃ (such as hydroxy(ethoxy)₁₋₂).

In some embodiments, each R³ is independently C₁₋₂₀alkyl, optionally substituted with any one or more independently selected from the group consisting of phenyl, 1-(methyl)piperazinyl, tetrahydropyranyl, N(C₁₋₃alkyl)₂, methoxy(ethoxy)₁₋₂, and hydroxy(ethoxy)₁₋₂.

In some embodiments, each R³ comprises fewer than 18 carbon atoms, e.g. no more than 12 carbon atoms.

In specific embodiments, each R³ is independently C₁₋₂₀alkyl, optionally substituted with any one or more independently selected from the group consisting of methoxy(ethoxy)₁₋₂ and hydroxy(ethoxy)₁₋₂.

As described above, R⁴ (of formula Id) is selected from the group consisting of C₂₋₂₀alkyl, C₂₋₂₀alkenyl, C₄₋₂₀dialkenyl and C₆₋₂₀trialkenyl, optionally substituted with any one or more independently selected from the group consisting of aryl, heterocyclyl, N(C₁₋₆ alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄ alkyl, haloC₁₋₄alkyl, C₁₋₄alkoxy and halo. Typically, R⁴ is optionally substituted C₂₋₂₀alkyl, such as optionally substituted C₂₋₁₀alkyl.

In some embodiments, R⁴ comprises a minimum of 5 or 6 carbon atoms, for example, R⁴ may be an optionally substituted C₅₋₂₀alkyl or C₆₋₂₀alkyl, such as an optionally substituted C₅₋₁₀alkyl or C₆₋₁₀alkyl. Alternatively, when R⁴ is a C₁₋₂alkyl or a C₁₋₄alkyl, it may be substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo.

Often, the optionally substituted aryl and heterocyclyl of R⁴ are monocyclic.

Sometimes, the optionally substituted aryl of R⁴ is optionally substituted phenyl (such as unsubstituted phenyl). Sometimes, the optionally substituted heterocyclyl of R⁴ is optionally substituted piperazinyl (such as 1-(C₁₋₆alkyl)piperazinyl, e.g. 1-methylpiperazinyl) or optionally substituted tetrahydropyranyl (such as unsubstituted tetrahydropyranyl).

Often, the optional substituents of the alkyl, alkenyl, dialkenyl and trialkenyl groups of R⁴ consist of any one or more independently selected from the group consisting of phenyl, 1-(C₁₋₆alkyl)piperazinyl (such as 1-(methyl)piperazinyl), tetrahydropyranyl, N(C₁₋₆alkyl)₂ (such as N(C₁₋₃alkyl)₂), methoxy(ethoxy)₁₋₃ (such as methoxy(ethoxy)₁₋₂), and hydroxy(ethoxy)₁₋₃ (such as hydroxy(ethoxy)₁₋₂).

In some embodiments, R⁴ is independently C₂₋₂₀alkyl, optionally substituted with any one or more independently selected from the group consisting of phenyl, 1-(methyl)piperazinyl, tetrahydropyranyl, N(C₁₋₃alkyl)₂, methoxy(ethoxy)₁₋₂, and hydroxy(ethoxy)₁₋₂.

In specific embodiments, R⁴ is unsubstituted C₂₋₁₀alkyl. Often, R⁴ is unsubstituted and unbranched C₂₋₁₀alkyl.

As described above, A-D is selected from the group consisting of formulae Ia to Id, i.e. in some embodiments A-D is Ia, in some embodiments A-D is Ib, in some embodiments A-D is Ic, and in some embodiments A-D is Id. For the avoidance of doubt, when A-D is Ic or Id, the nitrogen atom bonded to A and R³ or R⁴ is positively charged. The positive charge may be (and typically is) stabilised by a counterion, i.e. the compound is typically a cation that is stabilised by an anion to form a salt. An overview of pharmaceutical salts is provided by P. H. Stahl and C. G. Wermuth in Handbook of Pharmaceutical Salts: Properties, Selection and Use, Weinheim/Zürich: Wiley-VCH/VHCA, 2^{nd} Revised Edition, 2011. When the compound is a cation, it may be stabilised by one or more of the anions described in this review. The compounds may be isolated from reaction mixtures as pharmaceutically acceptable salts. The pharmaceutically acceptable salts may alternatively be prepared in situ during the isolation and purification of compounds or by treatment of the compound with a suitable acid, for example, trifluoroacetic acid, benzene sulfonic acid, hydrogen chloride, hydrogen bromide, hydrogen iodide, sulfuric acid, phosphoric acid, acetic acid, propionic acid, glycolic acid, maleic acid, malonic acid, methanesulfonic acid, fumaric acid, succinic acid, tartaric acid, citric acid, benzoic acid and ascorbic acid. When the compound is a cation, it may be stabilised by one or more of the conjugate bases of the acids listed above.

In some embodiments, A-D is selected from the group consisting of formulae Ia and Ib, such as Ia.

Sometimes, the compound is of formula II.

E of formula II is C₄₋₆alkylene or haloC₄₋₆alkylene (such as fluoroC₁₋₆alkylene). In some embodiments, E is C₄₋₆alkylene. Typically, E is unbranched such as any one selected from the group consisting of n-butylene, n-pentylene and n-hexylene. Sometimes, E is a C₄ or a C₅alkylene, such as n-butylene or n-pentylene.

Sometimes, the compound is of formula III.

As described above, each R¹ (of formula III) is independently selected from the group consisting of C₁₋₆alkyl, H and haloC₁₋₆alkyl, such as C₁₋₄alkyl, H and haloC₁₋₄alkyl (e.g. fluoroC₁₋₄alkyl), wherein at least one R¹ is H. Sometimes, one R¹ is H and the other is selected from the group consisting of methyl, H and fluoromethyl (such as trifluoromethyl). In some embodiments, one R¹ is H and the other is selected from the group consisting of C₁₋₄alkyl and H, such as methyl and H.

A-G (of formula III) is selected from the group consisting of formulae IIIa and IIIb, i.e. sometimes A-G is of formula IIIa and sometimes A-G is of formula IIIb. In some embodiments, A-G is of formulae IIIa. Sometimes, when A-G is of formula IIIb, Q^{a} is not dimethylaminophenyl.

Sometimes, the compound is of formula IV. In such cases, C(O)-J is often of formula IVa.

As described above, each R⁵ (of formula IVa) is independently selected from the group consisting of H, C₁₋₂₀alkyl, C₂₋₂₀alkenyl, C₄₋₂₀dialkenyl and C₆₋₂₀trialkenyl, wherein the C₂₋₂₀alkenyl, C₄₋₂₀dialkenyl and C₆₋₂₀trialkenyl are optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₆alkyl, pyrrolidinyl, piperidinyl, piperazinyl, morpholinyl, thiomorpholinyl, aryl, halo, cyano, C₁₋₆alkoxy, ═O and OH, and the C₁₋₂₀alkyl is optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl, halo, cyano, C₁₋₆alkoxy, ═O and OH, wherein the cycloC₃₋₈alkyl, pyrrolidinyl, piperidinyl, piperazinyl, morpholinyl, thiomorpholinyl, 1-(3-propyl)piperidine, 1-(3-propyl)piperazine, 4-(3-propyl)morpholine, 4-(3-propyl)thiomorpholine and aryl are optionally substituted with any one or more independently selected from the group consisting of C₁₋₄alkyl, haloC₁₋₄alkyl, C₁₋₄alkoxy and halo. Typically, the optionally substituted C₁₋₂₀alkyl, C₂₋₂₀alkenyl, C₄₋₂₀dialkenyl and C₆₋₂₀trialkenyl of R⁵ are optionally substituted C₁₋₁₂alkyl, C₂₋₁₂alkenyl, C₄₋₁₂dialkenyl and C₆₋₁₂trialkenyl.

In some embodiments, when the alkyl of R⁵ is substituted with di(C₁₋₄alkyl)amino, C₁₋₄alkylamino and amino substituents, the alkylene moiety comprises at least four carbon atoms, for example it is a substituted C₄₋₂₀alkyl or substituted C₇₋₂₀alkyl. In some embodiments, when the alkyl of R⁵ is a C₁₋₃alkyl or a C₁₋₆alkyl, it is not substituted with di(C₁₋₄alkyl)amino, C₁₋₄alkylamino and amino substituents, i.e. it is optionally substituted with any one or more independently selected from the group consisting of methoxy(ethoxy)₁₋₃, hydroxy(ethoxy)₁₋₃, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl, halo, cyano, C₁₋₆alkoxy, ═O and OH, wherein the cycloC₃₋₈alkyl, pyrrolidinyl, piperidinyl, piperazinyl, morpholinyl, thiomorpholinyl, 1-(3-propyl)piperidine, 1-(3-propyl)piperazine, 4-(3-propyl)morpholine, 4-(3-propyl)thiomorpholine and aryl are optionally substituted with any one or more independently selected from the group consisting of C₁₋₄alkyl, haloC₁₋₄alkyl, C₁₋₄alkoxy and halo.

In some embodiments, when the alkyl of one R⁵ is substituted with di(C₁₋₄alkyl)amino, C₁₋₄alkylamino and amino substituents, the other R⁵ is not H.

Often, each R⁵ is independently selected from the group consisting of H, optionally substituted C₁₋₁₂alkyl and optionally substituted C₄₋₁₂dialkenyl. Sometimes, the C₄₋₁₂dialkenyl is unsubstituted, i.e. each R⁵ is independently selected from group consisting of H, optionally substituted C₁₋₁₂alkyl and unsubstituted C₄₋₁₂dialkenyl.

As described above, the optional substituents of the alkenyl, dialkenyl and trialkenyl groups of R⁵ consist of any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, piperidinyl, piperazinyl, morpholinyl, thiomorpholinyl, aryl, halo, cyano, C₁₋₆alkoxy, ═O and OH, wherein the cycloC₃₋₈alkyl, pyrrolidinyl, piperidinyl, piperazinyl, morpholinyl, thiomorpholinyl and aryl are optionally substituted with any one or more independently selected from the group consisting of C₁₋₄alkyl, haloC₁₋₄alkyl, C₁₋₄alkoxy and halo.

Sometimes, the optional substituents of the alkenyl, dialkenyl and trialkenyl groups of R⁵ consist of any one or more independently selected from the group consisting of methoxy(ethoxy)₁₋₃, hydroxy(ethoxy)₁₋₃, di(C₁₋₆alkyl)amino, C₁₋₄alkylamino, amino and cycloC₃₋₈alkyl.

Often, the alkenyl, dialkenyl and trialkenyl groups of R⁵ are unsubstituted.

As described above, the optional substituents of the alkyl groups of R⁵ consist of any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl, halo, cyano, C₁₋₆alkoxy, ═O and OH, wherein the cycloC₃₋₈alkyl, pyrrolidinyl, 1-(3-propyl)piperidine, 1-(3-propyl)piperazine, 4-(3-propyl)morpholine, 4-(3-propyl)thiomorpholine and aryl are optionally substituted with any one or more independently selected from the group consisting of C₁₋₄alkyl, haloC₁₋₄alkyl, C₁₋₄alkoxy and halo.

In some embodiments, the alkyl groups of R⁵ are not substituted with di(C₁₋₄alkyl)amino, C₁₋₄alkylamino and amino substituents, i.e. the alkyl groups of R⁵ consist of any one or more independently selected from the group consisting of methoxy(ethoxy)₁₋₃, hydroxy(ethoxy)₁₋₃, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl, halo, cyano, C₁₋₆alkoxy, ═O and OH, wherein the cycloC₃₋₈alkyl, pyrrolidinyl, 1-(3-propyl)piperidine, 1-(3-propyl)piperazine, 4-(3-propyl)morpholine, 4-(3-propyl)thiomorpholine and aryl are optionally substituted with any one or more independently selected from the group consisting of C₁₋₄alkyl, haloC₁₋₄ alkyl, C₁₋₄alkoxy and halo. Sometimes, the optionally substituted aryl of R⁵ is optionally substituted phenyl (such as unsubstituted phenyl). Sometimes, the optionally substituted cycloC₃₋₈alkyl, pyrrolidinyl, 1-(3-propyl)piperidine, 1-(3-propyl)piperazine, 4-(3-propyl)morpholine, 4-(3-propyl)thiomorpholine and aryl are unsubstituted.

Often, the optional substituents of the alkyl group of R⁵ consist of any one or more independently selected from the group consisting of methoxy(ethoxy)₁₋₃, hydroxy(ethoxy)₁₋₃, di(C₁₋₄alkyl)amino (such as dimethylamino), C₁₋₄alkylamino (such as methylamino), amino, cycloC₃₋₈alkyl (such as cyclohexanyl or cyclopentanyl), pyrrolidinyl (such as N-pyrrolidinyl), 1-(3-propyl)piperidine, and C₁₋₆alkoxy (such as methoxy).

Often, the optional substituents of the alkyl group of R⁵ consist of any one or more independently selected from the group consisting of methoxy(ethoxy)₁₋₃, hydroxy(ethoxy)₁₋₃, di(C₁₋₄alkyl)amino (such as dimethylamino), C₁₋₄alkylamino (such as methylamino), amino, cycloC₃₋₈alkyl (such as cyclohexanyl or cyclopentanyl), pyrrolidinyl (such as N-pyrrolidinyl) and 1-(3-propyl)piperidine.

Sometimes, the optional substituents of the alkyl group of R⁵ consist of any one or more independently selected from the group consisting of methoxy(ethoxy)₁₋₂, hydroxy(ethoxy)₁₋₂, dimethylamino, methylamino, amino, cycloC₅₋₇alkyl (such as cyclohexanyl or cyclopentanyl), pyrrolidinyl (such as N-pyrrolidinyl) and 1-(3-propyl)piperidine, and C₁₋₃alkoxy (such as methoxy).

Sometimes, the optional substituents of the alkyl group of R⁵ consist of any one or more independently selected from the group consisting of methoxy(ethoxy)₁₋₂, hydroxy(ethoxy)₁₋₂, dimethylamino, methylamino, amino, cycloC₅₋₇alkyl (such as cyclohexanyl or cyclopentanyl), pyrrolidinyl (such as N-pyrrolidinyl) and 1-(3-propyl)piperidine.

In some embodiments, each R⁵ is independently selected from the group consisting of H, C₁₋₁₂alkyl, C₂₋₁₂alkenyl, C₄₋₁₂dialkenyl and C₆₋₁₂trialkenyl, wherein the C₂₋₁₂alkenyl, C₄₋₁₂dialkenyl and C₈₋₁₂trialkenyl are optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, piperidinyl, piperazinyl, morpholinyl, thiomorpholinyl and aryl, and the C₁₋₁₂alkyl is optionally substituted with any one or more independently selected from the group consisting of methoxy(ethoxy)₁₋₃, hydroxy(ethoxy)₁₋₃, di(C₁₋₄alkyl)amino, C₁₋₄alkyl)amino, amino, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl and C₁₋₃alkoxy.

In some embodiments, each R⁵ is independently selected from the group consisting of H, C₁₋₁₂alkyl, C₂₋₁₂alkenyl, C₄₋₁₂dialkenyl and C₆₋₁₂trialkenyl, wherein the C₂₋₁₂alkenyl, C₄₋₁₂dialkenyl and C₆₋₁₂trialkenyl are optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, piperidinyl, piperazinyl, morpholinyl, thiomorpholinyl and aryl, and the C₁₋₁₂alkyl is optionally substituted with any one or more independently selected from the group consisting of methoxy(ethoxy)₁₋₃, hydroxy(ethoxy)₁₋₃, di(C₁₋₄alkyl)amino, C₁₋₄alkyl)amino, amino, cycloC₃₋₈alkyl, pyrrolidinyl, 1-(3-propyl)piperidine, 1-(3-propyl)piperazine, 4-(3-propyl)morpholine, 4-(3-propyl)thiomorpholine, and aryl.

In some embodiments, each R⁵ is independently selected from the group consisting of H, C₁₋₁₂alkyl, C₂₋₁₂alkenyl, C₄₋₁₂dialkenyl and C₆₋₁₂trialkenyl, wherein the C₁₋₁₂ alkyl is optionally substituted with any one or more independently selected from the group consisting of methoxy(ethoxy)₁₋₂, hydroxy(ethoxy)₁₋₂, dimethylamino, -methylamino, amino, cycloC₅₋₇alkyl, pyrrolidinyl, 1-(3-propyl)piperidine, 1-(3-propyl)piperazine, 4-(3-propyl)morpholine, 4-(3-propyl)thiomorpholine, aryl and methoxy.

In some embodiments, each R⁵ is independently selected from the group consisting of H, C₁₋₁₂alkyl, C₂₋₁₂alkenyl, C₄₋₁₂dialkenyl and C₆₋₁₂trialkenyl, wherein the C₁₋₁₂ alkyl is optionally substituted with any one or more independently selected from the group consisting of methoxy(ethoxy)₁₋₂, hydroxy(ethoxy)₁₋₂, dimethylamino, -methylamino, amino, cycloC₅₋₇alkyl, pyrrolidinyl, 1-(3-propyl)piperidine, 1-(3-propyl)piperazine, 4-(3-propyl)morpholine, 4-(3-propyl)thiomorpholine, and aryl.

In some embodiments, each R⁵ is independently selected from the group consisting of H, C₁₋₁₂alkyl and C₄₋₁₂dialkenyl, wherein the C₁₋₁₂alkyl is optionally substituted with any one or more independently selected from the group consisting of methoxy(ethoxy)₁₋₂, hydroxy(ethoxy)₁₋₂, dimethylamino, methylamino, amino, cycloC₅₋₇alkyl, pyrrolidinyl, 1-(3-propyl)piperidine and methoxy.

In some embodiments, each R⁵ is independently selected from the group consisting of H, C₁₋₁₂alkyl and C₄₋₁₂dialkenyl, wherein the C₁₋₁₂alkyl is optionally substituted with any one or more independently selected from the group consisting of methoxy(ethoxy)₁₋₂, hydroxy(ethoxy)₁₋₂, dimethylamino, methylamino, amino, cycloC₅₋₇alkyl, pyrrolidinyl and 1-(3-propyl)piperidine.

As described above, n of formula IVb is 1 to 3. In some embodiments, n is 1 or 2, i.e. the heterocycle of IVb is 6- or 7-membered. In some embodiments, n is 2, i.e. the heterocycle of IVb is 7-membered.

In some embodiments, the compound is any one of the formulae defined in claim 21.

For the avoidance of doubt, when the compound is of formula I, it may be any one of formulae Ia1 to Id2. For example, when the compound is of formula I and A-D is of formula Ia, the compound may be any one of formulae Ia1 to Ia65, such as Ia1 to Ia47; when the compound is of formula I and A-D is of formula Ib, the compound may be any one of formulae Ib1 to Ib11; when the compound is of formula I and A-D is of formula Ic, the compound may be any one of formulae Ic1 to Ic23, such as Ic1 to Ic19; and when the compound is of formula I and A-D is of formula Id, the compound may be of formula Id1 or Id2.

When the compound is of formula II, it may be any one of formulae II1 to II4, such as of formula II1.

When the compound is of formula III, it may be any one of formulae IIIa1 to IIIb9. For example, when the compound is of formula III and A-G is of formula IIIa, the compound may be any one of formulae IIIa1 to IIIa9; and when the compound is of formula III and A-G is of formula IIIb, the compound may be any one of formulae IIIb1 to IIIb9.

When the compound is of formula IV, it may be any one of formulae IVa1 to IVb3. For example, when the compound is of formula IV and C(O)-J is of formula IVa, the compound may be any one of formulae IVa1 to IVa18, such as IVa1 to IVa15; and when the compound is of formula IV and C(O)-J is of formula IVb, the compound may be any one of formulae IVb1 to IVb3.

In some embodiments, the compound of the invention is in the form of a pharmaceutically acceptable salt, i.e. the compound may be isolated or prepared in the form of a pharmaceutically acceptable salt. The term “pharmaceutically acceptable salt” is intended to define salts that may be administered to a patient or used in pharmacy. The pharmaceutically acceptable salt may be prepared by reacting the compound with a suitable acid, such as hydrogen chloride, hydrogen bromide, hydrogen iodide, sulfuric acid, phosphoric acid, acetic acid, trifluoroacetic acid, benzene sulfonic acid, propionic acid, glycolic acid, maleic acid, malonic acid, methanesulfonic acid, fumaric acid, succinic acid, tartaric acid, citric acid, benzoic acid and ascorbic acid. Alternatively, the compound of the invention may be isolated as a pharmaceutically acceptable salt. As described above, when A-D (of formula I) is Ic or Id, the nitrogen atom bonded to A and R³ or R⁴ is positively charged. Thus, when A-D is Ic or Id, the resultant compounds are cations, and the positive charge of the cation is stabilised by a counterion (an anion) to form a salt. The counterion may be pharmaceutically acceptable, or the counterion may be exchanged for a pharmaceutically acceptable counterion, such that the resultant compound is a pharmaceutically acceptable salt. An overview of pharmaceutical salts is provided by P. H. Stahl and C. G. Wermuth in Handbook of Pharmaceutical Salts: Properties, Selection and Use, Weinheim/Zürich: Wiley-VCHNHCA, 2nd Revised Edition, 2011. When the compound is a cation, it may be stabilised by one or more of the anions described in this review.

The compounds of the invention may exhibit tautomerism. All tautomeric forms and mixtures thereof are included within the scope of the invention. The compounds may exist in different stereoisomeric forms. All stereoisomeric forms and mixtures thereof, including enantiomers and racemic mixtures, are included within the scope of the invention. Individual stereoisomers of compounds of the invention, i.e. compounds comprising less than 5% 2% or 1% (e.g. less than 1%) of the other stereoisomer, are included. Mixtures of stereoisomers in any proportion, for example a racemic mixture comprising substantially equal amounts of two enantiomers are also included within the invention.

Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which do not cause racemisation or epimerisation, or by derivatisation, for example with a homochiral acid followed by separation of the diastereomeric esters by conventional means (e.g. HPLC, chromatography over silica).

Also included are solvates and isotopically-enriched compounds of the invention. Isotopically-enriched compounds are identical to those described herein, with the exception that a quantity of the compound has a greater preponderance of an isotope of an element than that found naturally. Examples of such isotopes with which compounds of formula I may be enriched include particular isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl.

According to its second aspect, the invention provides a composition comprising one or more compounds (generally one compound) of the first aspect and a pharmaceutically acceptable excipient. An extensive overview of pharmaceutically acceptable excipients is described in the Handbook of Pharmaceutical Excipients, 6^{th} Edition; Editors R. C. Rowe, P. J. Sheskey and M. E. Quinn, The Pharmaceutical Press, London, American Pharmacists Association, Washington, 2009. Any suitable pharmaceutically acceptable excipient described within this document is within the scope of the invention.

The pharmaceutically acceptable excipient may be included within the composition for the purpose of long-term stabilization of the compound, bulking up solid formulations (often referred to as bulking agents, fillers, or diluents), or to enhance activity of the compound, such as by facilitating its absorption within the body, reducing its viscosity, or enhancing its solubility. The excipient may also enhance in vitro stability of the compound, such as prevention of denaturation or aggregation. Alternatively, the excipient may be used for identification purposes, or to make the compound more appealing to a patient, for example by improving its taste, smell and/or appearance. Typically, the excipient makes up the bulk of the composition.

Excipients include diluents or fillers, binders, disintegrants, lubricants, colouring agents and preservatives. Diluents or fillers are inert ingredients that may affect the chemical and physical properties of the final composition. If the dosage of the compound of the invention is small then more diluents will be required to produce a composition suitable for practical use. If the dosage of the compound of the invention is high then fewer diluents will be required.

Binders add cohesiveness to powders in order to form granules, which may form a tablet. The binder must also allow the tablet to disintegrate upon ingestion so that the compound of the invention dissolves. Disintegration of the composition after administration may be facilitated through the use of a disintegrant.

Of particular mention is a composition suitable for use as a disinfectant, e.g. for use in the prevention of microbial infection, such as compositions suitable for use as additives to contact lens solutions. Modern contact lens solutions typically comprise an antimicrobial, a surfactant, a chelating agent, a demulcent and propylene glycol (see, for example, Jones, L. and Senchyna, M. Optometry in Practice, 8, 2007, 45-56). According to a particular embodiment of the second aspect, a pharmaceutical composition is suitable for use as a contact lens solution. In some embodiments, the composition further comprises a pharmaceutically acceptable surfactant, chelating agent, demulcent and propylene glycol.

The antimicrobial disinfects the lens so that it is safe to be inserted into the eye, and also preserves the solution so that it is safe to be used after exposure to the environment. The antimicrobial is typically effective against a variety of pathogens and selective so that damage to ocular tissues is avoided. Often the antimicrobial is any one or a combination selected from the group consisting of hydrogen peroxide, polyhexamethylene biguanide, Polyquaternium-1, alexidine, and amidoamine. The compound of the invention may be included in a contact lens solution in addition to the antimicrobials typically used, or as an alternative to one or more of the antimicrobials typically used.

Surfactants are typically included in contact lens solutions to act as detergents or cleaners and remove loose debris and deposits from contact lenses by combining with these to form micelles. Surfactants also act to increase the wettability of hydrophobic surfaces, thus when contact lenses are formed from hydrophobic materials, such as silicone hydrogels, the hydrophobic surface is better able to contact the contact lens solution.

Chelating agents are typically included in contact lens solutions and may act synergistically with other components of the solution to improve disinfection efficacy or may aid in the removal of debris and deposits, such as proteins.

Demulcents are agents, usually water-soluble polymers, which are applied topically to the eye to protect and lubricate mucous membrane surfaces and relieve dryness and irritation. Demulcents are typically included in contact lens solutions to improve comfort, for example through modification of the lens surface.

Propylene glycol is typically included in contact lens solutions to enhance water retention on the surface of hydrogel lenses.

Examples of surfactants, chelating agents, and demulcents, and additional components suitable for inclusion in contact lens solutions are described in Jones, L. and Senchyna, M., 2007, supra.

For the avoidance of doubt, the embodiments described herein in relation to the compound defined in the first aspect of the invention apply mutatis mutandis to the second aspect. For example, the one or more compounds of the second aspect may be any one of the formulae defined herein.

As described above, the inventors have found that the compounds of the invention are able to bind to DNA and RNA, and are thus known as Nucleic Acid Binders (NABs). Compounds of the invention are thus able to displace, or inhibit the binding to DNA and RNA of enzymes or regulatory proteins. Enzymes that may be mentioned in this respect include those necessary for replication (thus providing the effect of inhibiting replication of the DNA or RNA) as well as those involved in transcription (thus providing the effect of inhibiting the expression of certain peptides (proteins, enzymes, etc.)).

Affinity to DNA or RNA may be measured by techniques known to those skilled in the art, such as capillary electrophoresis. Furthermore, affinity to certain sections of DNA or RNA may be determined by techniques known to those skilled in the art, such as RNA footprinting.

Due to their ability to inhibit DNA and/or RNA replication, compounds of the invention have utility in the treatment of diseases that rely upon DNA or RNA replication for their propagation. Such diseases include microbial infections and cancer. Such treatment may be particularly useful where the patient suffering from that disease is immunocompromised.

The third aspect of the invention provides a compound of the first aspect or a composition of the second aspect for use as a medicament.

The fourth aspect of the invention provides a compound of the first aspect or a composition of the second aspect for use in the treatment of any one or more selected from the group consisting of viral infection, bacterial infection, fungal infection, parasitic infection and cancer; and the fifth aspect of the invention provides a method of treatment of any one or more selected from the group consisting of a viral infection, bacterial infection, fungal infection, parasitic infection and cancer, said method comprising administering an effective amount of a compound of the first aspect or a composition of the second aspect to a patient in need thereof. According to another aspect of the invention, there is provided use of a compound of the first aspect or a composition of the second aspect, in the manufacture of a medicament for use in the treatment of any one or more conditions selected from the group consisting of viral infection, bacterial infection, fungal infection, parasitic infection and cancer. For the avoidance of doubt, the embodiments described herein in relation to the first and second aspects of the invention apply mutatis mutandis to these aspects. For example, the compound may be any one of the formulae defined herein and/or the composition may comprise one or more of the pharmaceutically acceptable excipients described in the Handbook of Pharmaceutical Excipients (supra).

Whilst the genetic material of bacteria, fungi and parasites is DNA, that of viruses is DNA or RNA.

When the disease treated by the compound of the invention is an RNA viral infection, the virus causing the infection may comprise RNA that is single-stranded (ssRNA) or double-stranded (dsRNA). Examples of double-stranded RNA viruses include reoviruses and rotaviruses. Typically, the RNA within the RNA virus is single-stranded.

Single-stranded RNA viruses may be positive-sense, negative-sense or ambisense. Positive-sense RNA viruses contain RNA that acts in a similar manner to messenger RNA (mRNA) and can be immediately translated by a host cell. Examples of a positive-sense single-stranded RNA virus include coronavirus, rhinovirus, poliovirus, hepatitis C and E virus and Zika virus.

Negative-sense RNA viruses contain RNA that must first be converted to positive-sense RNA by RNA-dependent RNA polymerase before they are translated by a host cell. Examples of a negative-sense single-stranded RNA virus include influenzavirus, measles virus, mumps virus, rabies virus and ebola virus. Ambisense RNA viruses resemble negative-sense viruses but contain at least one ambisense RNA segment that carries both positive-sense and negative-sense RNA. The RNA virus may be segmented or non-segmented. The genome of RNA viruses is often divided up into separate parts, i.e. separate RNA molecules, in which case it is called segmented. Each segment often codes for only one protein.

When the disease treated by the compound of the invention is a DNA viral infection, the virus causing the infection may comprise DNA that is double-stranded (dsDNA) or single-stranded (ssDNA). ssDNA viruses typically have positive sense genomes, which contain DNA that acts in a similar manner to mRNA and can be immediately translated by a host cell. When the DNA within the DNA virus is ssDNA, it is typically positive sense, often as a circular genome that is replicated via rolling circle replication. Typically, however, the DNA within the DNA virus is double-stranded. Examples of double-stranded DNA viruses include adenoviruses (e.g. Titi monkey adenovirus), herpes viruses (e.g. Macacine herpes virus) and polyomaviruses.

The RNA or DNA virus may be enveloped by a protein, i.e. a viral envelope. This protects the genetic material within the virus and is typically derived from portions of host cell membranes. Examples of enveloped RNA viruses include coronaviruses, hepatitis C viruses, zika viruses, influenza viruses, measles viruses, and rabies viruses. Examples of enveloped DNA viruses include the herpes viruses.

In some embodiments, the viral infection is caused by any one of the group consisting of Respiratory Syncytial virus, Human Rhino virus, Human Influenza virus, Influenza virus such as Influenza viruses A and B, Norovirus, Dengue virus, Yellow fever virus, West Nile virus, Zika Virus, Rift Valley fever virus, African swine fever virus, Japanese encephalitis virus, Nipah virus, coronavirus such as SARS-CoV-2, adenoviruses such as Titi monkey adenovirus, herpes viruses such as Macacine herpes virus, and polyomaviruses. Typically, the viral infection is caused by an RNA virus.

In some embodiments, the bacterial infection is caused by any one of the group consisting of Staphylococcus aureus, Enterococci, Streptococci, Clostridia, Corynebacteria, tubercular and non-tubercular mycobacteria, Enterobacteriaceae, Acinetobacter baumannii, Pseudomonas aeruginosa, Helicobacter ri, Campylobacter spp., Salmonellae, Neisseria gonorrhoeae, Haemophilus influenzae, and Shigella spp.

In some embodiments, the fungal infection is caused by any one of the group consisting of Candida albicans, Candida auris, Candida glabrata, Candida parapsilosis, Candida krusei, Candida guilliiermondii, Cryptococccus neoformans, Cryptococcus gattii, Aspergillus fumigatus, Aspergillus flavus, Rhizopus arrhizus, Fusarium oxysporum, Fusarium solani, Scedosporium prolificans, Lomentospora prolificans, Blastomyces dermatitidis, Paecilomyces variotii, Mucor spp., Pneumocystis jirovecii, Histoplasma capsulatum, Coccidioides immitis and Coccidioides posadasii.

In some embodiments, the parasitic infection is caused by any one of the group consisting of Trypanosoma brucei brucei, Trypanosoma brucei gambiense, Trypanosoma brucei rhodesiense, Trypanosoma congolense, Trypanosoma equiperdum, Trypanosoma evansi, Trypanosoma cruzi, Trichomonas vaginalis, Toxoplasma gondii, Plasmodia spp., Leishmania spp., and Acanthamoeba spp.

Owing to the different mode of action exhibited by the compounds of the invention to many conventional antimicrobial agents, compounds of the invention may be particularly useful in the treatment of microbial infections where the infective agent is resistant to one or more antimicrobial agents having a different mode of action. Accordingly, also provided herein is a method of treating a microbial infection, where the microbe causing the infection is resistant to one or more anti-microbial agents that do not act by inhibiting DNA and/or RNA replication, which method comprises administration of a therapeutically effective amount of a compound of the first aspect or a composition of the second aspect of the invention to a patient having that infection.

The compounds of the invention may be used in combination with one or more other compounds or treatment regimes that are used to treat diseases that rely upon DNA and/or RNA replication for their propagation. Accordingly, also provided herein is a method of treating a disease that relies upon DNA and/or RNA replication for its propagation (e.g. a microbial infection or cancer), which method comprises administering a therapeutically effective amount of a compound of the first aspect or a composition of the second aspect of the invention, in combination with one or more other agents that are known to be effective in treating that disease, to a patient.

When used herein, the term “in combination with” includes administration of the other agents that are known to be effective in treating the disease, before, during and/or following administration of a compound or composition of the invention. When more than one other agent is administered, the term also includes administration of the different other agents at different times relative to the time of administration of a compound or composition of the invention.

Agents that are known to be effective in treating diseases that rely upon DNA and/or RNA replication for their propagation (e.g. antimicrobial or anti-cancer agents) include those listed under the relevant headings in “Martindale: The Complete Drug Reference”, 40^{th} Edition, the Pharmaceutical Press, London (2020).

When a compound or composition of the invention is administered to a patient in combination with one or more other agents that are known to be effective in treating diseases that rely upon DNA and/or RNA replication for their propagation, the compound or composition of the invention and the other agents may be administered separately or, conveniently, as a single composition. Thus, there is also provided herein a combination product comprising a formulation comprising a compound of the invention; and a formulation comprising one or more other chemical agents that are known to be effective in treating diseases that rely upon DNA and/or RNA replication for their propagation.

The combination product may comprise the compound of the invention and the one or more other chemical agents as separate components or combined into a single formulation. When the compound of the invention and the one or more other chemical agents are separate components, the combination product is a kit-of-parts.

The compounds and compositions of the invention may be administered orally, subcutaneously, intravenously, intraarterially, transdermally, intranasally, by inhalation, or by any other enteral or parenteral route. The compounds and compositions are typically administered in the form of pharmaceutical preparations comprising the compound of the invention, e.g. as a free base or a non-toxic organic or inorganic acid addition salt, in a pharmaceutically acceptable dosage form. Depending upon the disease and patient to be treated, as well as the route of administration, the compounds and compositions of the invention may be administered at varying doses. Suitable daily doses of the compounds of the invention in therapeutic treatment of humans are about 0.1 to about 100 mg/kg, such as 0.15 to about 50 mg/kg.

The most effective mode of administration and dosage regimen for the compounds and compositions of the invention depends on several factors, including the particular condition being treated, the extent and localisation of that condition in the patient being treated, as well as the patient's state of health and their reaction to the compound being administered. Accordingly, the dosages of the compounds of the invention should be adjusted to suit the individual patient. Methods for determining the appropriate dose for an individual patient will be known to those skilled in the art.

In the sixth aspect, the invention provides the use of a compound of the first aspect or a composition of the second aspect in binding RNA or DNA, wherein said binding is ex vivo or in vitro. For the avoidance of doubt, the embodiments described herein in relation to the first and second aspects of the invention apply mutatis mutandis to the sixth aspect. For example, the compound may be any one of the formulae defined herein and/or the composition may comprise one or more of the pharmaceutically acceptable excipients described in the Handbook of Pharmaceutical Excipients (supra).

In a further aspect, there is provided a method of binding a compound of the first aspect or a composition of the second aspect to RNA or DNA, the method comprising contacting the compound or composition with RNA or DNA. The compound or composition may be contacted with the RNA or DNA ex vivo, in vitro or in vivo. Typically, the contacting takes place ex vivo or in vitro.

The compounds of the invention may have a particularly high affinity for at least one DNA and/or RNA sequence. When bound to at least one DNA and/or RNA oligomer or polymer, the compounds may have a dissociation constant of less than about 10⁻⁵ M, less than about 10⁻⁶ M (such as about 10⁻⁷ M) or less than about 10⁻⁸ M. In this respect, dissociation constants may be measured under conditions known to those skilled in the art, for example in water at room temperature (e.g. at or around 20° C.) in the presence of a buffer (e.g. a buffer that stabilises the pH at about 7.5, such as a borate (e.g. at about 0.02 M) or Tris/HCl (e.g. at about 0.01 M) buffer) and at a DNA or RNA concentration of between about 10 and about 30 mM (e.g. about 20 mM). Alternatively, dissociation constants may be estimated by a comparison of the binding affinity of a compound to a set DNA or RNA sequence with the binding affinity of a well-known compound to that same sequence.

Compounds of the invention may also be used in various assay methods based upon DNA or RNA binding. For example, it is known that compounds that bind to the minor groove of DNA have the ability to stabilise DNA duplexes, as well as to stabilise a fully matched (in terms of base pairs) DNA duplex to a greater extent than a mismatched DNA duplex, thereby enabling easier discrimination between the fully matched and mismatched duplexes (e.g. in terms of the melting temperatures of the duplexes).

Thus, according to a further aspect of the invention, there is provided a method of stabilising a DNA or RNA duplex formed between first and second single strands of DNA or RNA, respectively, which method comprises contacting that DNA or RNA duplex with a compound of the invention.

Further, there is also provided a method of enhancing the difference in melting temperatures between first and second DNA or RNA duplexes, wherein each DNA or RNA duplex is formed from a first single strand of DNA or RNA, respectively, that is the same in each duplex and a second single strand of DNA or RNA, respectively, that is different in each duplex, which method comprises contacting each DNA or RNA duplex with a compound of the invention. The first DNA or RNA duplex may have a greater degree of base-pair matching (e.g. it may be fully matched) than the second DNA or RNA duplex, which may have at least one base-pair mismatch.

Compounds that stabilise fully matched DNA or RNA duplexes to a greater extent than mismatched DNA or RNA duplexes may be used to reduce levels of “false positive” results in DNA or RNA hybridisation assay techniques, for example as described in U.S. Pat. No. 6,221,589. The reduction in “false positive” results may be achieved through the use of more stringent conditions (e.g. higher wash temperatures) following a hybridisation reaction in the presence of a duplex-stabilising compound than is possible following a reaction in the absence of such a compound. Thus, there is further provided herein a method of increasing the maximum temperature of a wash following a DNA or RNA hybridisation reaction, the method comprising the provision of a compound of the invention to the hybridisation reaction mixture. When used herein, the term “maximum temperature of a wash following a DNA or RNA hybridisation reaction” refers to the highest possible wash temperature that does not result in a substantial loss of the “true positive” results (i.e. the fully or most highly matched DNA or RNA duplexes).

When used herein in relation to the above-mentioned methods involving DNA or RNA duplexes, the term “contacting” includes admixing of a compound of the invention with a DNA or RNA duplex. However, the term also includes attaching (e.g. covalently bonding) a compound of the invention that bears a functional group that may be used to form a suitable attachment, to one or both of the single strands of DNA or RNA that form the duplex. Such “labelled” single strands of DNA or RNA may be used as primers, capture probes, or in a number of different assays (e.g. capture-detection assays, 5′-nuclease assays and Beacon assays).

Compounds of the invention may also possess fluorescence properties. Fluorescent compounds of the invention may be useful in various assay methods based upon DNA or RNA binding which involve or require fluorescence. Thus, according to a further aspect of the invention, there is provided a method of detecting dsDNA or dsRNA in a sample, said method comprising contacting a compound of the invention with the sample and comparing the fluorescence of said compound in contact with said sample with the fluorescence of said compound in isolation, a change in fluorescence indicating the presence of DNA or RNA in the sample.

In this embodiment of the invention, a change in fluorescence may be, for example, a change in the wavelength of light emitted by the compound of the invention, a change in the wavelength of light absorbed by said compound or a change in the intensity of light emitted by said compound. Further, the dsDNA or dsRNA may also be labelled with a fluorophore. When labelled in this way (and even when not so labelled), the dsDNA or dsRNA can act as a donor or acceptor in a “FRET’”-type assay for detecting the presence of dsDNA or dsRNA.

In an alternative aspect of the invention, there is provided a method of detecting and visualising dsDNA or dsRNA in a sample, said method comprising contacting the sample with a compound of the invention and irradiating the sample with ultraviolet light. In this embodiment of the invention, the sample might derive from agarose gel electrophoresis experiments or from DNA or RNA microarrays.

Any discussion herein of documents, acts, materials, devices, articles or the like is not to be taken as an admission that any or all of these matters form part of the prior art base or were common general knowledge in the field relevant to the present disclosure as it existed before the priority date of each claim of this application.

It will be appreciated by those skilled in the art that numerous variations and/or modifications may be made to the invention as described herein without departing from the scope of the invention as described. The present embodiments are therefore to be considered for descriptive purposes and are not restrictive, and are not limited to the extent of that described in the embodiment. The person skilled in the art is to understand that the present embodiments may be read alone, or in combination, and may be combined with any one or a combination of the features described herein.

The subject-matter of each patent and non-patent literature reference cited herein is hereby incorporated by reference in its entirety.

The invention may be further understood with reference to the following non-limiting clauses:
1. A compound of any one of formulae I, II, III and IV:

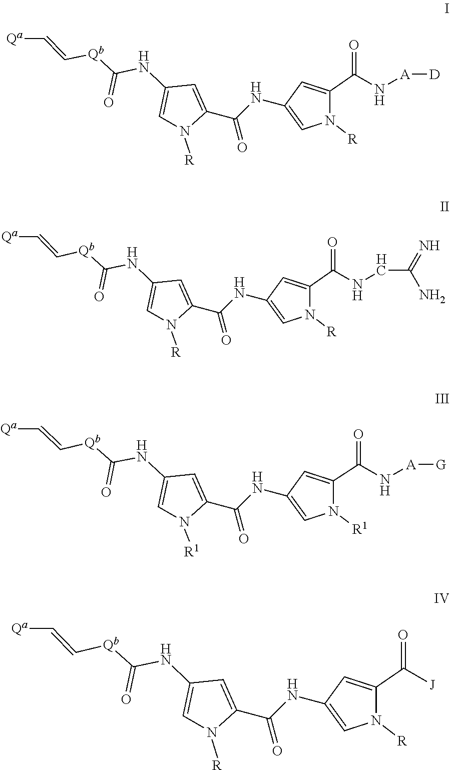

wherein:
   Q^{a} is optionally substituted aryl or optionally substituted heteroaryl and Q^{b} is optionally substituted arylene or optionally substituted heteroarylene, wherein the aryl, heteroaryl, arylene and heteroarylene are optionally substituted with any one or a combination selected from the group consisting of N(C₁₋₆alkyl)₂, haloC₁₋₆alkyl, cyano, C₁₋₆ alkyl, C₁₋₆alkoxy, NH(C₁₋₆alkyl), NH₂, halo and OH;
   each R is independently selected from the group consisting of C₁₋₆alkyl, H and haloC₁₋₆alkyl;
   each R¹ is independently selected from the group consisting of C₁₋₆alkyl, H and haloC₁₋₆alkyl, wherein at least one R¹ is H;
   A is C₁₋₆alkylene or haloC₁₋₆alkylene;
   E is C₄₋₆alkylene or haloC₄₋₆alkylene;
   A-D is selected from the group consisting of formulae Ia to Id:

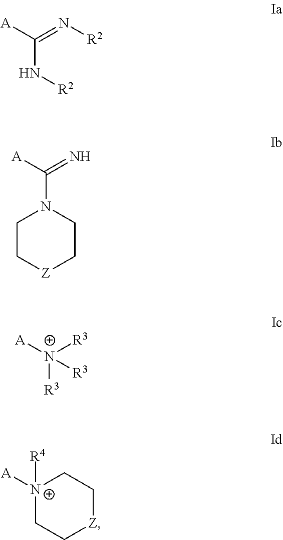

wherein:
Z is selected from the group consisting of O, CH₂, N(C₁₋₄alkyl) and S; and
each R² is independently selected from the group consisting of H, C₁₋₂₀alkyl, C₁₋₂₀alkoxy, C₂₋₂₀alkenyl, C₄₋₂₀dialkenyl and C₆₋₂₀trialkenyl, wherein at least one R² is not H, and wherein the C₁₋₂₀alkyl, C₁₋₂₀alkoxy, C₂₋₂₀alkenyl, C₄₋₂₀dialkenyl and C₆₋₂₀trialkenyl are optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo; each R³ is independently selected from the group consisting Of C₁₋₂₀alkyl, C₂₋₂₀ alkenyl, C₄₋₂₀dialkenyl and C₆₋₂₀trialkenyl, optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O, and OH, wherein the aryl and heterocyclyl are optionally substituted with any one or more independently selected from the group consisting of C₁₋₄alkyl, haloC₁₋₄alkyl, C₁₋₄alkoxy and halo;
R⁴ is selected from the group consisting of C₂₋₂₀alkyl, C₂₋₂₀alkenyl, C₄₋₂₀dialkenyl and C₆₋₂₀trialkenyl, optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo;
A-G is selected from the group consisting of formulae IIIa and IIIb:

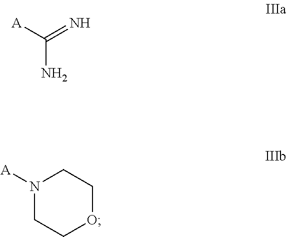

C(O)-J is any one selected from the group consisting of formulae IVa and IVb:

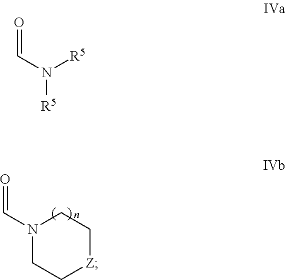

wherein:
each R⁵ is independently selected from the group consisting of H, C₁₋₂₀alkyl, C₂₋₂₀alkenyl, C₄₋₂₀dialkenyl and C₆₋₂₀trialkenyl, wherein the C₂₋₂₀alkenyl, C₄₋₂₀dialkenyl and C₆₋₂₀trialkenyl are optionally substituted with any one or more independently selected from the group consisting of methoxy(ethoxy)₁₋₃, hydroxy(ethoxy)₁₋₃, N(di(C₁₋₄ alkyl)aminoC₁₋₆alkyl)₂, N((C₁₋₄alkyl)aminoC₁₋₆alkyl)₂, N(aminoC₁₋₆alkyl)₂, cycloC₃₋₈alkyl, pyrrolidinyl, piperidinyl, piperazinyl, morpholinyl, thiomorpholinyl, aryl, halo, cyano, C₁₋₆ alkoxy, ═O and OH, and the C₁₋₂₀alkyl is optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl, halo, cyano, C₁₋₆alkoxy, ═O and OH, wherein the cycloC₃₋₈alkyl, pyrrolidinyl, piperidinyl, piperazinyl, morpholinyl, thiomorpholinyl, 1-(3-propyl)piperidine, 1-(3-propyl)piperazine, 4-(3-propyl)morpholine, 4-(3-propyl)thiomorpholine and aryl are optionally substituted with any one or more independently selected from the group consisting of C₁₋₄alkyl, haloC₁₋₄alkyl, C₁₋₄alkoxy and halo; and
n is 1 to 3.
2. The compound of clause 1, wherein the compound is in the form of a pharmaceutically acceptable salt.
3. The compound of clause 1 or clause 2, wherein Q^{a} and Q^{b} are optionally substituted with any one or a combination selected from the group consisting of N(C₁₋₆ alkyl)₂, haloC₁₋₆alkyl, cyano, C₁₋₆alkyl, C₁₋₆alkoxy and halo, for example wherein Q^{a} and Q^{b} are optionally substituted with any one or a combination selected from the group consisting of N(C₁₋₆alkyl)₂, haloC₁₋₆alkyl, cyano, C₁₋₆alkyl and C₁₋₆alkoxy.
4. The compound of clause 1 or clause 2, wherein Q^{a} and Q^{b} are optionally substituted with any one or a combination selected from the group consisting of N(C₁₋₄ alkyl)₂, haloC₁₋₄alkyl, cyano, C₁₋₄alkyl, C₁₋₄alkoxy and halo for example wherein Q^{a} and Q^{b} are optionally substituted with any one or a combination selected from the group consisting of N(C₁₋₄alkyl)₂, haloC₁₋₄alkyl, cyano, C₁₋₄alkyl and C₁₋₄alkoxy.
5. The compound of clause 1 or clause 2, wherein Q^{a} and Q^{b} are optionally substituted with any one or a combination selected from the group consisting of N(CH₃)₂, halomethyl, cyano, methyl, methoxy and fluoro, for example wherein Q^{a} and Q^{b} are optionally substituted with any one or a combination selected from the group consisting of N(CH₃)₂, halomethyl, cyano, methyl and methoxy.
6. The compound of any one of clauses 1 to 5, wherein Q^{a} is selected from the group consisting of optionally substituted phenyl, quinolinyl, benzoxadiazolyl, naphthalenyl, benzothiazolyl, isoquinolinyl, thiazolyl, pyridinyl, pyrimidinyl, thiophenyl, pyridazinyl, phthalazinyl, imidazolyl and pyrollyl.
7. The compound of any one of clauses 1 to 5, wherein Q^{a} is selected from the group consisting of optionally substituted phenyl, quinolinyl, benzoxadiazolyl, naphthalenyl and benzothiazolyl.
8. The compound of any one of clauses 1 to 7, wherein Q^{b} is monocyclic.
9. The compound of any one of clauses 1 to 7, wherein Q^{b} is selected from the group consisting of optionally substituted phendiyl and pyridindiyl.
10. The compound of any one of clauses 1 to 9, wherein Q^{b} is unsubstituted.
11. The compound of any one of clauses 1 to 10, wherein each R is independently selected from the group consisting of C₁₋₄alkyl and H.
12. The compound of any one of clauses 1 to 10, wherein each R is independently selected from the group consisting of methyl and H.
13. The compound of any one of clauses 1 to 12, wherein A is C₁₋₆alkylene.
14. The compound of any one of clauses 1 to 12, wherein A is C₂₋₅alkylene.
15. The compound of any one of clauses 1 to 14, wherein Z is selected from the group consisting of O, CH₂, N(CH₃) and S.
16. The compound of any one of clauses 1 to 15, wherein each R² is independently selected from the group consisting of H, C₁₋₆alkyl, C₁₋₆alkoxy, C₂₋₉alkenyl, C₄₋₉dialkenyl and C₆₋₉trialkenyl, wherein the C₁₋₆alkyl, C₁₋₆alkoxy, C₂₋₉alkenyl, C₄₋₉dialkenyl and C₆₋₉trialkenyl are optionally substituted with any one or more independently selected from the group consisting of phenyl, 1-(C₁₋₆alkyl)piperazinyl, tetrahydropyranyl, N(C₁₋₆alkyl)₂, methoxy(ethoxy)₁₋₃, hydroxy(ethoxy)₁₋₃, thiomorphinyl, C₁₋₃alkoxy and halo, for example wherein each R² is independently selected from the group consisting of H, C₁₋₆alkyl, C₁₋₉alkoxy, C₂₋₉alkenyl, C₄₋₉dialkenyl and C₆₋₉trialkenyl, wherein the C₁₋₉alkyl, C₁₋₉alkoxy, C₂₋₉alkenyl, C₄₋₉dialkenyl and C₆₋₉trialkenyl are optionally substituted with any one or more independently selected from the group consisting of phenyl, 1-(C₁₋₆alkyl)piperazinyl, tetrahydropyranyl, N(C₁₋₆alkyl)₂, methoxy(ethoxy)₁₋₃ and hydroxy(ethoxy)₁₋₃.
17. The compound of any one of clauses 1 to 15, wherein each R² is independently selected from the group consisting of H, C₁₋₉alkyl and C₁₋₉alkoxy, wherein the C₁₋₉alkyl and C₁₋₉alkoxy are optionally substituted with any one or more independently selected from the group consisting of phenyl, 1-(methyl)piperazinyl, tetrahydropyranyl, N(C₁₋₃ alkyl)₂, methoxy(ethoxy)₁₋₂, hydroxy(ethoxy)₁₋₂, thiomorphinyl, methoxy and fluoro, for example wherein each R² is independently selected from the group consisting of H, C₁₋₉ alkyl and C₁₋₉alkoxy, wherein the C₁₋₉alkyl and C₁₋₉alkoxy are optionally substituted with any one or more independently selected from the group consisting of phenyl, 1-(methyl)piperazinyl, tetrahydropyranyl, N(C₁₋₃alkyl)₂, methoxy(ethoxy)₁₋₂ and hydroxy(ethoxy)₁₋₂.
18. The compound of any one of clauses 1 to 17, wherein each R³ is independently C₁₋₂₀alkyl, optionally substituted with any one or more independently selected from the group consisting of phenyl, 1-(methyl)piperazinyl, tetrahydropyranyl, N(C₁₋₃alkyl)₂, methoxy(ethoxy)₁₋₂, and hydroxy(ethoxy)₁₋₂.
19. The compound of any one of clauses 1 to 17, wherein each R³ is independently C₁₋₂₀alkyl, optionally substituted with any one or more independently selected from the group consisting of methoxy(ethoxy)₁₋₂, and hydroxy(ethoxy)₁₋₂.
20. The compound of any one of clauses 1 to 19, wherein R⁴ is C₂₋₂₀alkyl, optionally substituted with any one or more independently selected from the group consisting of phenyl, 1-(methyl)piperazinyl, tetrahydropyranyl, N(C₁₋₃alkyl)₂, methoxy(ethoxy)₁₋₂, and hydroxy(ethoxy)₁₋₂.
21. The compound of any one of clauses 1 to 19, wherein R⁴ is C₂₋₁₀alkyl.
22. The compound of any one of clauses 1 to 17, wherein A-D is selected from the group consisting of formulae Ia and Ib.
23. The compound of any one of clauses 1 to 22, wherein E is C₄₋₆alkylene.
24. The compound of any one of clauses 1 to 23, wherein each R¹ is independently selected from the group consisting of C₁₋₄alkyl and H.
25. The compound of any one of clauses 1 to 23, wherein each R¹ is independently selected from the group consisting of methyl and H.
26. The compound of any one of clauses 1 to 25, wherein A-G is of formulae IIIa.
27. The compound of any one of clauses 1 to 26, wherein each R⁵ is independently selected from the group consisting of H, C₁₋₁₂alkyl, C₂₋₁₂alkenyl, C₄₋₁₂dialkenyl and C₆₋₁₂trialkenyl, wherein the C₂₋₁₂alkenyl, C₄₋₁₂dialkenyl and C₆₋₁₂trialkenyl are optionally substituted with any one or more independently selected from the group consisting of methoxy(ethoxy)₁₋₃, hydroxy(ethoxy)₁₋₃, di(C₁₋₆alkyl)amino, C₁₋₄alkylamino, amino, cycloC₃₋₈alkyl, pyrrolidinyl, piperidinyl, piperazinyl, morpholinyl, thiomorpholinyl and aryl, and the C₁₋₁₂alkyl is optionally substituted with any one or more independently selected from the group consisting of methoxy(ethoxy)₁₋₃, hydroxy(ethoxy)₁₋₃, di(C₁₋₄alkyl)amino, C₁₋₄alkylamino, aminoC₁₋₆alkyl, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl and C₁₋₃alkoxy, for example wherein each R⁵ is independently selected from the group consisting of H, C₁₋₁₂alkyl, C₂₋₁₂alkenyl, C₄₋₁₂dialkenyl and C₈₋₁₂trialkenyl, wherein the C₂₋₁₂alkenyl, C₄₋₁₂dialkenyl and C₆₋₁₂trialkenyl are optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, piperidinyl, piperazinyl, morpholinyl, thiomorpholinyl and aryl, and the C₁₋₁₂alkyl is optionally substituted with any one or more independently selected from the group consisting of methoxy(ethoxy)₁₋₃, hydroxy(ethoxy)₁₋₃, di(C₁₋₄alkyl)amino, C₁₋₄alkylamino, aminoC₁₋₆alkyl, cycloC₃₋₈alkyl, pyrrolidinyl, 1-(3-propyl)piperidine, 1-(3-propyl)piperazine, 4-(3-propyl)morpholine, 4-(3-propyl)thiomorpholine and aryl.
28. The compound of any one of clauses 1 to 26, wherein each R⁵ is independently selected from the group consisting of H, C₁₋₁₂alkyl, C₂₋₁₂alkenyl, C₄₋₁₂dialkenyl and C₆₋₁₂trialkenyl, wherein the C₁₋₁₂alkyl is optionally substituted with any one or more independently selected from the group consisting of methoxy(ethoxy)₁₋₂, hydroxy(ethoxy)₁₋₂, dimethylamino, methylamino, amino, cycloC₅₋₇alkyl, pyrrolidinyl, 1-(3-propyl)piperidine, 1-(3-propyl)piperazine, 4-(3-propyl)morpholine, 4-(3-propyl)thiomorpholine, aryl and C₁₋₃alkoxy, for example wherein each R⁵ is independently selected from the group consisting of H, C₁₋₁₂alkyl, C₂₋₁₂alkenyl, C₄₋₁₂dialkenyl and C₆₋₁₂trialkenyl, wherein the C₁₋₁₂alkyl is optionally substituted with any one or more independently selected from the group consisting of methoxy(ethoxy)₁₋₂, hydroxy(ethoxy)₁₋₂, dimethylamino, methylamino, amino, cycloC₅₋₇alkyl, pyrrolidinyl, 1-(3-propyl)piperidine, 1-(3-propyl)piperazine, 4-(3-propyl)morpholine, 4-(3-propyl)thiomorpholine and aryl.
29. The compound of any one of clauses 1 to 26, wherein each R⁵ is independently selected from the group consisting of H, C₁₋₁₂alkyl and C₄₋₁₂dialkenyl, wherein the C₁₋₁₂ alkyl is optionally substituted with any one or more independently selected from the group consisting of methoxy(ethoxy)₁₋₂, hydroxy(ethoxy)₁₋₂, dimethylamino, -methylamino, amino, cycloC₅₋₇alkyl, pyrrolidinyl, 1-(3-propyl)piperidine and methoxy, for example wherein each R⁵ is independently selected from the group consisting of H, C₁₋₁₂alkyl and C₄₋₁₂dialkenyl, wherein the C₁₋₁₂alkyl is optionally substituted with any one or more independently selected from the group consisting of methoxy(ethoxy)₁₋₂, hydroxy(ethoxy)₁₋₂, dimethylamino, methylamino, amino, cycloC₅₋₇alkyl, pyrrolidinyl and 1-(3-propyl)piperidine.
30. The compound of any one of clauses 1 to 29, wherein n is 1 or 2.
31. The compound of clause 1, which is any one of formulae Ia1 to IVb3 (depicted in claim 21).
32. A composition comprising one or more compounds of any one of clauses 1 to 31 and a pharmaceutically acceptable excipient.
33. A compound of any one of clauses 1 to 31 or a composition of clause 32 for use as a medicament.
34. A compound of any one of clauses 1 to 31 or a composition of clause 32 for use in the treatment of any one or more conditions selected from the group consisting of viral infection, bacterial infection, fungal infection, parasitic infection and cancer.
35. Use of a compound of any one of clauses 1 to 31 or a composition of clause 32 in the manufacture of a medicament for use in the treatment of any one or more conditions selected from the group consisting of viral infection, bacterial infection, fungal infection, parasitic infection and cancer.
36. A method of treatment of any one or more selected from the group consisting of a viral, bacterial, fungal and parasitic infection, and cancer, said method comprising administering an effective amount of a compound as defined in any one of clauses 1 to 31 or a composition as defined in clause 32 to a patient in need thereof.
37. The compound or composition for the use of clause 34; the use of clause 35; or the method of clause 36, wherein:
   (i) the viral infection is caused by any one of the group consisting of Respiratory Syncytial Virus, Human Rhino Virus, Human Influenza Virus, Influenza virus such as Influenza viruses A and B, Norovirus, Dengue virus, Yellow fever virus, West Nile virus, Zika Virus, Rift Valley fever virus, African swine fever virus, Japanese encephalitis virus, Nipah virus, coronavirus such as SARS-CoV-2, adenoviruses such as Titi monkey adenovirus, herpes viruses such as Macacine herpes virus, and polyomaviruses;
   (ii) the bacterial infection is caused by any one of the group consisting of Staphylococcus aureus, Enterococci, Streptococci, Clostridia, Corynebacteria, tubercular and non-tubercular mycobacteria, Enterobacteriaceae, Acinetobacter baumannii, Pseudomonas aeruginosa, Helicobacter pylon, Campylobacter spp., Salmonellae, Neisseria gonorrhoeae, Haemophilus influenzae, and Shigella spp;
   (iii) the fungal infection is caused by any one of the group consisting of Candida albicans, Candida auris, Candida glabrata, Candida parapsilosis, Candida krusei, Candida guilliiermondii, Cryptococccus neoformans, Cryptococcus gattii, Aspergillus fumigatus, Aspergillus flavus, Rhizopus arrhizus, Fusarium oxysporum, Fusarium solani, Scedosporium prolificans, Lomentospora prolificans, Blastomyces dermatitidis, Paecilomyces variotii, Mucor spp., Pneumocystis jirovecii, Histoplasma capsulatum, Coccidioides immitis and Coccidioides posadasii;
   (iv) the parasitic infection is caused by any one of the group consisting of Trypanosoma brucei brucei, Trypanosoma brucei gambiense, Trypanosoma brucei rhodesiense, Trypanosoma congolense, Trypanosoma equiperdum, Trypanosoma evansi, Trypanosoma cruzi, Trichomonas vaginalis, Toxoplasma gondii, Plasmodia spp., Leishmania spp., and Acanthamoeba spp.
38. Use of a compound of any one of clauses 1 to 31 or a composition of clause 32 in binding RNA or DNA, wherein said binding is ex vivo or in vitro.

### EXPERIMENTAL

The invention may be further understood with reference to the examples that follow.

### General Experimental Methods

Reagent Abbreviations: DMF, N,N-dimethylformamide; HBTU, 2-(1H-benzo[d][1,2,3]triazol-1-yl)-1,1,3,3-tetramethylisouronium hexafluorophosphate; TFA, trifluoroacetic acid; DIPEA, N,N-Diisopropylethylamine; HATU, 2-(3H-[1,2,3]triazolo[4,5-b]pyridin-3-yl)-1,1,3,3-tetramethylisouronium hexafluorophosphate; MeOH, methanol; MeCN, acetonitrile; DMSO, dimethyl sulfoxide; Et₂O, diethyl ether; THF, tetrahydrofuran; MgSO₄, magnesium sulfate; DCM, dichloromethane

### Purification Instrumentation:

### HPLC

Where indicated, compounds were purified using a Gilson PL C2250, fitted with a custom 5 μm particle size Nucleodur C18 (50×40-75 g) Gravity Preparative Column. A two solvent system was used: Solvent A (Water w/0.1% TFA), Solvent B (Acetonitrile w/0.1% TFA). The initial gradient method used from which purification was optimised was:

| Time (min) | Flow rate (mL/min) | % A | % B |
|---|---|---|---|
| 00 | 25 | 95 | 05 |
| 06:00 | 25 | 80 | 20 |
| 20:00 | 25 | 70 | 30 |
| 25:00 | 25 | 50 | 50 |
| 35:00 | 25 | 30 | 70 |
| 40:00 | 25 | 95 | 05 |

Note that the gradient was optimised during each run to maximise separation therefore the provided gradient method is only a guide. Fractions were analysed by low-resolution mass spectrometry (LRMS), and those with the desired material were combined and acetonitrile removed using a Smart Evaporator, ready for freeze drying. Upon reinjection, all final S-MGB were determined to be >95% purity. Due to The HPLC eluant containing TFA, all S-MGBs are isolated with TFA counterions as appropriate.

### Smart Evaporator

Acetonitrile was removed from fractions from HPLC purification using a BioChromato. Inc. Smart Evaporator C1. Samples were contained within an appropriately sized round bottomed flask for this procedure.

### Freeze Dryer

Samples contained within an appropriately sized round bottomed flask were frozen by submerging in liquid nitrogen before being lyophilized using an LTE Mini Lyotrap Freeze Dryer.

### Characterisation Instrumentation:

### Low-Resolution Mass Spectrometry (LRMS)

Samples were dissolved in MeOH/H₂O containing 0.1% formic acid. Low-resolution mass spectra were recorded using an Agilent Technologies 1200 series LC-MS instrument with a 6130 single Quadropole and a dual electrospray and atmospheric chemical ionisation source. LC traces were recorded at a wavelength of 254 nm using a Agilent poroshell 120, EC-C18 2.7 μm, 4.6×100 mm column at 1 mL per minute. Two solvent systems were used for chromatographic separation: Method A, using Solvent A (water with 0.1% formic acid), solvent B (MeCN with 0.1% formic acid); and Method B, using solvent C (water with 5 mM ammonium acetate), solvent D (MeCN with 5 mM ammonium acetate). Unless indicated otherwise, Method A was used.

| Time (min:sec) | Solvent A/C | Solvent B/D |
|---|---|---|
| 0 | 95 | 5 |
| 1:48 | 95 | 5 |
| 8:50 | 0 | 100 |
| 13:50 | 0 | 100 |
| 16:50 | 95 | 5 |
| 18:00 | 95 | 5 |

### High-Resolution Mass Spectrometry (HRMS)

This was carried out at NMSF at Swansea University. Samples were solvated in MeCN, then diluted into either MeOH (salts) or MeOH+30 mM ammonium acetate (neutrals) (to promote protonation and ammonium adduct formation rather than sodiation) for positive ion or diluted into either MeOH (salts) or MeOH+diethylamine (neutrals) for negative ion analysis. The Advion Triversa NanoMate (nano-electrospray) was used to deliver the appropriately diluted samples at a flow of approximately 0.25 μL/min. This inlet is used with a 96-well plate, corresponding transfer tips and 400-nozzle spray-chip. Applied nanoelectrospray settings include: spray voltage 1.4 kV, gas pressure 0.4 psi, transfer capillary temperature 200° C. and transfer capillary voltage 30 V. Mass spectrometric detection was via a Thermo Scientific LTQ Orbitrap XL in positive/negative ionization modes. The instrument is externally calibrated each day using an in-house solution of Caffeine, MRFA (Met-Arg-Phe-Ala), and Ultramark 1621 for positive ion and this solution with added Sodium Dodecyl Sulfate and Sodium Taurocholate for negative ion. Internal calibration is accomplished via ‘lockmass’ of known background ions. Spectra were recorded at Resolution 100,000 (FWHM) over the m/z range 150 to 2000 Da with a mass accuracy of <3 ppm RMS.

### Nuclear Magnetic Resonance (NMR)

All ¹H NMR spectral data for synthesised compounds were recorded using a Bruker DRX 500 spectrometer at 500 Hz, with console advance III HD and 4.0.7 topspin software, using the deuterated solvent specified. Chemical shift values (6) are expressed in parts per million (ppm). The following abbreviations are used for the multiplicity of the ¹H NMR signals: s (singlet), d (doublet), dd (doublet of doublets), dt (doublet of triplets), ddd doublet of doublets of doublets), m (multiplet), t (triplet), td (triplet of doublets), and q (quartet). Coupling constants are listed as J values, measured in Hz. Solvent references were DMSO-d₆ referenced at 2.50 (¹H).

### Infrared (IR)

IR spectra were run on a Shimadzu Corp. IRAffinity-1S Fourier Transform Infrared spectrophotometer fitted with a single refection ATR accessory.

### Definition of Structural Elements of Compounds of the Invention

The final step in the synthesis of compounds of the invention is generally achieved by one of two routes: (i) the coupling of a ‘head group dimer’ to a ‘tail group dimer’; or, (ii) the coupling of a ‘tetramer’ directly to a tail group. In some cases, compounds are modified further to generate other compounds of the invention.

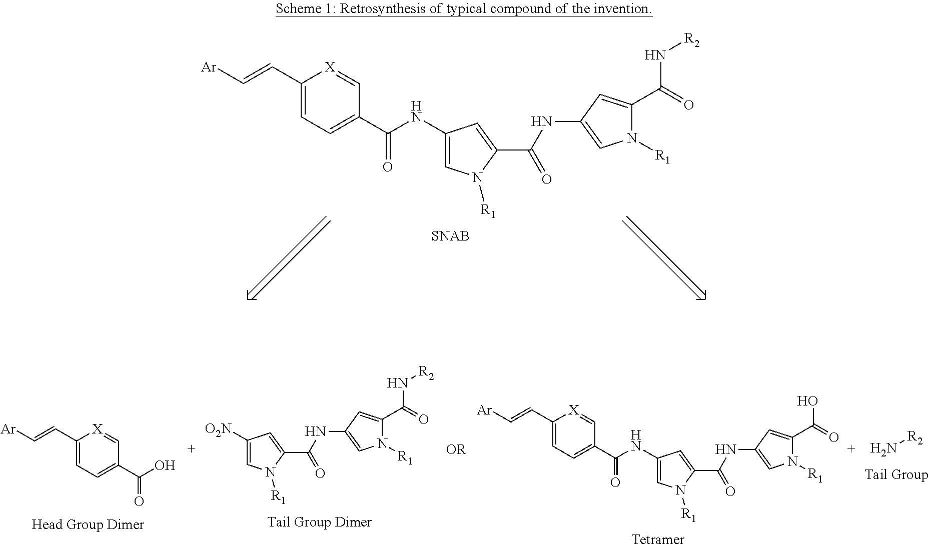

### Synthesis of Headgroup Dimers

In most cases, the head group dimers were prepared according to literature procedures, but the synthesis and characterisation of novel head group dimers is described in full. In most cases, the carboxylic acid form of the head group dimer was prepared from the corresponding methyl ester.

### 6-{(E)-2-[4-(dimethylamino)phenyl]ethenyl}nicotinic acid

Scott, Fraser J. et al., European Journal of Medicinal Chemistry, 2016, vol. 116, p. 116-125

### 4-[(E)-2-(3-quinolinyl)-ethenyl]benzoic acid

Anthony, Nahoum G.; et al., Journal of Medicinal Chemistry, 2007, vol. 50, #24, p. 6116-6125

### 4-[(E)-2-(2,1,3-benzoxadiazol-5-yl)ethenyl]benzoic acid

Scott, Fraser J., 2016, supra

### (E)-4-(2-(naphthalen-2-yl)vinyl)benzoic acid

Anthony, Nahoum G., 2007, supra

### 4-{(E)-2-[4-(trifluoromethyl)phenyl]ethenyl}benzoic acid

Scott, Fraser J., 2016, supra

### 4-[(E)-2-(1,3-benzothiazol-2-yl)ethenyl]benzoic acid

Anthony, Nahoum G., 2007, supra

### 6-[(E)-2-(4-methoxyphenyl)ethenyl]nicotinic acid

Anthony, Nahoum G., 2007, supra

### 4′-methoxy-trans-stilbene-4-carboxylic acid

This compound was prepared from its methyl ester, followed by hydrolysis.

### Methyl 3-[(E)-2-(4-methoxyphenyl)ethenyl]benzoate

To a solution of methyl 3-[(diethoxyphosphoryl)methyl]benzoate (1.02 g, 3.56 mmol) in THF (5 mL, dry) under nitrogen atmosphere was added sodium hydride (0.212 g, 60%, 18.8 mmol). After cooling the reaction mixture to 0° C., p-anisaldehyde (0.485 g, 3.56 mmol) in THE (10 mL, dry) was carefully added dropwise with stirring. The reaction mixture was stirred for 1 h at room temperature and then quenched with water. After neutralization with dilute hydrochloric acid, the two layers were separated. The water layer was extracted with ethyl acetate and the organic layers were combined, dried (MgSO₄), filtered and the solvent removed under reduced pressure. The product was purified using silica gel column chromatography to give a white solid (0.747 g, 78%), mp 92-94° C.

IR (cm⁻¹) 1710, 158, 1467, 1440, 1267, 1161, 792, 744, 686

¹H NMR (500 MHz, DMSO-d₆) δ (ppm) 8.16 (1H, s), 7.91 (1H, d, J=8.1 Hz), 7.86 (1H, d, J=8.1 Hz), 7.55-7.20 (6H, m), 6.87 (1H, dd, J=1.7 Hz, J=8.0 Hz), 3.88 (3H, s), 3.80 (3H, s).

HRMS m/z calculated for C₁₇H₁₆O₃ 268.1099 [M+H]⁺, found 268.1101 [M+H]⁺

### 3-[(E)-2-(4-Methoxyphenyl)ethenyl]benzoic acid

Methyl 3-[(E)-2-(4-methoxyphenyl)ethenyl]benzoate (0.740 mg, 2.75 mmol) was suspended in methanol (10 mL) and water (20 mL) to which lithium hydroxide solution (0.199 g, 8.27 mmol) in water (10 mL) was added with stirring. The reaction mixture was heated under reflux for 4 h. At the beginning, the starting material dissolved, and then a white precipitate appeared. The reaction mixture was cooled in an ice bath then dilute hydrochloric acid was added dropwise with vigorous stirring until pH 2. The product as a white solid material was filtered off, washed with water and dried under reduced pressure at 60° C. (0.235 g, 34%), mp 195-198° C.

IR (cm⁻¹) 1684, 1586, 1541, 1423, 1310, 1272, 961, 767, 687

¹H NMR (500 MHz, DMSO-d₆) δ (ppm) 8.12 (1H, s), 7.78 (1H, d, J=7.6 Hz), 7.63 (1H, d, J=7.6 Hz), 7.37-7.18 (7H, m), 6.83 (1H, dd, J=1.7 Hz, J=8.0 Hz), 3.80 (3H, s).

HRMS m/z calculated for C₁₆H₁₄O₃ 254.0943 [M+H]⁺, found 254.0935 [M+H]⁺

### 4-[(E)-2-(3-methoxyphenyl)ethenyl]benzoic acid

Anthony, Nahoum G., 2007, supra

### 6-[(E)-2-(3-Methoxyphenyl)ethenyl]nicotinic acid

This compound was prepared from its methyl ester, followed by hydrolysis.

### Methyl 6-[(E)-2-(3-methoxyphenyl)ethenyl]nicotinate

3-Methoxybenzaldehyde (210 mg, 1.52 mmol), methyl 6-methylnicotinate (230 mg, 1.52 mmol), acetic anhydride (310 mg, 3.04 mmol) and catalytic amount of zinc chloride were heated at 140° C. with stirring for 12 h. Ethyl acetate and brine were added to the cooled reaction mixture and the product was extracted. The organic layer was collected, dried (MgSO₄) and the solvent removed under reduced pressure. The crude product was applied to a silica gel column and the product was eluted with ethyl acetate/n-hexane 1:1. Fractions containing the required product were collected and the solvents removed under reduced pressure to give the desired material as a yellow solid (87 mg, 21%), mp 170-173° C.

IR (cm⁻¹) 1717, 1606, 1591, 1511, 1433, 1290, 1254, 1175, 1111, 1020, 844, 818, 760, 734

¹H NMR (500 MHz, DMSO-d₆) δ (ppm) 9.04 (1H, d, J=2.0 Hz), 8.25 (1H, dd, J=2.2 Hz, J=8.2 Hz), 7.81 (1H, d, J=16.0 Hz), 7.67-7.62 (3H, m), 7.28 (1H, d, J=16.0 Hz), 7.00 (2H, d, J=8.8 Hz), 3.88 (3H, s), 3.80 (3H, s).

HRMS m/z calculated for C₁₆H₁₆O₃N 270.1130 [M+H]⁺, found 270.1127 [M+H]⁺

### 6-[(E)-2-(3-Methoxyphenyl)ethenyl]nicotinic acid

Methyl 6-[(E)-2-(3-methoxyphenyl)ethenyl]nicotinate (80 mg, 0.297 mmol) was dissolved in methanol (5 mL) to which sodium hydroxide solution (145 mg) in water (10 mL) was added. The reaction mixture was heated under reflux for 3 h. Methanol was removed under reduced pressure and the remaining solution was cooled to 0° C. Hydrochloric acid (conc.) was added dropwise with vigorous stirring until pH 4. The yellow solid material was collected by filtration, washed with water and dried under reduced pressure at 50° C. to give the required material (59 mg, 78%), mp 230-233° C. (sublimed around 200° C.).

IR (cm⁻¹) 1717, 1681, 1635, 1595, 1513, 1292, 1250, 1173, 1023, 825

¹H NMR (500 MHz, DMSO-d₆) δ (ppm) 9.03 (1H, d, J=2.0 Hz), 8.29 (1H, dd, J=2.1 Hz, J=8.2 Hz), 7.90-7.65 (4H, m), 7.29 (1H, d, J=16.0 Hz), 7.01 (2H, m), 3.80 (3H, s) HRMS m/z calculated for C₁₅H₁₄O₃N 256.0974 [M+H]⁺, found 256.0972 [M+H]⁺

### Methyl (E)-6-(4-fluoro-3-methoxystyryl)nicotinate

4-Fluoro-3-methoxybenzaldehyde (1.02 g, 6.62 mmol), methyl 6-methyinicotinate (1.00 g, 6.62 mmol) and zinc chloride (5% by weight) were dissolved in acetic anhydride (3 mL). The reaction mixture was heated at 140° C. for 12 h with stirring. The reaction mixture was diluted with brine and ethyl acetate. Some of the product was filtered off since it was not soluble in methanol or ethyl acetate (247 mg). After the extraction, the organic layers were collected, dried (MgSO₄) and the solvent removed under reduced pressure to give the crude product which was purified further by column chromatography using ethyl acetate/n-hexane (1/4), R_{F}=0.20. The product was obtained as white crystalline (0.746 g, 39%), mp 150-153° C.

IR (cm⁻¹) 1711, 1638, 1590, 1515, 1458, 1436, 1418, 1343, 1275, 1157, 1124, 1114, 1031, 971, 850, 820

¹H NMR (500 MHz, DMSO-d₆) δ (ppm) 9.05 (1H, d, J=2.0 Hz), 8.29 (1H, dd, J=2.2 Hz & J=8.2 Hz), 7.83 (1H, d, J=16.1 Hz), 7.66 (1H, d, J=7.8 Hz), 7.56 (1H, dd, J=1.8 Hz & J=7.0 Hz), 7.44 (1H, d, J=16.1 Hz), 7.27-7.22 (2H, m), 3.91 (3H, s), 3.88 (3H, s).

HRMS m/z calculated for C₁₆H₁₅O₃NF 288.1036 [M+H]⁺, found 288.1039 [M+H]⁺

### (E)-6-(4-fluoro-3-methoxystyryl)nicotinic acid

Methyl 6-[(E)-2-(4-fluoro-3-methoxyphenyl)ethenyl]nicotinate (0.500 g, 1.74 mmol) was suspended in methanol (5 mL) to which sodium hydroxide solution (0.348 g, 8.71 mmol) was dissolved in water (20 mL) was added. The reaction mixture was heated under reflux for 3 h. Methanol was removed under reduced pressure and the water was cooled to 0° C. Hydrochloric acid (concentrated) was added dropwise with stirring at 0° C. until pH4. The pale yellow material formed was filtered, washed with water and dried under reduced pressure to give the required product as a pale yellow solid (0.457 g, 96%), mp>230° C.

IR (cm⁻¹) 1718, 1621, 1598, 1514, 1268, 1160, 1120, 1028, 961, 854, 801

¹H NMR (500 MHz, DMSO-d₆) δ (ppm) 9.04 (1H, d, J=2.0 Hz), 8.28 (1H, dd, J=2.2 Hz & J=8.2 Hz), 7.83 (1H, d, J=16.1 Hz), 7.67 (1H, d, J=8.2 Hz), 7.55 (1H, dd, J=1.6 Hz & J=7.4 Hz), 7.44 (1H, d, J=16.1 Hz), 7.29-7.22 (2H, m).

LRMS m/z calculated for C₁₅H₁₂O₃NF 273.08 [M]⁺, found 274.2 [M+H]⁺.

### Methyl (E)-4-(2-(pyrimidin-5-yl)vinyl)benzoate

4-Methylpyrimidine (250 mg, 2.66 mmol) and methyl 4-formylbenzoate (436 mg, 2.66 mmol) were dissolved in acetic anhydride (1 mL) to which zinc chloride (5% by weight) was added. The reaction mixture was heated under reflux overnight. The black mass was triturated with ethyl acetate and filtered. The solid material was washed with small amount of methanol to give pale yellow microcrystalline (236 mg, 37%), mp>230° C.

IR (cm⁻¹) 1717, 1599, 1466, 1399, 1274, 1104, 1021, 965, 852, 778

¹H NMR (500 MHz, DMSO-d₆) δ (ppm) 9.16 (1H, s), 8.81 (1H, d, J=5.2 Hz), 7.99 (2H, m), 7.88 (2H, d, J=4.8 Hz), 7.67 (1H, d, J=5.2 Hz), 7.48 (1H, d, J=16.0 Hz), 3.87 (3H, s)

HRMS m/z calculated for C₁₋₄H₁₃O₂N₂ 241.0977 [M+H]⁺, found 241.0974 [M+H]⁺

### (E)-4-(2-(pyrimidin-5-yl)vinyl)benzoic acid

Methyl 4-[(E)-2-(4-pyrimidinyl)ethenyl]benzoate (100 mg, 0.416 mmol) was suspended in methanol (2 mL) to which sodium hydroxide solution [NaOH (67 mg, 1.67 mmol) in water (10 mL)] was added. The reaction mixture was heated under reflux for 6 h. Solvents were removed to dryness and the white solid material was dissolved in water and cooled to 0° C. Concentrated HCl was added dropwise with stirring at 0° C. until pH4. The resultant “emulsion” was freeze dried to give the required product in quantitative yield (containing sodium chloride), mp>230° C.

IR (cm⁻¹) 2498, 1687, 1585, 1281, 1172, 1007, 853, 780

¹H NMR (500 MHz, DMSO-d₆) δ (ppm) 13.00 (1H, br), 9.16 (1H, d, J=0.8 Hz), 8.81 (1H, d, J=5.2 Hz), 8.04-7.97 (3H, m), 7.85 (2H, d, J=8.0 Hz), 7.67 (1H, dd, J=5.2 Hz & J=1.2 Hz), 7.46 (1H, d, J=16.0 Hz).

HRMS m/z calculated for C₁₃H₁₁O₂N2227.0821 [M+H]⁺, found 227.0822 [M+H]⁺

### Methyl (E)-4-(3-fluorostyryl)benzoate

Diethyl 3-fluorobenzylphosphonate (1.35 g, 5.50 mmol) was dissolved in THF (10 mL, dry) then cooled to 0° C. under nitrogen. Sodium hydride (300 mg, 60% suspension in oil, 7.50 mmol) was added portionwise with stirring. Methyl 4-formylbenzoate (0.903 g, 5.52 mmol) was dissolved in THE (10 mL, dry) then added to the reaction mixture at 0° C. under nitrogen with stirring. The reaction mixture was then left stirring at room temperature for 1 h. Water was added at 0° C. dropwise under nitrogen followed by dilute hydrochloric until pH6. The TH F layer was separated after the extraction and the water layer was extracted once more with ethyl acetate. The organic layers were combined, dried (MgSO4). TLC showed two spots: one corresponding to the product and the second one corresponding to the un-reacted methyl 4-formylbenzoate. The solvents were removed under reduced pressure then dissolved in DCM. Girard's reagent T (180 mg, 3.00 mmol) was dissolved in acetic acid (1.803 g, 30.00 mmol) was added. The reaction mixture was stirred for 2 h at room temperature and then water was added. After the extraction, the water layer was extracted with DCM (2×25 mL). The combined organic layers were washed with brine (3×15 mL), and aqueous Na₂CO₃ (3×15 mL), dried (MgSO₄), and evaporated under reduced pressure to give the required product as a white microcrystalline solid (0.490 g, 35%), mp 115-118° C., R_{F}=0.80 (1/2 ethyl acetate/n-hexane).

IR (cm⁻¹) 1718, 1609, 1580, 1438, 1281, 1264, 1112, 960, 864, 792, 757, 700

¹H NMR (500 MHz, DMSO-d₆) δ (ppm) 7.98 (2H, d, J=8.4 Hz), 7.76 (2H, d, J=8.4 Hz), 7.54-7.41 (5H, m), 7.16-7.12 (1H, m), 3.86 (3H, s).

HRMS m/z calculated for C₁₆H₁₃O₂F 256.0900 [M+H]⁺, found 256.0898 [M+H]⁺

### (E)-4-(3-fluorostyryl)benzoic acid

Methyl 4-[(E)-2-(3-fluorophenyl)ethenyl]benzoate (0.480 g, 1.87 mmol) was suspended in methanol (5 mL) to which sodium hydroxide solution (0.300 g, 7.50 mmol) was dissolved in water (15 mL) was added. The reaction mixture was heated under reflux for 5 h. Methanol was removed under reduced pressure and the water was cooled to 0° C. Hydrochloric acid (concentrated) was added dropwise with stirring at 0° C. until pH4. The white crystalline material formed was filtered, washed with water and dried under reduced pressure to give the required product as a white crystalline (0.420 g, 93%), mp>230° C.

IR (cm⁻¹) 3012, 1678, 1610, 1584, 1427, 1296, 1240, 1183, 1144, 950, 859, 774

¹H NMR (500 MHz, DMSO-d₆) δ (ppm) 7.66 (2H, d, J=8.2 Hz), 7.32 (2H, d, J=8.2 Hz), 7.26-7.18 (3H, m), 7.16 (1H, d, J=16.4 Hz), 7.08 (1H, d, J=16.4 Hz), 6.90-6.86 (1H, m).

LRMS m/z calculated for C₁₅H₁₁FO₂ 242.07 [M], found 241.1 [M−H]⁻

### Methyl (E)-6-(4-fluorostyryl)nicotinate

4-Fluorobenzaldehyde (0.821 g, 6.61 mmol), methyl 6-methylnicotinate (1.00 g, 6.61 mmol) and catalytic amount of zinc chloride were dissolved in acetic anhydride (3 mL). The reaction mixture was heated at 140° C. for 12 h with stirring. The reaction mixture was diluted with brine and ethyl acetate. After the extraction, the organic layers were collected, dried (MgSO₄) and the solvent removed under reduced pressure to give the crude product which was purified further by column chromatography using ethyl acetate/n-hexane (1/4), R_{F}=0.20. The product was obtained as white crystalline (0.400 g, 24%), mp 142-144° C.

IR (cm⁻¹) 1716, 1588, 1509, 1435, 1287, 1225, 1193, 1114, 993, 847, 827, 773, 736 cm

¹H NMR (500 MHz, DMSO-d₆) δ (ppm) 9.07 (1H, d, J=2.2 Hz), 8.29 (1H, dd, J=8.2 Hz & J=2.2 Hz), 7.87 (1H, d, J=16.1 Hz), 7.81-7.77 (2H, m), 7.68 (1H, d, J=8.2 Hz), 7.41 (1H, d, J=16.1 Hz), 7.27 (2H, t, J=8.8 Hz), 3.89 (3H, s)

HRMS m/z calculated for C₁₅H₁₃O₂NF 258.0930 [M+H]⁺, found 258.0933 [M+H]⁺

### (E)-6-(4-fluorostyryl)nicotinic acid

Methyl 6-[(E)-2-(4-Fluorophenyl)ethenyl]nicotinate (0.354 g, 1.38 mmol) was suspended in methanol (5 mL) to which sodium hydroxide solution (0.374 g, 9.35 mmol) was dissolved in water (10 mL) was added. The reaction mixture was heated under reflux for 3 h. Methanol was removed under reduced pressure and the water was cooled to 0° C. Hydrochloric acid (concentrated) was added dropwise with stirring at 0° C. until pH4. The pale yellow material formed was filtered, washed with water and dried under reduced pressure to give the required product as a pale yellow solid (0.300 g, 98%), mp>230° C.

IR (cm⁻¹) 2440, 1700, 1642, 1593, 1509, 1386, 1286, 1232, 1160, 965, 843, 822, 801, 731

¹H NMR (500 MHz, DMSO-d₆) δ (ppm) 13.32 (1H, br), 9.05 (1H, d, J=1.9 Hz), 8.26 (1H, dd, J=2.2 & J=8.1 Hz), 7.85 (1H, d, J=16.1 Hz), 7.80-7.77 (2H, m), 7.66 (1H, d, J=7.8 Hz), 7.41 (1H, d, J=16.1 Hz), 7.29 (2H, t, J=8.9 Hz)

HRMS m/z calculated for C₁₋₄H₁₁O₂NF 244.0774 [M+H]⁺, found 244.0773 [M+H]⁺

### (E)-6-(3-(trifluoromethyl)styryl)nicotinic acid

Scott, Fraser J. et al., Bioorganic and Medicinal Chemistry Letters, 2016, vol. 26, #15, p. 3478-3486

### Synthesis of Tail Group Dimers

Synthesis of Tail Group Dimers that Contain Alkyl Amidines or Amidines of

### Different Tail Group Lengths

Firstly, the appropriate cyano dimer is prepared by reacting the acid dimer with the appropriate amino and cyano functionalised alkane, using typical amide coupling conditions. The starting acid dimer is prepared as per Lown; Krowicki, Journal of Organic Chemistry, 1985, vol. 50, #20, p. 3774-3779.

The cyano dimers are then converted to their corresponding imidate analogues through typical Pinner conditions, after which the resultant imidate is used without isolation to yield the corresponding amidine dimer through reaction with the appropriate amine.

The ethyl imidate dimer is prepared as per Rao; Bathini; Lown, Journal of Organic Chemistry, 1990, vol. 55, #2, p. 728-737.

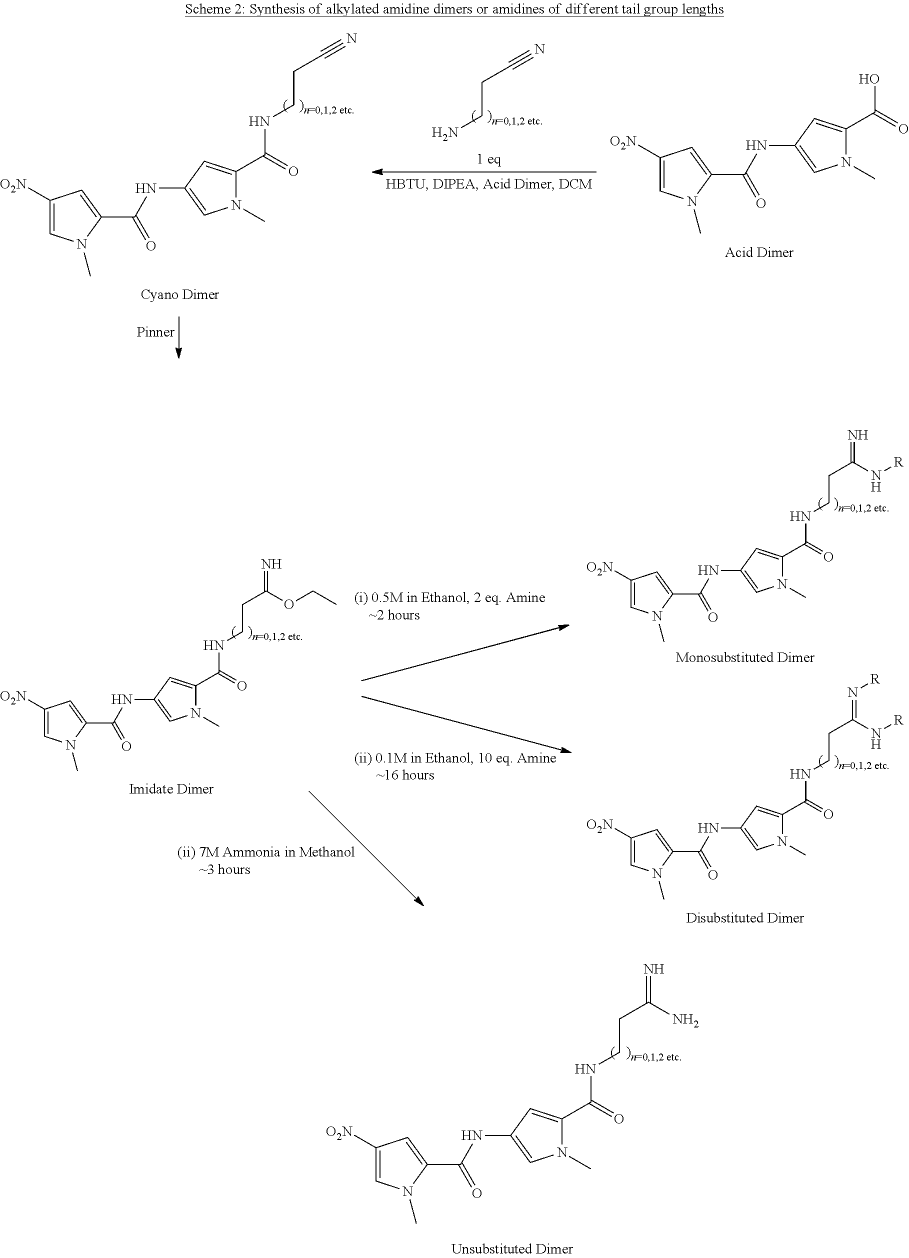

For cyano dimers, the acid dimer (1 eq) was dissolved in DMF with HATU (1.2 eq) and TEA (2.5 eq) and stirred at room temperature for 30 minutes. To this was added the appropriate cyanoalkylamine (1 eq) which was stirred overnight. Saturated brine solution was added to the reaction mixture and a precipitate was formed. The precipitate was collected by filtration, air dried and found to be the desired product.

For imidate dimers, the appropriate cyano tail group dimer (0.5-1 mmol, 1 eq) was suspended in ethanol (20 mL, dry) and cooled to −60° C. Dry hydrogen chloride gas was generated from concentrated sulfuric acid and concentrated hydrochloric acid and bubbled through the solution until saturation (approximately 30 mins-1 hour). The reaction mixture was left to warm up to room temperature and then stirred for 2 hours at room temperature. The solvent was removed by evaporation under reduced pressure. The resulting solid was triturated in dry diethyl ether and collected by vacuum filtration. The presence of an imidate was checked for by NMR and then the material was taken forward immediately.

For unsubstituted amidine dimers, the imidate dimer was dissolved in ammonia in methanol (7M, 10 mL) and the reaction mixture was stirred at 55° C. for 3 hours. The hot solution was filtered and the solid residue washed with dilute hydrochloric acid. The solid was allowed to air dry and found to be the product (˜80% yield).

For monosubstituted amidine dimers, the ethyl imidate dimer (0.30-0.65 mmol) was dissolved in ethanol to give a 0.5 M solution, then the amine (2.0 eq.) was added. The reaction mixture was stirred at 50° C. and followed by LCMS until completion (˜2 h). The resulting solid is then filtered and washed with Et₂O (10 mL). If the product does not crash out, the solvent is removed in vacuo and the resulting solid is washed with Et₂O (10 mL).

For disubstituted amidine dimers, the ethyl imidate dimer (1.0 eq.) was dissolved in dry ethanol (0.1 M), then the amine (10 eq.) was added. The reaction mixture was stirred at 50° C. (or reflux) overnight (or until reaction is over), about 16 hours. The solid is then filtered and washed with Et₂O (10 mL). If the product has not crashed out the solvent is removed in vacuo and the resulting solid is washed with Et₂O (10 mL).

HPLC purification was used to separate traces of undesired mono- or -di substituted dimer.

Where indicated, for instances where the mono- and di-substituted dimer mixture could not be adequately separated, the mixture was used without purification to form a mixture of full S-MGBs, which was separated by HPLC at that stage.

Characterisation Details of Dimers that Contain Alkyl Amidines or Amidines of Different Tail Group Lengths

### N-(2-cyanoethyl)-1-methyl-4-(1-methyl-4-nitro-1H-pyrrole-2-carboxamido)-1H-pyrrole-2-carboxamide

Debart et al., Journal of Medicinal Chemistry, 1989, vol. 32, #5, p. 1074-1083

### N-(4-cyanobutyl)-1-methyl-4-(1-methyl-4-nitro-1H-pyrrole-2-carboxamido)-1H-pyrrole-2-carboxamide

IR (cm⁻¹) 3128, 1635, 1498, 1305, 839

¹H NMR (500 MHz, DMSO-d₆) δ (ppm) 10.22 (s, 1H), 8.17 (d, J=2.1 Hz, 1H), 8.09 (t, J=5.8 Hz, 1H), 7.57 (d, J=2.1 Hz, 1H), 7.20 (d, J=2.0 Hz, 1H), 6.86 (d, J=2.0 Hz, 1H), 3.95 (s, 3H), 3.81 (s, 3H), 3.19 (a. J=7.1 Hz, 2H), 2.69-2.69 (m, 6H)

¹³C NMR (126 MHz, DMSO-d₆) δ (ppm) 128.2, 126.3, 117.9, 107.5, 103.9, 38.2, 37.4, 37.4, 35.9, 28.4, 22.4, 15.8

LRMS m/z calculated for C₁₇H₂₀N₆O₄ 372.39 [M], found 373.2 [M+H]⁺

### N-(cyanomethyl)-1-methyl-4-(1-methyl-4-nitro-1H-pyrrole-2-carboxamido)-1H-pyrrole-2-carboxamide

IR (cm⁻¹) 3338, 1762, 1390, 1207, 1114

¹H NMR (500 MHz, DMSO-d₆) δ (ppm) 10.28 (s, 1H), 8.71 (t, J=5.6 Hz, 1H), 8.18 (d, J=2.1 Hz, 1H), 7.58 (d, J=2.1 Hz, 1H), 7.30 (d, J=2.0 Hz, 1H), 6.95 (d, J=2.0 Hz, 1H), 4.20 (d, J=5.6 Hz, 2H), 3.95 (s, 3H), 3.85 (s, 3H)

¹³C NMR (126 MHz, DMSO-d₆) δ (ppm) 161.1, 156.9, 133.8, 128.2, 126.2, 121.7, 121.4, 119.3, 117.9, 107.6, 105.1, 37.4, 36.2, 27.1

LRMS m/z calculated for C₁₋₄H₁₄N₆O₄ 330.30 [M], found 331.3 [M+H]⁺

### N-(5-cyanopentyl)-1-methyl-4-(1-methyl-4-nitro-1H-pyrrole-2-carboxamido)-1H-pyrrole-2-carboxamide

IR (cm⁻¹) 3107, 1637, 1500, 1303, 769

¹H NMR (500 MHz, DMSO-d₆) δ (ppm) 10.26-10.22 (m, 1H), 8.17 (d, J=2.1 Hz, 1H), 8.05 (t, J=5.8 Hz, 1H), 7.59 (d, J=2.3 Hz, 1H), 7.20 (d, J=2.0 Hz, 1H), 6.85 (d, J=2.0 Hz, 1H), 3.95 (s, 3H), 3.80 (s, 3H), 3.17 (q, J=6.7 Hz, 2H), 2.49-2.46 (m, 2H), 1.58 (p, J=7.2 Hz, 2H), 1.50 (p, J=7.2 Hz, 2H), 1.42-1.33 (m, 2H)

LRMS m/z calculated for C₁₈H₂₂N₆O₄ 386.41 [M], found 387.2 [M+H]⁺

### N-(5-amino-5-iminopentyl)-1-methyl-4-(1-methyl-4-nitro-1H-pyrrole-2-carboxamido)-1H-pyrrole-2-carboxamide

IR (cm⁻¹) 3130, 1634, 1498, 1305, 840

¹H NMR (500 MHz, Acetone-d) δ (ppm) 11.02 (s, 1H), 9.94 (s, 1H), 8.68-8.64 (m, 1H), 8.38 (d, J=2.1 Hz, 1H), 7.98-7.94 (m, 1H), 7.82 (d, J=2.1 Hz, 1H), 7.68 (d, J=2.0 Hz, 1H), 7.34 (d, J=2.0 Hz, 1H), 4.52 (s, 3H), 4.36 (s, 3H), 3.83 (q, J=6.5 Hz, 2H), 3.12 (t, J=7.8 Hz, 2H), 2.30 (p, J=7.4 Hz, 2H), 2.13 (p, J=6.9 Hz, 2H) ¹³C NMR (126 MHz, DMSO-d₆) δ (ppm) 170.9, 161.1, 156.9, 133.8, 128.2, 126.3, 123.2, 121.3, 118.0, 107.6, 104.1, 37.7, 37.4, 36.0, 31.5, 28.6, 24.0

LRMS m/z calculated for C₁₇H₂₃N₇O₄ 389.42 [M], found 390.2 [M+H]⁺

### N-(2-amino-2-iminoethyl)-1-methyl-4-(1-methyl-4-nitro-1H-pyrrole-2-carboxamido)-1H-pyrrole-2-carboxamide

IR (cm⁻¹) 3115, 1643, 1442, 1309, 740

¹H NMR (500 MHz, DMSO-d₆) δ (ppm) 10.34 (s, 1H), 8.69 (s, 2H), 8.56 (t, J=6.1 Hz, 1H), 8.18 (d, J=2.1 Hz, 1H), 7.62 (d, J=2.1 Hz, 1H), 7.30 (d, J=2.0 Hz, 1H), 7.02 (d, J=2.0 Hz, 1H), 4.09 (d, J=5.3 Hz, 2H), 3.96 (s, 3H), 3.82 (s, 3H) ¹³C NMR (126 MHz, DMSO-d₆) δ (ppm) 168.6, 161.8, 156.9, 133.8, 128.3, 126.2, 121.9, 121.7, 118.9, 107.7, 105.3, 37.5, 36.2

LRMS m/z calculated for C₁₋₄H₁₇N₇O₄ 347.34 [M], found 348.3 [M+H]⁺

### N-(6-amino-6-iminohexyl)-1-methyl-4-(1-methyl-4-nitro-1H-pyrrole-2-carboxamido)-1H-pyrrole-2-carboxamide

¹H NMR (500 MHz, DMSO-d₆) δ (ppm) 10.29 (s, 1H), 8.18 (d, J=2.1 Hz, 1H), 8.06 (t, J=5.8 Hz, 1H), 7.62 (d, J=2.1 Hz, 1H), 7.20 (d, J=2.0 Hz, 1H), 6.87 (d, J=2.0 Hz, 1H), 3.95 (s, 3H), 3.80 (s, 3H), 3.17 (q, J=6.9 Hz, 2H), 2.40-2.35 (m, 2H), 1.68-1.59 (m, 2H), 1.54-1.48 (m, 2H), 1.34-1.29 (m, 2H)

LRMS m/z calculated for C₁₈H₂₅N₇O₄ 403.44 [M], found 404.3 [M+H]⁺

### N-(3-imino-3-(methylamino)propyl)-1-methyl-4-(1-methyl-4-nitro-1H-pyrrole-2-carboxamido)-1H-pyrrole-2-carboxamide

IR (cm⁻¹) 3302, 3243, 3207, 3109, 1684, 1640, 1613, 1568, 1501, 1416, 1387, 1302, 1277, 1256, 1204, 1152, 1132, 1113, 1092, 1074, 885, 847, 812, 779, 748

¹H NMR (500 MHz, DMSO-d₆) δ (ppm) 10.26 (s, 1H), 8.26 (t, J=5.75 Hz, 1H), 8.19 (d, J=1.75 Hz, 1H), 7.58 (d, J=1.75 Hz, 1H), 7.22 (d, J=1.75 Hz, 1H), 6.91 (d, J=1.75 Hz, 1H), 3.96 (s, 3H), 3.83 (s, 3H), 3.50 (m, 2H), 2.80 (s, 3H), 2.59 (t, J=6.8 Hz, 2H)

HRMS m/z calculated, C₁₆H₂₂N₇O₄ 376.1728 [M+H]⁺, found 376.1734 [M+H]⁺

### 1-methyl-4-(1-methyl-4-nitro-1H-pyrrole-2-carboxamido)-N-(3-(methylamino)-3-(methylimino)propyl)-1H-pyrrole-2-carboxamide

IR (cm⁻¹) 3452, 3300, 3254, 3213, 3123, 1663, 1632, 1566, 1545, 1535, 1499, 1435, 1395, 1306, 1289, 1256, 1213, 1194, 1148, 1119, 1092, 1026, 889, 856, 829, 812, 772, 748, 723

¹H NMR (500 MHz, DMSO-d₆) δ (ppm) 10.26 (s, 1H), 8.32 (t, J=5.75 Hz, 1H), 8.19 (d, J=1.75 Hz, 1H), 7.58 (d, J=1.75 Hz, 1H), 7.22 (d, J=1.75 Hz, 1H), 6.91 (d, J=1.75 Hz, 1H), 3.96 (s, 3H), 3.83 (s, 3H), 3.46 (m, 2H), 3.02 (s, 3H), 2.77 (s, 3H), 2.71 (t, J=6.8 Hz, 2H)

HRMS m/z calculated, C₁₇H₂₄N₇O₄ 390.1884 [M+H]⁺, found 390.1888 [M+H]⁺

### N-(3-(ethylamino)-3-iminopropyl)-1-methyl-4-(1-methyl-4-nitro-1H-pyrrole-2-carboxamido)-1H-pyrrole-2-carboxamide

IR (cm⁻¹) 3051, 1494, 1247, 750

¹H NMR (500 MHz, DMSO-d₆) δ (ppm) 10.3 (s, 1H), 9.38 (s, 1H), 9.03 (s, 1H), 8.55 (s, 1H), 8.23 (t, J=5.7 Hz, 1H), 8.18 (s, 1H), 7.57 (s, 1H), 7.21 (s, 1H), 6.91 (s, 1H), 3.95 (s, 3H), 3.82 (s, 3H), 3.50 (q, J=6.1 Hz, 2H), 3.23-3.17 (m, 2H), 1.13 (t, J=7.2 Hz, 3H)

LRMS m/z calculated for C₁₇H₂₃N₇O₄ 389.18 [M]⁺, found 390.3 [M+H]⁺

### N-(3-imino-3-(isopropylamino)propyl)-1-methyl-4-(1-methyl-4-nitro-1H-pyrrole-2-carboxamido)-1H-pyrrole-2-carboxamide

IR (cm⁻¹) 3419, 1689, 1022, 999

¹H NMR (500 MHz, DMSO-d₆) δ (ppm) 10.3 (s, 1H), 9.26 (d, J=7.9 Hz, 1H), 9.01 (s, 1H), 8.55 (s, 1H), 8.20 (t, J=5.8 Hz, 1H), 8.18 (d, J=1.3 Hz, 1H), 7.56 (d, J=1.8 Hz, 1H), 7.21 (d, J=1.3 Hz, 1H), 6.91 (d, J=1.8 Hz, 1H), 3.95 (s, 3H), 3.82 (s, 3H), 3.79-3.73 (m, 1H), 3.50 (q, J=6.1 Hz, 2H), 2.57 (t, J=6.3 Hz, 2H), 1.14 (d, J=6.4 Hz, 6H)

LRMS m/z calculated for C₁₈H₂₅N₇O₄ 403.20 [M]⁺, found 404.3 [M+H]⁺

### (Z)—N-(3-(ethylamino)-3-(ethylimino)propyl)-1-methyl-4-(1-methyl-4-nitro-1H-pyrrole-2-carboxamido)-1H-pyrrole-2-carboxamide

IR (cm⁻¹) 3263, 1629, 1305, 1118

¹H NMR (500 MHz, DMSO-d₆) δ (ppm) 10.2 (s, 1H), 9.26-9.20 (m, 1H), 8.59-8.53 (m, 1H), 8.28 (t, J=5.8 Hz, 1H), 8.19 (d, J=1.5 Hz, 1H), 7.56 (d, J=1.9 Hz, 1H), 7.21 (d, J=1.5 Hz, 1H), 6.92 (d, J=1.9 Hz, 1H), 3.95 (s, 3H), 3.82 (s, 3H), 3.49-3.41 (m, 2H), 3.23-3.16 (m, 2H), 2.71 (t, J=6.6 Hz, 2H), 1.18 (t, J=7.3 Hz, 3H), 1.14 (t, J=7.2 Hz, 3H)

LRMS m/z calculated for C₁₉H₂₇N₇O₄ 417.21 [M]⁺, found 418.4 [M+H]⁺

### N-(3-imino-3-(methoxyamino)propyl)-1-methyl-4-(1-methyl-4-nitro-1H-pyrrole-2-carboxamido)-1H-pyrrole-2-carboxamide

IR (cm⁻¹) 3315, 1641, 1494, 1257

¹H NMR (500 MHz, DMSO-d₆) δ (ppm) 10.2 (s, 1H), 8.17 (s, 1H), 7.97 (t, J=5.4 Hz, 1H), 7.59-7.56 (m, 1H), 7.23-7.20 (m, 1H), 6.84-6.82 (m, 1H), 3.95 (s, 3H), 3.81 (s, 3H), 3.58 (s, 3H), 3.37-3.33 (m, 2H), 2.20 (t, J=7.4 Hz, 2H)

LRMS m/z calculated for C₁₆H₂₁N₇O₅ 391.16 [M]⁺, found 392.3 [M+H]⁺

### N-(3-imino-3-(nonylamino)propyl)-1-methyl-4-(1-methyl-4-nitro-1H-pyrrole-2-carboxamido)-1H-pyrrole-2-carboxamide

IR (cm⁻¹) 3263, 3251, 3238, 3213, 3169, 3142, 3116, 2922, 2852, 1658, 1639, 1593, 1550, 1525, 1500, 1469, 1442, 1423, 1409, 1379, 1319, 1284, 1257, 1224, 1199, 1170, 1128, 1074, 1066, 1024, 1012, 989, 920, 889, 866, 829, 815, 800, 786, 752, 738, 717, 696, 667, 628

¹H NMR (500 MHz, d⁶-DMSO) δ (ppm) 10.24 (1H, s), 9.32 (1H, bs), 9.02 (1H, s), 8.54 (1H, s), 8.20-8.18 (2H, m), 7.57 (1H, s), 7.18 (1H, s), 6.93 (1H, s), 3.95 (3H, s), 3.81 (3H, s), 3.51-3.48 (2H, m), 3.15-3.11 (2H, m), 2.59 (2H, t, J=6.2 Hz), 1.51-1.45 (2H, m), 1.22-1.19 (12H, m), 0.83-0.80 (3H, m)

LRMS m/z calculated for C₂₄H₃₇N₇O₄ 487.6 [M]⁺, found 488.4 [M+H]⁺

### (E)-N-(3-(butylamino)-3-(butylimino)propyl)-1-methyl-4-(1-methyl-4-nitro-1H-pyrrole-2-carboxamido)-1H-pyrrole-2-carboxamide

IR (cm⁻¹) 3269.34, 3226.91, 3217.27, 3132.4, 3111.18, 2960.73, 2939.52, 2908.65, 2873.94, 1647.21, 1570.06, 1527.62, 1498.69, 1436.97, 1419.61, 1402.25, 1386.82, 1307.74, 1257.59, 1197.79, 1170.79, 1134.14, 1112.93, 1093.64, 1039.63, 1024.2, 1006.84, 985.62, 962.48, 947.05, 935.48, 889.18, 837.11, 812.03, 798.53, 775.38, 750.31, 738.74, 717.52, 705.95, 684.73, 667.37, 651.94, 638.44, 623.01

¹H NMR (500 MHz, d⁶-DMSO) δ 10.27 (1H, s), 9.30 (1H, t, J=4.5 Hz), 8.64 (1H, t, J=5.5 Hz), 8.31 (1H, t, J=6 Hz), 8.17 (1H, s), 7.20 (1H, s), 6.93 (1H, s), 3.95 (3H, s), 3.82 (3H, s), 3.48-3.45 (2H, m), 3.40-3.36 (2H, m), 3.18-3.14 (2H, m), 2.73 (2H, t, J=6.5 Hz), 1.57-1.47 (4H, m), 1.33-1.27 (4H, m), 0.89-0.85 (6H, m)

LRMS m/z calculated for C₂₃H₃₅N₇O₄ 473.28 [M]⁺, found 474.0 [M+H]⁺

### N-(3-imino-3-(pentylamino)propyl)-1-methyl-4-(1-methyl-4-nitro-1H-pyrrole-2-carboxamido)-1H-pyrrole-2-carboxamide

IR (cm⁻¹) 3278.99, 3109.25, 2935.66, 1637.56, 1406.11, 1315.45, 1199.72, 1178.51, 1128.36, 717.52

¹H NMR (500 MHz, d⁶-DMSO) δ 10.25 (1H, s), 9.38 (1H, s), 9.05 (1H, s), 8.58, (1H, s), 8.21-8.17 (2H, m), 7.56 (1H, s), 7.19 (1H, s), 6.92 (1H, s), 3.94 (3H, s), 3.81 (3H, s), 3.50 (2H, s), 3.15 (bs, 2H), 2.60 (2H, bs), 1.50 (2H, s), 1.25 (4H, s), 0.82 (3H, s)

HRMS m/z calculated, C₂₀H₃₀N₇O₄ 432.2354 [M+H]⁺, found 432.2355 [M+H]⁺

### (Z)—N-(3-(heptylamino)-3-(heptylimino)propyl)-1-methyl-4-(1-methyl-4-nitro-1H-pyrrole-2-carboxamido)-1H-pyrrole-2-carboxamide

IR (cm⁻¹) 3242.34, 3124.68, 2956.87, 2929.87, 2856.58, 1643.35, 1570.06, 1502.55, 1463.97, 1406.11, 1357.89, 1305.81, 1247.94, 1184.29, 1172.72, 1089.78, 1031.92, 773.46, 750.31, 721.38, 667.37, 650.01, 607.58

¹H NMR (500 MHz, d⁶-DMSO) δ 10.27 (1H, s), 9.28 (1H, s), 8.62 (1H, s), 8.29 (1H, s), 8.17 (1H, s), 7.56, (1H, s), 7.19 (1H, s), 6.94 (1H, s), 3.95 (3H, s), 3.82 (3H, s), 3.50 (2H, s), 3.15 (2H, s), 2.73 (2H, s), 1.54-1.50 (4H, m), 1.24 (18H, bs), 0.83 (6H, s)

LRMS m/z calculated for C₂₉H₄₇N₇O₄ 557.37 [M]⁺, found 558.4 [M+H]⁺

HRMS m/z calculated, C₂₉H₄₈N₇O₄ 558.3762 [M+H]⁺, found 558.3756 [M+H]⁺

### N-(3-((3-(dimethylamino)propyl)amino)-3-iminopropyl)-1-methyl-4-(1-methyl-4-nitro-1H-pyrrole-2-carboxamido)-1H-pyrrole-2-carboxamide

IR (cm⁻¹) 3242, 1639, 1305, 750

¹H NMR (500 MHz, DMSO-d₆) δ (ppm): 10.3 (s, 1H), 8.24-8.20 (m, 1H), 8.19 (s, 1H), 7.58 (s, 1H), 7.20 (s, 1H), 6.94 (s, 1H), 3.95 (s, 3H), 3.82 (s, 3H), 3.55-3.47 (m, 2H), 3.21-3.16 (m, 2H), 2.84-2.79 (m, 2H), 2.32-2.28 (m, 2H), 2.14 (s, 6H), 1.68-1.65 (m, 2H)

LRMS m/z calculated for C₂₀H₃₀N₈O₄ 446.51 [M]⁺, found 447.3 [M+H]⁺

HRMS m/z calculated for C₂₀H₃₁N₈O₄ 447.2463 [M+H]⁺, found 447.2462 [M+H]⁺

### N-(3-imino-3-thiomorpholinopropyl)-1-methyl-4-(1-methyl-4-nitro-1H-pyrrole-2-carboxamido)-1H-pyrrole-2-carboxamide

IR (cm⁻¹) 3271, 1641, 1307, 750

¹H NMR (500 MHz, DMSO-d₆) δ (ppm) 10.3 (s, 1H), 9.38 (s, 1H), 8.87 (s, 1H), 8.36 (t, J=5.8 Hz, 1H), 8.18 (d, J=2.0 Hz, 1H), 7.62 (d, J=2.0 Hz, 1H), 7/24 (d, J=2.0 Hz, 1H), 6.95 (d, J=2.0 Hz, 1H), 3.97-3.92 (m, 5H), 3.88-3.82 (m, 5H), 3.45 (d, J=6.3 Hz, 2H), 2.88-2.76 (m, 6H)

LRMS m/z calculated for C₁H₂₅N₇O₄S 447.17 [M]⁺, found 448.2 [M+H]⁺

HRMS m/z calculated for C₁₉H₂₆N₇O₄S 448.1761 [M+H]⁺, found 448.1759 [M+H]⁺

### N-(3-imino-3-morpholinopropyl)-1-methyl-4-(1-methyl-4-nitro-1H-pyrrole-2-carboxamido)-1H-pyrrole-2-carboxamide

IR (cm⁻¹) 3140, 1654, 1307, 1116

¹H NMR (500 MHz, DMSO-d₆) δ (ppm) 10.32 (s, 1H), 8.36 (s, 1H), 8.18 (s, 1H), 7.61 (s, 1H), 7.23 (s, 1H), 6.95 (s, 1H), 3.95, 3.83 (s, 3H), 3.71-3.69 (m, 4H), 3.49-3.43 (m, 2H), 2.97-2.95 (m, 4H), 2.85-2.79 (m, 2H)

LRMS m/z calculated for C₁H₂₅N₇O₅431.45 [M]⁺, found 432.2 [M+H]⁺

HRMS m/z calculated for C₁₉H₂₅N₇O₅432.1990 [M+H]⁺, found 432.1987 [M+H]⁺

### N-(3-imino-3-(piperidin-1-yl)propyl)-1-methyl-4-(1-methyl-4-nitro-1H-pyrrole-2-carboxamido)-1H-pyrrole-2-carboxamide

IR (cm⁻¹) 3082, 1639, 1305, 750

¹H NMR (500 MHz, DMSO-d₆) δ (ppm) 10.3 (s, 1H), 9.03 (s, 1H), 8.51 (s, 1H), 8.30 (s, 1H), 8.18 (d, J=1.7 Hz, 1H), 7.58 (s, 1H), 7.21 (s, 1H), 6.93 (s, 1H), 3.95 (s, 3H), 3.82 (s, 3H), 3.67-3.63 (m, 2H), 3.54-3.49 (m, 2H), 3.47-3.40 (m, 2H), 3.03-2.98 (m, 1H), 2.80-2.71 (m, 2H), 1.68-1.52 (m, 7H)

LRMS m/z calculated for C₂₀H₂₇N₇O₄ 429.48 [M]⁺, found 430.3 [M+H]⁺

HRMS m/z calculated for C₂₀H₂₈N₇O₄ 430.2197 [M+H]⁺, found 430.2196 [M+H]⁺

### N-(3-imino-3-(4-methylpiperazin-1-yl)propyl)-1-methyl-4-(1-methyl-4-nitro-1H-pyrrole-2-carboxamido)-1H-pyrrole-2-carboxamide

IR (cm⁻¹) 3091, 1490, 1309, 798

¹H NMR (500 MHz, DMSO-d₆) δ (ppm) 10.31 (m, 1H), 9.27 (s, 1H), 8.36-8.31 (m, 1H), 8.20-8.18 (m, 1H), 7.60 (s, 1H), 7.23 (s, 1H), 6.94 (s, 1H), 3.95 (s, 3H), 3.83 (s, 3H), 3.70-3.66 (m, 2H), 3.60-3.54 (m, 2H), 3.06-2.99 (m, 8H), 2.20 (s, 3H)

LRMS m/z calculated for C₂₀H₂₈N₈O₄ 444.50 [M]⁺, found 445.3 [M+H]⁺

HRMS m/z calculated for C₂₀H₂₉N₈O₄445.2306 [M+H]⁺, found 445.2304 [M+H]⁺

### N-(3-imino-3-((2-(tetrahydro-2H-pyran-4-yl)ethyl)amino)propyl)-1-methyl-4-(1-methyl-4-nitro-1H-pyrrole-2-carboxamido)-1H-pyrrole-2-carboxamide

IR (cm⁻¹) 2931, 1631, 1303, 748

¹H NMR (500 MHz, DMSO-d₆) δ (ppm) 10.3 (s, 1H), 9.38 (s, 1H), 9.08 (s, 1H), 8.58 (s, 1H), 8.23 (s, 1H), 8.19 (s, 1H), 7.59 (s, 1H), 7.20 (s, 1H), 6.97 (s, 1H), 3.96 (s, 3H), 3.83 (3H), 3.81-3.75 (m, 2H), 3.56-3.48 (m, 2H), 1.60-1.43 (m, 7H), 1.20-1.09 (m, 3H)

LRMS m/z calculated for C₂₂H₃₁N₇O₅ 473.53 [M]⁺, found 474.3 [M+H]⁺

HRMS m/z calculated for C₂₂H₃₂N₇O₅ 474.2459 [M+H]⁺, found 474.2456 [M+H]⁺

### N-(5-((12-imino-2,5,8-trioxa-11-azatetradecan-14-yl)carbamoyl)-1-methyl-1H-pyrrol-3-yl)-1-methyl-4-nitro-1H-pyrrole-2-carboxamide

IR (cm⁻¹) 2873, 1647, 1307, 1093

¹H NMR (500 MHz, DMSO-d₆) δ (ppm) 10.30 (s, 1H), 9.56 (s, 1H), 9.17 (s, 1H), 8.69 (s, 1H), 8.23 (t, J=5.9 Hz, 1H), 8.19-8.17 (m, 1H), 7.61-7.59 (m, 1H), 7.23-7.21 (m, 1H), 6.95-6.93 (m, 1H), 3.95 (s, 3H), 3.82 (s, 3H), 3.48-3.36 (m, 14H), 3.22 (s, 3H), 2.66-2.62 (m, 2H)

LRMS m/z calculated for C₂₂H₃₃N₇O₇ 507.55 [M]⁺, found 508.3 [M+H]⁺

HRMS m/z calculated for C₂₂H₃₃N₇O₇ 508.2514 [M+H]⁺, found 508.2500 [M+H]⁺

### N-(3-imino-3-((2-(4-methylpiperazin-1-yl)ethyl)amino)propyl)-1-methyl-4-(1-methyl-4-nitro-1H-pyrrole-2-carboxamido)-1H-pyrrole-2-carboxamide

IR (cm⁻¹) 2939, 1641, 1305, 750

¹H NMR (500 MHz, DMSO-d₆) δ (ppm) 10.35 (s, 1H), 8.27 (t, J=5.4 Hz, 1H), 8.19-8.17 (m, 1H), 7.65-7.62 (m, 1H), 7.24-7.22 (m, 1H), 6.98-6.95 (m, 1H), 3.95 (s, 3H), 3.82 (s, 3H), 3.54-3.49 (m, 2H), 3.31 (t, J=6.2 Hz, 2H), 2.87 (t, J=6.2 Hz, 2H), 2.28-2.17 (m, 8H), 2.13-2.05 (m, 5H)

LRMS m/z calculated for C₂₂H₃₃N₉O₄487.57 [M]⁺, found 488.1 [M+H]⁺

HRMS m/z calculated for C₂₂H₃₄N₉O₄488.2728 [M+H]⁺, found 488.2723 [M+H]⁺

### N-(3-imino-3-(phenethylamino)propyl)-1-methyl-4-(1-methyl-4-nitro-1H-pyrrole-2-carboxamido)-1H-pyrrole-2-carboxamide

IR (cm⁻¹) 2964, 1307, 740, 694

¹H NMR (500 MHz, DMSO-d₆) δ (ppm) 10.40 (s, 1H), 9.78 (s, 1H), 9.29 (s, 1H), 8.84 (s, 1H), 8.32 (t, J=5.7 Hz, 1H), 8.17 (d, J=1.7 Hz, 1H), 7.66 (d, J=1.7 Hz, 1H), 7.30-7.25 (m, 6H), 6.99 (d, J=1.7 Hz, 1H), 3.94 (s, 3H), 3.82 (s, 3H), 3.54-3.50 (m, 2H), 3.48-3.43 (m, 2H), 2.88-2.83 (m, 2H), 2.67 (t, J=6.4 Hz, 2H)

LRMS m/z calculated for C₂₃H₂₇N₇O₄ 465.51 [M]⁺, found 466.3 [M+H]⁺

HRMS m/z calculated for C₂₃H₂₈N₇O₄ 466.2197 [M+H]⁺, found 466.2193 [M+H]⁺

### N-(3-imino-3-((2-thiomorpholinoethyl)amino)propyl)-1-methyl-4-(1-methyl-4-nitro-1H-pyrrole-2-carboxamido)-1H-pyrrole-2-carboxamide

IR (cm⁻¹) 3120, 1635, 1305, 752

¹H NMR (500 MHz, DMSO-d₆) δ (ppm) 10.36 (s, 1H), 8.28 (t, J=5.7 Hz, 1H), 8.18-8.16 (m, 1H), 7.64-7.62 (m, 1H), 7.23-7.21 (m, 1H), 6.99-6.97 (m, 1H), 3.95 (s, 3H), 3.82 (s, 3H), 2.62-2.59 (m, 8H), 2.56-2.52 (m, 8H)

LRMS m/z calculated for C₂₁H₃₀N₈O₄S 490.58 [M]⁺, found 491.3 [M+H]⁺

HRMS m/z calculated for C₂₁H₃₁N₈O₄S 491.2183 [M+H]⁺, found 491.2179 [M+H]⁺

### N-(3-((2,2-difluoroethyl)amino)-3-iminopropyl)-1-methyl-4-(1-methyl-4-nitro-1H-pyrrole-2-carboxamido)-1H-pyrrole-2-carboxamide

Adequate separation of the mono- and disubstituted dimers could not be achieved, and therefore the mixture was used without purification. The mixture was 50:50 of the mono-(m/z 426.2 [M+H]⁺) and di-substituted (m/z 490.2 [M+H]⁺) amidine by LCMS.

### (Z)—N-(3-((2,2-difluoroethyl)amino)-3-((2,2-difluoroethyl)imino)propyl)-1-methyl-4-(1-methyl-4-nitro-1H-pyrrole-2-carboxamido)-1H-pyrrole-2-carboxamide

Adequate separation of the mono- and disubstituted dimers could not be achieved, and therefore the mixture was used without purification. The mixture was 50:50 of the mono-(m/z 426.2 [M+H]⁺) and di-substituted (m/z 490.2 [M+H]⁺) amidine by LCMS.

### N-(3-imino-3-((2-methoxyethyl)amino)propyl)-1-methyl-4-(1-methyl-4-nitro-1H-pyrrole-2-carboxamido)-1H-pyrrole-2-carboxamide

IR (cm⁻¹) 3132, 1637, 1494, 1309, 752

¹H NMR (500 MHz, DMSO-d₆) δ (ppm) 10.35 (s, 1H), 9.63 (s, 1H), 9.24 (s, 1H), 8.76 (s, 1H), 8.26 (t, J=5.8 Hz, 1H), 8.18 (d, J=2.0 Hz, 1H), 7.63 (d, J=2.1 Hz, 1H), 7.23 (d, J=1.8 Hz, 1H), 6.95 (d, J=2.0 Hz, 1H), 3.95 (s, 3H), 3.82 (s, 3H), 3.54-3.49 (m, 2H), 3.47 (t, J=5.3 Hz, 2H), 3.41-3.37 (m, 2H), 3.25 (s, 3H), 2.67 (t, J=6.5 Hz, 2H)

LRMS m/z calculated for C₁₈H₂₅N₇O₅ 419.44 [M], found 420.3 [M+H]⁺

### (Z)—N-(3-((2-methoxyethyl)amino)-3-((2-methoxyethyl)imino)propyl)-1-methyl-4-(1-methyl-4-nitro-1H-pyrrole-2-carboxamido)-1H-pyrrole-2-carboxamide

¹H NMR (500 MHz, CDCl₃) δ (ppm) 8.92 (s, 1H), 7.58 (d, J=2.0 Hz, 1H), 7.49 (d, J=2.0 Hz, 1H), 7.29 (d, J=2.0 Hz, 1H), 6.78 (t, J=6.0 Hz, 1H), 6.41 (d, J=1.8 Hz, 1H), 6.23 (s, 1H), 4.03 (s, 3H), 3.87 (s, 3H), 3.61 (q, J=6.0 Hz, 3H), 3.51-3.41 (m, 7H), 3.39-3.29 (m, 7H), 2.54-2.48 (m, 2H)

LRMS m/z calculated for C₂₁H₃₁N₇O₆ 477.52 [M], found 478.3 [M+H]⁺

### N-(5-((12-imino-2,5,8-trioxa-11-azatetradecan-14-yl)carbamoyl)-1-methyl-1H-pyrrol-3-yl)-1-methyl-4-nitro-1H-pyrrole-2-carboxamide

IR (cm⁻¹) 2873, 1647, 1307, 1093

¹H NMR (500 MHz, DMSO-d₆) δ (ppm) 10.30 (s, 1H), 9.56 (s, 1H), 9.17 (s, 1H), 8.69 (s, 1H), 8.23 (t, J=5.9 Hz, 1H), 8.19-8.17 (m, 1H), 7.61-7.59 (m, 1H), 7.23-7.21 (m, 1H), 6.95-6.93 (m, 1H), 3.95 (s, 3H), 3.82 (s, 3H), 3.48-3.36 (m, 14H), 3.22 (s, 3H), 2.66-2.62 (m, 2H)

LRMS m/z calculated for C₂₂H₃₃N₇O₇ 507.55 [M], found 508.3 [M+H]⁺

HRMS m/z calculated for C₂₂H₃₃N₇O₇ 508.2514 [M+H]⁺, found 508.2500 [M+H]⁺

Synthesis of Tail Group Dimers that Contain NH Pyrroles

The tail group dimers were prepared through an amide coupling of an additional nitropyrrole carboxylic acid, either NH or NMe, to the appropriate tail group monomer.

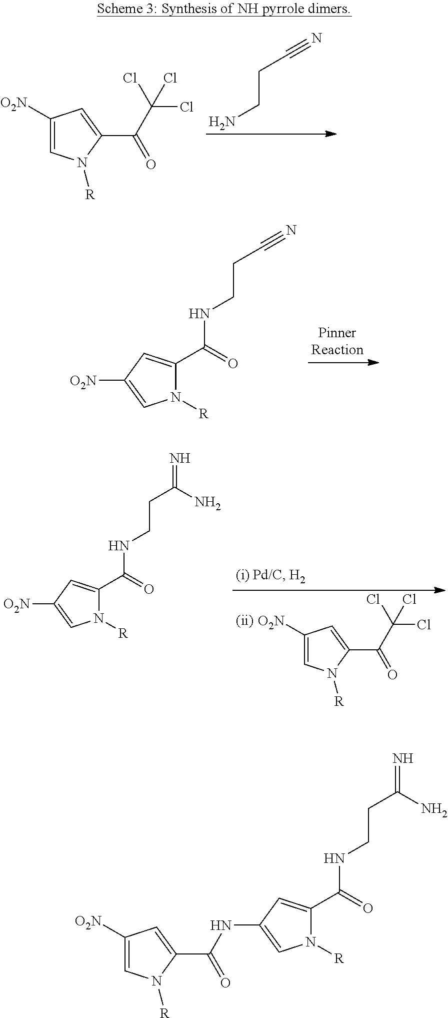

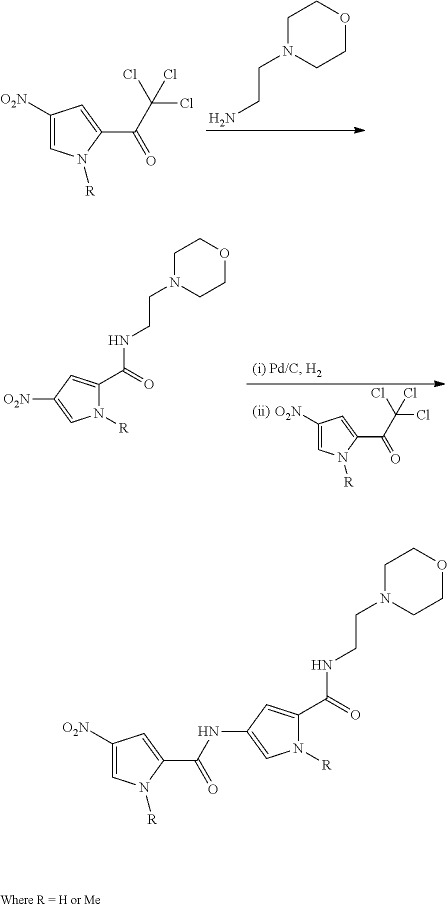

### For Amidine Containing Tail Group Monomers:

Step 1: The appropriate trichloroacetyl pyrrole (10.48 mmol) was dissolved in DMF (15 mL) and 3-Aminopropionitrile (1.5 mL, 20.97 mmol) in DMF (15 mL) was added dropwise and the reaction was stirred at room temperature overnight. Upon completion, saturated brine solution (50 mL) was added and a precipitate was observed which was collected by filtration, air dried and found to be the desired product (˜90% yield).

Step 2: The appropriate nitrile tail group monomer (3.60 mmol) was suspended in ethanol (50 mL, dry) and cooled to −60° C. Dry hydrogen chloride gas was generated from concentrated sulfuric acid and concentrated hydrochloric acid and bubbled through the solution until saturation (approximately 30 mins-1 hour). The reaction mixture was left to warm up to room temperature and then stirred for 2 hours at room temperature. The solvent was removed by evaporation under reduced pressure. The resulting solid was triturated in dry diethyl ether and collected by vacuum filtration. The presence of an ethyl imidate was checked for by NMR and then the material was taken forward immediately. The ethyl imidate was dissolved in ammonia in methanol (7M, 10 mL) and the reaction mixture was stirred at 55° C. for 3 hours. The hot solution was filtered and the solid residue washed with dilute hydrochloric acid. The solid was allowed to air dry and found to be the product (˜80% yield).

### For Morpholine Containing Tail Group Monomers:

The appropriate trichloroacetyl pyrrole (1.16 mmol) was dissolved in DCM (10 mL) to which was added 2-morpholinoethan-1-amine (151 mg, 153 μL, 1.16 mmol). The reaction was left stirring at room temperature overnight. The reaction was then cooled to 0° C. to encourage precipitation of the product. The product was isolated by Buchner filtration and allowed to air dry (˜75% yield).

For amidine or morpholine containing tail group dimers (steps (i) and (ii) of Scheme 3):

The appropriate amidine/morpholine monomer (0.25 mmol) was dissolved in methanol (25 mL) and cooled with ice-water and then Pd/C-10% (50 mg) was added portion-wise with stirring under hydrogen. The reaction mixture was hydrogenated overnight at room temperature and atmospheric pressure. The catalyst was removed over celite and the solvent removed under reduced pressure. The amine so formed was dissolved in DMF (1 mL, dry) and was treated with drop-wise addition of the trichloroacetyl derivative of either NH or NMe monomer (depending on the final expected version of dimer) in DMF (2-3 mL) and the reaction was stirred overnight and the solvent was concentrated by evaporation under reduced pressure. The resulting mixture was diluted with a minimum quantity of methanol, and was the treated with drop-wise addition of diethyl ether until the product precipitated. This was filtered and allowed to air dry to obtain the desired product (20-50% yield).

### N-(2-cyanoethyl)-4-nitro-1H-pyrrole-2-carboxamide

IR (cm⁻¹) 3340, 3165, 3138, 3099, 1643, 1568, 1537, 1494, 1475, 1440, 1413, 1384, 1361, 1311, 1261, 1234, 1215, 1139, 1109, 1070, 1041, 1010, 997, 972, 950, 921,877, 848, 835, 813, 804, 785, 752, 719, 700, 682, 644, 605

¹H NMR (500 MHz, DMSO-d₆) δ (ppm) 12.28 (1H, s), 8.78 (1H, t, J=5.2 Hz), 7.92 (1H, s), 7.45 (1H, s), 3.49 (2H, m), 2.75 (2H, t, J=6.5 Hz)

LRMS m/z calculated for C₈H₈N₄O₃ 208.1 [M]⁺, found 207.2 [M−H]⁻

### N-(2-cyanoethyl)-1-methyl-4-nitro-1H-pyrrole-2-carboxamide

Prepared as described in Federico et al., Gazzetta Chimica Italiana, 97, 1110-15, 1967

### N-(3-amino-3-iminopropyl)-4-nitro-1H-pyrrole-2-carboxamide

IR (cm⁻¹) 3215, 3099, 2158, 2029, 1685, 1639, 1535, 1492, 1469, 1415, 1352, 1311, 1253, 1226, 1205, 1143, 1107, 842, 783, 752, 705

¹H NMR (500 MHz, DMSO-d₆) δ (ppm) 12.78 (1H, s), 9.01 (2H, s), 8.74 (1H, s), 8.60 (2H, s), 7.92-7.91 (1H, m), 7.49 (1H, s), 3.58-3.54 (2H, m), 2.63 (2H, t, J=6 Hz)

LRMS m/z calculated for C₈H₁₁N₅O₃ 225.2 [M]⁺, found 226.1 [M+H]⁺

### N-(3-amino-3-iminopropyl)-1-methyl-4-nitro-1H-pyrrole-2-carboxamide Prepared as described in Berta et al., Journal of Medicinal Chemistry, 2004, Vol. 47, No. 10

### N-(2-morpholinoethyl)-4-nitro-1H-pyrrole-2-carboxamide

IR (cm⁻¹) 3402, 3215, 3142, 3101, 2970, 2862, 2810, 2783, 2160, 2032, 1629, 1618, 1575, 1533, 1500, 1475, 1438, 1415, 1355, 1319, 1280, 1261, 1226, 1209, 1139, 1112, 1066, 1026, 935, 916, 860, 842, 802, 771, 746, 613

¹H NMR (500 MHz, DMSO-d₆) δ (ppm) 12.69 (1H, s), 8.38-8.35 (1H, m), 7.88 (1H, s), 7.42 (1H, s), 3.57-3.55 (4H, m), 3.37-3.33 (2H, m), 2.45-2.40 (6H, m);

LRMS m/z calculated for C₁₁H₁₆N₄O₄ 268.1 [M]⁺, found 269.3 [M+H]⁺

### 1-methyl-N-(2-morpholinoethyl)-4-nitro-1H-pyrrole-2-carboxamide

Prepared as described in Vooturi, Sunil K. et al., Journal of Medicinal Chemistry, 2009, vol. 52, #16, p. 5020-5031

### N-(3-amino-3-iminopropyl)-4-(4-nitro-1H-pyrrole-2-carboxamido)-1H-pyrrole-2-carboxamide

IR (cm⁻¹) 3257, 3134, 3113, 3080, 3061, 2983, 2918, 2854, 1685, 1637, 1581, 1475, 1406, 1375, 1313, 1288, 1257, 1211, 1136, 1097, 1045, 964, 825, 810, 750, 636

¹H NMR (500 MHz, DMSO-d₆) δ (ppm) 12.80 (1H, s), 11.29 (1H, s), 10.24 (1H, s), 8.9 (2H, bs), 8.7 (2H, bs), 8.31 (1H, t, J=6 Hz), 7.96 (1H, bs), 7.61-7.58 (1H, m), 7.19 (1H, bs), 6.90-6.88 (1H, m), 3.57-3.53 (2H, m), 2.64-2.60 (2H, m)

LRMS m/z calculated for C₁₃H₁₅N₇O₄ 333.31 [M]⁺, found 334.2 [M+H]⁺

HRMS m/z calculated for C₁₃H₁₆N₇O₄ 334.1258 [M+H]⁺, found 334.1262 [M+H]⁺

### N-(3-amino-3-iminopropyl)-1-methyl-4-(4-nitro-1H-pyrrole-2-carboxamido)-1H-pyrrole-2-carboxamide

IR (cm⁻¹) 3387, 3136, 2987, 2949, 1631, 1587, 1550, 1531, 1504, 1469, 1435, 1419, 1402, 1373, 1309, 1284, 1259, 1199, 1180, 1130, 1095, 1008, 964, 839, 812, 796, 771, 746, 721, 707, 684, 634

¹H NMR (500 MHz, DMSO-d₆) δ (ppm) 12.77 (1H, s), 10.23 (1H, s), 8.9 (2H, bs), 8.49 (2H, bs), 8.25 (1H, t, J=6 Hz), 7.98-7.95 (1H, m), 7.61-7.58 (1H, m), 7.23 (1H, s), 6.86 (1H, s), 3.83 (3H, s), 3.53-3.48 (2H, m), 2.62-2.58 (2H, m)

LRMS m/z calculated for C₁₋₄H₁₇N₇O₄ 347.13 [M]⁺, found 348.5 [M+H]⁺

HRMS m/z calculated for C₁₋₄H₁₈N₇O₄ 348.1415 [M+H]⁺, found 348.1419 [M+H]⁺

### N-(5-((3-amino-3-iminopropyl)carbamoyl)-1H-pyrrol-3-yl)-1-methyl-4-nitro-1H-pyrrole-2-carboxamide

IR (cm⁻¹) 3138, 1639, 1633, 1591, 1575, 1550, 1535, 1506, 1469, 1438, 1419, 1402, 1377, 1311, 1286, 1259, 1199, 1180, 1134, 1097, 871, 839, 812, 798, 773, 746, 723, 709, 636, 603

¹H NMR (500 MHz, DMSO-d₆) δ (ppm) 11.26 (1H, s), 10.21 (1H, s), 8.91 (2H, bs), 8.49 (2H, bs), 8.26 (1H, t, J=6 Hz), 8.18 (1H, bs), 3.95 (3H, s), 3.56-3.51 (2H, m), 2.63-2.59 (2H, m)

LRMS m/z calculated for C₁₋₄H₁₇N₇O₄ 347.13 [M]⁺, found 348.2 [M+H]⁺

### N-(2-morpholinoethyl)-4-(4-nitro-1H-pyrrole-2-carboxamido)-1H-pyrrole-2-carboxamide

IR (cm⁻¹) 3244, 2972, 2574, 1637, 1508, 1436, 1394, 1369, 1311, 1261, 1220, 1134, 1099, 979, 854, 812, 775, 752

¹H NMR (500 MHz, DMSO-d₆) δ (ppm) 12.89 (1H, s), 11.39 (1H, s), 10.26 (1H, s), 8.36 (1H, t, J=5.5), 7.96 (1H, s), 7.60 (1H, s), 7.20 (1H, s), 6.92 (1H, s), 4.05-3.95 (2H, m), 3.71-3.62 (2H, m), 3.60-3.53 (4H, m), 3.29-3.27 (2H, m), 3.20-3.08 (2H, m)

LRMS m/z calculated for C₁₆H₂₀N₆O₅ 376.15 [M]⁺, found 377.2 [M+H]⁺

### 1-methyl-N-(2-morpholinoethyl)-4-(4-nitro-1H-pyrrole-2-carboxamido)-1H-pyrrole-2-carboxamide

IR (cm⁻¹) 3392, 3186, 3128, 2868, 1629, 1577, 1570, 1533, 1492, 1413, 1390, 1375, 1305, 1267, 1251, 1205, 1105, 985, 908, 848, 813, 775, 750, 709, 653

¹H NMR (500 MHz, DMSO-d₆) δ (ppm) 12.78 (1H, s), 10.26 (1H, s), 8.30 (1H, t, J=5.6 Hz), 7.97 (1H, bs), 7.60 (1H, s), 7.25 (1H, s), 6.90 (1H, s), 4.01-3.98 (2H, m), 3.84 (3H, s), 3.68-3.64 (2H, m), 3.56-3.52 (2H, m), 3.28 (2H, t, J=5.9 Hz), 3.15-3.12 (2H, m)

LRMS m/z calculated for C₁₇H₂₂N₆O₅ 390.17 [M]⁺, found 391.2 [M+H]⁺

HRMS m/z calculated for C₁₇H₂₃N₆O₅ 391.1724 [M+H]⁺, found 391.1726 [M+H]⁺

### 1-methyl-N-(5-((2-morpholinoethyl)carbamoyl)-1H-pyrrol-3-yl)-4-nitro-1H-pyrrole-2-carboxamide

IR (cm⁻¹) 3350, 3232, 2804, 1664, 1631, 1595, 1544, 1512, 1489, 1334, 1307, 1109, 1068, 858, 821, 746, 713

¹H NMR (500 MHz, DMSO-d₆) δ (ppm) 11.21 (1H, s), 10.20 (1H, s), 8.17 (1H, s), 7.99-7.96 (1H, m), 7.56 (1H, s), 7.13 (1H, s), 6.88 (1H, s), 3.95 (3H, s), 3.58-3.56 (4H, m), 3.37-3.34 (2H, m), 2.44-2.40 (4H, m), 2.36-2.32 (2H, m)

LRMS m/z calculated for C₁₇H₂₂N₆O₅ 390.17 [M]⁺, found 391.4 [M+H]⁺

HRMS m/z calculated for C₁₇H₂₂N₆O₅Na 413.1544 [M+Na]⁺, found 413.1545 [M+H]⁺

### Synthesis of Quaternary Ammonium Tail Group Dimers

Quaternary ammonium tail group dimers can be prepared directly from their corresponding tertiary amine tail group dimer by reacting with an appropriate alkylhalide.

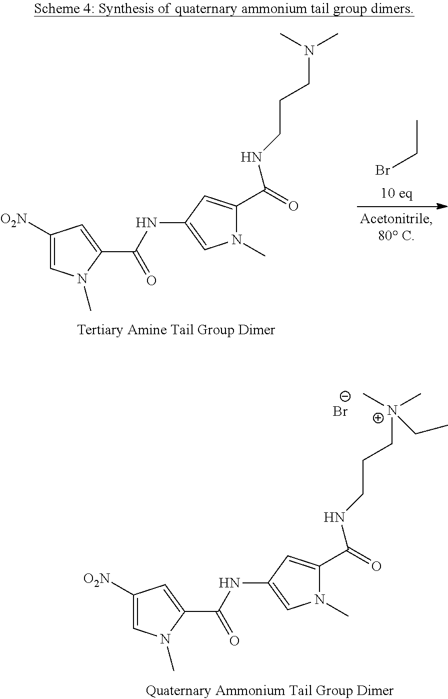

In the preparation of the compounds of the invention, the specific quaternary ammonium tail group dimer, N-ethyl-N,N-dimethyl-3-(1-methyl-4-(1-methyl-4-nitro-1H-pyrrole-2-carboxamido)-1H-pyrrole-2-carboxamido)propan-1-aminium bromide, was synthesised by reacting N-(3-(dimethylamino)propyl)-1-methyl-4-(1-methyl-4-nitro-1H-pyrrole-2-carboxamido)-1H-pyrrole-2-carboxamide with 10 equivalents of bromoethane in acetonitrile (3 mL) at 80° C. for 20 hours. The solid which precipitates upon reduction in volume of the reaction mixture is the desired material.

### N-ethyl-N,N-dimethyl-3-(1-methyl-4-(1-methyl-4-nitro-1H-pyrrole-2-carboxamido)-1H-pyrrole-2-carboxamido)propan-1-aminium bromide

IR cm⁻¹ 3495.01, 3325.28, 3240.41, 3113.11, 2358.94, 2160.27, 1978.97, 1647.21, 1595.13, 1570.06, 1533.41, 1516.05, 1436.97, 1421.54, 1402.25, 1363.67, 1311.59, 1259.52, 1213.23, 1203.58, 1118.71, 1118.71

¹H NMR (500 MHz, DMSO-d₆) δ (ppm) 10.31 (s, 1H), 8.22 (t, J=5.9 Hz, 1H), 8.19 (d, J=2.0 Hz, 1H), 7.63 (d, J=2.0 Hz, 1H), 7.22 (d, J=1.9 Hz, 1H), 6.94 (d, J=1.9 Hz, 1H), 3.96 (s, 3H), 3.83 (s, 3H), 3.35 (q, J=7.2 Hz, 3H), 3.32-3.23 (m, 4H), 3.01 (s, 6H), 1.91 (p, J=6.5 Hz, 2H), 1.24 (t, J=7.3 Hz, 3H).

¹³C NMR (126 MHz, DMSO) δ (ppm) 161.87, 157.40, 134.27, 128.71, 126.76, 123.39, 121.92, 118.75, 108.15, 104.84, 61.09, 59.02, 50.05, 40.62, 37.95, 36.56, 36.08, 23.19, 8.26.

LCMS m/z calculated for C₁₉H₂₉N₆O₄⁺ 405.22 [M]⁺, found 405.3 [M]⁺

### Synthesis of Compounds of the Invention

### Synthesis of Compounds Containing Alkyl Amidines or Amidines of Different Tail Group Lengths

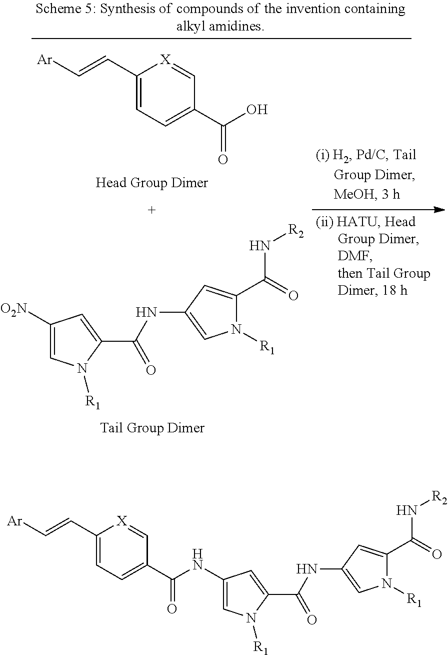

The nitro tail group dimer (or mixture of mono and di-substituted dimers) (0.3 mmol) was dissolved in methanol (25 mL) to which 10% palladium on carbon (10% by weight) was added at 0° C. under nitrogen with stirring.

The reaction mixture was hydrogenated for at least 5 h at room temperature and atmospheric pressure, monitoring for full reduction by LC-MS. The catalyst was removed over celite and the methanol was removed under reduced pressure at 50° C. to give the amine tail group dimer, which was used in the next step without purification.

The carboxylic acid head group dimer (1 eq.) was dissolved in DMF (0.5 mL, dry) to which was added HATU (2 eq.) and DIPEA (1 eq.), and then left to stir for 30 mins to form the activated ester. The amine formed in the previous step was dissolved in DMF* (0.5 mL, dry) and slowly added to the activated ester at 0° C. The stirring was continued at room temperature overnight. The product was purified by HPLC and fractions containing the required material were combined and freeze dried to give the desired product.
For those dimers that were obtain by HPLC purification, and hence were the di TFA salt, an extra two equivalents of DIPEA was added to this DMF solution.

### Characterisation Details of Compounds of the Invention Containing Alkyl Amidines or Amidines of Different Tail Group Lengths

### Ia1 (E)-6-(4-(dimethylamino)styryl)-N-(5-((5-((3-(ethylamino)-3-iminopropyl)carbamoyl)-1-methyl-1H-pyrrol-3-yl)carbamoyl)-1-methyl-1H-pyrrol-3-yl)nicotinamide

IR (cm⁻¹) 3273, 3041, 2951, 2870, 1654, 1570, 1294, 1244, 1201, 1174, 1130, 1109, 1014, 831, 698, 638

¹H NMR (500 MHz, DMSO-d₆) δ (ppm) δ 10.43 (1H, s), 9.95 (1H, s), 9.34 (2H, s), 9.01 (2H, d, J=17.2 Hz), 8.52 (1H, s), 8.23 (2H, d, J=8.05 Hz), 8.17 (1H, t, J=5.5 Hz), 7.70 (1H, d, J=15.85 Hz), 7.61-7.60 (1H, d, J=8.65 Hz), 7.53 (2H, d, J=8.5 Hz), 7.33 (1H, s), 7.18 (1H, s), 7.13-7.10 (2H, m), 6.95 (1H, s), 6.75 (2H, d, J=8.6 Hz), 3.87 (3H, s, under the water peak), 3.18 (3H, s, under water peak), 3.55-3.40 (2H, m) 3.21-3.17 (2H, m), 2.97 (6H, s), 2.60-2.57 (2H, m). 1.10-1.20 (3H, m)

LRMS m/z calculated for C₃₃H₃₉N₉O₃ 609.3 [M]⁺, found 610.4 [M+H]⁺

HRMS m/z calculated for C₃₃H₄₀O₉N₃ 610.3249 [M+H]⁺ found 610.3245 [M+H]⁺

### Ia2 (E)-N-(3-imino-3-(methylamino)propyl)-1-methyl-4-(1-methyl-4-(4-(4-(trifluoromethyl)styryl)benzamido)-1H-pyrrole-2-carboxamido)-1H-pyrrole-2-carboxamide

IR (cm⁻¹) 3310, 3201, 3003, 1638, 1580, 1526, 1464, 1435, 1404, 1323, 1265, 1199, 1165, 1109, 1065, 1015, 964, 949, 843, 799, 777, 758, 720

¹H NMR (500 MHz, DMSO-d₆) δ (ppm) 10.35 (s, 1H), 9.96 (s, 1H), 9.42 (m, 1H), 9.06 (s, 1H), 8.52 (s, 1H), 8.26 (t, J=5.60 Hz, 4H), 7.99 (d, J=8.4 Hz, 2H), 7.87 (d, J=8.15 Hz, 2H), 7.76-7.81 (m, 4H), 7.52 (s, 2H), 7.34 (d, J=1.75 Hz, 1H), 7.19 (d, J=1.75 Hz, 1H), 7.12 (d, J=1.75 Hz, 1H), 6.91 (d, J=1.75 Hz, 1H), 3.89 (s, 3H), 3.83 (s, 3H), 3.50 (m, 2H), 2.80 (d, J=4.9 Hz, 3H), 2.56 (t, J=6.45 Hz, 2H)

HRMS m/z calculated for C₃₂H₃₃N₇O₃F₃ 620.2591 [M+H]⁺, found 620.2579 [M+H]⁺

### Ia3 (E)-4-(4-(2-(benzo[c][1,2,5]oxadiazol-5-yl)vinyl)benzamido)-N-(5-((3-imino-3-(methylamino)propyl)carbamoyl)-1-methyl-1H-pyrrol-3-yl)-1-methyl-1H-pyrrole-2-carboxamide

IR (cm⁻¹) 3309, 3003, 2990, 1626, 1537, 1503, 1468, 1435, 1408, 1289, 1265, 1199, 1179, 1125, 1007, 957, 878, 849, 829, 800, 754, 720

¹H NMR (500 MHz, DMSO-d₆) δ (ppm) 10.38 (s, 1H), 9.96 (s, 1H), 9.41 (m, 1H), 9.06 (s, 1H), 8.52 (s, 1H), 8.21 (t, J=5.60 Hz, 4H), 8.11 (s, 3H), 8.02 (d, J=8.4 Hz, 2H), 7.82 (d, J=8.4 Hz, 2H), 7.70 (d, J=16.5 Hz, 1H), 7.62 (d, J=16.5 Hz, 1H), 7.34 (d, J=1.75 Hz, 1H), 7.19 (d, J=1.75 Hz, 1H), 7.13 (d, J=1.75 Hz, 1H), 6.95 (d, J=1.75 Hz, 1H), 3.89 (s, 3H), 3.83 (s, 3H), 3.50 (m, 2H), 2.80 (d, J=4.9 Hz, 3H), 2.59 (t, J=6.45 Hz, 2H)

HRMS m/z calculated for C₃₁H₃₂N₉O₄ 594.2572 [M+H]⁺, found 594.2570 [M+H]⁺

### Ia4 (E)-N-(3-imino-3-(methylamino)propyl)-1-methyl-4-(1-methyl-4-(4-(2-(naphthalen-2-yl)vinyl)benzamido)-1H-pyrrole-2-carboxamido)-1H-pyrrole-2-carboxamide

IR (cm⁻¹) 3296, 3103, 1670, 1638, 1580, 1533, 1464, 1435, 1404, 1267, 1199, 1182, 1130, 1063, 1011, 959, 895, 833, 801, 772, 745, 721

¹H NMR (500 MHz, DMSO-d₆) δ (ppm) 10.34 (s, 1H), 9.96 (s, 1H), 9.41 (m, 1H), 9.06 (s, 1H), 8.52 (s, 1H), 8.21 (t, J=5.60 Hz, 1H), 8.08 (s, 1H), 8.01 (d, J=8.4 Hz, 2H), 7.90-7.99 (m, 4H), 7.80 (d, J=8.4 Hz, 2H), 7.45-7.51 (m, 4H), 7.35 (d, J=1.75 Hz, 1H), 7.20 (d, J=1.75 Hz, 1H), 7.13 (d, J=1.75 Hz, 1H), 6.95 (d, J=1.75 Hz, 1H), 3.89 (s, 3H), 3.83 (s, 3H), 3.50 (m, 2H), 2.80 (d, J=4.9 Hz, 3H), 2.60 (t, J=6.45 Hz, 2H)

HRMS m/z calculated for C₃₅H₃₆N₇O₃ 602.2874 [M+H]⁺, found 602.2866 [M+H]⁺

### Ia5 1-methyl-4-(1-methyl-4-(4-((E)-4-(trifluoromethyl)styryl)benzamido)-1H-pyrrole-2-carboxamido)-N—((Z)-3-(methylamino)-3-(methylimino)propyl)-1H-pyrrole-2-carboxamide

IR (cm⁻¹) 3269, 3115, 2961, 1643, 1634, 1614, 1520, 1504, 1435, 1404, 1385, 1325, 1265, 1200, 1169, 1111, 1067, 1015, 968, 951, 845, 833, 818, 799, 773, 758, 719, 692, 675, 667, 627, 608

¹H NMR (500 MHz, DMSO-d₆) δ (ppm) 10.36 (s, 1H), 9.97 (s, 1H), 9.34 (m, 1H), 8.60 (m, 1H), 8.27 (t, J=5.60 Hz, 4H), 7.99 (d, J=8.4 Hz, 2H), 7.87 (d, J=8.15 Hz, 2H), 7.76-7.81 (m, 4H), 7.52 (s, 2H), 7.34 (d, J=1.75 Hz, 1H), 7.20 (d, J=1.75 Hz, 1H), 7.12 (d, J=1.75 Hz, 1H), 6.95 (d, J=1.75 Hz, 1H), 3.89 (s, 3H), 3.83 (s, 3H), 3.47 (m, 2H), 3.03 (d, J=4.75 Hz, 3H), 2.79 (d, J=4.9 Hz, 3H), 2.72 (t, J=6.45 Hz, 2H)

HRMS m/z calculated for C₃₂H₃₃N₇O₃F₃ 620.2591 [M+H]⁺, found 620.2579 [M+H]⁺

### Ia6 4-(4-((E)-2-(benzo[c][1,2,5]oxadiazol-5-yl)vinyl)benzamido)-1-methyl-N-(1-methyl-5-(((Z)-3-(methylamino)-3-(methylimino)propyl)carbamoyl)-1H-pyrrol-3-yl)-1H-pyrrole-2-carboxamide

IR (cm⁻¹) 3309, 3003, 2990, 1626, 1537, 1503, 1468, 1435, 1408, 1289, 1265, 1199, 1179, 1125, 1007, 957, 878, 849, 829, 800, 754, 720

¹H NMR (500 MHz, DMSO-d₆) δ (ppm) 10.38 (s, 1H), 9.97 (s, 1H), 9.35 (m, 1H), 8.60 (m, 1H), 8.27 (t, J=5.60 Hz, 4H), 8.11 (s, 3H), 8.02 (d, J=8.4 Hz, 2H), 7.82 (d, J=8.4 Hz, 2H), 7.70 (d, J=16.5 Hz, 1H), 7.62 (d, J=16.5 Hz, 1H), 7.34 (d, J=1.75 Hz, 1H), 7.20 (d, J=1.75 Hz, 1H), 7.13 (d, J=1.75 Hz, 1H), 6.95 (d, J=1.75 Hz, 1H), 3.89 (s, 3H), 3.83 (s, 3H), 3.47 (m, 2H), 3.03 (d, J=4.6 Hz, 3H), 2.79 (d, J=4.9 Hz, 3H), 2.72 (t, J=6.45 Hz, 2H)

HRMS m/z calculated for C₃₁H₃₂N₉O₄ 594.2572 [M+H]⁺, found 594.2570 [M+H]⁺

### Ia7 1-methyl-4-(1-methyl-4-(4-((E)-2-(naphthalen-2-yl)vinyl)benzamido)-1H-pyrrole-2-carboxamido)-N—((Z)-3-(methylamino)-3-(methylimino)propyl)-1H-pyrrole-2-carboxamide

IR (cm⁻¹) 3279, 3127, 2955, 1653, 1578, 1522, 1437, 1404, 1364, 1265, 1200, 1179, 1126, 1015, 962, 897, 860, 818, 773, 745, 719

¹H NMR (500 MHz, DMSO-d₆) δ (ppm) 10.34 (s, 1H), 9.97 (s, 1H), 9.34 (m, 1H), 8.60 (m, 1H), 8.27 (t, J=5.60 Hz, 1H), 8.08 (s, 1H), 8.00 (d, J=8.4 Hz, 2H), 7.90-7.99 (m, 4H), 7.80 (d, J=8.4 Hz, 2H), 7.45-7.51 (m, 4H), 7.35 (d, J=1.75 Hz, 1H), 7.20 (d, J=1.75 Hz, 1H), 7.13 (d, J=1.75 Hz, 1H), 6.95 (d, J=1.75 Hz, 1H), 3.89 (s, 3H), 3.83 (s, 3H), 3.48 (m, 2H), 3.03 (d, J=4.6 Hz, 3H), 2.79 (d, J=4.9 Hz, 3H), 2.72 (t, J=6.45 Hz, 2H)

HRMS m/z calculated for C₃₅H₃₆N₇O₃ 602.2874 [M+H]⁺, found 602.2866 [M+H]⁺

### Ia8 6-((E)-4-(dimethylamino)styryl)-N-(1-methyl-5-((1-methyl-5-(((E)-3-(methylamino)-3-(methylimino)propyl)carbamoyl)-1H-pyrrol-3-yl)carbamoyl)-1H-pyrrol-3-yl)nicotinamide

IR (cm⁻¹) 3045, 1571, 1166, 702

¹H NMR (500 MHz, DMSO-d₆) δ (ppm) 10.5 (s, 1H), 9.97 (s, 1H), 9.39-9.33 (m, 1H), 9.06-9.03 (m, 1H), 8.65-8.59 (m, 1H), 8.35-8.30 (m, 1H), 8.28 (t, J=5.9 Hz, 1H), 7.78-7.69 (m, 2H), 7.55 (d, J=8.8 Hz, 2H), 7.35 (d, J=1.5 Hz, 1H), 7.20 (d, J=1.7 Hz, 1H), 7.17-7.10 (m, 2H), 6.94 (d, J=1.7 Hz, 1H), 6.78 (d, J=8.8 Hz, 2H), 3.88 (s, 3H), 3.82 (s, 3H), 3.50-3.44 (m, 2H), 3.04-3.02 (m, 3H), 2.99 (s, 6H), 2.78 (d, J=4.9 Hz, 3H), 2.74-2.69 (m, 2H)

LRMS m/z calculated for C₃₃H₃₉N₉O₃ 609.74 [M]⁺, found 610.4 [M+H]⁺

HRMS m/z calculated for C₃₃H₄₀N₉O₃ 610.3249 [M+H]⁺, found 610.3243 [M+H]⁺

### Ia9 (E)-6-(4-(dimethylamino)styryl)-N-(5-((5-((3-imino-3-(methylamino)propyl)carbamoyl)-1-methyl-1H-pyrrol-3-yl)carbamoyl)-1-methyl-1H-pyrrol-3-yl)nicotinamide

IR (cm⁻¹) 3363, 1653, 1124, 954

¹H NMR (500 MHz, DMSO-d₆) δ (ppm) 10.5 (s, 1H), 9.97 (s, 1H), 9.46-9.40 (m, 1H), 9.08-9.04 (m, 2H), 8.53 (s, 1H), 8.33 (dd, J=8.3, 2.2 Hz, 1H), 8.21 (t, J=5.8 Hz, 1H), 7.79-7.72 (m, 2H), 7.55 (d, J=8.8 Hz, 2H), 7.35 (d, J=1.5 Hz, 1H), 7.20 (d, J=1.5 Hz, 1H), 7.16-7.09 (m, 2H), 6.94 (d, J=1.5 Hz, 1H), 6.78 (d, J=8.8 Hz, 2H), 3.88 (s, 3H), 3.82 (s, 3H), 3.52-3.47 (m, 2H), 2.99 (6H), 2.80 (d, J=4.9 Hz, 3H), 2.61-2.56 (m, 2H)

LRMS m/z calculated for C₃₂H₃₇O₃N₉ 595.71 [M]⁺, found 596.4 [M+H]⁺

HRMS m/z calculated for C₃₂H₃₈O₃N₉ 596.3092 [M+H]⁺, found 596.3084 [M+H]⁺

### Ia10 1-methyl-4-(1-methyl-4-(4-((E)-2-(quinolin-3-yl)vinyl)benzamido)-1H-pyrrole-2-carboxamido)-N—((Z)-3-(methylamino)-3-(methylimino)propyl)-1H-pyrrole-2-carboxamide

IR (cm⁻¹) 3300, 1633, 1172, 1128

¹H NMR (500 MHz, DMSO-d₆) δ (ppm) 10.4 (s, 1H), 9.97 (s, 1H), 9.37-9.32 (m, 1H), 9.29 (s, 1H), 8.60 (s, 1H), 8.27 (t, J=5.9 Hz, 1H), 8.06-8.00 (m, 4H), 7.82 (d, J=8.5 Hz, 2H), 7.80-7.76 (m, 1H), 7.71-7.62 (m, 3H), 7.34 (d, J=1.6 Hz, 1H), 7.20 (d, J=1.6 Hz, 1H), 7.12 (d, J=1.6 Hz, 1H), 6.95 (d, J=1.6 Hz, 1H), 3.88 (s, 3H), 3.82 (s, 3H), 3.47 (q, J=6.3 Hz, 2H), 3.03 (d, J=4.7 Hz, 3H), 2.78 (d, J=4.9 Hz, 2H), 2.72 (t, J=6.5 Hz, 2H)

LRMS m/z calculated for C₃₅H₃₆N₈O₃ 616.73 [M]⁺, found 617.4 [M+H]⁺

HRMS m/z calculated for C₃₅H₃₇N₈O₃ 617.2983 [M+H]⁺, found 617.2979 [M+H]⁺

### Ia11 4-(4-((E)-2-(benzo[d]thiazol-2-yl)vinyl)benzamido)-1-methyl-N-(1-methyl-5-(((Z)-3-(methylamino)-3-(methylimino)propyl)carbamoyl)-1H-pyrrol-3-yl)-1H-pyrrole-2-carboxamide

IR (cm⁻¹) 3215, 1640, 1184, 1124, 721

¹H NMR (500 MHz, DMSO-d₆) δ (ppm) 10.4 (s, 1H), 9.98 (s, 1H), 9.38-9.33 (m, 1H), 8.63-8.59 (m, 1H), 8.31-8.26 (m, 1H), 8.14 (d, J=8.1 Hz, 1H), 8.04-8.00 (m, 3H), 7.97-7.93 (m, 2H), 7.79-7.76 (m, 2H), 7.58-7.54 (m, 1H), 7.50-7.46 (m, 1H), 7.37-7.35 (m, 1H), 7.22-7.20 (m, 1H), 7.15-7.13 (m, 1H), 6.97-6.95 (m, 1H), 3.89 (s, 3H), 3.83 (s, 3H), 3.49-3.47 (m, 2H), 3.04 (d, J=4.7 Hz, 3H), 2.79 (d, J=4.7 Hz, 3H), 2.72 (t, J=6.6 Hz, 2H).

LRMS m/z calculated for C₃₃H₃₄N₈O₃S 622.25 [M]⁺, found 623.0 [M+H]⁺

HRMS m/z calculated for C₃₃H₃₄N₈O₃S 622.2575 [M]⁺, found 623.2547 [M+H]⁺

### Ia12 N—((Z)-3-amino-3-(ethylimino)propyl)-4-(4-(4-((E)-2-(benzo[c][1,2,5]oxadiazol-5-yl)vinyl)benzamido)-1-methyl-1H-pyrrole-2-carboxamido)-1-methyl-1H-pyrrole-2-carboxamide

IR (cm⁻¹) 3084, 1610, 1261, 621

¹H NMR (500 MHz, DMSO-d₆) δ (ppm) 10.4, (s, 1H), 9.97 (s, 1H), 9.40 (s, 1H), 9.05 (s, 1H), 8.56 (s, 1H), 8.22-8.18 (m, 1H), 8.12 (s, 3H), 8.03 (d, J=8.4 Hz, 2H), 7.83 (d, J=8.4 Hz, 2H), 7.71 (d, J=16 Hz, 1H), 7.62 (d, J=16 Hz, 1H), 7.36 (d, J=1.7 Hz, 1H), 7.20 (d, J=1.7 Hz, 1H), 7.13 (d, J=1.7 Hz, 1H), 6.97 (s, J=1.7 Hz, 1H), 3.89 (s, 3H), 3.83 (s, 3H), 3.54-3.49 (m, 2H), 3.25-3.18 (m, 2H), 2.60 (t, J=6.2 Hz, 2H), 1.15 (t, J=7.2 Hz, 3H)

LRMS m/z calculated for C₃₂H₃₃N₉O₄ 607.27 [M]⁺, found 608.5 [M+H]⁺

HRMS m/z calculated for C₃₂H₃₄O₄N₉ 608.2728 [M+H]⁺, found 608.2722 [M+H]⁺

### Ia13 N—((Z)-3-amino-3-(isopropylimino)propyl)-4-(4-(4-((E)-2-(benzo[c][1,2,5]oxadiazol-5-yl)vinyl)benzamido)-1-methyl-1H-pyrrole-2-carboxamido)-1-methyl-1H-pyrrole-2-carboxamide

IR (cm⁻¹) 3080, 1627, 1199, 800

¹H NMR (500 MHz, DMSO-d₆) δ (ppm) 10.37 (s, 1H, NH), 9.96 (s, 1H, NH), 9.22 (d, J=8.55, 1H, NH), 8.97 (s, 1H, NH), 8.52 (s, 1H, NH), 8.16-8.12 (m, 1H, NH), 8.11 (m, 3H, ArH), 8.01 (d, J=8.39 Hz, 2H, ArH), 7.82 (d, J=8.39 Hz, 2H, ArH), 7.70 (d, J=16.60, 1H, ArCH═CHR), 7.61 (d, J=16.60, 1H, ArCH═CHR), 7.34 (d, J=1.67 Hz, 1H, pyrroleH), 7.18 (d, J=1.67 Hz, 1H, pyrroleH), 7.11 (d, J=1.67 Hz, 1H, pyrroleH), 6.97 (d, J=1.67 Hz, 1H, pyrroleH), 3.88 (s, 3H, pyrroleCH₃), 3.82 (s, 3H, pyrroleCH₃), 3.79-3.72 (m, 1H, RCH)CH₃)₂), 3.53-3.48 (m, 2H, RNHCH₂CH₂R), 2.59-2.54 (m, 2H, RNHCH₂CH₂R), 1.16 (d, J=6.41 Hz, 6H, RCH)CH₃)₂)

LRMS m/z calculated for C₃₃H₃₅N₉O₄ 621.70 [M]⁺, found 622.0 [M+H]⁺

### Ia14 N—((Z)-3-amino-3-(ethylimino)propyl)-1-methyl-4-(1-methyl-4-(4-((E)-2-(quinolin-3-yl)vinyl)benzamido)-1H-pyrrole-2-carboxamido)-1H-pyrrole-2-carboxamide

IR (cm⁻¹) 3265, 3096, 1670, 1630, 1528, 1506, 1466, 1437, 1404, 1265, 1198, 1180, 1130, 1063, 862, 831, 799, 772, 750, 719, 685, 660, 625, 610

¹H NMR (500 MHz, DMSO-d₆) δ (ppm) 10.4 (s, 1H), 9.96 (s, 1H), 9.35 (s, 1H), 9.27 (s, 1H), 9.00 (s, 1H), 8.56 (s, 1H), 8.52 (s, 1H), 8.18 (t, J=6.0 Hz, 1H), 8.05-7.99 (m, 5H), 7.84-7.80 (m, 2H), 7.79-7.74 (m, 2H), 7.68-7.63 (m, 3H), 7.34 (d, J=1.7 Hz, 1H), 7.19 (d, J=1.7 Hz, 1H), 7.12 (d, J=1.7 Hz, 1H), 6.96 (d, J=1.7 Hz, 1H), 3.88 (s, 3H), 3.82 (s, 3H), 3.52-3.48 (m, 2H), 3.23-3.17 (m, 2H), 2.59 (t, J=6.5 Hz, 2H), 1.14 (t, J=7.3 Hz, 3H).

LRMS m/z calculated for C₃₅H₃₆N₈O₃ 616.29 [M]⁺, found 617.1 [M+H]⁺

HRMS m/z calculated for C₃₅H₃₇O₃N₈ 617.2983 [M+H]⁺, found 617.2978 [M+H]⁺

### Ia15 (E)-N-(5-((5-((3-imino-3-(isopropylamino)propyl)carbamoyl)-1-methyl-1H-pyrrol-3-yl)carbamoyl)-1-methyl-1H-pyrrol-3-yl)-6-(4-methoxystyryl)nicotinamide

IR (cm⁻¹) 3394, 2941, 1341, 1022

¹H NMR (500 MHz, DMSO-d₆) δ (ppm) 10.5 (s, 1H), 9.96 (s, 1H), 9.26-9.21 (m, 1H), 9.08-9.06 (m, 1H), 8.98 (s, 1H), 8.53 (s, 1H), 8.26 (dd, J=8.2, 2.3 Hz, 1H), 8.18-8.12 (m, 1H), 7.76 (d, J=16 Hz, 1H), 7.69-7.63 (m, 3H), 7.35 (d, J=1.7 Hz, 1H), 7.25 (d, J=16 Hz, 1H), 7.18 (d, J=1.7 Hz, 1H), 7.10 (d, J=1.8 Hz, 1H), 7.00 (d, J=8.9 Hz, 2H), 6.96 (d, J=1.8 Hz, 1H), 3.88 (s, 3H), 3.82 (s, 3H), 3.81 (s, 3H), 3.77-3.75 (m, 1H), 3.52-3.49 (m, 2H), 2.59-2.55 (m, 2H), 1.15 (d, J=6.4 Hz, 6H)

LRMS m/z calculated for C₃₃H₃₈N₈O₄ 610.30 [M]⁺, found 611.4 [M+H]⁺

### Ia16 (E)-4-(4-(4-cyanostyryl)benzamido)-N-(5-((3-(ethylamino)-3-iminopropyl)carbamoyl)-1-methyl-1H-pyrrol-3-yl)-1-methyl-1H-pyrrole-2-carboxamide

IR (cm⁻¹) 3128, 1631, 1195, 704

¹H NMR (500 MHz, DMSO-d₆) δ (ppm) 10.35 (s, 1H, NH), 9.50 (s, 1H, NH), 9.35 (s, 1H, NH), 9.00 (s, 1H, NH), 8.52 (s, 1H, NH), 8.18 (t, J=5.52 Hz, 1H, NH), 8.01-7.97 (m, 2H, ArH), 7.89-7.82 (m, 4H, ArH), 7.81-7.77 (m, 2H, ArH), 7.56 (d, J=16.5 Hz, 1H, ArCH═CHAr), 7.49 (d, J=16.5 Hz, 1H, ArCH═CHAr), 7.33 (d, J=1.67 Hz, 1H, pyrroleH), 7.18 (d, J=1.67 Hz, 1H, pyrroleH), 7.11 (d, J=1.67 Hz, 1H, pyrroleH), 6.95 (d, J=1.67 Hz, pyrroleH), 3.88 (s, 3H, NCH₃), 3.82 (s, 3H, NCH₃), 3.52-3.48 (m, 2H, NHCH₂), 3.23-3.16 (m, 2H, CH₃CH₂NH), 2.61-2.56 (m, 2H, NHCH₂CH₂NH), 1.16-1.12 (t, J=7.24 Hz, 3H, CH₃CH₂NH)

LRMS m/z calculated, found C₃₃H₃₄N₈O₃ 590.69 [M]⁺, found 591.0 [M+H]⁺

HRMS m/z calculated for C₃₃H₃₅O₃N₈ 591.2827 [M+H]⁺, found 591.2819 [M+H]⁺

### Ia17 (E)-4-(4-(4-cyanostyryl)benzamido)-N-(5-((3-imino-3-(isopropylamino)propyl)carbamoyl)-1-methyl-1H-pyrrol-3-yl)-1-methyl-1H-pyrrole-2-carboxamide

IR (cm⁻¹) 3091, 1643, 1199, 759

¹H NMR (500 MHz, DMSO-d₆) δ (ppm) 10.32 (s, 1H, NH), 9.96 (s, 1H, NH), 9.28-9.22 (m, 1H, NH), 8.99 (s, 1H, NH), 8.54 (s, 1H, NH), 8.20-8.13 (m, 1H, NH), 7.99 (d, J=8.09 Hz, 2H, ArH), 7.88-7.82 (m, 4H, ArH), 7.81-7.77 (m, 2H, ArH), 7.56 (d, J=16.51 Hz, 1H, ArCH═CHAr), 7.49 (d, J=16.51 Hz, 1H, ArCH═CHAr), 7.37-7.32 (m, 1H, pyrroleH), 7.22-7.17 (m, 1H, pyrroleH), 7.13-7.09 (m, 1H, pyrroleH), 6.98-6.94 (m, 1H, pyrroleH), 3.87 (s, 3H, pyrroleCH₃), 3.82 (s, 3H, pyrroleCH₃), 3.79-3.71 (m, 1H, RCH(CH₃)₃), 3.54-3.47 (m, 2H, RNHCH₂CH₂R); 2.60-2.55 (m, 2H, RNHCH₂CH₂R), 1.16 (s, 3H, RCH(CH₃)₃), 1.15 (s, 3H, RCH(CH₃)₃)

LRMS m/z calculated C₃₄H₃₈N₈O₃ 604.72 [M]⁺, found 605.1 [M+H]⁺

HRMS m/z calculated for C₃₄H₃₇O₃N₈ 605.2983 [M+H]⁺, found 605.2976 [M+H]⁺

### Ia18 (E)-N-(3-imino-3-(isopropylamino)propyl)-4-(4-(4-(4-methoxystyryl)benzamido)-1-methyl-1H-pyrrole-2-carboxamido)-1-methyl-1H-pyrrole-2-carboxamide

IR (cm⁻¹) 3269, 1633, 1201, 719

¹H NMR (500 MHz, DMSO-d₆) δ (ppm) 10.3 (s, 1H), 9.95 (s, 1H), 9.21 (d, J=7.0 Hz, 1H), 8.96 (s, 1H), 8.51 (s, 1H), 8.14 (t, J=5.0 Hz, 1H), 7.97 (d, J=8.4 Hz, 2H), 7.74 (d, J=8.4 Hz, 2H), 7.37-7.36 (m, 2H), 7.34-7.30 (m, 2H), 7.23-7.20 (m, 2H), 7.18 (d, J=1.7 Hz, 1H), 7.11 (d, J=1.7 Hz, 1H), 6.96 (d, J=1.5 Hz, 1H), 6.91-6.87 (m, 1H), 3.87 (s, 3H), 3.82 (s, 3H), 3.81 (S, 3H), 3.77-3.70 (m, 1H), 3.51-3.49 (m, 2H), 2.58-2.55 (m, 2H), 1.15 (d, J=6.5 Hz, 6H)

LRMS m/z calculated for C₃₄H₃₉N₇O₄ 609.31 [M]⁺, found 610.1 [M+H]⁺

HRMS m/z calculated for C₃₅H₄₀O₄N₇ 610.3136 [M+H]⁺, found 610.3137 [M+H]⁺

### Ia19 (E)-N-(3-imino-3-(isopropylamino)propyl)-4-(4-(4-(3-methoxystyryl)benzamido)-1-methyl-1H-pyrrole-2-carboxamido)-1-methyl-1H-pyrrole-2-carboxamide

IR (cm⁻¹) 3080, 1629, 1199, 719

¹H NMR (500 MHz, DMSO-d₆) δ (ppm) 10.3 (s, 1H), 9.95 (s, 1H), 9.22 (d, J=8.0 Hz, 1H), 8.96 (s, 1H), 8.51 (s, 1H), 8.14 (t, J=6.0 Hz, 1H), 7.97 (d, J=8.5 Hz, 2H), 7.74 (d, J=8.5 Hz, 2H), 7.38-7.30 (m, 4H), 7.24-7.21 (m, 2H), 7.18 (d, J=1.7 Hz, 1H), 7.11 (d, J=1.7 Hz, 1H), 6.96 (d, J=1.7 Hz), 6.91-6.87 (m, 1H), 3.87 (s, 3H), 3.82 (s, 3H), 3.81 (s, 3H), 3.52-3.49 (m, 2H), 2.58-2.55 (m, 2H), 6.37 (d, J=6.4 Hz, 6H)

LRMS m/z calculated for C₃₄H₃₉N₇O₄ 609.31 [M]⁺, found 610.1 [M+H]⁺

HRMS m/z calculated for C₃₄H₄₀O₄N₇ 610.3136 [M+H]⁺, found 610.3130 [M+H]⁺

### Ia20 (E)-N-(3-(ethylamino)-3-iminopropyl)-4-(4-(4-(4-methoxystyryl)benzamido)-1-methyl-1H-pyrrole-2-carboxamido)-1-methyl-1H-pyrrole-2-carboxamide

IR (cm⁻¹) 3255, 1629, 1199, 719

¹H NMR (500 MHz, DMSO-d₆) δ (ppm) 10.3 (s, 1H), 9.95 (s, 1H), 9.35 (s, 1H), 9.01 (s, 1H), 8.53 (s, 1H), 8.18 (t, J=8.5 Hz, 1H), 7.97 (d, J=8.3 Hz, 2H), 7.74 (d, J=8.3 Hz, 2H), 7.39-7.29 (m, 4H), 7.24-7.18 (m, 3H), 7.11 (d, J=1.4 Hz, 1H), 6.95 (d, J=1.5 Hz, 1H), 6.91-6.87 (m, 1H), 3.88 (s, 3H), 8.83-3.80 (m, 6H), 3.50 (t, J=6.1 Hz, 2H), 3.23-3.17 (m, 2H), 2.59 (t, J=6.1 Hz, 2H), 1.14 (t, J=7.2 Hz, 3H)

LRMS m/z calculated for C₃₃H₃₇N₇O₄ 595.29 [M]⁺, found 596.3 [M+H]⁺

HRMS m/z calculated for C₃₃H₃₈N₇O₄ 596.2980 [M+H]⁺, found 596.2972 [M+H]⁺

### Ia21 (E)-N-(3-imino-3-(isopropylamino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3265, 1670, 1197, 719

¹H NMR (500 MHz, DMSO-d₆) δ (ppm): 10.4 (s, 1H), 9.96 (s, 1H), 9.28-9.26 (m, 1H), 9.24-9.19 (m, 1H), 8.97 (s, 1H), 8.56 (s, 1H), 8.51 (s, 1H), 8.17-8.12 (m, 1H), 8.05-7.99 (m, 4H), 7.81 (d, J=8.4 Hz, 2H), 7.79-7.74 (m, 1H), 7.71-7.59 (m, 4H), 7.34 (d, J=1.7 Hz, 1H), 7.18 (d, J=1.7 Hz, 1H), 7.12 (d, J=1.7 Hz, 1H), 6.97 (d, J=1.7 Hz, 1H), 3.88 (s, 3H), 3.82 (s, 3H), 3.79-3.73 (m, 2H), 3.53-3.47 (m, 2H), 2.59-2.55 (m, 1H), 1.16 (d, J=6.4 Hz, 6H)

LRMS m/z calculated for C₃₆H₃₈N₈O₃ 630.31 [M]⁺, found 631.0 [M+H]⁺

HRMS m/z calculated for C₃₆H₉₇O₃N₈ 631.3140 [M+H]⁺, found 631.3135 [M+H]⁺

### Ia22 (E)-N-(3-(ethylamino)-3-iminopropyl)-4-(4-(4-(3-methoxystyryl)benzamido)-1-methyl-1H-pyrrole-2-carboxamido)-1-methyl-1H-pyrrole-2-carboxamide

IR (cm⁻¹) 3307, 1377, 1128, 950

¹H NMR (500 MHz, DMSO-d₆) δ (ppm) 10.4 (s, 1H), 9.98 (s, 1H), 9.29 (d, J=1.8 Hz, 1H), 8.60 (s, 1H), 8.29 (t, J=5.7 Hz, 1H), 7.07-7.00 (m, 4H), 7.84-7.76 (m, 3H), 7.71-7.60 (m, 4H), 7.35 (d, J=1.4 Hz, 1H), 7.21 (d, J=1.4 Hz, 1H), 7.12 (d, J=1.4 Hz, 1H), 7.00 (d, J=1.4 Hz, 1H), 3.88 (s, 3H), 3.84 (s, 6H), 3.60-3.56 (m, 2H), 3.44-3.40 (m, 2H), 3.35-3.30 (m, 2H), 0.90-0.87 (m, 3H)

LRMS m/z calculated for C₃₃H₃₇N₇O₄ 595.29 [M]⁺, found 596.3 [M+H]⁺

HRMS m/z calculated for C₃₃H₃₈O₄N₇ 596.2980 [M+H]⁺, found 596.2975 [M+H]⁺

### Ia23 (E)-N-(5-((5-((3-imino-3-(isopropylamino)propyl)carbamoyl)-1-methyl-1H-pyrrol-3-yl)carbamoyl)-1-methyl-1H-pyrrol-3-yl)-6-(3-methoxystyryl)nicotinamide

IR (cm⁻¹) 3342, 1379, 950, 653

¹H NMR (500 MHz, DMSO-d₆) δ (ppm) 10.5 (s, 1H), 9.96 (s, 1H), 9.22 (d, J=7.7 Hz, 1H), 9.09 (s, J=2.1 Hz, 1H), 8.30-8.27 (m, 1H), 8.15 (t, J=5.7 Hz, 1H), 7.77 (d, J=16 Hz, 1H), 7.70 (d, J=8.2 Hz, 1H), 7.43 (d, J=16 Hz, 1H), 7.37-7.32 (m, 2H), 7.30-7.26 (m, 2H), 7.18 (d, J=1.8 Hz, 1H), 7.10 (d, J=1.8 Hz, 1H), 6.96 (d, J=1.7 Hz, 1H), 6.95-6.92 (m, 1H), 3.88 (s, 3H), 3.82 (s, 6H), 3.78-3.73 (m, 1H), 3.53-3.48 (m, 2H), 2.57 (t, J=6.3 Hz, 2H), 1.15 (d, J=6.4 Hz, 6H)

LRMS m/z calculated for C₃₃H₃₈N₈O₄ 610.30 [M], found 611.7 [M+H]⁺

HRMS m/z calculated for C₃₃H₃₉O₄N₈ 611.3089 [M+H]⁺, found 611.3086 [M+H]⁺

### Ia24 (E)-N-(5-((5-((3-(ethylamino)-3-iminopropyl)carbamoyl)-1-methyl-1H-pyrrol-3-yl)carbamoyl)-1-methyl-1H-pyrrol-3-yl)-6-(4-methoxystyryl)nicotinamide

IR (cm⁻¹) 3120, 1498, 1257, 655

¹H NMR (500 MHz, DMSO-d₆) δ (ppm) 10.5 (s, 1H), 9.96 (s, 1H), 9.06 (d, J=2.2 Hz, 1H), 9.00 (s, 1H), 8.52 (s, 1H), 8.27-8.24 (m, 1H), 8.18 (t, J=5.7 Hz, 1H), 7.75 (d, J=16 Hz, 1H), 7.68-7.60 (m, 4H), 7.34 (d, J=1.7 Hz, 1H), 7.25 (d, J=16 Hz, 1H), 7.19 (d, J=1.7 Hz, 1H), 7.11 (d, J=1.7 Hz, 1H), 7.00 (d, J=8.9 Hz, 1H), 6.95 (d, J=1.7 Hz, 1H), 3.88 (s, 3H), 3.82 (s, 3H), 3.81 (s, 3H), 3.52-3.47 (m, 2H), 3.22-3.18 (m, 2H), 2.60-2.58 (m, 2H), 1.14 (t, J=7.2 Hz, 1H)

LRMS m/z calculated for C₃₂H₃₆N₈O₄ 596.29 [M]⁺, found 597.3 [M+H]⁺

### Ia25 4-(4-((E)-2-(benzo[c][1,2,5]oxadiazol-5-yl)vinyl)benzamido)-N-(5-(((Z)-3-(ethylamino)-3-(ethylimino)propyl)carbamoyl)-1-methyl-1H-pyrrol-3-yl)-1-methyl-1H-pyrrole-2-carboxamide

IR (cm⁻¹) 3317, 2968, 1379, 1128, 950

¹H NMR (500 MHz, DMSO-d₆) δ (ppm) 10.4 (s, 1H), 9.98 (s, 1H), 9.26-9.22 (m, 1H), 8.59-8.55 (m, 1H), 8.28-8.23 (m, 1H), 8.02 (d, J=8.4 Hz, 2H), 7.83 (d, J=8.4 Hz, 2H), 7.71 (d, J=17 Hz, 1H), 7.62 (d, J=17 Hz, 1H), 7.35 (d, J=1.7 Hz, 1H), 7.20 (d, J=1.7 Hz, 1H), 7.19 (s, 1H), 7.13 (d, J=1.7 Hz, 1H), 7.09 (s, 1H), 6.99 (s, 1H), 6.97 (d, J=1.7 Hz, 1H), 3.89 (s, 3H), 3.83 (s, 3H), 3.49-3.45 (m, 4H), 3.23-3.18 (m, 2H), 2.75-2.71 (m, 2H), 1.21-1.16 (m, 6H)

LRMS m/z calculated for C₃₄H₃₇N₉O₄ 635.30 [M]⁺, found 636.3 [M+H]⁺

HRMS m/z calculated for C₃₄H₃₈O₄N₉ 636.3041 [M+H]⁺, found 636.3028 [M+H]⁺

### Ia26 N-(5-((5-(((Z)-3-(ethylamino)-3-(ethylimino)propyl)carbamoyl)-1-methyl-1H-pyrrol-3-yl)carbamoyl)-1-methyl-1H-pyrrol-3-yl)-6-((E)-3-methoxystyryl)nicotinamide

IR (cm⁻¹) 2968, 1377, 1128, 950

¹H NMR (500 MHz, DMSO-d₆) δ (ppm) 10.5 (s, 1H), 9.96 (s, 1H), 9.35 (s, 1H), 9.09 (s, 1H), 9.01 (s, 1H), 8.53 (s, 1H), 8.31-8.27 (m, 1H), 8.20-8.16 (m, 1H), 7.77 (d, J=16 Hz, 1H), 7.69 (d, J=8.0 Hz, 1H), 7.43 (d, J=16 Hz, 1H), 7.37-7.32 (m, 2H), 7.30-7.27 (m, 2H), 7.19 (d, J=1.7 Hz, 1H), 7.11 (d, J=1.7 Hz, 1H), 6.96-6.92 (m, 2H), 3.88 (s, 1H), 3.82 (s, 6H), 3.52-3.47 (m, 2H), 3.22-3.18 (m, 2H), 2.59 (t, J=6.1 Hz, 2H), 1.14 (t, J=7.2 Hz, 3H)

LRMS m/z calculated for C₃₂H₃₆N₈O₄ 596.29 [M]⁺, found 597.0 [M+H]⁺

HRMS m/z calculated for C₃₂H₃₇O₄N₈ 597.2932 [M+H]⁺, found 597.2924 [M+H]⁺

### Ia27 (E)-N-(3-imino-3-(methylamino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3091, 1490, 1309, 775

¹H NMR (500 MHz, DMSO-d₆) δ (ppm) 10.4 (s, 1H), 9.96 (s, 1H), 9.44-9.39 (m, 1H), 9.28 (d, J=1.9 Hz, 1H), 8.20 (t, J=5.8 Hz, 1H), 8.06-7.97 (m, 4H), 7.87-7.81 (m, 2H), 7.79-7.76 (m, 1H), 7.68-7.63 (m, 3H), 7.34 (d, J=1.6 Hz, 1H), 7.19 (d, J=1.6 Hz, 1H), 7.12 (d, J=1.6 Hz, 1H), 6.95 (d, J=1.6 Hz, 1H), 3.88 (s, 3H), 3.82 (s, 3H), 3.49-3.48 (m, 2H, under H₂O peak), 2.80 (d, J=4.9 Hz, 3H), 2.61-2.56 (m, 2H)

LRMS m/z calculated for C₃₄H₃₄N₈O₃ 602.70 [M]⁺, found 603.3 [M+H]⁺

HRMS m/z calculated for C₃₄H₃₅N₈O₃ 603.2827 [M+H]⁺, found 603.2821 [M+H]⁺

### Ia28 (E)-N-(3-imino-3-(methoxyamino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3258, 3076, 2938, 2818, 2690, 2160, 2037, 1979, 1773, 1667, 1632, 1580, 1535, 1464, 1435, 1404, 1348, 1263, 1196, 1182, 1130, 1061, 1015, 968, 951, 895, 833, 797, 770, 750, 719, 704.

¹H NMR (500 MHZ, DMSO-d₆) δ (ppm) 10.37 (s, 1H), 9.97 (s, 1H), 9.29 (d, 1H, J=1.8 Hz), 8.60 (d, 1H, J=1.35 Hz), 8.23 (t, 1H, 5.35 Hz) 8.08-8.00 (m, 5H), 7.86-7.77 (m, 4H), 7.83 (d, 2H, J=8.35 Hz), 7.79 (t, 1H, J=7.68 Hz), 7.73-7.61 (m, 3H), 7.36 (d, 1H, J=1.1 Hz), 7.21 (d, 1H, J=1.41 Hz), 7.13 (d, 1H, J=1.36), 6.96 (d, 1H, J=1.0 Hz), 3.89 (s, 3H), 3.83 (s, 3H), 3.73 (s, 1H), 3.49 (q, 2H, J=5.9 Hz), 2H triplet can't be seen as hidden underneath DMSO peak at 2.53-2.50 ppm

LRMS m/z calculated for C₃₄H₃₄N₈O₄ 618.70 [M]⁺, found 619.4 [M+H]⁺

### Ia29 (E)-N-(3-imino-3-(methoxyamino)propyl)-4-(4-(4-(3-methoxystyryl)benzamido)-1-methyl-1H-pyrrole-2-carboxamido)-1-methyl-1H-pyrrole-2-carboxamide

IR (cm⁻¹) 3280, 1670, 1199, 721

¹H NMR (500 MHz, DMSO-d₆) δ (ppm) 10.3 (s, 1H), 9.95 (s, 1H), 7.98 (d, J=8.3 Hz, 2H), 7.74 (d, J=8.3 Hz, 2H), 7.38-7.30 (m, 4H), 7.24-7.18 (m, 3H), 7.12-7.09 (m, 1H), 6.94-6.87 (m, 2H), 3.87 (s, 3H), 3.81 (s, 6H), 3.67 (s, 3H), 3.54-3.50 (m, 2H), 2.44-2.37 (m, 2H)

LRMS m/z calculated for C₃₂H₃₅N₇O₅ 597.68 [M]⁺, found 598.6 [M+H]⁺

### Ia30 (E)-4-(4-(2-(benzo[c][1,2,5]oxadiazol-5-yl)vinyl)benzamido)-N-(5-((3-imino-3-(methoxyamino)propyl)carbamoyl)-1-methyl-1H-pyrrol-3-yl)-1-methyl-1H-pyrrole-2-carboxamide

IR (cm⁻¹) 3313, 1637, 1199, 798

¹H NMR (500 MHz, DMSO-d₆) δ (ppm) 10.4 (s, 1H), 9.95 (s, 1H), 8.11 (s, 3H), 8.02 (d, J=8.3 Hz, 2H), 7.82 (d, J=8.3 Hz, 2H), 7.70 (d, J=16 Hz, 1H), 7.61 (d, J=16 Hz, 1H), 7.36-7.34 (m, 1H), 7.21-7.19 (m, 1H), 7.12-7.09 (m, 1H), 6.93-6.91x (m, 1H), 3.88 (s, 3H), 3.82 (s, 3H), 3.68 (s, 3H), 3.54-3.49 (m, 2H), 2.46-3.37 (m, 2H)

LRMS m/z calculated for C₃₁H₃₁N₉O₅ 609.65 [M]⁺, found 610.5 [M+H]⁺

### Ia31 (E)-6-(4-(dimethylamino)styryl)-N-(5-((5-((3-imino-3-(methoxyamino)propyl)carbamoyl)-1-methyl-1H-pyrrol-3-yl)carbamoyl)-1-methyl-1H-pyrrol-3-yl)nicotinamide

IR (cm⁻¹) 3111, 1664, 1128, 721

¹H NMR (500 MHz, DMSO-d₆) δ (ppm) 10.5 (s, 1H), 9.97 (s, 1H), 9.06 (s, 1H), 8.28 (d, J=8.2 Hz, 1H), 8.17 (s, 1H), 7.73 (d, J=16 Hz, 1H), 7.64 (d, J=7.8 Hz, 1H), 7.54 (d, J=8.6 Hz, 2H), 7.35 (s, 1H), 7.21 (s, 1H), 7.16-7.10 (m, 2H), 6.92 (s, 1H), 6.76 (d, J=8.6 Hz, 2H), 3.88 (s, 3H), 3.81 (s, 3H), 3.69 (s, 1H), 3.47-3.44 (m, 2H), 2.98 (s, 6H), 2.46-2.43 (m, 2H); LRMS m/z calculated for C₃₂H₃₇N₉O₄ 611.71 [M]⁺, found 612.7 [M+H]⁺

### Ia32 (E)-6-(4-(dimethylamino)styryl)-N-(5-((5-((3-imino-3-(nonylamino)propyl)carbamoyl)-1-methyl-1H-pyrrol-3-yl)carbamoyl)-1-methyl-1H-pyrrol-3-yl)nicotinamide

IR (cm⁻¹) 2926, 2856, 1664, 1577, 1527, 1433, 1365, 1288, 1168, 1128, 798, 721

¹H NMR (500 MHz, DMSO-d₆) δ (ppm) 10.43 (1H, s), 9.94 (1H, s), 9.30 (2H, bs), 9.04-8.99 (3H, m), 8.50 (2H, s), 8.24-8.20 (2H, m), 8.14 (1H, t, J=6 Hz), 7.71-7.52 (6H, m), 7.33 (1H, s), 7.17-7.07 (5H, m), 6.97 (1H, s), 6.77-6.73 (2H, m), 3.87 (3H, s), 3.81 (3H, s), 3.51-3.48 (2H, m), 3.16-3.13 (2H, m), 2.97 (6H, s), 2.62-2.60 (2H, m), 1.51-1.48 (2H, m), 1.24-1.20 (12H, m), 0.85-0.81 (3H, m)

LRMS m/z calculated for C₄₀H₅₃N₉O₃ 707.4, found 708.1 [M+H]⁺

HRMS m/z calculated for C₄₀H₅₄N₉O₃ 708.4344 [M+H]⁺, found 715.4068 [M+H]⁺

### Ia33 6-((E)-4-(dimethylamino)styryl)-N-(5-((5-(((E)-3-(heptylamino)-3-(heptylimino)propyl)carbamoyl)-1-methyl-1H-pyrrol-3-yl)carbamoyl)-1-methyl-1H-pyrrol-3-yl)nicotinamide

IR (cm⁻¹) 3248, 3088, 2927, 2856, 1643, 1573, 1529, 1435, 1402, 1367, 1313, 1286, 1265, 1199, 1170, 1128, 1064, 1002,831, 775, 719

¹H NMR (500 MHz, DMSO-d₆) δ (ppm) 10.45 (1H, s), 9.96, (1H, s), 9.24-9.20 (2H, m), 9.04 (1H, s), 8.56-8.51 (3H, m), 8.25-8.21−(4H, m), 7.72-7.61 (2H, m), 7.55-7.51 (3H, m), 7.34 (1H, s), 7.17-7.07 (5H, m), 6.98 (1H, s), 6.77-6.74 (3H, m), 3.87 (3H, s), 3.81 (3H, s), 3.49-3.45 (2H, m), 3.39-3.35 (2H, m), 3.16-3.12 (2H, m), 2.97 (6H, s), 2.74-2.70 (2H, m), 1.54-1.50 (4H, m), 1.30-1.23 (16H, m), 0.88-0.83 (6H)

LRMS m/z calculated for C₄₅H₆₃N₉O₃ 777.5 [M]⁺, found 778.0 [M+H]⁺

HRMS m/z calculated for C₄₅H₆₄N₉O₃ 778.5127 [M+H]⁺, found 778.5118 [M+H]⁺

### Ia34 (E)-N-(3-(butylamino)-3-iminopropyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 2962.66, 2872.01, 1670.35, 1635.64, 1577.77, 1533.41, 1462.04, 1435.04, 1402.25, 1390.68, 1261.45, 1197.79, 1170.79, 1126.43, 1062.78, 1014.56, 975.98, 960.55, 894.97, 873.75, 837.11, 792.74, 756.1, 721.38, 704.02, 692.44, 607.58

¹H NMR (500 MHz, DMSO-d₆) δ (ppm) 10.36 (1H, s), 9.96 (1H, s), 9.32 (1H, s), 9.29 (1H, bs), 9.01 (1H, bs), 8.60-8.54 (3H, m), 8.18 (1H, t, J=6 Hz), 8.08-7.99 (5H, m), 7.84-7.77 (4H, m), 7.71-7.61 (4H, m), 7.35 (1H, s), 7.18 (1H, s), 7.13 (1H, s), 6.98 (1H, s), 3.89 (3H, s), 3.83 (3H, s), 3.52-3.49 (2H, m), 3.17-3.14 (2H, m), 2.62-2.59 (2H, m), 1.52-1.49 (2H, m), 1.34-1.29 (2H, m), 0.90-0.89 (3H, m);

LRMS m/z calculated for C₃₇H₄₀N₈O₃ 644.3 [M]⁺, found 645.8 [M+H]⁺

HRMS m/z calculated for C₃₇H₄₁N₈O₃ 645.3296 [M+H]⁺, found 645.3285 [M+H]⁺

### Ia35 (E)-N-(3-imino-3-(pentylamino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3259, 3093, 2964, 1670, 1637, 1577, 1560, 1521, 1458, 1436, 1382, 1265, 1195, 1176, 1128, 1062, 966, 894, 831, 798, 771, 750, 719, 628

¹H NMR (500 MHz, DMSO-d₆) δ (ppm) 10.36 (1H, s), 9.95 (1H, s), 9.33 (1H, s), 9.28 (1H, bs), 9.01 (1H, bs), 8.59-8.54 (3H, m), 8.16 (1H, t, J=6 Hz), 8.05-7.97 (5H, m), 7.82-7.75 (4H, m), 7.70-7.60 (4H, m), 7.34 (1H, s), 7.17 (1H, s), 7.11 (2H, s), 6.97 (1H, s), 3.88 (3H, s), 3.82 (3H, s), 3.52-3.48 (2H, m), 3.17-3.13 (2H, m), 2.62-2.59 (2H, m), 1.52-1.50 (2H, m), 1.27-1.25 (4H, m), 0.85-0.82 (3H, m)

LRMS m/z calculated for C₃₈H₄₂N₈O₃ 658.3 [M]⁺, found 659.0 [M+H]⁺

HRMS m/z calculated for C₃₈H₄₂N₈O₃ 659.3453 [M+H]⁺, found 659.3443 [M+H]⁺

### Ia36 (E)-N-(3-imino-3-(nonylamino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3259, 3088, 2929, 2856, 1633, 1579, 1531, 1462, 1433, 1402, 1263, 1197, 1178, 1130, 1064, 968, 894, 831, 798, 773, 748, 719

¹H NMR (500 MHz, DMSO-d₆) δ (ppm) 10.35 (1H, s), 9.94 (1H, s), 9.31-9.26 (3H, m), 8.99 (2H, s), 8.56-8.51 (4H, m), 8.15 (2H, t, J=6 Hz), 8.04-8.00 (5H, m), 7.82-7.74 (5H, m), 7.66-7.63 (3H, m), 7.34 (1H, s), 7.17 (2H, s), 7.12 (1H, s), 6.98 (1H, s), 3.88 (3H, s), 3.82 (3H, s), 3.51-3.48 (2H, m), 3.16-3.13 (2H, m), 2.62-2.60 (2H, m), 1.51-1.48 (2H, m), 1.24-1.20 (12H, m), 0.85-0.81 (3H, m)

LRMS m/z calculated for C₄₂H₅₀N₈O₃ 714.4 [M]⁺, found 715.1 [M+H]⁺

HRMS m/z calculated for C₄₂H₅₁N₈O₃ 715.4079, found 715.4068 [M+H]⁺

### Ia37 N—((Z)-3-(heptylamino)-3-(heptylimino)propyl)-1-methyl-4-(1-methyl-4-(4-((E)-2-(quinolin-3-yl)vinyl)benzamido)-1H-pyrrole-2-carboxamido)-1H-pyrrole-2-carboxamide

IR (cm⁻¹) 3253, 3088, 2929, 2858, 1633, 1581, 1462, 1435, 1402, 1263, 1197, 1174, 1128, 1060, 1016, 968, 894, 829, 771, 750, 719, 624

¹H NMR (500 MHz, DMSO-d₆) δ (ppm) 10.36, (1H, s), 9.27-9.21 (3H, m), 8.56-8.51 (4H, m), 8.224-8.21 (1H, m), 8.05-8.01 (6H, m), 7.84-7.75 (4H, m), 7.68-7.64 (4H, m), 7.35 (1H, s), 7.19-7.09 (4H, m), 7.01-6.98 (2H, m), 3.89 (3H, s), 3.83 (3H, s), 3.49-3.45 (2H, m), 3.40-3.36 (2H, m), 3.17-3.14 (2H, m), 2.75-2.71 (2H, m), 1.56-1.52 (4H, m), 1.29-1.24 (16H), 0.87-0.84 (6H, m)

LRMS m/z calculated for C₄₇H₆₀N₈O₃ 784.4 [M]⁺, found 785.0 [M+H]⁺

HRMS m/z calculated for C₄₇H₆₁N₈O₃ 785.4861 [M+H]⁺, found 785.4851 [M+H]⁺

### Ia38 N—((E)-3-(butylamino)-3-(butylimino)propyl)-1-methyl-4-(1-methyl-4-(4-((E)-2-(quinolin-3-yl)vinyl)benzamido)-1H-pyrrole-2-carboxamido)-1H-pyrrole-2-carboxamide

IR (cm⁻¹) 3265, 3080, 2956, 1633, 1531, 1435, 1402, 1384, 1263, 1197, 1178, 1130, 1062, 968, 831, 798, 771, 750, 719, 707, 605

¹H NMR (500 MHz, DMSO-d₆) δ (ppm) 10.35 (1H, s), 9.96 (1H, s), 9.27 (1H, s), 9.22 (1H, bs), 8.56-8.52 (3H, m), 8.24 (1H, t, J=6 Hz), 8.05-8.00 (5H, m), 7.82-7.75 (4H, m), 7.69-7.60 (4H, m), 7.34 (1H, s), 7.17 (1H, s), 7.11 (1H, s), 6.98 (1H, s), 3.88 (3H, s), 3.82 (3H, s), 3.48-3.46 (2H, m), 3.41-3.38 (2H, m), 3.17-3.14 (2H, m), 2.74-2.70 (2H, m), 1.56-1.49 (4H, m), 1.35-1.28 (4H, m), 0.91-0.86 (6H, m)

LRMS m/z calculated for C₄₁H₄₈N₈O₃ 700.8 [M]⁺, found 701.9 [M+H]⁺

HRMS m/z calculated for C₄₁H₄₉N₈O₃ 701.3922 [M+H]⁺, found 701.3910 [M+H]⁺

### Ia39 N-(5-((5-(((E)-3-(butylamino)-3-(butylimino)propyl)carbamoyl)-1-methyl-1H-pyrrol-3-yl)carbamoyl)-1-methyl-1H-pyrrol-3-yl)-6-((E)-4-(dimethylamino)styryl)nicotinamide

IR (cm⁻¹) 3251, 3091, 2956, 2933, 1653, 1573, 1527, 1458, 1435, 1404, 1365, 1313, 1288, 1265, 1166, 1126, 1064, 1002, 964, 945, 831, 812, 798, 777, 719, 661, 623

¹H NMR (500 MHz, DMSO-d₆) δ (ppm) 10.48 (1H, s), 9.96, (1H, s), 9.24-9.22 (2H, m), 9.04 (1H, s), 9.56-9.54 (1H, m), 8.29-8.23 (2H, m), 7.75-7.66 (2H, m), 7.55-7.52 (2H, m), 7.34 (1H, s), 7.18-7.10 (3H, m), 6.97 (1H, s), 6.78-6.75 (2H, m), 3.88 (3H, s), 3.82 (3H, s), 3.48-3.44 (2H, m), 3.40-3.36 (2H, m), 3.17-3.13 (2H, m), 2.98 (6H, s), 2.74-2.70 (2H, m), 1.57-1.48 (4H, m), 1.36-1.25 (4H, m), 0.90-0.84 (6H, m)

LRMS m/z calculated for C₃₉H₅₁N₉O₃ 693.4 [M]⁺, found 694.0 [M+H]⁺

HRMS m/z calculated for C₃₉H₅₂N₉O₃ 694.4188 [M+H]⁺, found 694.4178 [M+H]⁺

### Ia40 (E)-4-(4-(2-(benzo[c][1,2,5]oxadiazol-5-yl)vinyl)benzamido)-N-(5-((12-imino-2,5,8-trioxa-11-azatetradecan-14-yl)carbamoyl)-1-methyl-1H-pyrrol-3-yl)-1-methyl-1H-pyrrole-2-carboxamide

IR (cm⁻¹) 3109, 1629, 1199, 721

¹H NMR (500 MHz, DMSO-d₆) δ (ppm) 10.4 (s, 1H), 9.95 (s, 1H), 9.48 (s, 1H), 9.07 (s, 1H), 8.60 (s, 1H), 8.15 (t, J=5.5 Hz, 1H), 8.11 (s, 3H), 8.02 (d, J=8.3 Hz, 2H), 7.82 (d, J=8.3 Hz, 2H), 7.70 (d, J=17 Hz, 1H), 7.63 (d, J=17 Hz, 1H), 7.34 (d, J=1.5 Hz, 1H), 7.18 (d, J=1.6 Hz, 1H), 7.12 (d, J=1.6 Hz, 1H), 6.97 (d, J=1.5 Hz, 1H), 3.88 (s, 3H), 3.82 (s, 3H), 3.58-3.48 (m, 14H), 3.23 (s, 3H), 2.63-2.59 (m, 2H)

LRMS m/z calculated for C₃₇H₄₃N₉O₇ 725.33 [M]⁺, found 726.7 [M+H]⁺

HRMS m/z calculated for C₃₇H₄₄N₉O₇ 726.3358 [M+H]⁺, found 726.3354 [M+H]⁺

### Ia41 (E)-N-(3-imino-3-((2-(4-methylpiperazin-1-yl)ethyl)amino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3097, 1629, 1182, 719

¹H NMR (500 MHz, DMSO-d₆) δ (ppm) 10.4 (s, 1H), 9.96 (s, 1H), 9.27 (s, 1H), 9.08 (s, 1H), 8.62 (s, 1H), 8.56 (s, 1H), 8.18 (t, J=5.8 Hz, 1H), 8.04-8.00 (m, 4H), 7.82 (d, J=8.5 Hz, 2H), 7.78-7.74 (m, 1H), 7.68-7.62 (m, 3H), 7.33 (d, J=1.3 Hz, 1H), 7.17 (d, J=1.5 Hz, 1H), 7.14 (d, J=1.3 Hz, 1H), 7.01-6.99 (m, 1H), 3.88 (s, 3H), 3.82 (s, 3H), 3.52-3.49 (m, 7H), 3.40-3.34 (m, 5H), 3.31-3.28 (m, 2H), 2.99-2.93 (m, 2H), 2.76 (s, 3H)

LRMS m/z calculated for C₄₀H₄₆N₁₀O₃ 714.38 [M]⁺, found 715.0 [M+H]⁺

HRMS m/z calculated for C₄₀H₄₇N₁₀O₃ 715.3827 [M+H]⁺, found 715.3825 [M+H]⁺

### Ia42 (E)-4-(4-(2-(benzo[c][1,2,5]oxadiazol-5-yl)vinyl)benzamido)-N-(5-((3-imino-3-((2-(4-methylpiperazin-1-yl)ethyl)amino)propyl)carbamoyl)-1-methyl-1H-pyrrol-3-yl)-1-methyl-1H-pyrrole-2-carboxamide

IR (cm⁻¹) 3032, 1629, 1180, 705

¹H NMR (500 MHz, DMSO-d₆) δ (ppm) 10.4 (s, 1H), 9.27 (s, 1H), 9.10 (s, 1H), 8.64 (s, 1H), 8.18 (t, J=5.5 Hz, 1H), 8.11 (s, 3H), 8.01 (d, J=8.3 Hz, 2H), 7.82 (d, J=8.3 Hz, 2H), 7.70 (d, J=17 Hz, 1H), 7.61 (d, J=17 Hz, 1H), 7.33 (d, J=1.5 Hz, 1H), 7.17 (d, J=1.6 Hz, 1H), 7.14 (d, J=1.6 Hz, 1H), 6.99 (d, J=1.5 Hz, 1H), 3.88 (s, 3H), 3.82 (s, 3H), 3.39-3.33 (m, 5H), 3.32-3.28 (m, 3H), 3.00-2.94 (m, 2H), 2.91-2.83 (m, 2H), 2.76 (s, 3H), 2.65-2.60 (m, 3H)

LRMS m/z calculated for C₃₇H₄₃N₁₁O₄ 705.35 [M]⁺, found 705.9 [M+H]⁺

HRMS m/z calculated for C₃₇H₄₄N₁₁O₄ 706.3572 [M+H]⁺, found 706.3564 [M+H]⁺

### Ia43 (E)-4-(4-(2-(benzo[c][1,2,5]oxadiazol-5-yl)vinyl)benzamido)-N-(5-((3-imino-3-(phenethylamino)propyl)carbamoyl)-1-methyl-1H-pyrrol-3-yl)-1-methyl-1H-pyrrole-2-carboxamide

IR (cm⁻¹) 3255, 1627, 1201, 698

¹H NMR (500 MHz, DMSO-d₆) δ (ppm) 10.4 (s, 1H), 9.27 (s, 1H), 9.10 (s, 1H), 8.64 (s, 1H), 8.18 (t, J=5.5 Hz, 1H), 8.11 (s, 3H), 8.01 (d, J=8.3 Hz, 2H), 7.82 (d, J=8.3 Hz, 2H), 7.70 (d, J=17 Hz, 1H), 7.61 (d, J=17 Hz, 1H), 7.33 (d, J=1.5 Hz, 1H), 7.17 (d, J=1.6 Hz, 1H), 7.14 (d, J=1.6 Hz, 1H), 6.99 (d, J=1.5 Hz, 1H), 3.88 (s, 3H), 3.82 (s, 3H), 3.39-3.33 (m, 5H), 3.32-3.28 (m, 3H), 3.00-2.94 (m, 2H), 2.91-2.83 (m, 2H), 2.76 (s, 3H), 2.65-2.60 (m, 3H)

LRMS m/z calculated for C₃₈H₃₇N₉O₄ 683.30 [M]⁺, found 684.4 [M+H]⁺

HRMS m/z calculated for C₃₈H₃₈N₉O₄ 684.3041 [M+H]⁺, found 684.3038 [M+H]⁺

### Ia44 (E)-N-(3-imino-3-((2-(tetrahydro-2H-pyran-4-yl)ethyl)amino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 2933, 1674, 1201, 835

¹H NMR (500 MHz, DMSO-d₆) δ (ppm) 10.4 (s, 1H), 9.95 (s, 1H), 9.29 (d, J=1.9 Hz, 1H), 9.03 (s, 1H), 8.60 (s, 1H), 8.55 (s, 1H), 8.16 (t, J=5.8 Hz, 1H), 8.07-7.99 (m, 5H), 7.84-7.76 (m, 4H), 7.69-7.61 (m, 3H), 7.34 (d, J=1.5 Hz, 1H), 7.16 (d, J=1.6 Hz, 1H), 7.11 (d, J=1.6 Hz, 1H), 7.00 (d, J=1.5 Hz, 1H), 3.88 (s, 3H), 3.82 (s, 3H), 3.53-3.49 (m, 2H), 3.22-3.16 (m, 4H), 2.62-2.58 (m, 2H), 1.55-1.50 (m, 3H), 1.49-1.42 (m, 3H), 1.19-1.09 (m, 3H)

LRMS m/z calculated for C₄₀H₄₄N₈O₄700.35 [M]⁺, found 701.4 [M+H]⁺

HRMS m/z calculated for C₄₀H₄₅O₄N₈ 701.3558 [M+H]⁺, found 701.3548 [M+H]⁺

### Ia45 (E)-4-(4-(2-(benzo[c][1,2,5]oxadiazol-5-yl)vinyl)benzamido)-N-(5-((3-imino-3-((2-(tetrahydro-2H-pyran-4-yl)ethyl)amino)propyl)carbamoyl)-1-methyl-1H-pyrrol-3-yl)-1-methyl-1H-pyrrole-2-carboxamide

IR (cm⁻¹) 3084, 1629, 1199, 798

¹H NMR (500 MHz, DMSO-d₆) δ (ppm) 10.4 (s, 1H), 9.95 (s, 1H), 9.31 (s, 1H), 9.03 (s, 1H), 8.54 (s, 1H), 8.16 (t, J=5.9 Hz, 1H), 8.10 (s, 3H), 8.01 (d, J=8.3 Hz, 2H), 7.81 (d, J=8.3 Hz, 2H), 7.70 (d, J=16 Hz, 1H), 7.61 (d, J=16 Hz, 1H), 7.34 (d, J=1.2 Hz, 1H), 7.15 (d, J=1.5 Hz, 1H), 7.11 (d, J=1.2 Hz, 1H), 7.00 (d, J=1.5 Hz, 1H), 3.88 (s, 3H), 3.82 (s, 3H), 3.80-3.77 (m, 2H), 3.52-3.49 (m, 2H), 3.20-3.18 (m, 2H), 2.62-2.58 (m, 2H), 1.56-1.49 (m, 4H), 1.49-1.43 (m, 3H), 1.25-1.11 (m, 4H)

LRMS m/z calculated for C₃₇H₄₁N₉O₅ 691.32 [M]⁺, found 692.3 [M+H]⁺

HRMS m/z calculated for C₃₇H₄₂O₅N₉ 692.3303 [M+H]⁺, found 692.3300 [M+H]⁺

### Ia46 (E)-4-(4-(2-(benzo[c][1,2,5]oxadiazol-5-yl)vinyl)benzamido)-N-(5-((3-((3-(dimethylamino)propyl)amino)-3-iminopropyl)carbamoyl)-1-methyl-1H-pyrrol-3-yl)-1-methyl-1H-pyrrole-2-carboxamide

IR (cm⁻¹) 3290, 1676, 1448, 1022

¹H NMR (500 MHz, DMSO-d₆) δ (ppm) 10.4 (s, 1H), 9.95 (s, 1H), 9.18 (s, 1H), 8.68 (s, 1H), 8.22 (s, 1H), 8.01 (d, J=8.5 Hz, 2H), 7.82 (d, J=8.5 Hz, 2H), 7.70 (d, J=16 Hz, 2H), 7.61 (d, J=16 Hz, 2H), 7.33 (s, 1H), 7.20-7.12 (m, 2H), 7.03-6.97 (m, 1H), 3.88 (s, 3H), 3.82 (s, 3H), 3.57-3.50 (m, 2H), 3.25-3.24 (m, 2H), 3.06-3.03 (m, 2H), 2.76-2.75 (m, 6H), 2.61-2.58 (m, 2H), 1.89-1.86 (m, 2H)

LRMS m/z calculated for C₃₅H₄₀N₁₀O₄ 664.32 [M]⁺, found 665.3 [M+H]⁺

HRMS m/z calculated for C₃₅H₄₁O₄N₁₀ 665.3307 [M+H]⁺, found 665.3297 [M+H]⁺

### Ia47 (E)-N-(3-imino-3-((2-thiomorpholinoethyl)amino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3319, 1629, 1305, 831

¹H NMR (500 MHz, DMSO-d₆) δ (ppm) 10.4 (s, 1H), 9.96 (s, 1H), 9.54 (s, 1H), 9.37 (s, 1H), 9.28 (s, 1H), 8.84 (s, 1H), 8.58 (s, 1H), 8.25 (s, 1H), 8.05-7.99 (m, 4H), 7.82 (d, J=8.2 Hz, 2H), 7.79-7.75 (m, 1H), 7.69-7.60 (3H), 7.34 (s, 1H), 7.16 (s, 1H), 7.13 (s, 1H), 7.01 (s, 1H), 3.92-3.90 (m, 2H), 3.88 (s, 3H), 3.83 (s, 3H), 3.74-3.72 (m, 2H), 3.61-3.57 (m, 2H), 3.55-3.48 (m, 4H), 2.95-2.81 (m, 4H), 2.64-2.60 (m, 2H)

LRMS m/z calculated for C₃₉H₄₃N₉O₃S 717.32 [M]⁺, found 718.6 [M+H]⁺

HRMS m/z calculated for C₃₉H₄₄N₉O₃S 718.3282 [M+H]⁺, found 718.3283 [M+H]⁺

### Ia48 (E)-N-(3-imino-3-(phenethylamino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3072, 1629, 1178, 719

¹H NMR (500 MHz, DMSO-d₆) δ (ppm) 10.37 (s, 1H), 9.97 (s, 1H), 9.51 (s, 1H), 9.29-9.25 (m, 1H), 9.09 (s, 1H), 8.65 (s, 1H), 8.59-8.55 (m, 1H), 8.23-8.17 (m, 1H), 8.06-7.99 (m, 4H), 7.82 (d, J=8.1 Hz, 2H), 7.80-7.73 (m, 1H), 7.72-7.59 (m, 3H), 7.36-7.20 (m, 6H), 7.20-7.17 (m, 1H), 7.14-7.10 (m, 1H), 7.02-6.98 (m, 1H), 3.87 (s, 3H), 3.83 (s, 3H), 3.52-3.40 (m, 4H), 2.89-2.82 (m, 2H), 2.64-2.57 (m, 2H)

LRMS m/z calculated for C₄₁H₄₀N₈O₃ 692.82 [M], found 693.7 [M+H]⁺

HRMS m/z calculated for C₄₁H₄₁N₈O₃ 693.3296 [M+H]⁺, found 693.3291 [M+H]⁺

### Ia49 (E)-6-(4-(dimethylamino)styryl)-N-(5-((5-((3-imino-3-(phenethylamino)propyl)carbamoyl)-1-methyl-1H-pyrrol-3-yl)carbamoyl)-1-methyl-1H-pyrrol-3-yl)nicotinamide

IR (cm⁻¹) 3213, 2922, 1676, 1074

¹H NMR (500 MHz, DMSO-d₆) δ (ppm) 10.45 (s, 1H), 9.96 (s, 1H), 9.48 (bs, 1H), 9.09-9.02 (m, 2H), 8.63 (s, 1H), 8.29-8.23 (m, 1H), 8.19 (t, J=5.9 Hz, 1H), 7.72 (d, J=16.0 Hz, 1H), 7.65 (d, J=8.3 Hz, 1H), 7.54 (d, J=8.5 Hz, 2H), 7.36-7.20 (m, 6H), 7.20-7.16 (m, 1H), 7.15-7.06 (m, 2H), 7.02-6.99 (m, 1H), 6.76 (d, J=8.6 Hz, 2H), 3.87 (s, 3H), 3.82 (s, 3H), 3.49 (d, J=6.4 Hz, 2H), 3.43 (d, J=7.5 Hz, 2H), 2.98 (s, 6H), 2.87-2.83 (m, 2H), 2.61-2.57 (m, 2H)

LRMS m/z calculated for C₃₉H₄₃N₉O₃ 685.83 [M], found 686.6 [M+H]⁺

### Ia50 N—((Z)-3-(ethylamino)-3-(ethylimino)propyl)-1-methyl-4-(1-methyl-4-(4-((E)-2-(quinolin-3-yl)vinyl)benzamido)-1H-pyrrole-2-carboxamido)-1H-pyrrole-2-carboxamide

IR (cm⁻¹) 3244, 1674, 1174, 1126, 1026, 719

¹H NMR (500 MHz, DMSO-d₆) δ (ppm): 10.36 (s, 1H), 9.97 (s, 1H), 9.26 (d, J=2.3 Hz, 1H), 9.25-9.22 (m, 1H), 8.59-8.53 (m, 2H), 8.24 (t, J=6.1 Hz, 1H), 8.06-7.98 (m, 4H), 7.82 (d, J=8.5 Hz, 2H), 7.78-7.73 (m, 1H), 7.71-7.58 (m, 3H), 7.35 (d, J=2.0 Hz, 1H), 7.19 (d, J=2.0 Hz, 1H), 7.12 (d, J=2.0 Hz, 1H), 6.96 (d, J=1.8 Hz, 1H), 3.88 (s, 3H), 3.82 (s, 3H), 3.48-3.42 (m, 4H), 2.73-2.69 (m, 2H), 2.52-2.51 (m, 2H), 1.19 (t, J=7.2 Hz, 3H), 1.15 (t, J=7.2 Hz, 3H),

LRMS m/z calculated for C₃₇H₄₀N₈O₃ 644.78 [M], found 645.4 [M+H]⁺

### Ia51 6-((E)-4-(dimethylamino)styryl)-N-(5-((5-(((Z)-3-(ethylamino)-3-(ethylimino)propyl)carbamoyl)-1-methyl-1H-pyrrol-3-yl)carbamoyl)-1-methyl-1H-pyrrol-3-yl)nicotinamide

IR (cm⁻¹) 3234, 1637, 1575, 1126, 719

¹H NMR (500 MHz, DMSO) δ 10.48 (s, 1H), 9.98 (s, 1H), 9.29 (s, 1H), 9.05 (s, 1H), 8.63 (s, 1H), 8.28-8.25 (m, 2H), 7.72 (d, J=15.1 Hz, 1H), 7.63 (d, J=8.5 Hz, 1H), 7.54 (d, J=8.9 Hz, 2H), 7.37-7.33 (m, 1H), 7.22-7.18 (m, 1H), 7.15-7.09 (m, 2H), 6.96 (d, J=2.0 Hz, 1H), 6.76 (d, J=8.7 Hz, 2H), 3.88 (s, 3H), 3.82 (s, 3H), 3.48-3.43 (m, 4H), 3.21 (t, J=6.3 Hz, 2H), 2.75-2.71 (m, 2H), 1.20-1.13 (m, 6H).

LRMS m/z calculated for C₃₅H₄₃N₉O₃ 637.79 [M], found 638.4 [M+H]⁺

### Ia52 (E)-6-(4-(dimethylamino)styryl)-N-(5-((5-((3-imino-3-((2-thiomorpholinoethyl)amino)propyl)carbamoyl)-1-methyl-1H-pyrrol-3-yl)carbamoyl)-1-methyl-1H-pyrrol-3-yl)nicotinamide

¹H NMR (500 MHz, DMSO-d₆) δ (ppm) 10.45 (s, 1H), 9.96 (s, 1H), 9.54-9.44 (m, 1H), 9.36 (s, 1H), 9.06-9.02 (m, 1H), 8.83 (s, 1H), 8.28-8.21 (m, 2H), 7.72 (d, J=15.9 Hz, 1H), 7.64 (d, J=8.2 Hz, 1H), 7.54 (d, J=8.6 Hz, 2H), 7.35-7.31 (m, 1H), 7.18-7.08 (m, 3H), 7.02-6.99 (m, 1H), 6.76 (d, J=8.6 Hz, 2H), 3.88 (s, 3H), 3.83 (s, 3H), 3.74-3.72 (m, 2H), 3.61-3.48 (m, 6H), 2.98 (s, 6H), 2.87-2.82 (m, 4H), 2.62-2.58 (m, 2H)

LRMS m/z calculated for C₃₇H₄₆N₁₀O₃S 710.90 [M], found 711.9 [M+H]⁺

### Ia53 4-(4-((E)-3-methoxystyryl)benzamido)-1-methyl-N-(1-methyl-5-(((E)-3-(methylamino)-3-(methylimino)propyl)carbamoyl)-1H-pyrrol-3-yl)-1H-pyrrole-2-carboxamide

IR (cm⁻¹) 3277, 1637, 1433, 1199

¹H NMR (500 MHz, DMSO-d₆) δ (ppm) 10.32 (s, 1H), 9.96 (s, 1H), 9.36-9.32 (m, 1H), 8.62-8.57 (m, 1H), 8.28-8.25 (m, 1H), 7.97 (d, J=8.5 Hz, 2H), 7.74 (d, J=8.5 Hz, 2H), 7.39-7.29 (m, 4H), 7.25-7.18 (m, 3H), 7.12 (s, 1H), 6.95 (s, 1H), 6.89 (d, J=7.8 Hz, 1H), 3.88 (s, 3H), 3.84-3.79 (m, 6H), 3.49-3.44 (m, 2H), 3.02 (d, J=4.9 Hz, 3H), 2.78 (d, J=4.9 Hz, 3H), 2.72 (t, J=6.9 Hz, 2H)

LRMS m/z calculated for C₃₃H₃₇N₇O₄ 595.29 [M], found 596.3 [M+H]⁺

### Ia54 6-((E)-4-methoxystyryl)-N-(1-methyl-5-((1-methyl-5-(((E)-3-(methylamino)-3-(methylimino)propyl)carbamoyl)-1H-pyrrol-3-yl)carbamoyl)-1H-pyrrol-3-yl)nicotinamide

IR (cm⁻¹) 3088, 1662, 1197, 1172, 1126, 719

¹H NMR (500 MHz, DMSO-d₆) δ (ppm) 10.48 (s, 1H), 9.97 (s, 1H), 9.37-9.33 (m, 1H), 9.09-9.05 (m, 1H), 8.63-8.59 (m, 1H), 8.30-8.24 (m, 2H), 7.76 (d, J=16.0 Hz, 1H), 7.66 (m, 3H), 7.36-7.32 (m, 1H), 7.25 (d, J=16.2 Hz, 1H), 7.22-7.18 (m, 1H), 7.13-7.09 (m, 1H), 7.00 (d, J=8.9 Hz, 2H), 6.96-6.93 (m, 1H), 3.88 (s, 3H), 3.82 (s, 3H), 3.81 (s, 3H), 3.51-3.43 (m, 2H), 3.02 (d, J=4.7 Hz, 3H), 2.78 (d, J=5.0 Hz, 3H), 2.72 (t, J=6.6 Hz, 2H)

LRMS m/z calculated for C₃₂H₃₆N₈O₄ 596.29 [M], found 597.4 [M+H]⁺

### Ia55 (E)-N-(5-((5-((3-imino-3-(methylamino)propyl)carbamoyl)-1-methyl-1H-pyrrol-3-yl)carbamoyl)-1-methyl-1H-pyrrol-3-yl)-6-(4-methoxystyryl)nicotinamide

IR (cm⁻¹) 3190, 1688, 1172, 1126, 719

¹H NMR (500 MHz, DMSO-d₆) δ (ppm) 10.47 (s, 1H), 9.96 (s, 1H), 9.41 (s, 1H), 9.09-9.04 (m, 2H), 8.51 (s, 1H), 8.28-8.23 (m, 1H), 8.22-8.19 (m, 1H), 7.75 (d, J=16.0 Hz, 1H), 7.69-7.62 (m, 3H), 7.36-7.32 (m, 1H), 7.25 (d, J=16.0 Hz, 1H), 7.21-7.18 (m, 1H), 7.12-7.09 (m, 1H), 7.00 (d, J=8.9 Hz, 2H), 6.96-6.92 (m, 1H), 3.89 (s, 3H), 3.83 (s, 3H), 3.82 (s, 3H), 3.50-3.48 (m, 2H), 2.80 (d, J=4.9 Hz, 3H), 2.59 (t, J=6.6 Hz, 2H)

LRMS m/z calculated for C₃₁H₃₄N₈O₄ 582.27 [M], found 583.3 [M+H]⁺

### Ia56 (E)-N-(3-imino-3-(methylamino)propyl)-4-(4-(4-(3-methoxystyryl)benzamido)-1-methyl-1H-pyrrole-2-carboxamido)-1-methyl-1H-pyrrole-2-carboxamide

IR (cm⁻¹) 3250, 1629, 1431, 1199, 686

¹H NMR (500 MHz, DMSO-d₆) δ (ppm) 10.32 (s, 1H), 9.95 (s, 1H), 9.42-9.39 (m, 1H), 9.05 (s, 1H), 8.51 (s, 1H), 8.20 (t, J=5.7 Hz, 1H), 7.97 (d, J=8.5 Hz, 2H), 7.74 (d, J=8.5 Hz, 2H), 7.39-7.29 (m, 4H), 7.25-7.17 (m, 3H), 7.14-7.10 (m, 1H), 6.96-6.92 (m, 1H), 6.92-6.86 (m, 1H), 3.88 (s, 3H), 3.84-3.79 (m, 6H), 3.51-3.48 (m, 2H), 2.80 (d, J=5.0 Hz, 3H), 2.59 (t, J=6.6 Hz, 2H)

LRMS m/z calculated for C₃₂H₃₅N₇O₄ 581.68 [M], found 582.3 [M+H]⁺

### Ia57 N—((Z)-3-((2,2-difluoroethyl)amino)-3-((2,2-difluoroethyl)imino)propyl)-1-methyl-4-(1-methyl-4-(4-((E)-2-(quinolin-3-yl)vinyl)benzamido)-1H-pyrrole-2-carboxamido)-1H-pyrrole-2-carboxamide

IR (cm⁻¹) 3209, 1651, 1118, 719

¹H NMR (500 MHz, DMSO-d₆) δ (ppm) 10.36 (s, 1H), 10.17 (t, J=6.3 Hz, 1H), 9.97 (s, 1H), 9.44 (t, J=5.8 Hz, 1H), 9.28 (d, J=2.3 Hz, 1H), 8.60 (s, 1H), 8.27 (t, J=6.3 Hz, 1H), 8.07-7.99 (m, 4H), 7.85-7.74 (m, 3H), 7.69-7.62 (m, 3H), 7.35 (d, J=2.0 Hz, 1H), 7.20 (d, J=2.0 Hz, 1H), 7.12 (d, J=2.0 Hz, 1H), 6.97 (d, J=2.0 Hz, 1H), 6.40-6.13 (m, 2H), 4.04-3.94 (m, 4H), 3.88 (s, 3H), 3.82 (s, 3H), 3.47 (q, J=6.6 Hz, 2H), 2.84 (t, J=6.9 Hz, 2H)

¹⁹F NMR (471 MHz, DMSO-d₆) δ (ppm) −74.38 (TFA), −122.15 (dt, J=54.6, 15.6 Hz), −122.90 (dt, J=54.6, 15.6 Hz)

LRMS m/z calculated for C₃₇H₃₆F₄N₈O₃ 716.74 [M], found 717.3 [M+H]⁺

### Ia58 (E)-N-(3-imino-3-((2-methoxyethyl)amino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 2360, 1193, 1116, 839

¹H NMR (500 MHz, DMSO-d₆) δ (ppm) 10.35 (s, 1H), 9.95 (s, 1H), 9.48 (s, 1H), 9.26 (d, J=2.1 Hz, 1H), 9.06 (s, 1H), 8.60 (s, 1H), 8.55 (s, 1H), 8.16 (m, 1H), 8.06-7.98 (m, 4H), 7.82 (d, J=8.4 Hz, 2H), 7.76 (t, J=7.6 Hz, 1H), 7.71-7.58 (m, 3H), 7.34 (d, J=2.0 Hz, 1H), 7.18 (d, J=2.0 Hz, 1H), 7.12 (d, J=2.0 Hz, 1H), 6.97 (d, J=2.0 Hz, 1H), 3.88 (s, 3H), 3.82 (s, 3H), 3.5-3.45 (m, 4H, underneath water peak), 3.37-3.35 (m, 2H), 3.26 (s, 3H), 2.62-2.59 (m, 2H)

LRMS m/z calculated for C₃₆H₃₈N₈O₄ 646.75 [M], found 647.3 [M+H]⁺

### Ia59 6-((E)-4-fluoro-3-methoxystyryl)-N-(1-methyl-5-((1-methyl-5-(((Z)-3-(methylamino)-3-(methylimino)propyl)carbamoyl)-1H-pyrrol-3-yl)carbamoyl)-1H-pyrrol-3-yl)nicotinamide

IR (cm⁻¹) 3252, 3103, 2951, 1653, 1588, 1517, 1465, 1437, 1407, 1318, 1264, 1184, 1122, 1031, 1009, 964, 800, 722

¹H NMR (500 MHz, DMSO-d₆) δ (ppm) 10.52 (s, 1H), 9.99 (s, 1H), 9.38 (q, J=5.0 Hz, 1H), 9.10 (d, J=2.3 Hz, 1H), 8.64 (q, J=4.8 Hz, 1H), 8.34-8.26 (m, 2H), 7.79 (d, J=16.0 Hz, 1H), 7.69 (d, J=8.2 Hz, 1H), 7.59-7.53 (m, 1H), 7.43 (d, J=16.1 Hz, 1H), 7.36 (d, J=1.8 Hz, 1H), 7.27-7.19 (m, 2H), 7.20 (d, J=1.9 Hz, 1H), 7.12 (d, J=1.9 Hz, 1H), 6.95 (d, J=1.9 Hz, 1H), 3.94 (s, 3H), 3.89 (s, 3H), 3.83 (s, 3H), 3.48 (q, J=6.5 Hz, 2H), 3.04 (d, J=4.9 Hz, 3H), 2.79 (d, J=4.9 Hz, 3H), 2.73 (t, J=6.7 Hz, 2H).

LRMS m/z calculated for C₃₂H₃₅FN₈O₄ 614.68 [M], found 615.3 [M+H]⁺

### Ia60 1-methyl-4-(1-methyl-4-(4-((E)-2-(pyrimidin-5-yl)vinyl)benzamido)-1H-pyrrole-2-carboxamido)-N—((Z)-3-(methylamino)-3-(methylimino)propyl)-1H-pyrrole-2-carboxamide

IR (cm⁻¹) 1074.35, 1128.36, 1186.22, 1271.09, 1388.75, 1404.18, 1436.97, 1465.9, 1560.41, 1570.06, 1610.56, 1647.21, 1653, 1978.97, 2042.62, 2160.27, 2360.87, 2945.3, 3105.39, 3263.56 cm-1

¹H NMR (500 MHz, DMSO-d₆) δ (ppm) 10.41 (s, 1H), 9.98 (s, 1H), 9.35 (m, 1H), 9.18 (s, 1H), 8.82 (d, J=5.2 Hz, 1H), 8.62 (m, 1H), 8.28 (m, 1H), 8.05-7.98 (m, 3H), 7.89 (d, J=8.1 Hz, 2H), 7.70-7.65 (m, 1H), 7.46 (d, J=16.1 Hz, 1H), 7.35 (d, J=1.9 Hz, 1H), 7.21 (d, J=1.8 Hz, 1H), 7.13 (d, J=2.0 Hz, 1H), 6.96 (d, J=1.9 Hz, 1H), 3.89 (s, 3H), 3.83 (s, 3H), 3.41-3.48 (m, 2H), 3.04 (d, J=4.7 Hz, 3H), 2.79 (d, J=4.9 Hz, 3H), 2.73 (t, J=6.8 Hz, 2H)

LRMS m/z calculated for C₃₁H₃₃N₈O₃ 567.27 [M], found 568.4 [M+H]⁺

### Ia61 N-(1-methyl-5-((1-methyl-5-(((Z)-3-(methylamino)-3-(methylimino)propyl)carbamoyl)-1H-pyrrol-3-yl)carbamoyl)-1H-pyrrol-3-yl)-6-((E)-3-(trifluoromethyl)styryl)nicotinamide

IR (cm⁻¹) 3275.13, 2166.06, 1656.85, 1587.42, 1535.34, 1435.04, 1406.11, 1327.03, 1294.24, 1265.3, 1197.79, 1118.71

¹H NMR (500 MHz, DMSO-d₆) δ (ppm) 10.52 (s, 1H), 9.97 (s, 1H), 9.34 (m, 1H), 9.11 (s, 1H), 8.66-8.58 (m, 1H), 8.34-8.24 (m, 2H), 8.10-8.01 (m, 2H), 7.86 (d, 16.0 Hz, 1H), 7.75-7.56 (m, 4H), 7.35 (d, J=1.9 Hz, 1H), 7.20 (d, J=1.8 Hz, 1H), 7.11 (d, J=1.9 Hz, 1H), 6.94 (d, J=1.9 Hz, 1H), 3.89 (s, 3H), 3.82 (s, 3H), 3.44-3.50 (m, 2H, underneath water peak), 3.03 (d, J=4.7 Hz, 3H), 2.78 (d, J=5.0 Hz, 3H), 2.72 (t, J=6.7 Hz, 2H)

LRMS m/z calculated for C₃₂H₃₃F₃N₈O₃ 634.26 [M], found 635.3 [M+H]⁺

### Ia62 (E)-N-(3-((2,2-difluoroethyl)amino)-3-iminopropyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3228, 1629, 1197, 1122, 719

¹H NMR (500 MHz, DMSO-d₆) δ (ppm) 10.36 (s, 1H), 9.96 (s, 1H), 9.77 (m, 1H), 9.42 (s, 1H), 9.27 (d, J=2.3 Hz, 1H), 8.99 (s, 1H), 8.56 (s, 1H), 8.19 (t, J=5.8 Hz, 1H), 8.03-7.99 (m, 4H), 7.84-7.80 (m, 2H), 7.78-7.74 (m, 1H), 7.69-7.60 (m, 3H), 7.34 (d, J=1.8 Hz, 1H), 7.19 (d, J=1.8 Hz, 1H), 7.12 (d, J=2.0 Hz, 1H), 6.96 (d, J=2.0 Hz, 1H), 6.37-6.05 (m, 1H), 3.88 (s, 3H), 3.82 (s, 3H), 3.78-3.73 (m, 2H), 3.52-3.49 (m, 2H), 2.66 (t, J=6.3 Hz, 2H)

LRMS m/z calculated for C₃₅H₃₄F₂N₈O₃ 652.71 [M], found 653.3 [M+H]⁺

### Ia63 (E)-N-(5-((5-((3-((2,2-difluoroethyl)amino)-3-iminopropyl)carbamoyl)-1-methyl-1H-pyrrol-3-yl)carbamoyl)-1-methyl-1H-pyrrol-3-yl)-6-(4-(dimethylamino)styryl)nicotinamide

IR (cm⁻¹) 3269, 1570, 1527, 1166, 1122, 719

¹H NMR (500 MHz, DMSO-d₆) δ (ppm) 10.46 (s, 1H), 9.96 (s, 1H), 9.79-9.76 (m, 1H), 9.42 (s, 1H), 9.04 (d, J=2.6 Hz, 1H), 9.00 (s, 1H), 8.26 (dd, J=8.1, 2.4 Hz, 1H), 8.19 (t, J=5.8 Hz, 1H), 7.72 (d, J=16.0 Hz, 1H), 7.65 (d, J=8.2 Hz, 1H), 7.54 (d, J=8.9 Hz, 2H), 7.34 (d, J=2.0 Hz, 1H), 7.19 (d, J=2.0 Hz, 1H), 7.15-7.08 (m, 2H), 6.95 (d, J=1.8 Hz, 1H), 6.76 (d, J=9.2 Hz, 2H), 6.35-6.09 (m, 1H), 3.88 (s, 3H), 3.82 (s, 3H), 3.78-3.71 (m, 2H), 3.51 (q, J=6.3 Hz, 2H), 2.98 (s, 6H), 2.66 (t, J=6.6 Hz, 2H)

LRMS m/z calculated for C₃₃H₃₇F₂N₉O₃ 645.72 [M], found 646.4 [M+H]⁺

### Ia64 4-(4-((E)-3-fluorostyryl)benzamido)-1-methyl-N-(1-methyl-5-(((Z)-3-(methylamino)-3-(methylimino)propyl)carbamoyl)-1H-pyrrol-3-yl)-1H-pyrrole-2-carboxamide

IR (cm⁻¹) 3253.91, 3116.97, 2953.02, 2358.94, 2331.94, 2166.06, 2050.33, 1978.97, 1651.07, 1581.63, 1523.76, 1465.90, 1435.04, 1402.25, 1267.23, 1199.72, 1128.36

¹H NMR (500 MHz, DMSO-d₆) δ (ppm) 10.34 (s, 1H), 9.97 (s, 1H), 9.34 (m, 1H), 8.60 (m, 1H), 8.27 (t, J=5.2 Hz, J=5.8 Hz, 1H), 7.98 (d, J=8.3 Hz, 2H), 7.75 (d, J=8.3 Hz, 2H), 7.52 (d, J=10.9 Hz, 1H), 7.42-7.50 (m, 4H), 7.34 (d, J=1.6 Hz, 1H), 7.20 (d, J=1.6 Hz, 1H), 7.11-7.18 (m, 2H), 6.95 (d, J=1.5 Hz, 1H), 3.89 (s, 3H), 3.82 (s, 3H), 3.47 (q, J=6.5 Hz, 2H), 3.03 (d, J=4.6 Hz, 3H), 2.79 (d, J=5.0 Hz, 3H), 2.72 (t, J=6.5 Hz, 2H)

LRMS m/z calculated for C₃₂H₃₄FN₇O₃ 583.7 [M], found 584.3 [M+H]⁺

### Ia65 6-((E)-4-fluorostyryl)-N-(1-methyl-5-((1-methyl-5-(((Z)-3-(methylamino)-3-(methylimino)propyl)carbamoyl)-1H-pyrrol-3-yl)carbamoyl)-1H-pyrrol-3-yl)nicotinamide

IR (cm⁻¹) 3265.49, 3122.75, 2953.02, 2162.20, 2036.83, 1978.97, 1654.92, 1637.56, 1591.27, 1527.62, 1508.33, 1458.18, 1436.97, 1406.11, 1292.31, 1267.23, 1240.23, 1190.08, 1128.36

¹H NMR (500 MHz, DMSO-d₆) δ (ppm) 10.50 (s, 1H), 9.98 (s, 1H), 9.34 (m, 1H), 9.10 (d, J=2.0 Hz, 1H), 8.61 (m, 1H), 8.26-8.32 (m, 2H), 7.78 (m, 3H), 7.69 (d, J=8.2 Hz, 1H), 7.38 (d, J=16.8 Hz, 1H), 7.35 (d, J=1.5 Hz, 1H), 7.28 (t, J=9 Hz, 2H), 7.20 (d, J=1.6 Hz, 1H), 7.12 (d, J=1.4 Hz, 1H), 6.95 (d, J=1.5 Hz, 1H), 3.89 (s, 3H), 3.82 (s, 3H), 3.3-35 (m, 2H, underneath water peak), 3.03 (d, J=4.5 Hz, 3H), 2.79 (d, J=4.5 Hz, 3H), 2.72 (t, J=6.5 Hz, 2H)

LRMS m/z calculated for C₃₁H₃₃FN₈O₃ 584.7 [M], found 585.3 [M+H]⁺

### Ib1 (E)-N-(3-imino-3-(piperidin-1-yl)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3226, 1670, 1199, 839

¹H NMR (500 MHz, DMSO-d₆) δ (ppm) 10.4 (s, 1H), 9.97 (s, 1H), 9.27 (d, J=1.9 Hz, 1H), 8.99 (s, 1H), 8.58 (s, 1H), 8.48 (s, 1H), 8.23 (t, J=5.9 Hz, 1H), 8.03-7.00 (m, 4H), 7.82 (d, J=8.3 Hz, 2H), 7.79-7.74 (m, 1H), 7.68-7.62 (m, 3H), 7.34 (d, J=1.5 Hz, 1H), 7.19 (d, J=1.5 Hz, 1H), 7.12 (d, J=1.5 Hz, 1H), 6.96 (d, J=1.5 Hz, 1H), 3.88 (s, 3H), 3.82 (s, 3H), 3.68-3.66 (m, 2H), 3.51-3.50 (m, 2H), 2.80-2.75 (m, 2H), 1.71-1.58 (m, 8H)

LRMS m/z calculated for C₃₈H₄₀N₈O₃ 656.32 [M]⁺, found 657.3 [M+H]⁺

HRMS m/z calculated C₃₈H₄₁O₃N₈ 657.3296 [M+H]⁺, found 657.3292 [M+H]⁺

### Ib2 (E)-4-(4-(2-(benzo[c][1,2,5]oxadiazol-5-yl)vinyl)benzamido)-N-(5-((3-imino-3-(piperidin-1-yl)propyl)carbamoyl)-1-methyl-1H-pyrrol-3-yl)-1-methyl-1H-pyrrole-2-carboxamide

IR (cm⁻¹) 3097, 1627, 1199, 798

¹H NMR (500 MHz, DMSO-d₆) δ (ppm) 10.4 (s, 1H), 9.97 (s, 1H), 9.00 (s, 1H), 8.49 (s, 1H), 8.24 (t, J=5.9 Hz, 1H), 8.10 (s, 3H), 8.02 (d, J=8.3 Hz, 2H), 7.82 (d, J=8.3 Hz, 2H), 7.70 (d, J=16 Hz, 1H), 7.61 (d, J=16 Hz, 1H), 7.34 (d, J=1.5 Hz, 1H), 7.19 (d, J=1.5 Hz, 1H), 7.12 (d, J=1.5 Hz, 1H), 6.96 (d, J=1.5 Hz, 1H), 3.88 (s, 3H), 3.82 (s, 3H), 3.67-3.63 (m, 2H), 3.52-3.49 (m, 2H), 2.79-2.75 (m, 2H), 1.70-1.56 (m, 8H)

LRMS m/z calculated for C₃₅H₃₇N₉O₄ 647.30 [M]⁺, found 648.3 [M+H]⁺

HRMS m/z calculated C₃₅H₃₈O₄N₉ 648.3041 [M+H]⁺, found 648.3036 [M+H]⁺

### Ib3 (E)-N-(3-imino-3-(4-methylpiperazin-1-yl)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 2993, 1629, 1199, 835

¹H NMR (500 MHz, DMSO-d₆) δ (ppm) 10.4 (s, 1H), 9.98 (s, 1H), 9.53 (s, 1H), 9.32 (s, 1H), 8.92 (s, 1H), 8.67 (s, 1H), 8.28 (t, J=6.0 Hz, 1H), 8.08-8.00 (m, 4H), 7.84-7.81 (m, 3H), 7.70-7.61 (m, 3H), 7.36-7.32 (m, 1H), 7.20-7.17 (m, 1H), 7.15-7.13 (m, 1H), 7.03-7.00 (m, 1H), 3.88 (s, 3H), 3.83 (s, 3H), 3.83-3.81 (m, 2H), 3.50-3.43 (m, 4H), 2.89-2.81 (m, 5H)

LRMS m/z calculated for C₃₈H₄₁N₉O₃ 671.31 [M]⁺, found 672.3 [M+H]⁺

HRMS m/z calculated for C₃₈H₄₂O₃N₉ 672.3405 [M+H]⁺, found 672.3401 [M+H]⁺

### Ib4 (E)-4-(4-(2-(benzo[c][1,2,5]oxadiazol-5-yl)vinyl)benzamido)-N-(5-((3-imino-3-(4-methylpiperazin-1-yl)propyl)carbamoyl)-1-methyl-1H-pyrrol-3-yl)-1-methyl-1H-pyrrole-2-carboxamide

IR (cm⁻¹) 3051, 1668, 1199, 837

¹H NMR (500 MHz, DMSO-d₆) δ (ppm) 10.4 (s, 1H), 9.98 (s, 1H), 9.52 (s, 1H), 8.94 (s, 1H), 8.28 (t, J=5.6 Hz, 1H), 8.11 (s, 3H), 8.01 (d, J=8.3 Hz, 2H), 7.82 (d, J=8.3 Hz, 2H), 7.69-7.62 (m, 2H), 7.34 (d, J=1.4 Hz, 1H), 7.19 (d, J=1.4 Hz, 1H), 7.13 (d, J=1.5 Hz, 1H), 7.01-6.99 (m, 1H), 3.88 (s, 3H), 3.83 (s, 3H), 3.82-3.79 (m, 2H), 3.14-3.08 (m, 2H), 2.83 (s, 3H), 2.83-2.75 (m, 6H) (2H missing)

LRMS m/z calculated for C₃₅H₃₈N₁₀O₄ 662.31 [M]⁺, found 663.3 [M+H]⁺

HRMS m/z calculated for C₃₅H₃₉O₄N₁₀663.3150 [M+H]⁺, found 663.3145 [M+H]⁺

### Ib5 (E)-N-(3-imino-3-morpholinopropyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3280, 1670, 1197, 719

¹H NMR (500 MHz, DMSO-d₆) δ (ppm) 10.4 (s, 1H), 9.97 (s, 1H), 9.30 (d, J, =2.1 Hz, 1H), 9.21 (s, 1H), 8.64 (s, 1H), 8.56 (s, 1H), 8.25 (t, J=6.6 Hz, 1H), 8.05-7.99 (m, 4H), 7.84-7.81 (m, 2H), 7.75-7.74 (m, 1H), 7.68-7.63 (m, 3H), 7.35 (d, J=1.5 Hz, 1H), 7.20 (d, J=1.5 Hz, 1H), 7.12 (d, J=1.5 Hz, 1H), 6.98 (d, J=1.5 Hz, 1H), 3.88 (s, 3H), 3.83 (s, 3H), 3.74-3.71 (m, 6H), 3.56-3.53 (m, 3H), 3.48-3.46 (m, 2H)

LRMS m/z calculated for C₃₇H₃₈N₈O₄ 658.30 [M]⁺, found 659.3 [M+H]⁺

HRMS m/z calculated for C₃₇H₃₉O₄N₈ 659.3089 [M+H]⁺, found 659.3082 [M+H]⁺

### Ib6 (E)-4-(4-(2-(benzo[c][1,2,5]oxadiazol-5-yl)vinyl)benzamido)-N-(5-((3-imino-3-morpholinopropyl)carbamoyl)-1-methyl-1H-pyrrol-3-yl)-1-methyl-1H-pyrrole-2-carboxamide

IR (cm⁻¹) 3138, 1627, 1199, 798

¹H NMR (500 MHz, DMSO-d₆) δ (ppm) 10.4 (s, 1H), 9.97 (s, 1H), 9.23 (s, 1H), 8.65 (s, 1H), 8.25 (t, J=6.0 Hz, 1H), 8.11 (s, 3H), 8.01 (d, J=8.4 Hz, 2H), 7.81 (d, J=8.4 Hz, 2H), 7.69 (d, J=16 Hz, 1H), 7.61 (d, J=16 Hz, 1H), 7.34 (d, J=1.5 Hz, 1H), 7.21-7.19 (m, 1H), 7.12 (d, J=1.5 Hz, 1H), 6.97 (d, J=1.7 Hz, 1H), 3.88 (s, 3H), 3.83 (s, 3H), 3.75-3.70 (m, 8H), 3.47-3.45 (m, 2H), 2.82-2.72 (m, 2H)

LRMS m/z calculated for C₃₄H₃₅N₉O₅ 649.28 [M]⁺, found 650.3 [M+H]⁺

HRMS m/z calculated for C₃₄H₃₆O₅N₉ 650.2834 [M+H]⁺, found 650.2833 [M+H]⁺

### Ib7 (E)-N-(3-imino-3-thiomorpholinopropyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3263, 1629, 1197, 719

¹H NMR (500 MHz, DMSO-d₆) δ (ppm) 10.4 (s, 1H), 9.97 (s, 1H), 9.27 (d, J=2.0 Hz, 1H), 9.22 (s, 1H), 8.66 (s, 1H), 8.57 (s, 1H), 8.24 (t, J, =5.8 Hz, 1H), 8.05-7.99 (m, 5H), 7.82 (d, J=8.6 Hz, 2H), 7.79-7.75 (m, 1H), 7.68-7.63 (m, 3H), 7.35 (d, J=1.5 Hz, 1H), 7.20 (d, J=1.5 Hz, 1H), 7.13 (d, J=1.5 Hz, 1H), 6.98 (d, J=1.5 Hz, 1H), 3.98-3.93 (m, 3H), 3.88 (s, 3H), 3.83 (s, 3H), 3.47-3.42 (m, 2H), 2.90-2.86 (m, 2H), 2.81-2.76 (m, 5H)

LRMS m/z calculated for C₃₇H₃₈N₈O₃S 674.28 [M]⁺, found 675.3 [M+H]⁺

HRMS m/z calculated for C₃₇H₃₉O₃N₈S 675.2860 [M+H]⁺, found 675.2861 [M+H]⁺

### Ib8 (E)-4-(4-(2-(benzo[c][1,2,5]oxadiazol-5-yl)vinyl)benzamido)-N-(5-((3-imino-3-thiomorpholinopropyl)carbamoyl)-1-methyl-1H-pyrrol-3-yl)-1-methyl-1H-pyrrole-2-carboxamide

IR (cm⁻¹) 3371, 1379, 1128, 950

¹H NMR (500 MHz, DMSO-d₆) δ (ppm) 10.4 (s, 1H), 9.97 (s, 1H), 9.23 (s, 1H), 8.67 (s, 1H), 8.24 (t, J=6.3 Hz, 1H), 8.11 (s, 3H), 8.02 (d, J=8.4 Hz, 2H), 7.82 (d, J=8.4 Hz, 2H), 7.70 (d, J=16 Hz, 1H), 7.61 (d, J=16 Hz, 1H), 7.34 (d, J=1.7 Hz, 1H), 7.20 7.34 (d, J=1.7 Hz, 1H), 7.12 (d, J=1.7 Hz, 1H), 6.97 (d, J=1.7 Hz, 1H), 3.97-3.93 (m, 4H), 3.88 (s, 3H), 3.83 (s, 3H), 3.46-3.43 (m, 2H), 2.89-2.86 (m, 2H), 2.80-2.78 (m, 4H)

LRMS m/z calculated for C₃₄H₃₅N₉O₄S 665.25 [M]⁺, found 666.3 [M+H]⁺

HRMS m/z calculated for C₃₄H₃₆O₄N₉S 666.2605 [M+H]⁺, found 666.2601 [M+H]⁺

### Ib9 (E)-6-(4-(dimethylamino)styryl)-N-(5-((5-((3-imino-3-thiomorpholinopropyl)carbamoyl)-1-methyl-1H-pyrrol-3-yl)carbamoyl)-1-methyl-1H-pyrrol-3-yl)nicotinamide

IR (cm⁻¹) 3105, 1670, 1575, 1166, 717

¹H NMR (500 MHz, DMSO-d₆) δ (ppm) 10.4 (s, 1H), 9.97 (s, 1H), 9.31 (s, 1H), 9.03 (s, 1H), 8.65 (s, 1H), 8.27-8.21 (m, 2H), 7.71 (d, J=16 Hz, 1H), 7.63 (d, J=8.2 Hz, 1H), 7.54 (d, J=8.8 Hz, 2H), 7.34 (s, 1H), 7.19 (s, 1H), 7.14-7.09 (m, 2H), 6.97 (s, 1H), 6.76 (d, J=8.8 Hz, 2H), 3.97-3.94 (m, 4H), 3.88 (s, 3H), 3.83 (s, 3H), 3.45-3.43 (m, 2H), 2.98 (s, 6H), 2.89-2.86 (m, 2H), 2.81-2.77 (m, 4H)

LRMS m/z calculated for C₃₅H₄₁N₉O₃S 667.31 [M]⁺, found 668.4 [M+H]⁺

HRMS m/z calculated for C₃₅H₄₂N₉O₃S 668.3126 [M+H]⁺, found 668.3118 [M+H]⁺

### Ib10 (E)-6-(4-(dimethylamino)styryl)-N-(5-((5-((3-imino-3-(4-methylpiperazin-1-yl)propyl)carbamoyl)-1-methyl-1H-pyrrol-3-yl)carbamoyl)-1-methyl-1H-pyrrol-3-yl)nicotinamide

IR (cm⁻¹) 2970, 1670, 1128, 798

¹H NMR (500 MHz, DMSO-d₆) δ (ppm) 10.5 (s, 1H), 9.98 (s, 1H), 9.53 (s, 1H), 9.05-9.03 (m, 1H), 8.94 (s, 1H), 8.31-8.22 (m, 2H), 7.72 (d, J=16 Hz, 1H), 7.64 (d, J=8.2 Hz, 1H), 7.54 (d, J=8.8 Hz, 2H), 7.33 (s, 1H), 7.19 (s, 1H), 7.14-7.08 (m, 2H), 7.00 (s, 1H), 6.76 (d, J=8.8 Hz, 2H), 3.88 (s, 3H), 3.83 (s, 3H), 3.49-3.42 (m, 3H), 2.98 (s, 6H), 2.87-2.81 (m, 5H), 2.09-2.07 (m, 5H)

LRMS m/z calculated for C₃₆H₄₄N₁₀O₃ 664.36 [M]⁺, found 665.6 [M+H]⁺

HRMS m/z calculated for C₃₆H₄₅N₁₀O₃ 665.3671 [M+H]⁺, found 665.3665 [M+H]⁺

### Ib11 (E)-6-(4-(dimethylamino)styryl)-N-(5-((5-((3-imino-3-morpholinopropyl)carbamoyl)-1-methyl-1H-pyrrol-3-yl)carbamoyl)-1-methyl-1H-pyrrol-3-yl)nicotinamide

IR (cm⁻¹) 3307, 1653, 1570, 1168

¹H NMR (500 MHz, DMSO-d₆) δ (ppm) 10.4 (s, 1H), 9.97 (s, 1H), 9.21 (s, 1H), 9.04 (s, 1H), 8.64 (s, 1H), 8.28-8.22 (m, 2H), 7.71 (d, J=16 Hz, 1H), 7.63 (d, J=8.2 Hz, 1H), 7.54 (d, J=8.8 Hz, 2H), 7.34 (s, 1H), 7.20 (s, 1H), 7.14-7.09 (m, 2H), 6.97 (s, 1H), 6.76 (s, 1H), 3.88 (s, 3H), 3.83 (s, 3H), 3.76-3.66 (m, 8H), 3.55-3.53 (m, 2H), 2.98 (s, 6H), 2.81-2.77 (m, 2H)

LRMS m/z calculated for C₃₅H₄₁O₄N₉ 651.33 [M]⁺, found 652.8 [M+H]⁺

HRMS m/z calculated for C₃₅H₄₂O₄N₉ 652.3354 [M+H]⁺, found 652.3349 [M+H]⁺

### Synthesis of Compounds of the ‘Quaternary’ Set

Compounds of this set are synthesised by reaction of a compound of the invention or otherwise, which contains a tertiary amine, with an appropriate alkyl halide (Scheme 6, route A) or reaction of an appropriate quaternary ammonium tail group dimer with an appropriate head group dimer (Scheme 6, route B).

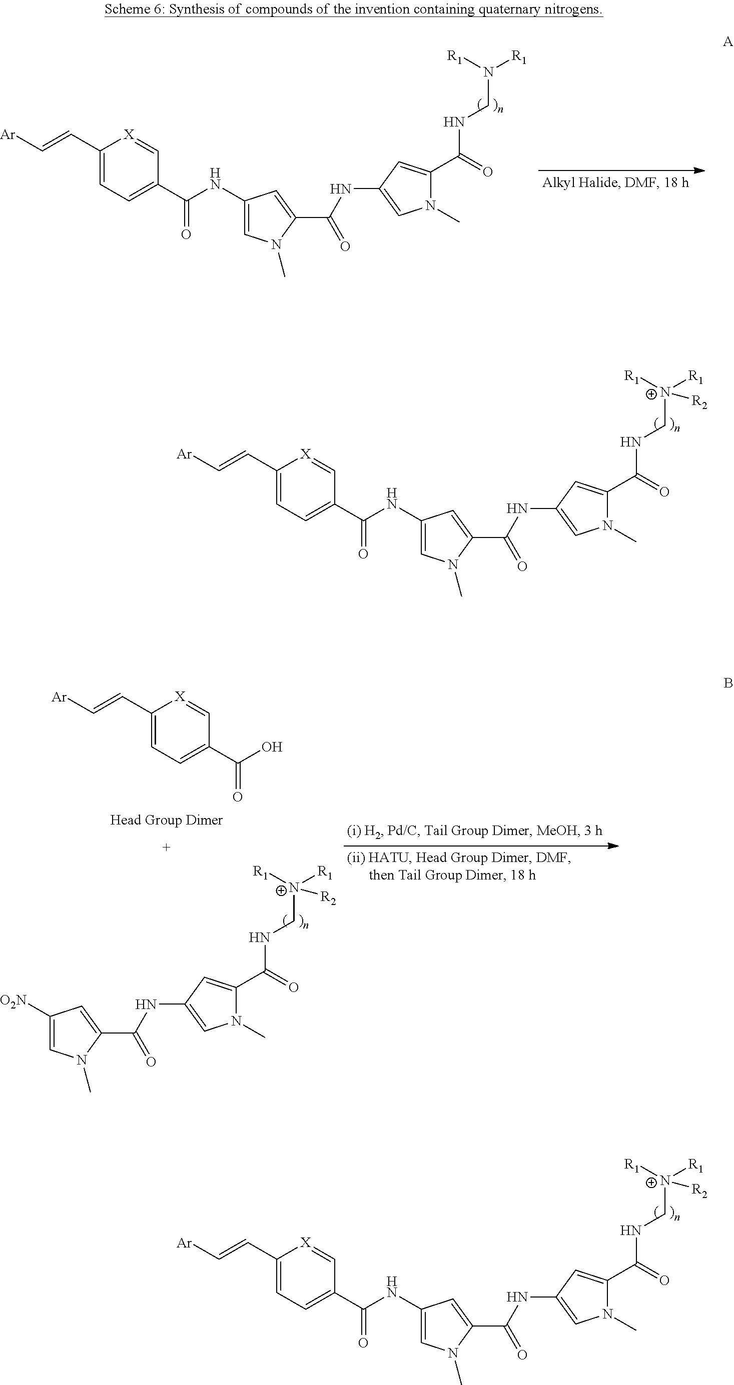

For route A, the appropriate starting compound (0.058 mmol) was dissolved in DMF (1 mL) along with the appropriate amine (2 eq, 0.116 mmol) and stirred at 50° C. for 4-24 hours. The reaction was returned to room temperature once there was no more starting material remaining (monitored by LCMS). After completion the reaction was purified by HPLC and the fractions containing the desired compound were freeze dried (˜40% yield).

For route B, the appropriate quaternary ammonium tail group dimer (0.1-0.2 mmol, 1.5 eq.) in methanol (20 mL) was treated with Pd/C (10% w/w) and hydrogenated at room temperature overnight via a balloon. Following this, the reaction mixture was filtered through celite and the methanol removed by evaporation under reduced pressure. The resulting residue was then re-dissolved in DMF (1.0 mL). The appropriate headgroup (1 eq) in DMF (1.0 mL) was treated with HBTU (1.5 eq.) and DIPEA (1.5 eq.) and stirred for 30 min before combining with the DMF solution of the amine. The reaction mixture was stirred at room temperature overnight and then purified directly by HPLC and the appropriate fractions freeze dried to obtain the desired compound (˜50% yield).

For the compounds of the invention exemplified below, the synthesis and characterisation details of the starting compounds (Ref-7 and Ref-3) are also provided.

### Ref-7 (E)-N-(2-(dimethylamino)ethyl)-1-methyl-4-(1-methyl-4-(4-(2-(quinolin-3-yl)vinyl)benzamido)-1H-pyrrole-2-carboxamido)-1H-pyrrole-2-carboxamide

F. Giordani et al., Journal of Medicinal Chemistry 2019 62 (6), 3021-3035, DOI: 10.1021/acs.jmedchem.8b01847

### Ref-3 (E)-1-methyl-4-(1-methyl-4-(4-(2-(quinolin-3-yl)vinyl)benzamido)-1H-pyrrole-2-carboxamido)-N-(2-morpholinoethyl)-1H-pyrrole-2-carboxamide

Nahoum G., 2007, supra

### Characterisation Details of Compounds of the Invention Containing Quaternary Nitrogen Atoms

### Ic1 (E)-N-ethyl-N,N-dimethyl-2-(1-methyl-4-(1-methyl-4-(4-(2-(quinolin-3-yl)vinyl)benzamido)-1H-pyrrole-2-carboxamido)-1H-pyrrole-2-carboxamido)ethan-1-aminium

IR (cm⁻¹) 3325, 1676, 1199, 719

¹H NMR (500 MHz, DMSO-d₆) δ (ppm) 9.98 (s, 1H), 9.30 (s, 1H), 8.62 (s, 1H), 8.28 (t, J=5.6 Hz, 1H), 8.08-7.99 (m, 4H), 7.85-7.76 (m 3H), 7.72-7.60 (m, 3H), 7.35 (d, J=1.6 Hz, 1H), 7.22 (d, J=1.6 Hz, 1H), 7.13 (d, J=1.7 Hz, 1H), 6.99 (d, J=1.7 Hz, 1H), 3.88 (s, 3H), 3.84 (s, 3H), 3.61-3.55 (m, 2H), 3.45-3.36 (m, 4H), 3.07 (s, 6H), 1.28 (t, J=7.2 Hz, 3H)

LRMS m/z calculated for C₃₆H₄₀N₇O₃⁺ 618.32, found 618.4 [M]⁺

HRMS m/z calculated for C₃₆H₄₀N₇O₃ 618.3187, found 618.3178 [M]⁺

### Ic2 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)propan-1-aminium

IR (cm⁻¹) 3138, 1627, 1199, 1128

¹H NMR (500 MHz, DMSO-d₆) δ (ppm) 10.4 (s, 1H), 9.98 (s, 1H), 9.28 (d, J=1.9 Hz, 1H), 8.60 (s, 1H), 8.28 (t, J=5.6 Hz, 1H), 8.06-8.00 (m, 4H), 7.82 (d, J=8.4 Hz, 2H), 7.78 (t, J=6.9 Hz, 1H), 7.71-7.60 (m, 3H), 7.35 (d, J=1.7 Hz, 1H), 7.21 (d, J=1.7 Hz, 1H), 7.13 (d, J=1.7 Hz, 1H) 7.00 (d, J=1.7 Hz, 1H), 3.88 (s, 3H), 3.84 (s, 3H), 3.59 (q, J=6.1 Hz, 2H), 3.41 (t, J=6.6 Hz, 2H), 3.32-3.27 (m, 2H), 3.09 (s, 6H), 1.77-1.68 (m, 2H)

LRMS m/z calculated for C₃₇H₄₂N₇O₃⁺ 632.33, found 632.4 [M]⁺

HRMS m/z calculated for C₃₇H₄₂N₇O₃ 632.3344, found 632.3336 [M]⁺

### Ic3 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)butan-1-aminium

IR (cm⁻¹) 3296, 1637, 1197, 719

¹H NMR (500 MHz, DMSO-d₆) δ (ppm) 10.4 (s, 1H), 9.98 (s, 1H), 9.27 (d, J=2.1 Hz, 1H), 8.57 (s, 1H), 8.27 (t, J=5.5 Hz, 1H), 8.05-7.99 (m, 4H), 7.82 (d, J=8.4 Hz, 2H), 7.79-7.74 (m, 1H), 7.68-7.65 (m, 3H), 7.35 (d, J=1.5 Hz, 1H), 7.21 (d, J=1.5 Hz, 1H), 7.12 (d, J=1.5 Hz, 1H), 6.99 (d, J=1.5 Hz, 1H), 3.88 (s, 3H), 3.84 (s, 3H), 3.61-3.56 (m, 2H), 3.44-3.40 (m, 2H), 3.36-3.31 (m, 2H), 3.09 (s, 6H), 1.73-1.65 (m, 2H), 1.35-1.27 (m, 2H), 0.93 (t, J=7.3 Hz, 3H)

LRMS m/z calculated for C₃₈H₄₄N₇O₃⁺ 646.35, found 646.6 [M]⁺

HRMS m/z calculated for C₃₈H₄₄O₃N₇ 646.3500, found 646.3496 [M]⁺

### Ic4 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)pentan-1-aminium

IR (cm⁻¹) 3288, 1639, 1197, 748

¹H NMR (500 MHz, DMSO-d₆) δ (ppm) 10.4 (s, 1H), 9.98 (s, 1H), 9.29 (d, J=1.8 Hz, 1H), 8.60 (s, 1H), 8.29 (t, J=5.7 Hz, 1H), 8.07-8.00 (m, 4H), 7.82 (d, J=8.2 Hz, 2H), 7.80-7.76 (m, 1H), 7.71-7.60 (m, 3H), 7.35 (d, J=1.4 Hz, 1H), 7.21 (d, J=1.4 Hz, 1H), 7.12 (d, J=1.4 Hz, 1H), 7.00 (d, J=1.4 Hz, 1H), 3.88 (s, 3H), 3.84 (s, 3H), 3.61-3.56 (m, 2H), 3.44-3.40 (m, 2H), 3.35-3.30 (m, 2H), 3.09 (s, 6H), 1.71-1.63 (m, 2H), 1.34-1.25 (m, 4H), 0.90-0.87 (m, 3H)

LRMS m/z calculated for C₃₉H₄₆N₇O₃⁺ 660.37, found 660.6 [M]⁺

HRMS m/z calculated for C₃₉H₄₆O₃N₇⁺ 660.3657, found 660.3653 [M]⁺

### Ic5 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)hexan-1-aminium

IR (cm⁻¹) 3282, 1627, 1199, 842

¹H NMR (500 MHz, DMSO-d₆) δ (ppm) 10.4 (s, 1H), 9.99 (s, 1H), 9.31 (s, 1H), 8.32 (s, 1H), 8.10-7.99 (m, 4H), 7.85-7.78 (m, 3H), 7.73-7.59 (m, 3H), 7.35 (m, 1H), 7.21 (s, 1H), 7.13 (s, 1H), 7.02 (s, 1H), 3.88 (s, 3H), 3.84 (s, 3H), 3.62-3.55 (m, 2H), 3.45-3.39 (m, 2H), 3.37-3.30 (m, 2H), 3.10 (s, 6H), 1.74-1.64 (m, 2H), 1.39-1.25 (m, 2H), 0.86 (t, J=6.5 Hz, 3H)

LRMS m/z calculated for C₄₀H₄₈N₇O₃⁺ 674.38, found 674.4 [M]⁺

HRMS m/z calculated for C₄₀H₄₈O₃N₇⁺ 674.3813, found 674.3816 [M]⁺

### Ic6 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)heptan-1-aminium

IR (cm⁻¹) 3271, 1627, 1122, 719

¹H NMR (500 MHz, DMSO-d₆) δ (ppm) 10.4 (s, 1H), 9.98 (s, 1H), 9.27 (s, 1H), 8.58 (s, 1H), 8.27 (t, J=5.5 Hz, 1H), 8.05-8.00 (m, 4H), 7.82 (d, J=8.1 Hz, 2H), 7.79-7.75 (m, 1H), 7.71-7.60 (m, 3H), 7.36-7.34 (m, 1H), 7.21-7.20 (m, 1H), 7.13-7.12 (m, 1H), 7.02-7.00 (m, 1H), 3.88 (s, 3H), 3.84 (s, 3H), 3.62-3.55 (m, 2H), 3.44-3.39 (m, 2H), 3.36-3.30 (m, 2H), 3.08 (s, 6H), 1.73-1.65 (m, 2H), 1.30-1.22 (m, 8H), 0.86 (t, J=7.0 Hz, 3H)

LRMS m/z calculated for C₄₁H₅₀N₇O₃⁺ 688.40, found 688.6 [M]⁺

HRMS m/z calculated for C₄₁H₅₀N₇O₃ 688.3970, found 688.3967 [M]⁺

### Ic7 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)octan-1-aminium

IR (cm⁻¹) 3298, 1641, 1197, 719

¹H NMR (500 MHz, DMSO-d₆) δ (ppm) 10.4 (s, 1H), 9.98 (s, 1H), 9.29 (d, J=1.8 Hz, 1H), 8.61 (s, 1H), 8.28 (t, J=5.5 Hz, 1H), 8.07-8.00 (m, 4H), 7.82 (d, J=8.3 Hz, 2H), 7.81-7.76 (m, 1H), 7.71-7.60 (m, 3H), 7.35-7.34 (m, 1H), 7.22-7.20 (m, 1H), 7.14-7.11 (m, 1H), 7.02-7.00 (m, 1H), 3.88 (s, 3H), 3.84 (s, 3H), 3.61-3.56 (m, 2H), 3.44-3.39 (m, 2H), 3.34-3.30 (m, 2H), 3.09 (s, 6H), 1.73-1.63 (m, 2H), 1.33-1.17 (m, 1H), 0.84 (t, J=6.8 Hz, 3H)

LRMS m/z calculated for C₄₂H₅₂N₇O₃⁺ 702.41, found 702.5 [M]⁺

HRMS m/z calculated for C₄₂H₅₂O₃N₇⁺ 702.4126, found 702.4109 [M]⁺

### Ic8 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)nonan-1-aminium

IR (cm⁻¹) 3275, 1637, 1197, 719

¹H NMR (500 MHz, DMSO-d₆) δ (ppm) 10.4 (s, 1H), 9.98 (s, 1H), 9.28 (d, J=2.0 Hz, 1H), 8.61 (s, 1H), 8.28 (t, J=5.5 Hz, 1H), 8.06-7.98 (m, 4H), 7.82 (d, J=8.2 Hz, 2H), 7.80-7.75 (m, 1H), 7.71-7.60 (m, 3H), 7.34 (d, J=1.5 Hz, 1H), 7.20 (d, J=1.5 Hz, 1H), 7.12 (d, J=1.5 Hz, 1H), 7.01 (d, J=1.5 Hz, 1H), 3.88 (s, 3H), 3.84 (s, 3H), 3.61-3.56 (m, 2H), 3.44-3.39 (m, 2H), 3.35-3.30 (m, 2H), 3.08 (s, 6H), 1.73-1.64 (m, 2H), 1.31-1.19 (m, 12H), 0.84 (t, J=6.8 Hz, 3H)

LRMS m/z calculated for C₄₃H₅₄N₇O₃⁺ 716.43, found 716.5 [M]⁺

HRMS m/z calculated for C₄₃H₅₅O₃N₇⁺ 358.7178, found 358.7180 [(M+H)/2]²⁺.

### Ic9 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)decan-1-aminium

IR (cm⁻¹) 3288.63, 2926.01, 1670.35, 1641.42, 1608.63, 1529.55, 1465.9, 1433.11, 1404.18, 1382.96, 1261.45, 1197.79, 1172.72, 1128.36, 1060.85, 1014.56, 1006.84, 966.34, 952.84, 894.97, 864.11, 829.39, 798.53, 771.53, 750.31, 719.45, 705.95

¹H NMR (500 MHz, DMSO-d₆) δ (ppm) 10.36 (s, 1H), 9.97 (s, 1H), 9.27 (d, 1H, J=2.08 Hz), 8.56 (d, 1H), 8.27 (t, 1H, J=5.34 Hz), 8.05-7.99 (m, 4H), 7.82 (d, 2H, J=8.39 Hz), 7.76 (td, 1H, J=7.01 Hz), 7.68 (d, 1H, J=16.78 Hz), 7.64 (td, 1H), 7.61 (d, 1H, J=16.42 Hz), 7.34 (d, 1H, J=1.67 Hz), 7.20 (d, 1H, J=1.67 Hz), 7.12 (d, 1H, J=1.89 Hz), 7.01 (d, 1H, J=1.67 Hz), 3.88 (s, 3H), 3.83 (s, 3H), 3.58 (q, 2H, J=8.10 Hz), 3.41 (t, 2H, J=8.10 Hz), 3.32 (m, 2H), 3.08 (s, 6H), 1.29-1.14 (m, 18H), 0.84 (t, 3H, J=6.71 Hz)

LRMS m/z calculated for C₄₄H₅₆N₇O₃⁺ 730.44, found 730.0 [M]⁺

### Ic10 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)undecan-1-aminium

IR (cm⁻¹) 3288, 1639, 1199, 831

¹H NMR (500 MHz, DMSO-d₆) δ (ppm) 10.4 (s, 1H), 9.97 (s, 1H), 9.28 (d, J=1.9 Hz, 1H), 8.58 (s, 1H), 8.28 (t, J=5.6 Hz, 1H), 8.06-8.00 (m, 4H), 7.82 (d, J=8.2 Hz, 2H), 7.80-7.75 (m, 1H), 7.71-7.59 (m, 3H), 7.35 (d, J=1.4 Hz, 1H), 7.21 (d, J=1.4 Hz, 1H), 7.13 (d, J=1.4 Hz, 1H), 7.01 (d, J=1.4 Hz, 1H), 3.88 (s, 3H), 3.84 (s, 3H), 3.59-3.58 (m, 2H), 3.44-3.39 (m, 2H), 3.35-3.28 (m, 2H), 3.08 (s, 6H), 1.72-1.63 (m, 2H), 1.29-1.19 (m, 18H), 0.84 (t, J=7.0 Hz, 3H)

LRMS m/z calculated for C₄₅H₅₈N₇O₃⁺ 744.46, found 744.4 [M]⁺

HRMS m/z calculated for C₄₅H₅₈O₃N₇⁺ 744.4596, found 744.4596 [M]⁺

### Ic11 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)dodecan-1-aminium

IR (cm⁻¹) 3271, 2926, 2854, 1672, 1637, 1554, 1433, 1384, 1263, 1199, 1122, 1060

¹H NMR (500 MHz, DMSO-d₆) δ (ppm) 10.37 (s, 1H), 9.99 (s, 1H), 9.28 (d, 1H, J=1.85 Hz), 8.58 (d, 1H, J=1.37 Hz), 8.28 (t, 1H, J=5.95 Hz), 8.05 (dd, 1H, J=8.69 Hz), 8.02 (dd, 1H), 8.02 (d, 2H), 7.83 (d, 2H, J=8.26 Hz), 7.78 (td, 1H, J=7.47 Hz), 7.69 (d, 1H, J=16.25 Hz), 7.66 (td, 1H), 7.63 (d, 1H, J=16.59 Hz), 7.35 (d, 1H, J=1.06 Hz), 7.21 (d, 1H, J=1.09 Hz), 7.13 (d, 1H, J=1.67 Hz), 7.02 (d, 1H, J=1.31 Hz), 3.89 (s, 3H), 3.85 (s, 3H), 3.59 (q, 2H, J=5.95 Hz), 3.31 (t, 2H, J=8.08 Hz), 3.09 (s, 6H), 1.23 (m, 22H), 0.85 (t, 3H, J=6.86 Hz)

HRMS m/z calculated for C₄₆H₆₀N₇O₃⁺ 758.4756 [M+H]⁺, found 758.4752 [M+H]⁺

### Ic12 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)tridecan-1-aminium

IR (cm⁻¹) 2931, 1635, 1498, 1305, 840

¹H NMR (500 MHz, DMSO-d₆) δ (ppm): 10.4 (s, 1H), 9.97 (s, 1H), 9.28 (s, 1H), 8.58 (s, 1H), 8.27 (t, J=5.6 Hz, 1H), 8.08 (d, J 17 Hz, 1H), 8.06-8.00 (m, 4H), 7.82 (d, J=8.3 Hz, 2H), 7.80-7.76 (m, 1H), 7.71-7.60 (m, 3H), 7.34 (d, J=1.5 Hz, 1H), 7.20 (d, J=1.5 Hz, 1H), 7.13 (d, J=1.5 Hz, 1H), 7.02 (d, J=1.5 Hz, 1H), 3.88 (s, 3H), 3.84 (s, 3H), 3.60-3.56 (m, 2H), 3.43-3.50 (m, 2H), 3.35-3.29 (m, 2H), 3.08 (s, 6H), 1.74-1.62 (m, 2H), 1.28-1.21 (m, 20H), 0.84 (t, J=7.0 Hz, 3H)

LRMS m/z calculated for C₄₇H₆₂N₇O₅ 772.49, found 772.5 [M]⁺

### Ic13 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)tetradecan-1-aminium

IR (cm⁻¹) 2922, 1672, 1122, 719

¹H NMR (500 MHz, DMSO-d₆) δ (ppm) 10.4 (s, 1H), 9.98 (s, 1H), 9.30 (d, J=2.0, 1H), 8.63 (s, 1H), 8.28 (t, J=5.6 Hz, 1H), 8.07-8.00 (m, 4H), 7.87-7.77 (m, 3H), 7.71-7.60 (m, 3H), 7.35 (d, J=1.5 Hz, 1H), 7.20 (d, J=1.5 Hz, 1H), 7.13 (d, J=1.5 Hz, 1H), 7.01 (d, J=1.5 Hz, 1H), 3.88 (s, 3H), 3.84 (s, 3H), 3.61-3.55 (m, 2H), 3.44-3.39 (m, 2H), 3.35-3.28 (m, 2H), 3.08 (s, 6H), 1.73-1.63 (m, 2H), 1.25-1.20 (m, 22H), 0.84 (t, J=7.0 Hz, 3H)

HRMS m/z calculated for C₄₈H₆₄N₇O₃⁺ 786.5065, found 786.5053 [M]⁺.

### Ic14 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)pentadecan-1-aminium

IR (cm⁻¹) 3286, 2924, 2852, 1670, 1637, 1558, 1433, 1406, 1263, 1199, 1124

¹H NMR (500 MHz, DMSO-d₆) δ (ppm): 10.37 (s, 1H), 9.99 (s, 1H), 9.28 (d, 1H, J=1.85 Hz), 8.58 (d, 1H, J=1.37 Hz), 8.28 (t, 1H, J=5.95 Hz), 8.05 (dd, 1H, J=8.69 Hz), 8.02 (dd, 1H), 8.02 (d, 2H), 7.83 (d, 2H, J=8.26 Hz), 7.78 (td, 1H, J=7.47 Hz), 7.69 (d, 1H, J=16.25 Hz), 7.66 (td, 1H), 7.63 (d, 1H, J=16.59 Hz), 7.35 (d, 1H, J=1.06 Hz), 7.21 (d, 1H, J=1.09 Hz), 7.13 (d, 1H, J=1.67 Hz), 7.02 (d, 1H, J=1.31 Hz), 3.89 (s, 3H), 3.85 (s, 3H), 3.59 (q, 2H, J=5.95 Hz), 3.31 (t, 2H, J=8.08 Hz), 3.09 (s, 6H), 1.23 (m, 22H), 0.85 (t, 3H, J=6.86 Hz)

HRMS m/z calculated for C₄₉H₆₆N₇O₃⁺ 800.5222 [M+H]⁺, found 800.5222 [M+H]⁺

### Ic15 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)hexadecan-1-aminium

IR (cm⁻¹) 3518, 2922, 2850, 1672, 1643, 1554, 1433, 1404, 1261, 1199, 1176, 1118

¹H NMR (500 MHz, DMSO-d₆) δ (ppm): 10.37 (s, 1H), 9.98 (s, 1H), 9.28 (d, 1H, J=2.13 Hz), 8.58 (d, 1H, J=2.27 Hz), 8.28 (t, 1H, J=5.77 Hz), 8.04 (dd, 1H, J=8.59 Hz), 8.02 (dd, 1H), 8.02 (d, 2H), 7.83 (d, 2H, J=8.44 Hz), 7.77 (td, 1H, J=8.33 Hz), 7.69 (d, 1H, J=16.41 Hz), 7.65 (td, 1H), 7.62 (d, 1H, J=16.31 Hz), 7.35 (d, 1H, J=1.50 Hz), 7.21 (d, 1H, J=1.75 Hz), 7.14 (d, 1H, J=1.77 Hz), 7.02 (d, 1H, J=1.79 Hz), 3.89 (s, 3H), 3.85 (s, 3H), 3.59 (q, 2H, J=6.02 Hz), 3.32 (t, 2H), 3.09 (s, 6H), 1.23 (m, 30H), 0.85 (t, 3H, J=6.52 Hz)

### Ic16 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)heptadecan-1-aminium

IR (cm⁻¹) 2922, 1644, 1197, 1168

¹H NMR (500 MHz, DMSO-d₆) δ (ppm) 10.4 (s, 1H), 9.98 (s, 1H), 9.30 (s, 1H), 8.63 (s, 1H), 8.29 (t, J=5.5 Hz, 1H), 8.08-8.00 (m, 4H), 7.84-7.77 (m, 3H), 7.72-7.60 (m, 3H), 7.35 (d, J=1.5 Hz, 1H), 7.21 (d, J=1.5 Hz, 1H), 7.13 (d, J=1.5 Hz, 1H), 7.01 (d, J=1.5 Hz, 1H), 3.88 (s, 3H), 3.84 (s, 3H), 3.62-3.56 (m, 2H), 3.44-3.40 (m, 2H), 3.35-3.30 (m, 2H), 3.08 (s, 6H), 1.71-1.63 (m, 2H), 1.26-1.19 (m, 28H), 0.83 (t, J=7.0 Hz, 3H)

LRMS m/z calculated for C₅₁H₇₀N₇O₃⁺ 828.55, found 828.3 [M]⁺

HRMS m/z calculated for C₅₁H₇₀N₇O₃⁺ 828.5535, found 828.5543 [M]⁺

### Ic17 (E)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)octadecan-1-aminium

IR (cm⁻¹) 3269.34, 2929.87, 2858.51, 2160.27, 2017.54, 1978.97, 1670.35, 1637.56, 1523.76, 1436.97, 1388.75, 1265.3, 1197.79, 1172.72, 1120.64, 1060.85, 970.19, 827.46, 756.1, 719.45

¹H NMR (500 MHz, d⁶-DMSO) δ (ppm) 10.35 (1H, s), 9.97 (1H, s), 9.25 (1H, s), 8.54 (1H, s), 8.29-8.26 (1H, m), 8.03-7.99 (5H, m), 7.83-7.81 (2H, m), 7.77-7.74 (1H, m), 7.66-7.63 (3H, m), 7.34 (1H, s), 7.19 (2H, s), 7.17 (1H, s), 7.12 (1H, s), 7.07 (1H, s), 7.01 (1H, s), 6.97 (1H, s), 3.88 (3H, s), 3.83 (3H, s), 3.44-3.40 (2H, m), 3.34-3.28 (2H, m), 3.08 (6H, s), 1.67 (2H, bs), 1.26-1.21 (32H, m), 0.85-0.82 (3H, m)

HRMS m/z calculated for C₅₂H₇₂N₇O₃⁺ 842.5691, found 842.5683 [M]⁺

### Ic18 (E)-2-(2-methoxyethoxy)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)ethan-1-aminium

IR (cm⁻¹) 3298, 1637, 1116, 717

¹H NMR (500 MHz, DMSO-d₆) δ (ppm) 10.4 (s, 1H), 9.98 (s, 1H), 9.29 (d, J=1.8 Hz, 1H), 8.62 (s, 1H), 8.28 (t, J=5.7 Hz, 1H), 8.08 (d, J 17 Hz, 1H), 8.05-8.00 (m, 4H), 7.83-7.78 (m, 3H), 7.72-7.60 (m, 4H), 7.35 (d, J=1.4 Hz, 1H), 7.22 (d, J=1.4 Hz, 1H), 7.13 (d, J=1.4 Hz, 1H), 6.99 (d, J=1.4 Hz, 1H), 3.88 (s, 3H), 3.84 (s, 3H), 3.64-3.57 (m, 7H), 3.53-3.46 (m, 5H), 3.26 (s, 3H), 3.15 (s, 6H)

LRMS m/z calculated for C₃₉H₄₆N₇O₅⁺ 692.36, found 692.4 [M]⁺

HRMS m/z calculated for C₃₉H₄₆O₅N₇⁺ 692.3555, found 692.3555 [M]⁺

### Ic19 (E)-2-(2-(2-methoxyethoxy)ethoxy)-N,N-dimethyl-N-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)ethan-1-aminium

IR (cm⁻¹) 3263, 1643, 1116, 717

¹H NMR (500 MHz, DMSO-d₆) δ (ppm) 10.4 (s, 1H), 9.98 (s, 1H), 9.27 (d, J=2.0 Hz, 1H), 8.58 (s, 1H), 8.27 (t, J=5.5 Hz, 1H), 8.06-7.99 (m, 4H), 7.82 (d, J=8.1 Hz, 2H), 7.79-7.75 (m, 1H), 7.71-7.60 (m, 3H), 7.34 (d, J=1.5 Hz, 1H), 7.21 (d, J=1.5 Hz, 1H), 7.13 (d, J=1.5 Hz, 1H), 6.99 (d, J=1.5 Hz, 1H), 3.88 (s, 6H), 3.84 (s, 3H), 3.73 (t, J=5.8 Hz, 3H), 3.64-3.60 (m, 5H), 3.54-3.50 (m, 8H), 3.45-3.41 (m, 5H), 3.24 (s, 3H), 3.23 (s, 3H)

LRMS m/z calculated for C₄₁H₅₀N₇O₆⁺ 736.38, found 736.4 [M]⁺

HRMS m/z calculated for C₄₁H₅₀N₇O₆⁺ 736.3817, found 736.3820 [M]⁺

### Ic20 (E)-N-ethyl-N,N-dimethyl-3-(1-methyl-4-(1-methyl-4-(6-(3-(trifluoromethyl)styryl)nicotinamido)-1H-pyrrole-2-carboxamido)-1H-pyrrole-2-carboxamido)propan-1-aminium

IR (cm⁻¹) 3261.63, 3116.97, 3034.03, 2943.37, 2160.27, 1978.97, 1774.51, 1774.51, 1735.93, 1637.56, 1577.77, 1529.55, 1463.97, 1435.04, 1406.11, 1327.03, 1294.24, 1263.37, 1201.65, 1163.08, 1120.64, 1097.50, 1070.49, 1006.84

¹H NMR (500 MHz, DMSO-d₆) δ (ppm) 10.57 (s, 1H), 9.97 (s, 1H), 9.14 (s, 1H), 8.34 (dd, J=6.20 Hz, J=2.10 Hz, 1H), 8.14 (t, J=8.80 Hz, 1H), 8.09 (s, 1H), 8.04 (d, J=7.50 Hz, 1H), 7.91 (d, J=15.95 Hz, 1H), 7.76-7.66 (m, 3H), 7.61 (d, J=16.05 Hz, 1H), 7.36 (d, J=1.50 Hz, 1H), 7.19 (d, J=1.50 Hz, 1H), 7.13 (d, J=1.50 Hz, 1H), 6.96 (d, J=1.55 Hz, 1H), 3.89 (s, 3H), 3.83 (s, 3H), 3.34 (q, J=7.20 Hz, 2H), 3.30-3.24 (m, 4H), 3.00 (s, 6H), 1.94-1.87 (m, 2H), 1.24 (t, J=7.05 Hz, 3H)

LRMS m/z calculated for C₃₄H₃₉F₃N₇O₃⁺ 650.7 [M]⁺, found 650.3 [M]⁺

### Ic21 (E)-N-ethyl-3-(4-(4-(6-(4-fluoro-3-methoxystyryl)nicotinamido)-1-methyl-1H-pyrrole-2-carboxamido)-1-methyl-1H-pyrrole-2-carboxamido)-N,N-dimethylpropan-1-aminium

IR (cm⁻¹) 3292.49, 3105.39, 2322.29, 2166.06, 2050.33, 1978.97, 1654.92, 1637.56, 1587.42, 1514.12, 1460.11, 1436.97, 1406.11, 1261.45, 1197.79, 1120.64

¹H NMR (500 MHz, DMSO-d₆) δ (ppm) 10.52 (s, 1H), 9.97 (s, 1H), 9.10 (s, 1H), 8.31 (d, J=8.1 Hz, 1H), 8.14 (s, 1H), 7.79 (d, J=15.7 Hz, 1H), 7.69 (d, J=7.9 Hz, 1H), 7.56 (d, J=8.0 Hz, 1H), 7.43 (d, J=15.9 Hz, 1H), 7.36 (s, 1H), 7.30-7.25 (m, 2H), 7.20 (s, 1H), 7.12 (s, 1H), 6.96 (s, 1H), 3.94 (s, 3H), 3.90 (s, 3H), 3.83 (s, 3H), 3.42-3.22 (m, 6H), 3.00 (s, 6H), 1.91 (s, 2H), 1.25 (t, J=7.3 Hz, 3H).

LCMS m/z calculated for C₃₄H₄₁FN₇O₄ 630.32 [M], found 630.4 [M]+

### Ic22 (E)-3-(4-(4-(4-(2-(benzo[c][1,2,5]oxadiazol-5-yl)vinyl)benzamido)-1-methyl-1H-pyrrole-2-carboxamido)-1-methyl-1H-pyrrole-2-carboxamido)-N-ethyl-N,N-dimethylpropan-1-aminium

IR (cm⁻¹) 3284.77, 2943.37, 2164.13, 1978.97, 1685.79, 1641.42, 1577.77, 1535.34, 1463.97, 1436.97, 1402.25, 1261.45, 1199.72, 1172.72, 1122.57, 1006.84

¹H NMR (500 MHz, DMSO-d₆) δ (ppm) 10.38 (s, 1H), 9.96 (s, 1H), 8.13 (m, 4H), 8.03 (d, J=7.9 Hz, 2H), 7.83 (d, J=8.2 Hz, 2H), 7.71 (d, J=16.2 Hz, 1H), 7.62 (d, J=16.6 Hz, 1H), 7.35 (s, 1H), 7.19 (s, 1H), 7.13 (s, 1H), 6.96 (s, 1H), 3.89 (s, 3H), 3.83 (s, 3H), 3.4-3.2 (m, 6H, underneath water peak), 3.00 (s, 6H), 1.91 (m, 2H), 1.22 (m, 3H).

LCMS m/z calculated for C₃₄H₃₉N₈O₄⁺ 623.3 [M]⁺, found 623.3 [M]⁺ Ic23 (E)-N-ethyl-N,N-dimethyl-3-(1-methyl-4-(1-methyl-4-(4-(2-(quinolin-3-yl)vinyl)benzamido)-1H-pyrrole-2-carboxamido)-1H-pyrrole-2-carboxamido)propan-1-aminium

IR (cm⁻¹) 3284.77, 3124.68, 2162.2, 1978.97, 1637.56, 1577.77, 1560.41, 1533.41, 1508.33, 1498.69, 1465.9, 1436.97, 1404.18, 1301.95, 1259.52, 1201.65

¹H NMR (500 MHz, DMSO-d₆) δ (ppm) 10.35 (s, 1H), 9.94 (s, 1H), 9.26 (d, J=2.2 Hz, 1H), 8.55 (d, J=2.3 Hz, 1H), 8.12 (t, J=6.0 Hz, 1H), 8.05-7.95 (m, 4H), 7.84-7.72 (m, 3H), 7.71-7.58 (m, 3H), 7.34 (d, J=1.8 Hz, 1H), 7.18 (d, J=1.9 Hz, 1H), 7.12 (d, J=1.9 Hz, 1H), 6.95 (d, J=1.9 Hz, 1H), 3.88 (s, 3H), 3.82 (s, 3H), 3.33 (q, J=7.2 Hz, 2H), 3.29-3.23 (m, 4H), 2.98 (s, 6H), 1.93-1.86 (m, 2H), 1.23 (t, J=7.1 Hz, 3H).

LCMS m/z calculated for C₃₇H₄₂N₇O₃⁺ 632.3 [M]⁺, found 632.4 [M]+

### Id1 (E)-4-hexyl-4-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)morpholin-4-ium

IR (cm⁻¹) 3304, 3188, 2956, 2933, 2870, 2160, 1670, 1637, 1608, 1577, 1523, 1506, 1436, 1400, 1386, 1263, 1197, 1174, 1122, 1058, 1016, 1006, 970, 956,894, 866, 829

¹H NMR (500 MHz, DMSO-d₆) δ (ppm) 10.49 (s, 1H), 10.05 (s, 1H), 10.00 (s, 1H), 9.47 (s, 1H), 8.67 (d, J=8.9 Hz, 1H), 8.50 (d, J=8.2 Hz, 1H), 8.29 (t, J=7.6 Hz, 2H), 8.14-8.09 (m, 3H), 7.91-7.88 (m, 3H), 7.73 (d, J=16.7 Hz, 1H), 7.41 (s, 1H), 7.28 (s, 1H), 7.20 (s, 1H), 7.07 (s, 1H), 5.13 (t, J=6.64 Hz, 2H), 4.07 (d, J=10.59 Hz, 2H), 3.95 (s, 3H), 3.90 (s, 3H), 3.74 (t, J=8.31 Hz, 3H), 3.67-3.58 (m, 3H), 3.25-3.17 (m, 2H), 2.14-2.04 (m, 2H), 1.58-1.50 (m, 2H), 1.44-1.29 (m, 6H), 0.95 (t, J=6.92 Hz, 3H)

LRMS m/z calculated for C₄₂H₅₀N₇O₄⁺ 716.9, found 716.5 [M]+

### Id2 (E)-4-heptyl-4-(2-(1-methyl-4-(1-methyl-4-(4-(2-(quinolin-3-yl)vinyl)benzamido)-1H-pyrrole-2-carboxamido)-1H-pyrrole-2-carboxamido)ethyl)morpholin-4-ium

IR (cm⁻¹) 3290, 2929, 2864, 2160, 1637, 1577, 1521, 1433, 1400, 1382, 1263, 1195, 1172, 1122, 1056, 1006, 974, 894, 866, 827, 798

¹H NMR (500 MHz, DMSO-d₆) δ (ppm) 10.42 (s, 1H), 9.99 (s, 1H), 9.93 (s, 1H), 9.40 (s, 1H), 8.61 (d, J=9.4 Hz, 1H), 8.43 (d, J=8.4 Hz), 8.23 (t, J=7.3 Hz, 2H), 8.08-8.04 (m, 3H), 7.85-7.81 (m, 3H), 7.67 (d, J=17.0 Hz, 1H), 7.35 (s, 1H), 7.21 (s, 1H), 7.14 (s, 1H), 7.02 (s, 1H), 5.07 (t, J=8.4 Hz, 2H), 4.01 (d, J=12.9 Hz, 2H), 3.89 (s, 3H), 3.84 (s, 3H), 3.69-3.64 (m, 3H), 3.30-3.26 (m, 2H), 3.19-3.10 (m, 2H), 2.07-2.02 (m, 2H), 1.47-1.44 (m, 2H), 1.37-1.34 (m, 2H), 1.30-1.26 (m, 6H), 0.87 (t, J=6.9 Hz, 3H)

LRMS m/z calculated for C₄₃H₅₂N₇O₄⁺ 730.9, found 730.4 [M]⁺

### Characterisation Details of Compounds of the Invention Containing Amidine Groups (of Formula II)

### II1 (E)-N-(5-amino-5-iminopentyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3307, 1670, 1174, 839

¹H NMR (500 MHz, DMSO-d₆) δ (ppm) 10.4 (s, 1H), 9.93 (s, 1H), 9.27 (s, 1H), 8.86 (s, 2H), 8.57 (s, 1H), 8.43 (s, 2H), 8.05-8.01 (m, 4H), 7.82 (d, J=8.3 Hz, 2H), 7.78-7.75 (m, 1H), 7.68-7.63 (m, 3H), 7.34 (d, J=1.5 Hz, 1H), 7.17 (d, J=1.6 Hz, 1H), 7.12 (d, J=1.6 Hz, 1H), 6.92 (d, J=1.5 Hz, 1H), 3.88 (s, 3H), 3.81 (s, 3H), 3.21-3.18 (m, 2H), 2.41-2.38 (m, 2H), 1.66-1.60 (m, 2H), 1.54-1.48 (m, 2H)

LRMS m/z calculated for C₃₅H₃₆O₃N₈ 616.73 [M]⁺, found 617.8 [M+H]⁺

### 112 (E)-N-(5-((5-((5-amino-5-iminopentyl)carbamoyl)-1-methyl-1H-pyrrol-3-yl)carbamoyl)-1-methyl-1H-pyrrol-3-yl)-6-(4-(dimethylamino)styryl)nicotinamide

IR (cm⁻¹) 3053, 1170, 1124, 719

¹H NMR (500 MHz, DMSO-d₆) δ (ppm) 10.45 (s, 1H), 9.93 (s, 1H), 9.03 (m, 1H), 8.86 (s, 2H), 8.42 (s, 2H), 8.28-8.22 (m, 1H), 8.06-8.03 (m, 1H), 7.71 (d, J=15.9 Hz, 1H), 7.63 (d, J=8.2 Hz, 1H), 7.54 (d, J=8.9 Hz, 2H), 7.35-7.32 (m, 1H), 7.18-7.15 (m, 1H), 7.15-7.08 (m, 2H), 6.92-6.89 (m, 1H), 6.76 (d, J=8.9 Hz, 2H), 3.88 (s, 3H), 3.81 (s, 3H), 3.21-3.18 (m, 2H), 2.98 (s, 6H), 2.40-2.37 (m, 2H), 1.65-1.61 (m, 2H), 1.53-1.48 (m, 2H)

LRMS m/z calculated for C₃₃H₃₉N₉O₃ 609.74 [M], found 610.3 [M+H]⁺

### 113 (E)-N-(6-amino-6-iminohexyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3263, 1670, 1199, 719

¹H NMR (500 MHz, DMSO-d₆) δ (ppm) 10.35 (s, 1H), 9.92 (s, 1H), 9.29-9.25 (m, 1H), 8.84 (bs, 2H), 8.56 (s, 1H), 8.41 (bs, 2H), 8.04-8.00 (m, 5H), 7.83-7.74 (m, 3H), 7.68-7.62 (m, 3H), 7.36-7.32 (m, 1H), 7.18-7.14 (m, 1H), 7.13-7.10 (m, 1H), 6.92-6.89 (m, 1H), 3.88 (s, 3H), 3.81 (s, 3H), 3.20-3.17 (m, 2H), 2.36-2.35 (m, 2H), 1.67-1.60 (m, 2H), 1.55-1.48 (m, 2H), 1.35-1.30 (m, 2H)

LRMS m/z calculated for C₃₆H₃₉N₉O₃ 5630.75 [M], found 631.3 [M+H]⁺

### 114 (E)-N-(5-((5-((6-amino-6-iminohexyl)carbamoyl)-1-methyl-1H-pyrrol-3-yl)carbamoyl)-1-methyl-1H-pyrrol-3-yl)-6-(4-(dimethylamino)styryl)nicotinamide

IR (cm⁻¹) 3275, 1670, 1570, 1170, 833

¹H NMR (500 MHz, DMSO-d₆) δ (ppm) 10.47 (s, 1H), 9.93 (s, 1H), 9.04 (s, 1H), 8.85 (s, 2H), 8.43 (s, 2H), 8.29 (d, J=9.9 Hz, 1H), 8.00 (t, J=5.6 Hz, 1H), 7.77-7.66 (m, 2H), 7.54 (d, J=9.0 Hz, 2H), 7.36-7.32 (m, 1H), 7.16-7.10 (m, 3H), 6.91-6.88 (m, 1H), 6.77 (d, J=9.0 Hz, 2H), 3.88 (s, 3H), 3.80 (s, 3H), 3.19-3.16 (m, 2H), 2.98 (s, 6H), 2.37-2.35 (m, 2H), 1.68-1.58 (m, 2H), 1.56-1.48 (m, 2H), 1.36-1.28 (m, 2H)

LRMS m/z calculated for C₃₄H₄₁N₉O₃ 623.76 [M], found 624.4 [M+H]⁺

### Synthesis of Compounds of the Invention Containing NH Pyrroles

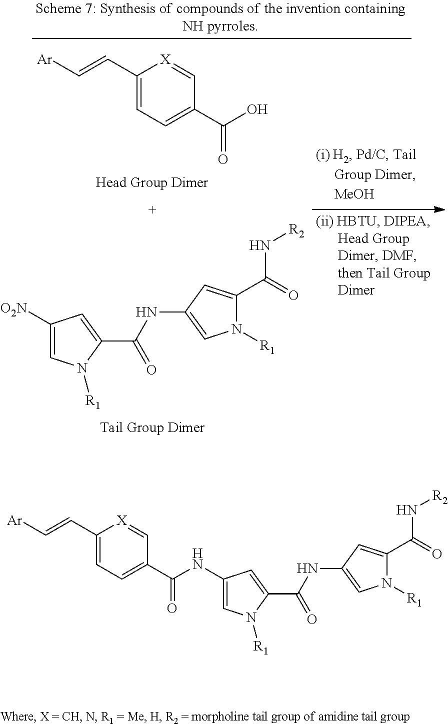

The appropriate tail group dimer (0.105 mmol, 1.5 eq.) in methanol (20 mL) was treated with Pd/C (10% w/w) and hydrogenated at room temperature overnight via a balloon. Following this, the reaction mixture was filtered through celite and the methanol removed by evaporation under reduced pressure. The resulting residue was then re-dissolved in DMF (1.0 mL).

The appropriate headgroup (0.070 mmol, 1 eq.) in DMF (1.0 mL) was treated with HBTU (40 mg, 0.105 mmol. 1.5 eq.) and DIPEA (1.5 eq.) and stirred for 30 min before combining with the DMF solution of the amine. The reaction mixture was stirred at room temperature overnight and then purified directly by HPLC and the appropriate fractions freeze dried (5-35% yield).

### IIIa1 (E)-N-(3-amino-3-iminopropyl)-4-(4-(4-(2-(benzo[c][1,2,5]oxadiazol-5-yl)vinyl)benzamido)-1H-pyrrole-2-carboxamido)-1-methyl-1H-pyrrole-2-carboxamide

IR (cm⁻¹) 719, 750, 796, 833, 966, 1008, 1122, 1180, 1267, 1288, 1313, 1363, 1525, 1662, 3240

¹H NMR (500 MHz, d⁶-DMSO) δ (ppm) 11.28 (1H, s), 10.37 (1H, s) 9.95 (1H, s), 8.90 (2H, s), 8.50 (2H, s), 8.23-8.21 (1H, t, J=6 Hz), 8.11 (3H, s), 8.03-8.02 (2H, m), 7.83-7.81 (2H, m), 7.72-7.60 (2H, m), 7.29 (1H, bs), 7.23 (1H, bs), 7.19 (1H, bs), 6.90 (1H, bs), 3.83 (3H, s), 3.53-3.50 (2H, m), 2.63-2.60 (2H, m)

LRMS m/z calculated for C₂₉H₂₇N₉O₄ 565.22 [M]⁺, found 566.3 [M+H]⁺

HRMS m/z calculated for C₂₉H₂₈N₉O₄ 566.2259 [M+H]⁺, found 566.2249 [M+H]⁺

### IIIa2 (E)-N-(5-((3-amino-3-iminopropyl)carbamoyl)-1H-pyrrol-3-yl)-1-methyl-4-(4-(2-(quinolin-3-yl)vinyl)benzamido)-1H-pyrrole-2-carboxamide

IR (cm⁻¹) 3269, 3145, 2160, 1676, 1608, 1577, 1560, 1541, 1521, 1481, 1408, 1386, 1323, 1298, 1267, 1195, 1182, 1116, 1072, 1053, 1039, 1014, 985, 972, 862, 846, 827, 819, 800, 792, 750, 736, 719, 690, 678, 657, 651, 638, 630, 617, 607

¹H NMR (500 MHz, de-DMSO) δ (ppm) 11.16 (1H, s), 10.35 (1H, s), 9.93 (1H, s), 9.26 (1H, s), 8.91 (2H, s), 8.54 (1H, s), 8.44 (2H, s), 8.22-8.20 (1H, t, J=6 Hz), 8.03-7.97 (5H, m), 7.82-7.81 (2H, m), 7.77-7.75 (2H, m), 7.64-7.60 (4H, m), 7.34 (1H, s), 7.15-7.12 (2H, m), 7.01 (1H, bs), 3.88 (3H, s) 3.56-3.53 (2H, m, underneath water peak), 2.63-2.61 (2H, m, underneath DMSO peak)

LRMS m/z calculated for C₃₂H₃₀N₈O₃ 574.24 [M]⁺, found 575.3 [M+H]⁺

HRMS m/z calculated for C₃₂H₃₁O₃N₈ 575.2514 [M+H]⁺, found 575.2507 [M+H]⁺

### IIIa3 (E)-N-(5-((3-amino-3-iminopropyl)carbamoyl)-1H-pyrrol-3-yl)-4-(4-(2-(benzo[c][1,2,5]oxadiazol-5-yl)vinyl)benzamido)-1-methyl-1H-pyrrole-2-carboxamide

IR (cm⁻¹) 3305, 1672, 1643, 1625, 1537, 1519, 1502, 1438, 1402, 1267, 1201, 1184, 1122, 1006, 952, 837, 798, 756, 721, 688, 669, 644, 624, 609

¹H NMR (500 MHz, d⁶-DMSO) δ (ppm) 11.16 (1H, s), 10.36, (1H, s), 9.93 (1H, s), 8.91 (2H, s), 8.45 (2H, s), 8.22-8.20 (1H, t, J=6 Hz), 8.10 (3H, s), 8.02-8.00 (2H, m), 7.82-7.80 (2H, m), 7.71-7.59 (2H, m), 7.34 (1H, s), 7.13-7.12 (2H, m), 7.10 (1H, s), 3.88 (3H, s), 3.56-3.52 (2H, m), 2.62-2.60 (2H, m)

LRMS m/z calculated for C₂₉H₂₇N₉O₄ 565.22 [M]⁺, found 566.3 [M+H]⁺

HRMS m/z calculated for C₂₉H₂₈O₄N₉ 566.2259 [M+H]⁺, found 566.2252 [M+H]⁺

### IIIa4 (E)-N-(3-amino-3-iminopropyl)-1-methyl-4-(4-(4-(2-(quinolin-3-yl)vinyl)benzamido)-1H-pyrrole-2-carboxamido)-1H-pyrrole-2-carboxamide

IR (cm⁻¹) 3265, 3080, 1629, 1527, 1404, 1388, 1361, 1340, 1288, 1263, 1193, 1184, 1124, 1014, 968, 894, 833, 798, 748, 719, 688, 655, 628

¹H NMR (500 MHz, d⁶-DMSO) δ (ppm) 11.27 (1H, s), 8.58 (1H, bs), 8.52 (2H, bs), 8.22 (1H, t, J=6 Hz), 8.04-8.00 (4H, m), 7.82-7.78 (3H, m), 7.66-7.63 (3H, m), 7.29 (1H, bs), 7.22 (1H, bs), 7.18 (1H, bs), 6.89 (1H, s), 3.82 (3H, s), 3.56-3.53 (2H, m, underneath water peak), 2.63-2.61 (2H, m, underneath DMSO peak)

LRMS m/z calculated for C₃₂H₃₀N₈O₃ 574.24 [M]⁺, found 575.3 [M+H]⁺

HRMS m/z calculated for C₃₂H₃₁N₈O₃ 575.2514 [M+H]⁺, found 575.2507 [M+H]⁺

### IIIa5 (E)-N-(5-((5-((3-amino-3-iminopropyl)carbamoyl)-1-methyl-1H-pyrrol-3-yl)carbamoyl)-1H-pyrrol-3-yl)-6-(4-(dimethylamino)styryl)nicotinamide

IR (cm⁻¹) 3253.91, 3035.96, 1629.85, 1525.69, 1433.11, 1365.6, 1315.45, 1284.59, 1176.58, 1124.5, 968.27, 900.76, 833.25, 798.53, 750.31, 719.45

¹H NMR (500 MHz, d⁶-DMSO) δ (ppm) 11.31 (1H, s), 10.45 (1H, s), 9.95 (1H, s), 9.04 (1H, s), 8.9 (2H, s), 8.52 (2H, s), 8.29-8.27 (1H, m), 8.22 (1H, t, J=Hz), 7.74 (1H, s), 7.71 (1H, s), 7.67-7.66 (1H, m), 7.54-7.53 (2H, d, J=Hz), 7.28 (1H, bs), 7.22 (1H, s), 7.16-7.09 (2H, m), 6.88 (1H, s), 6.76 (2H, d, J=Hz), 3.82 (3H, s), 3.52-3.49 (2H, m), 2.98 (6H, s), 2.63-2.61 (2H, m)

LRMS m/z calculated for C₃₀H₃₃N₉O₃ 567.27 [M]⁺, found 568.3 [M+H]⁺

HRMS m/z calculated for C₃₀H₃₄O₃N₉ 568.2779 [M+H]⁺, found 568.2771 [M+H]⁺

### IIIa6 (E)-N-(3-amino-3-iminopropyl)-4-(4-(4-(2-(quinolin-3-yl)vinyl)benzamido)-1H-pyrrole-2-carboxamido)-1H-pyrrole-2-carboxamide

IR (cm⁻¹) 719, 748, 798, 835, 964, 1124, 1190, 1290, 1398, 1629, 1670, 3248, 3332

¹H NMR (500 MHz, d⁶-DMSO) δ (ppm) 11.30 (1H, s), 11.18 (1H, s), 10.34 (1H, s), 9.94 (1H, s), 9.27 (1H, s), 8.93 (2H, s), 8.56-8.53 (3H, m), 8.26-8.24 (1H, m), 8.04-8.00 (4H, m), 7.82-7.81 (2H, m), 7.78-7.74 (1H, m), 7.69-7.60 (3H, m), 7.28 (1H, bs), 7.20-7.18 (2H, m), 6.92 (1H, bs), 3.57-3.53 (2H, m), 2.63-2.60 (2H, m)

LRMS m/z calculated for C₃₁H₂₈N₈O₃ 560.23 [M]⁺, found 561.3 [M+H]⁺

HRMS m/z calculated for C₃₁H₂₉O₃N₈ 561.2357 [M+H]⁺, found 561.2350 [M+H]⁺

### IIIa7 (E)-N-(5-((5-((3-amino-3-iminopropyl)carbamoyl)-1H-pyrrol-3-yl)carbamoyl)-1-methyl-1H-pyrrol-3-yl)-6-(4-(dimethylamino)styryl)nicotinamide

IR (cm⁻¹) 2980, 1660, 1631, 1564, 1529, 1436, 1369, 1321, 1286, 1255, 1180, 1168, 1151, 1124, 1064, 958, 945, 837, 796, 723

¹H NMR (500 MHz, de-DMSO) δ (ppm) 11.16 (1H, s), 10.43 (1H, s), 9.93 (1H, s), 9.03 (1H, s), 8.91 (2H, s), 8.45 (2H, s), 8.24-8.20 (2H, m), 7.72-7.68 (1H, m), 7.61-7.60 (1H, m), 7.54-7.52 (2H, m), 7.33 (1H, s), 7.12-7.09 (3H, m), 7.00 (1H, bs), 6.76-6.74 (2H, m), 3.88 (3H, s), 3.56-3.50 (2H, m), 2.97 (6H, s), 2.63-2.60 (2H, m)

LRMS m/z calculated for C₃₀H₃₃N₉O₃ 567.27 [M]⁺, found 568.4 [M+H]⁺

HRMS m/z calculated for C₃₀H₃₄O₃N₉ 568.2779 [M+H]⁺, found 568.2780 [M+H]⁺

### IIIa8 (E)-N-(3-amino-3-iminopropyl)-4-(4-(4-(2-(benzo[c][1,2,5]oxadiazol-5-yl)vinyl)benzamido)-1H-pyrrole-2-carboxamido)-1H-pyrrole-2-carboxamide

IR (cm⁻¹) 1670, 1610, 1591, 1519, 1404, 1263, 1201, 1184, 1126, 962, 837, 800, 746, 721

¹H NMR (500 MHz, de-DMSO) δ (ppm) 11.31 (1H, s), 11.19 (1H, s), 10.36 (1H, s), 9.94 (1H, s), 8.93 (2H, s), 8.54 92H, s), 8.26 (1H, t, J=6 Hz), 8.10 (3H, s), 8.06-8.01 (2H, m), 7.82-7.80 (2H, m), 7.71-7.59 (2H, m), 7.28 (1H, bs), 7.21-7.17 (2H, m), 6.92 (1H, bs), 3.56-3.53 (2H, m), 2.63-2.61 (2H, m)

LRMS m/z calculated for C₂₈H₂₅N₉O₄ 551.20 [M]⁺, found 552.0 [M+H]⁺

HRMS m/z calculated for C₂₉H₂₆O₄N₉ 552.2102 [M+H]⁺, found 552.2096 [M+H]⁺

### IIIa9 (E)-N-(5-((5-((3-amino-3-iminopropyl)carbamoyl)-1H-pyrrol-3-yl)carbamoyl)-1H-pyrrol-3-yl)-6-(4-(dimethylamino)styryl)nicotinamide

IR (cm⁻¹) 3240, 1629, 1521, 1406, 1363, 1315, 1288, 1182, 1126, 1070, 945, 835, 812, 798, 750, 721

¹H NMR (500 MHz, d⁶-DMSO) δ (ppm) 11.32 (1H, s), 11.18 (1H, s), 10.44 (1H, s), 9.94 (1H, s), 9.04 (1H, s), 8.92 (2H, s), 8.50 (2H, s), 8.27-8.23 (2H, m), 7.71 (1H, d, J=16.0 Hz), 7.64 (1H, d, J=8.2 Hz), 7.54 (2H, d, J=8.8 Hz), 7.27 (1H, s), 7.49 (1H, s), 7.16 (1H, s), 7.11 (1H, d, J=16.0 Hz), 6.91 (1H, bs), 6.76 (2H, d, J=8.8 Hz), 3.56-3.52 (2H, m, under the water peak), 2.97 (6H, s), 2.63-2.60 (2H, m)

LRMS m/z calculated for C₂₉H₃₁N₉O₃ 553.25 [M]⁺, found 554.0 [M+H]⁺

HRMS m/z calculated for C₂₉H₃₂N₉O₃ 554.2623 [M+H]⁺, found 554.2616 [M+H]⁺

### IIIb1 (E)-4-(4-(2-(benzo[c][1,2,5]oxadiazol-5-yl)vinyl)benzamido)-1-methyl-N-(5-((2-morpholinoethyl)carbamoyl)-1H-pyrrol-3-yl)-1H-pyrrole-2-carboxamide

IR (cm⁻¹) 3267, 1670, 1527, 1436, 1394, 1265, 1197, 1180, 1122, 831

¹H NMR (500 MHz, d⁶-DMSO) δ (ppm) 11.27 (1H, s), 10.38 (1H, s), 9.98 (1H, s), 9.56 (1H, bs), 8.27 (1H, m), 8.1 (2H, s), 8.02-8.00 (3H, m), 7.82-7.80 (3H, m), 7.33 (1H, s), 7.16-7.13 (1H, m), 7.12-7.10 (1H, m), 7.04-7.01 (1H, bs), 4.02-4.00 (2H, m), 3.88 (3H, s), 3.68-3.63 (2H, m), 3.59-3.55 (4H, m), 3.33 (6H, s), 3.14-3.08 (4H, m)

LRMS m/z calculated for C₃₂H₃₂N₈O₅ 608.25 [M]⁺, found 609.3.3 [M+H]⁺

HRMS m/z calculated for C₃₂H₃₃N₈O₅ 609.2568 [M+H]⁺, found 609.2562 [M+H]⁺

### IIIb2 (E)-6-(4-(dimethylamino)styryl)-N-(5-((1-methyl-5-((2-morpholinoethyl)carbamoyl)-1H-pyrrol-3-yl)carbamoyl)-1H-pyrrol-3-yl)nicotinamide

IR (cm⁻¹) 3253, 1666, 1641, 1529, 1438, 1402, 1288, 1197, 1182, 1124, 968, 831, 752

¹H NMR (500 MHz, d⁶-DMSO) δ (ppm) 11.27 (1H, s), 10.47 (1H, s), 9.96 (1H, s), 9.04 (1H, bs), 8.37-8.27 (2H, m), 7.75-7.66 (2H, m), 7.54-7.53 (2H, m), 7.33 (1H, s), 7.15-7.10 (3H, m), 7.03 (1H, bs), 6.77-6.75 (2H, m), 3.99-3.88 (2H, m), 3.67 (3H, s), 3.59-3.55 (6H, m), 3.28 (2H, s), 3.10-3.08 (3H, m)

LRMS m/z calculated for C₃₃H₃₈N₈O₄ 610.30 [M]⁺, found 611.4 [M+H]⁺

HRMS m/z calculated for C₃₃H₃₉N₈O₄ 611.3089 [M+H]⁺, found 611.3083 [M+H]⁺

### IIIb3 (E)-6-(4-(dimethylamino)styryl)-N-(1-methyl-5-((5-((2-morpholinoethyl)carbamoyl)-1H-pyrrol-3-yl)carbamoyl)-1H-pyrrol-3-yl)nicotinamide

IR (cm⁻¹) 3290, 1531, 1435, 1367, 1315, 1286, 1267, 1168, 1124, 1064, 904, 831, 796, 719, 704

¹H NMR (500 MHz, de-DMSO) δ (ppm) 11.27 (1H, s), 10.47 (1H, s), 9.96 (1H, s), 9.04 (1H, bs), 8.37-8.27 (2H, m), 7.75-7.66 (2H, m), 7.54-7.53 (2H, m), 7.33 (1H, s), 7.15-7.10 (3H, m), 7.03 (1H, bs), 6.77-6.75 (2H, m), 4.02-3.88 (2H, m), 3.88 (3H, s), 3.72-3.65 (2H, m), 3.60-3.55 (4H, m), 3.31-3.28 (2H, m), 3.14-3.06 (2H, m), 2.96 (6H, s)

LRMS m/z calculated for C₃₃H₃₈N₈O₄ 610.30 [M]⁺, found 611.4 [M+H]⁺

HRMS m/z calculated for C₃₃H₃₉N₈O₄ 611.3089 [M+H]⁺, found 611.3084 [M+H]⁺

### IIIb4 (E)-1-methyl-N-(5-((2-morpholinoethyl)carbamoyl)-1H-pyrrol-3-yl)-4-(4-(2-(quinolin-3-yl)vinyl)benzamido)-1H-pyrrole-2-carboxamide

IR (cm⁻¹) 3255, 1670, 1535, 1436, 1388, 1259, 1197, 1126, 968, 833, 719

¹H NMR (500 MHz, d⁶-DMSO) δ (ppm) 11.27 (1H, s), 10.35 (1H, s), 9.96 (1H, s), 9.27 (1H, s), 8.57 (1H, s), 8.29-8.27 (1H, m), 8.04-8.00 (4H, m), 7.83-7.81 (2H, m), 7.78-7.71 (1H, m), 7.66-7.60 (3H, m), 7.34 (1H, bs), 7.15 (1H, bs), 7.12-7.11 (1H, m), 7.04 (1H, bs), 4.03-4.00 (2H, m), 3.88 (3H, s), 3.68-3.63 (2H, m), 3.59-3.56 (4H, m), 3.31-3.26 (2H, m), 3.17-3.13 (2H, m)

LRMS m/z calculated for C₃₅H₃₅N₇O₄ 617.28 [M]⁺, found 618.3 [M+H]⁺

HRMS m/z calculated for C₃₅H₃₆N₇O₄ 618.2323 [M+H]⁺, found 618.2815 [M+H]⁺

### IIIb5 (E)-N-(2-morpholinoethyl)-4-(4-(4-(2-(quinolin-3-yl)vinyl)benzamido)-1H-pyrrole-2-carboxamido)-1H-pyrrole-2-carboxamide

IR (cm⁻¹) 2980, 1583, 1583, 1519, 1384, 1259, 1182, 1120, 1045, 966, 952, 831, 798, 719

¹H NMR (500 MHz, de-DMSO) δ (ppm) 11.30-11.28 (2H, m), 10.34 (1H, s), 9.96 (1H, s), 9.27 (1H, s), 8.56 (1H, s), 8.31-8.29 (1H, m), 8.04-8.00 (4H, m), 7.82-7.80 (2H, m), 7.77-7.74 (1H, m), 7.69-7.60 (3H, m), 7.28-7.27 (1H, m), 7.22-7.19 (2H, m), 6.96-6.95 (1H, m), 4.02-4.00 (2H, m), 3.68-3.57 (8H, m, underneath water peak), 3.15 (2H, bs)

LRMS m/z calculated for C₃₄H₃₃N₇O₄ 603.26 [M]⁺, found 604.3 [M+H]⁺

HRMS m/z calculated for C₃₄H₃₄O₄N₇ 604.2667 [M+H]⁺, found 604.2658 [M+H]⁺

### IIIb6 (E)-4-(4-(2-(benzo[c][1,2,5]oxadiazol-5-yl)vinyl)benzamido)-N-(5-((2-morpholinoethyl)carbamoyl)-1H-pyrrol-3-yl)-1H-pyrrole-2-carboxamide

IR (cm⁻¹) 3236, 1629, 1591, 1533, 1400, 1284, 1199, 1184, 1128, 879, 831, 800, 761, 719

¹H NMR (500 MHz, d⁶-DMSO) δ (ppm) 11.30 (2H, bs), 10.35 (1H, s), 9.96 (1H, s), 9.62 (1H, s), 8.30 (1H, s), 8.1 (4H, s), 8.02-8.01 (2H, m), 7.94-7.88 (1H, m), 7.82-7.80 (2H, m), 7.71-7.68 (1H, m), 7.63-7.59 (1H, m), 7.27 (1H, bs), 7.21-7.17 (2H, m), 6.95 (1H, m), 4.02-4.00 (2H, m), 3.68-3.57 (8H, m), 3.15 (2H, bs)

LRMS m/z calculated for C₃₁H₃₀N₈O₅ 594.23 [M]⁺, found 595.3 [M+H]⁺

HRMS m/z calculated for C₃₁H₃₁O₅N₈ 595.2412 [M+H]⁺, found 595.2403 [M+H]⁺

### IIIb7 (E)-6-(4-(dimethylamino)styryl)-N-(5-((5-((2-morpholinoethyl)carbamoyl)-1H-pyrrol-3-yl)carbamoyl)-1H-pyrrol-3-yl)nicotinamide

IR (cm⁻¹) 1529, 1436, 1369, 1321, 1286, 1255, 1180, 1168, 1151, 1124, 1064, 958, 945, 837, 796, 723

¹H NMR (500 MHz, d⁶-DMSO) δ 11.36 (1H, s), 11.29 (1H, s), 10.52 (1H, s), 9.98 (1H, s), 9.06 (1H, s), 8.39-8.37 (1H, m), 8.32 (1H, t, J=5.4 Hz), 7.80-7.77 (2H, m), 7.57, 7.54 (2H, m), 7.28 (1H, s), 7.11 (1H, s), 7.18-7.15 (2H, m), 6.94 (1H, s), 6.780-6.77 (2H, m), 4.03-3.99 (2H, m), 3.66 (2H, t, J=12 Hz), 3.60-3.57 (4H, m), 3.30-3.28 (2H, m), 3.15-3.12 (2H, m), 2.99 (6H, s)

LRMS m/z calculated for C₃₂H₃₆N₈O₄ 596.29 [M]⁺, found 597.4 [M+H]⁺

HRMS m/z calculated for C₃₂H₃₇O₄N₈ 597.2932 [M+H]⁺, found 597.2929 [M+H]⁺

### IIIb8 (E)-1-methyl-N-(2-morpholinoethyl)-4-(4-(4-(2-(quinolin-3-yl)vinyl)benzamido)-1H-pyrrole-2-carboxamido)-1H-pyrrole-2-carboxamide

IR (cm⁻¹) 3253, 1670, 1643, 1583, 1529, 1438, 1402, 1286, 1265, 1195, 1178, 1120, 1055, 1016, 968, 862, 831, 798, 748, 719

¹H NMR (500 MHz, d⁶-DMSO) δ (ppm) 11.28 (1H, s), 10.35 (1H, s), 9.97 (1H, s), 9.63 (1H, bs), 9.28 (1H, s), 8.59 (1H, s), 8.25 (1H, t, J=6 Hz), 8.05-8.01 (4H, m), 7.82-7.76 (3H, m), 7.67-7.60 (3H, m), 7.29 (1H, bs), 7.25 (1H, bs), 7.19 (1H, bs), 6.94 (1H, s), 4.02-4.00 (2H, m), 3.84 (3H, s), 3.75-3.44 (6H, m), 3.29-3.27 (2H, m), 3.19-3.08 (2H, m)

LRMS m/z calculated for C₃₅H₃₅N₇O₄ 617.28 [M]⁺, found 618.3 [M+H]⁺

HRMS m/z calculated for C₃₅H₃₆N₇O₄ 618.2823 [M+H]⁺, found 618.2817 [M+H]⁺

### IIIb9 (E)-4-(4-(4-(2-(benzo[c][1,2,5]oxadiazol-5-yl)vinyl)benzamido)-1H-pyrrole-2-carboxamido)-1-methyl-N-(2-morpholinoethyl)-1H-pyrrole-2-carboxamide

IR (cm⁻¹) 3253, 1666, 1641, 1529, 1438, 1402, 1288, 1197, 1182, 1124, 968, 831, 752

¹H NMR (500 MHz, de-DMSO) δ (ppm) 11.28 (1H, s), 10.36 (1H, s), 9.97 (1H, s), 9.55 (1H, bs), 8.24 (1H, bs), 8.10 (3H, s), 8.02-8.01 (2H, m), 7.82-7.80 (2H, m), 7.71-7.59 (2H, m), 7.28-7.19 (3H, m), 6.93 (1H, s), 4.03-4.00 (2H, m), 3.84 (3H, s), 3.68-3.63 (2H, m), 3.57-3.53 (6H, m), 3.16-3.13 (2H, m)

LRMS m/z calculated for C₃₂H₃₂N₈O₅ 608.25 [M]⁺, found 609.3 [M+H]⁺

HRMS m/z calculated for C₃₂H₃₃N₈O₅ 609.2568 [M+H]⁺, found 609.2560 [M+H]⁺

### Synthesis of Compounds of the Invention of the ‘Amide’ Set

Firstly, the appropriate ‘tetramer ester’ is synthesised through an amide coupling between a head group dimer and a dimer ester. Following this, the ‘tetramer acid’ is formed via hydrolysis. Finally, compounds of the invention are formed via amide coupling of the appropriate amine ‘tail group’ to the tetramer acid.

It should be noted that the final amide coupling has been carried out at a standard scale to afford enough compound for purification and isolation from the reaction mixture, and also a near quantitative 96-well plate format that can be used for directly for biological evaluation without purification. The characterisation entry for compounds synthesised via a 96-well plate method are indicated.

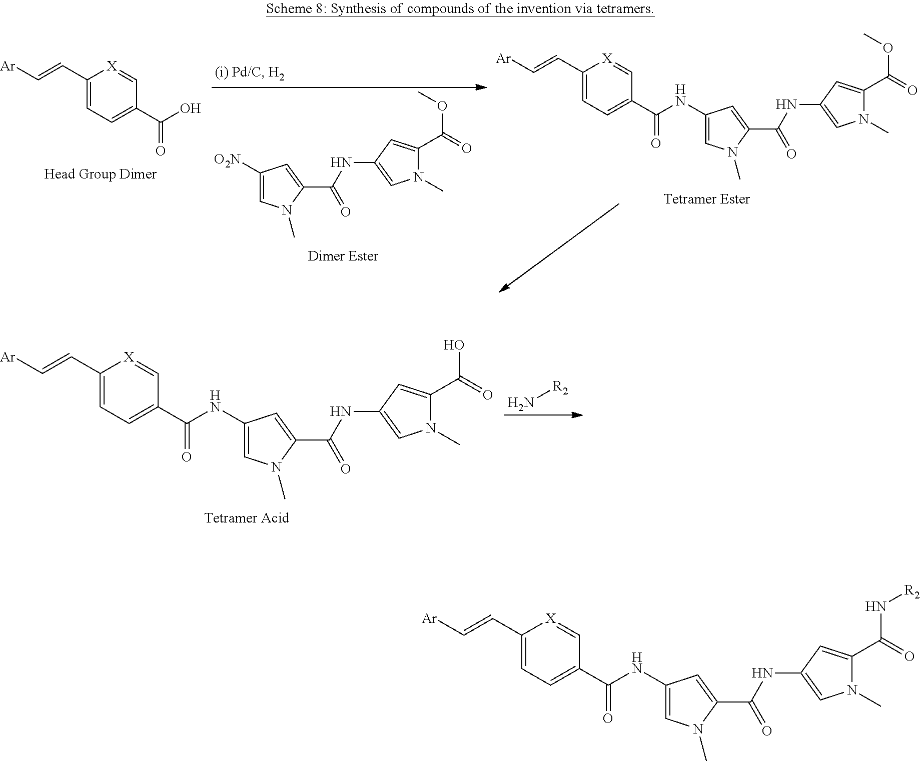

### Synthesis Details of Tetramer Esters

Methyl-1-methyl-4-(1-methyl-4-nitro-1H-pyrrole-2-carboxamido)-1H-pyrrole-2-carboxylate (0.300 g, 1.5 eq, 0.98 mmol) was added to a round bottomed flask along with palladium on carbon (0.150 g) and a stirrer bar. The reactants were wetted with a small quantity of ethyl acetate (to prevent ignition) then dissolved in methanol. The solution was hydrogenated using hydrogen gas via a balloon and left to stir for 3 h. Simultaneously, the appropriate head group dimer (0.65 mmol) was suspended in thionyl chloride (2 mL) and refluxed at 75° C. with stirring for 5 hours. The excess thionyl chloride was removed by evaporation under reduced pressure. The resulting acid chloride was then dissolved in DCM (5 mL). After the hydrogenation was complete, the mixture containing the newly formed amine was filtered through celite, and the methanol removed by evaporation under reduced pressure. The amine was then dissolved in DCM (10 mL) along with triethylamine (0.41 mL, 2.16 mmol) and cooled to −20° C. The acid chloride solution was slowly added to the cooled amine solution. The reaction was then allowed to warm-up to room temperature and stirred overnight. The solvent was removed in vacuo then triturated in sodium bicarbonate solution to remove any excess acid. Clean product was obtained after drying (˜70% yield).

### Synthesis Details of Tetramer Acids

The appropriate tetramer ester (0.73 mmol) was dissolved in methanol (20 ml). NaOH (10.95 mmol, 15 eq, 0.443 mg) was dissolved in water (20 ml) then added and heated to reflux (˜100° C., 3 h). The methanol removed by evaporation under reduced pressure and to the remaining solution HCl (1 mol) was added dropwise until the desired compound precipitated, which was then isolated by filtration (˜50% yield)

### methyl (E)-1-methyl-4-(1-methyl-4-(4-(2-(quinolin-3-yl)vinyl)benzamido)-1H-pyrrole-2-carboxamido)-1H-pyrrole-2-carboxylate

IR (cm⁻¹) 3275.13, 3207.62, 2951.09, 2922.16, 2852.72, 2158.35, 1631.78, 1606.7, 1566.2, 1556.55, 1504.48, 1435.04, 1386.82, 1338.6, 1278.81, 1259.52, 1201.65, 1155.36, 1111, 1056.99, 1039.63, 1016.49, 1006.84, 966.34

¹H NMR (500 MHz, DMSO-d₆) δ (ppm) 10.388 (s, 1H), 9.99 (s, 1H), 9.269 (d, 1H, J=2.39 Hz), 8.55 (d, 1H, J=1.89 Hz), 8.04 (dd, 1H), 8.03 (d, 2H), 8.00 (dd, 1H), 7.82 (d, 2H, J=8.67 Hz), 7.76 (td, 1H, J=8.19 Hz), 7.68 (d, 1H, J=17.40 Hz), 7.77 (td, 1H), 7.63 (d, 1H, J=16.35 Hz), 7.49 (d, 1H, J=1.72 Hz), 7.37 (d, 1H, J=2.12 Hz), 7.14 (d, 1H, J=2.23 Hz), 6.93 (d, 1H, J=), 3.89 (s, 3H), 3.86 (s, 3H), 3.75 (s, 3H)

LRMS m/z calculated for C₃₁H₂₇N₅O₄ 533.21 [M]⁺, found 534.3 [M+H]⁺

### methyl (E)-4-(4-(6-(4-(dimethylamino)styryl)nicotinamido)-1-methyl-1H-pyrrole-2-carboxamido)-1-methyl-1H-pyrrole-2-carboxylate

¹H NMR (500 MHz, DMSO-d₆) δ (ppm) 10.71 (s, 1H), 10.03 (s, 1H), 9.13 (d, J=1.93 Hz, 1H), 8.54 (d, J=8.53 Hz, 1H), 7.95 (m, 2H), 7.57 (d, J=8.78 Hz, 2H), 7.49 (d, J=1.98 Hz 1H), 7.39 (d, J=1.71 Hz, 1H), 7.23 (d, J=16.00 Hz, 1H), 7.15 (d, J=1.76 Hz, 1H), 6.94 (d, J=1.93 Hz, 1H), 6.84 (d, J=8.28 Hz, 2H), 3.89 (s, 3H), 3.29 (s, 3H), 3.71 (s, 3H), 3.02 (s, 6H).

LRMS m/z calculated for C₂₉H₃₀N₆O₄ 526.23 [M]⁺, found 527 [M+H]⁺

### (E)-1-methyl-4-(1-methyl-4-(4-(2-(quinolin-3-yl)vinyl)benzamido)-1H-pyrrole-2-carboxamido)-1H-pyrrole-2-carboxylic acid

IR (cm⁻¹) 3255.84, 2953.02, 2922.16, 2852.72, 2588.47, 1637.56, 1633.71, 1568.13, 1537.27, 1435.04, 1384.89, 1321.24, 1253.73, 1242.16, 1203.58, 1151.50, 1111.00, 1056.99, 1035.77, 1004.91, 966.34

¹H NMR (500 MHz, DMSO-d₆) δ (ppm) ˜12.2 (br, 1H), 10.41 (s, 1H), 9.96 (d, 1H, J=1.65 Hz), 9.29 (d, 1H), 8.20 (dd, 1H, J=8.46 Hz), 8.16 (dd, 1H, J=8.03 Hz), 8.05 (d, 2H, J=8.37 Hz), 7.93 (td, 1H, J=8.04 Hz), 7.82 (d, 2H, J=8.146 Hz), 7.80 (td, 1H), 7.77 (d, 1H), 7.67 (d, 1H, J=16.5 Hz), 7.44 (d, 1H, J=2.46 Hz), 7.37 (d, 1H, J=1.69 Hz), 7.134 (d, 1H, J=2.08 Hz), 6.87 (d, 1H, J=2.46 Hz) 3.89 (s, 3H), 3.840 (s, 3H).

LRMS m/z calculated for C₃₀H₂₅N₅O₄ 519.19 [M]⁺, found 520.2 [M+H]⁺

### (E)-4-(4-(6-(4-(dimethylamino)styryl)nicotinamido)-1-methyl-1H-pyrrole-2-carboxamido)-1-methyl-1H-pyrrole-2-carboxylic acid

IR (cm⁻¹) 3255.84, 2953.02, 2920.23, 2852.72, 1629.85, 1570.06, 1521.84, 1436.97, 1400.32, 1363.67, 1278.81, 1261.45, 1240.23, 1205.51, 1182.36, 1122.57, 1091.71, 1058.92, 1002.98, 968.27, 945.12, 893.04, 867.97, 804.32, 781.17, 752.24, 721.38.

¹H NMR (500 MHz, DMSO-d₆) δ (ppm) 10.46 (s, 1H), 9.95 (s, 1H), 9.05 (d, J=2.04 Hz, 1H), 8.25 (dd, J=8.08 Hz, 1.93 Hz, 1H), 7.72 (d, 15.72 Hz. 1H), 7.62 (d, J=8.29 Hz, 1H), 7.55 (d, 8.84 Hz, 2H), 7.44 (d, J=1.85 Hz, 1H), 7.36 (d, J=1.53 Hz, 1H), 7.12 (m, 2H), 6.87 (d, J=1.83 Hz, 1H), 6.77 (d, J=8.85 Hz, 2H), 3.89 (s, 3H), 3.84 (s, 3H), 2.96 (s, 6H).

LRMS m/z calculated for C₂₈H₂₆N₆O₄ 512.22 [M]⁻, found 511 [M−H]⁻

### Synthesis Details of Compounds of the ‘Amide’ Set

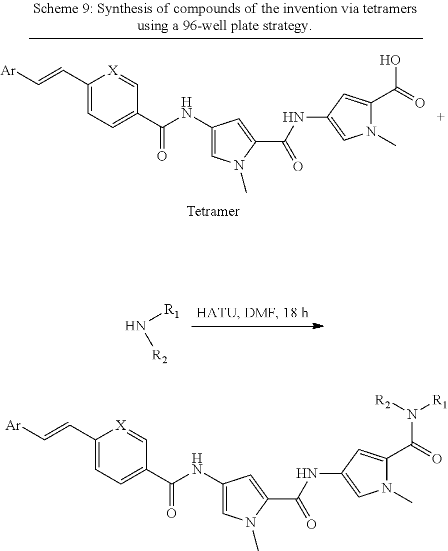

### Standard Procedure

The appropriate tetramer carboxylic acid (0.058 mmol), was dissolved in DMF (1 mL) along with HATU (2 eq, 0.116 mmol, 43.8 mg), and DIPEA (2 eq, 20 μL, 0.116 mmol) were stirred at room temperature for 10 minutes. The appropriate amine (2 eq, 0.116 mmol) was added and the reaction was stirred at room temperature. After completion the reaction was purified by HPLC and the fractions containing the desired compound were freeze dried.

### 96 Well Plate Procedure

Amine stock solutions of 60 mM were prepared in 100 uL DMF. The appropriate tetramer acid was dissolved in DMF to give a 25 mM stock solution. HATU was dissolved in DMF to yield a 60 mM stock solution. DIPEA was also prepared to 60 mM stock solution in DMF. Appropriate volumes of the HATU and DIPEA stock solutions were combined to give a single stock solution of 30 mM in both HATU and DIPEA. This solution was then combined with the appropriate volume of tetramer acid stock solution to yield a solution of HATU (15 mM), DIPEA (15 mM) and tetramer acid (12.5 mM). 80 μL of the HATU, DIPEA and tetramer acid solution was added to the wells of a 96-well plate. Added to this was 20 μL of amine (60 mM stock solution) to yield a final solution containing 10 mM tetramer acid, 12 mM HATU, 12 mM DIPEA and 12 mM amine. The plate containing the resulting solutions was left to react via amide coupling for 48 hrs at room temperature, without stirring. LCMS analysis, after 48 hrs confirmed the near quantitative conversion to final product, resulting in a solution containing the appropriate compound at 10 mM.

### Characterisation Details of Compounds of the ‘Amide’ Set

### IVa1 (E)-N-(5-carbamoyl-1-methyl-1H-pyrrol-3-yl)-1-methyl-4-(4-(2-(quinolin-3-yl)vinyl)benzamido)-1H-pyrrole-2-carboxamide

IR (cm⁻¹) 3261, 1651, 1568, 1436, 1402, 1197, 1128, 968

¹H NMR (500 MHz, DMSO-d₆) δ (ppm) 10.36 (s, 1H), 9.94 (s, 1H), 9.27 (d, 1H, J=2.07 Hz), 8.56 (d, 1H, J=1.98 Hz), 8.033 (m, 4H), 7.83 (d, 2H, J=8.5 Hz), 7.77 (td, 1H, J=1.35 Hz), 7.68 (d, 1H, J=16.86 Hz), 7.65 (td, 1H), 7.64 (d, 1H, J=16.48 Hz), 7.36 (d, 1H, J=1.67 Hz), 7.24 (d, 1H, J=1.67 Hz), 7.11 (d, 1H, J=1.83 Hz), 6.87 (d, 1H, J=1.83 Hz), 3.89 (s, 3H), 3.81 (s, 3H)

N.B Primary amide potentially exchanging and so cannot elucidate on NMR spectrum

LRMS m/z calculated for C₃₀H₂₆N₆O₃ 518.21 [M], found 519.3 [M+H]⁺

HRMS m/z calculated for C₃₀H₂₇N₆O₃ 519.2133 [M+H]⁺, found 591.2139 [M+H]⁺

### IVa2 (E)-N,1-dimethyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3030, 1670, 1627, 1527, 1436, 1400, 1271, 1197, 1126, 970

¹H NMR (500 MHz, DMSO-d₆) δ (ppm) 10.36 (s, 1H), 9.96 (s, 1H), 9.27 (d, 1H, J=2.07 Hz), 8.56 (d, 1H, J=1.62 Hz), 8.033 (m, 4H), 7.94 (t, 1H, J=4.57 Hz), 7.83 (d, 2H, J=8.4 Hz), 7.77 (td, 1H, J=1.06 Hz), 7.68 (d, 1H, J=16.78 Hz), 7.65 (td, 1H), 7.62 (d, 1H, J=16.63 Hz), 7.36 (d, 1H, J=1.71 Hz), 7.22 (d, 1H, J=1.70 Hz), 7.11 (d, 1H, J=1.83 Hz), 6.83 (d, 1H, J=1.77 Hz), 3.89 (s, 3H), 3.81 (s, 3H), 2.707 (d, 3H, J=4.57 Hz)

LRMS m/z calculated for C₃₁H₂₈N₆O₃ 532.22 [M], found 533.3 [M+H]⁺

HRMS m/z calculated for C₃₁H₂₉N₆O₃ 533.2990 [M+H]⁺, found 533.2296 [M+H]⁺

### IVa3 (E)-N-(2-cyclohexylethyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3267, 2922, 2848, 1637, 1529, 1435, 1402, 1261, 1195, 1130, 964

¹H NMR (500 MHz, DMSO-d₆) δ (ppm) 10.37 (s, 1H), 9.94 (s, 1H), 9.30 (d, 1H, J=2.22 Hz), 8.62 (d, 1H, J=1.83 Hz), 8.057 (dd, 1H, J=8.69 Hz), 8.04 (dd, 1H, J=5.64 Hz), 8.027 (d, 2H, J=8.23 Hz), 7.96 (t, 1H, J=5.34 Hz), 7.83 (d, 2H, J=8.39 Hz), 7.79 (td, 1H, J=8.23 Hz), 7.69 (d, 1H, J=16.47 Hz), 7.67 (td, 1H), 7.63 (d, 1H, J=16.78 Hz), 7.36 (d, 1H, J=1.98 Hz), 7.21 (d, 1H, J=2.13 Hz), 7.11 (d, 1H, J=1.63 Hz), 6.86 (d, 1H, J=1.83 Hz), 3.89 (s, 3H), 3.81 (s, 3H), 3.20 (q, 2H, J=6.71 Hz), 1.67 (m, 8H), 1.39 (q, 2H, J=6.86 Hz), 0.91 (m, 2H)

LRMS m/z calculated for C₃₈H₄₀N₆O₃ 628.32 [M], found 629.4 [M+H]⁺

HRMS m/z calculated for C₃₈H₄₁N₆O₃ 629.3228 [M+H]⁺, found 629.3235 [M+H]⁺

### IVa4 (E)-N-isopropyl-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 1676, 1629, 1521, 1436, 1400, 1261, 1201, 1128

¹H NMR (500 MHz, DMSO-d₆) δ (ppm) 10.36 (s, 1H), 9.91 (s, 1H), 9.29 (d, 1H, J=2.07 Hz), 8.60 (d, 1H, J=1.59 Hz), 8.052 (dd, 1H, J=8.39 Hz), 8.03 (dd, 1H), 8.03 (d, 2H, J=8.39 Hz), 7.83 (d, 2H, J=8.39 Hz), 7.78 (td, 1H, J=1.22 Hz), 7.69 (d, 1H, J=16.78 Hz), 7.66 (td, 1H), 7.63 (d, 1H, J=16.48 Hz), 7.36 (d, 1H, J=1.67 Hz), 7.16 (d, 1H, J=1.53 Hz), 7.10 (d, 1H, J=1.67 Hz), 6.92 (d, 1H, J=1.52 Hz), 3.89 (s, 3H), 3.81 (s, 3H), 1.15 (s, 3H), 1.13 (s, 3H)

LRMS m/z calculated for C₃₃H₃₂N₆O₃ 560.25 [M], found 561.3 [M+H]⁺

HRMS m/z calculated for C₃₃H₃₃N₆O₃ 561.2603 [M+H]⁺, found 561.2609 [M+H]⁺

### IVa5 (E)-N-butyl-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3280.92, 2956.87, 2933.73, 2162.2, 1670.35 1635.64, 1608.63, 1527.62, 1463.97, 1435.04, 1402.25, 1263.37, 1199.72, 1130.29, 1060.85, 1014.56, 964.41, 894.97, 831.32, 798.53, 771.53

¹H NMR (500 MHz, DMSO-d₆) δ (ppm) 10.36 (s, 1H), 9.94 (s, 1H), 9.28 (d, 1H, J=2.13 Hz), 8.57 (d, 1H), 8.06-8.00 (m, 4H), 7.98 (t, 1H, J=5.49 Hz), 7.83 (d, 2H, J=8.23 hz), 7.77 (td, 1H, J=8.10 Hz), 7.69 (d, 1H, J=16.6 Hz), 7.65 (td, 1H), 7.63 (d, 1H, J=16.32 Hz), 7.36 (d, 1H, J=1.37 Hz), 7.19 (d, 1H, J=1.83 Hz), 7.11 (d, 1H, J=1.67 Hz), 6.87 (d, 1H, J=1.67 Hz), 3.89 (s, 3H), 3.81 (s, 3H), 3.18 (q, 2H, J=6.86 Hz), 1.48 (m, 2H, J=7.01 Hz), 1.32 (m, 2H, J=7.66 Hz), 0.91 (t, 2H, J=7.01 Hz)

LRMS m/z calculated for C₃₄H₃₄N₆O₃ 574.27 [M], found 575.4 [M+H]⁺

### IVa6 (E)-N-ethyl-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3155, 2960, 1633, 1435, 1259, 1184, 1087, 1014, 796

¹H NMR (500 MHz, DMSO-d₆) δ (ppm) 10.37 (s, 1H), 9.94 (s, 1H), 9.29 (d, 1H, J=2.07 Hz), 8.60 (d, 1H, J=1.37 Hz), 8.056 (dd, 1H, J=8.39), 8.031 (dd, 1H), 8.031 (d, 2H, J=8.39), 8.00 (t, 1H, J=), 7.83 (d, 2H, J=8.39 Hz), 7.78 (td, 1H, J=1.22 Hz), 7.69 (d, 1H, J=16.78 Hz), 7.66 (td, 1H), 7.63 (d, 1H, J=16.48 Hz), 7.36 (d, 1H, J=1.67 Hz), 7.19 (d, 1H, J=1.53 Hz), 7.11 (d, 1H, J=1.67 Hz), 6.87 (d, 1H, J=1.52 Hz), 3.89 (s, 3H), 3.82 (s, 3H), 3.49 (d, 2H, J=11.23), 3.31 (t, 2H, J=11.73 Hz), 3.09 (q, 2H, J=12.35 Hz), 2.86 (s, 3H)

LRMS m/z calculated for C₃₂H₃₀N₆O₃ 546.24 [M], found 547.3 [M+H]⁺

### IVa7 (E)-N-(2-(2-(2-hydroxyethoxy)ethoxy)ethyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3321, 2858, 1633, 1537, 1435, 1404, 1263, 1199, 1118, 1062

¹H NMR (500 MHz, DMSO-d₆) δ (ppm) 10.36 (s, 1H), 9.95 (s, 1H), 9.30 (d, 1H, J=1.52 Hz), 8.62 (d, 1H, J=2.28 Hz), 8.06 (dd, 1H, J=8.39 Hz), 8.04 (dd, 1H), 8.02 (d, 2H, J=8.23 Hz), 7.83 (d, 2H, J=7.98 Hz), 7.79 (td, 1H, J=8.20 Hz), 7.70 (d, 1H, J=16.46 Hz), 7.67 (td, 1H), 7.63 (d, 1H, J=16.51 Hz), 7.36 (d, 1H, J=1.22 Hz), 7.22 (d, 1H), 7.11 (d, 1H), 6.88 (d, 1H), 3.89 (s, 3H), 3.81 (s, 3H), 3.54 (s, 1H), 3.50 (m, 2H), 3.43 (m, 6H), 3.34 (q, 4H)

LRMS m/z calculated for C₃₆H₃₈N₆O₆ 650.29 [M], found 651.3 [M+H]⁺

### IVa8 (E)-N-(2-(2-(2-methoxyethoxy)ethoxy)ethyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3282, 2866, 1641, 1527, 1463, 1435, 1382, 1259, 1199, 1112, 966

¹H NMR (500 MHz, DMSO-d₆) δ (ppm) 10.36 (s, 1H), 9.95 (s, 1H), 9.30 (d, 1H, J=1.91 Hz), 8.02 (d, 1H, J=2.13 Hz), 8.0 (dd, 1H, J=8.69 Hz), 8.03 (dd, 1H), 8.02 (d, 2H, J=8.39 Hz), 7.83 (d, 2H, J=8.23 Hz), 7.79 (td, 1H, J=8.23 Hz), 7.70 (d, 1H, J=16.42 Hz), 7.67 (td, 1H), 7.63 (d, 1H, J=16.78 Hz), 7.36 (d, 1H, J=1.52 Hz), 7.22 (d, 1H, J=1.95 Hz), 7.11 (d, 1H, J=1.67 Hz), 6.88 (d, 1H, J=2.25 Hz), 3.89 (s, 3H), 3.82 (s, 3H), 3.54 (m, 2H), 3.34 (m, 6H), 3.24 (m, 7H)

LRMS m/z calculated for C₃₇H₄₀N₆O₆ 644.30 [M], found 645.3 [M+H]⁺

### IVa9 (E)-N,N-bis(3-(dimethylamino)propyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3037, 2723, 1672, 1435, 1390, 1182, 1132, 837

¹H NMR (500 MHz, DMSO-d₆) δ (ppm) 10.36 (s, 1H), 9.92 (s, 1H), 9.28 (d, 1H, J=2.05 Hz), 8.56 (d, 1H, J=1.52 Hz), 8.05 (dd, 1H, J=8.54 Hz), 8.01 (dd, 1H), 8.01 (d, 2H, J=8.23 Hz), 7.78 (d, 2H, J=8.57 Hz), 7.77 (td, 1H, J=8.64 Hz), 7.69 (d, 1H, J=16.81 Hz), 7.65 (td, 1H), 7.62 (d, 1H, J=16.47 Hz), 7.32 (d, 1H, J=2.13 Hz), 7.24 (d, 1H, J=1.54 Hz), 7.16 (d, 1H, J=1.83 Hz), 6.61 (d, 1H, J=1.67 Hz), 3.89 (s, 3H), 3.65 (s, 3H), 3.52 (t, 4H, J=6.10 Hz), 3.05 (m, 4H, J=10.07 Hz), 2.83 (s, 6H), 2.82 (s, 6H), 1.95 (m, 4H, J=8.69 Hz)

LRMS (ES+APCI) m/z calculated for C₄₀H₄₈N₈O₃ 688.88 [M], found 689.4; [M+H]⁺

### IVa10 N—((E)-3,7-dimethylocta-2,6-dien-1-yl)-1-methyl-4-(1-methyl-4-(4-((E)-2-(quinolin-3-yl)vinyl)benzamido)-1H-pyrrole-2-carboxamido)-1H-pyrrole-2-carboxamide

IR (cm⁻¹) 3327, 1979, 1672, 1521, 1436, 1400, 1201, 1134, 837

¹H NMR (500 MHz, DMSO-d₆) δ (ppm) 10.37 (s, 1H), 9.95 (s, 1H), 9.28 (d, 1H, J=2.01 Hz), 8.57 (d, 1H, J=1.75 Hz), 8.09 (t, 1H, J=5.52 Hz), 8.04 (dd, 1H, J=7.02 Hz), 8.02 (dd, 1H), 8.01 (d, 2H, J=6.77 Hz), 7.83 (d, 2H, J=8.28 Hz), 7.76 (td, 1H, J=6.72 Hz), 7.68 (d, 1H, J=16.28 Hz), 7.65 (td, 1H), 7.63 (d, 1H, J=16.70 Hz), 7.36 (d, 1H, J=1.75 Hz), 7.20 (d, 1H, J=1.87 Hz), 7.10 (d, 1H, J=1.75 Hz), 6.88 (d, 1H, J=2.00 Hz), 5.22 (t, 1H, J=7.02 Hz), 5.10 (t, 1H, J=7.06 Hz), 3.89 (s, 3H), 3.81 (s, 3H), 3.79 (t, 2H, J=5.52 Hz), 2.05 (m, 2H), 1.98 (m, 2H), 1.67 (s, 3H), 1.65 (s, 3H), 1.58 (s, 3H)

LRMS m/z calculated for C₄₀H₄₂N₆O₃ 654.33 [M], found 655.3; [M+H]⁺

HRMS m/z calculated for C₄₀H₄₃N₆O₃ 655.3384 [M+H]⁺, found 655.3391 [M+H]⁺

### IVa16 (E)-N,N,1-trimethyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3278.99, 3088.03, 2160.27, 2023.33, 1978.97, 1672.28, 1643.35, 1438.90, 1394.53, 1259.52, 1199.72, 1182.36, 1128.36, 1058.92, 970.19, 831.32, 773.46, 752.24, 721.38

¹H NMR (500 MHz, DMSO-d₆) δ (ppm) 10.36 (s, 1H), 9.90 (s, 1H), 9.29 (d, 1H, J=2.2 Hz), 8.60 (s, 1H), 8.04 (m, 4H), 7.83 (d, 2H, J=8.2 Hz), 7.78 (td, 1H, J=1.4 Hz, 1.7 Hz), 7.69 (d, 1H, J=16.5 Hz), 7.66 (m, 1H), 7.64 (d, 1H, J=16.9 Hz), 7.34 (d, 1H, J=1.8 Hz), 7.26 (d, 1H, J=1.8 Hz), 7.12 (d, 1H, J=1.9 Hz), 6.47 (d, 1H, J=1.8 Hz), 3.89 (s, 3H), 3.66 (s, 3H), 3.06 (s, 6H).

LRMS m/z calculated for C₃₂H₃₀N₆O₃ 546.63 [M], found 547.3 [M+H]⁺

### IVa17 (E)-1-methyl-4-(1-methyl-4-(4-(2-(quinolin-3-yl)vinyl)benzamido)-1H-pyrrole-2-carboxamido)-N-propyl-1H-pyrrole-2-carboxamide

IR (cm⁻¹) 3657.04, 2980.02, 2970.38, 2887.44, 2160.27, 1977.04, 1670.35, 1637.56, 1390.68, 1197.79, 1176.58, 1122.57, 1070.49, 956.69, 833.25, 719.45,

¹H NMR (500 MHz, DMSO-d₆) δ (ppm) 10.37 (s, 1H), 9.94 (s, 1H), 9.28 (s, 1H), 8.57 (s, 1H), 8.07-7.99 (m, 5H), 7.83 (d, J=8.1 Hz, 2H), 7.77 (dd, J=7.7, 0.7 Hz, 1H), 7.71-7.60 (m, 3H), 7.37 (s, 1H), 7.20 (s, 1H), 7.11 (s, 1H), 6.88 (s, 1H), 3.89 (s, 3H), 3.82 (s, 3H), 3.15 (d, J=6.3 Hz, 2H), 1.51 (q, J=7.1 Hz, 2H), 0.89 (t, J=7.3 Hz, 3H).

LRMS m/z calculated for C₃₃H₃₂N₆O₃ 560.25 [M], found 561.3 [M+H]⁺

### IVa18 (E)-N-(2-methoxyethyl)-1-methyl-4-(1-methyl-4-(4-(2-(quinolin-3-yl)vinyl)benzamido)-1H-pyrrole-2-carboxamido)-1H-pyrrole-2-carboxamide

IR (cm⁻¹) 3302.13, 2939.52, 1637.56, 1527.62, 1436.97, 1400.32, 1388.75, 1263.37, 1197.79, 1118.71, 829.39, 798.53, 771.53, 750.31, 719.45, 682.80, 609.51

¹H NMR (500 MHz, DMSO-d₆) δ (ppm) 10.37 (s, 1H), 9.95 (s, 1H), 9.28 (d, J=2.2 Hz, 1H), 8.58 (s, 1H), 8.07-8.00 (m, 5H), 7.83 (d, J=8.2 Hz, 2H), 7.78 (t, J=7.9 Hz, 1H), 7.73-7.60 (m, 3H), 7.37 (d, J=1.8 Hz, 1H), 7.22 (d, J=1.9 Hz, 1H), 7.11 (d, J=1.8 Hz, 1H), 6.90 (d, J=1.8 Hz, 1H), 3.89 (s, 3H), 3.82 (s, 3H), 3.43 (q, J=5.9 Hz, 2H, underneath water peak), 3.28 (s, 3H), 2.55 (m, 2H).

LRMS m/z calculated for C₃₃H₃₂N₆O₄ 576.66 [M], found 577.4 [M+H]⁺

### IVb1 (E)-1-methyl-N-(1-methyl-5-(piperidine-1-carbonyl)-1H-pyrrol-3-yl)-4-(4-(2-(quinolin-3-yl)vinyl)benzamido)-1H-pyrrole-2-carboxamide

IR (cm⁻¹) 2939, 2924, 2358, 1676, 1571, 1438, 1402, 1257, 1197, 1132, 1001

¹H NMR (500 MHz, DMSO-d₆) δ (ppm) 10.36 (s, 1H), 9.89 (s, 1H), 9.28 (d, 1H, J=2.07 Hz), 8.57 (d, 1H, J=2.29 Hz), 8.05 (dd, 1H, J=8.54 Hz), 8.025 (dd, 1H), 8.025 (d, 2H, J=8.39 Hz), 7.83 (d, 2H, J=8.54 Hz), 7.78 (td, 1H, J=1.83 Hz), 7.69 (d, 1H, J=16.43 Hz), 7.66 (td, 1H), 7.63 (d, 1H, J=16.63 Hz), 7.34 (d, 1H, J=1.67 Hz), 7.26 (d, 1H, J=1.67 Hz), 7.12 (d, 1H, J=1.76 Hz), 6.39 (d, 1H, J=1.83 Hz), 3.89 (s, 3H), 3.63 (s, 3H), 3.59 (m, 4H), 1.65 (m, 2H), 1.54 (m, 4H)

LRMS m/z calculated for C₃₅H₃₄N₆O₃ 586.27 [M], found 587.7 [M+H]⁺

HRMS m/z calculated for C₃₅H₃₅N₆O₃ 587.2759 [M+H]⁺, found 587.2762 [M+H]⁺

### IVb2 (E)-1-methyl-N-(1-methyl-5-(thiomorpholine-4-carbonyl)-1H-pyrrol-3-yl)-4-(4-(2-(quinolin-3-yl)vinyl)benzamido)-1H-pyrrole-2-carboxamide

IR (cm⁻¹) 3294, 2362, 2160, 1670, 1627, 1608, 1436, 1388, 1253, 1126

¹H NMR (500 MHz, DMSO-d₆) δ (ppm) 10.36 (s, 1H), 9.90 (s, 1H), 9.28 (d, 1H, J=2.01 Hz), 8.58 (d, 1H, J=1.52 Hz), 8.033 (m, 4H), 7.83 (d, 2H, J=8.3 Hz), 7.77 (td, 1H, J=1.18 Hz), 7.68 (d, 1H, J=16.53 Hz), 7.65 (td, 1H), 7.63 (d, 1H, J=16.41 Hz), 7.34 (d, 1H, J=1.67 Hz), 7.29 (d, 1H, J=1.67 Hz), 7.128 (d, 1H, J=1.81 Hz), 6.418 (d, 1H, J=1.54 Hz), 3.89 (s, 3H), 3.66 (m, 8H), 3.64 (s, 3H)

LRMS m/z calculated for C₃₄H₃₂N₆O₃S 604.23 [M], found 605.0 [M+H]⁺

HRMS m/z calculated for C₃₄H₃₃N₆O₃S 605.2324 [M+H]⁺, found 605.2329 [M+H]⁺

### IVb3 (E)-N-(5-(azepane-1-carbonyl)-1-methyl-1H-pyrrol-3-yl)-1-methyl-4-(4-(2-(quinolin-3-yl)vinyl)benzamido)-1H-pyrrole-2-carboxamide

IR (cm⁻¹) 3331, 2926, 1670, 1525, 1514, 1436, 1278, 1199, 1130, 970

¹H NMR (500 MHz, DMSO-d₆) δ (ppm) 10.36 (s, 1H), 9.88 (s, 1H), 9.30 (d, 1H, J=1.52 Hz), 8.62 (d, 1H, J=2.28 Hz), 8.06 (dd, 1H, J=8.39 Hz), 8.04 (dd, 1H), 8.02 (d, 2H, J=8.23 Hz), 7.83 (d, 2H, J=7.98 Hz), 7.80 (td, 1H, J=8.20 Hz), 7.70 (d, 1H, J=16.46 Hz), 7.66 (td, 1H), 7.64 (d, 1H, J=16.51 Hz), 7.34 (d, 1H, J=1.22 Hz), 7.26 (d, 1H), 7.12 (d, 1H), 6.42 (d, 1H), 3.89 (s, 3H), 3.63 (s, 3H), 1.71 (m, 6H), 1.56 (m, 6H)

LRMS m/z calculated for C₃₆H₃₆N₆O₃ 600.28 [M], found 601.4 [M+H]⁺

HRMS m/z calculated for C₃₆H₃₇N₆O₃ 601.2915 [M+H]⁺, found 601.2922 [M+H]⁺

The following compounds were prepared through the 96-well plate strategy:

The ˜10 mM stock solutions of these compounds were used directly in biological evaluation.

### IVa11 (E)-1-methyl-4-(1-methyl-4-(4-(2-(quinolin-3-yl)vinyl)benzamido)-1H-pyrrole-2-carboxamido)-N-(4-(piperidin-1-yl)butyl)-1H-pyrrole-2-carboxamide

LRMS m/z calculated for C₃₉H₄₃N₇O₃ 657.34, found 658.4 [M+H]⁺

Purity based on conversion from starting material: >90%

### IVa12 (E)-1-methyl-4-(1-methyl-4-(4-(2-(quinolin-3-yl)vinyl)benzamido)-1H-pyrrole-2-carboxamido)-N-(4-(pyrrolidin-1-yl)butyl)-1H-pyrrole-2-carboxamide

LRMS m/z calculated for C₃₈H₄₁N₇O₃ 643.33, found 644.4 [M+H]⁺

Purity based on conversion from starting material: >90%

### IVa13 (E)-6-(4-(dimethylamino)styryl)-N-(1-methyl-5-((1-methyl-5-(methylcarbamoyl)-1H-pyrrol-3-yl)carbamoyl)-1H-pyrrol-3-yl)nicotinamide

LRMS m/z calculated for C₂₉H₃₁N₇O₃ 525.25, found 526 [M+H]⁺

Purity based on conversion from starting material: >50%

### IVa14 (E)-6-(4-(dimethylamino)styryl)-N-(1-methyl-5-((1-methyl-5-((4-(piperidin-1-yl)butyl)carbamoyl)-1H-pyrrol-3-yl)carbamoyl)-1H-pyrrol-3-yl)nicotinamide

LRMS m/z calculated for C₃₇H₄₆N₈O₃ 650.37, found 651 [M+H]⁺

Purity based on conversion from starting material: >90%

### IVa15 (E)-6-(4-(dimethylamino)styryl)-N-(1-methyl-5-((1-methyl-5-((4-(pyrrolidin-1-yl)butyl)carbamoyl)-1H-pyrrol-3-yl)carbamoyl)-1H-pyrrol-3-yl)nicotinamide

LRMS m/z calculated for C₃₆H₄₄N₈O₃ 636.35, found 637 [M+H]⁺

Purity based on conversion from starting material: >90%

### Anti-Infective Data

Compounds of the invention have been assessed for their antibacterial, antifungal, antiparasitic and antiviral activities.

### Antibacterial Activity

To demonstrate antibacterial activity, the compounds were evaluated against Staphylococcus aureus (ATCC 43300), Enterococcus faecalis (ATCC 51299) and Escherichia coli (ATCC 25922). The Minimum Inhibitory Concentration (MIC, at 80% inhibition) of the compounds was determined using the broth microdilution method as recommended by the CLSI (Clinical and Laboratory Standards Institute, Methods for Dilution Antimicrobial Susceptibility Tests for Bacteria that Grow Aerobically—11th Edition: Approved Standard M07, 2018).

### Antifungal Activity

To demonstrate antifungal activity, the compounds were evaluated against C. albicans (ATCC 90028), C. neoformans (F10025), A. fumigatus A1160 (MFIG001), A. fumigatus TR34/L98H (F10017, an azole resistant strain), A. flavus (CBS 128.202), Rhizopus arrhizus (NEQAS12.35321), Candida auris (H.17.157), Candida glabrata (ACPF3309), Fusarium oxysporum (F7098), and Scedosporium prolificans (F2746). The Minimum Inhibitory Concentration (MIC, at 99% inhibition) of the compounds was determined using the broth microdilution method as recommended by EUCAST (EUCAST Definitive Document E.DEF 9.3.2, Method for the determination of broth dilution minimum inhibitory concentrations of antifungal agents for conidia forming moulds and EUCAST Definitive Document E.DEF 7.3.2 Method for the determination of broth dilution minimum inhibitory concentrations of antifungal agents for yeasts).

Four of the most active antifungal compounds were further assessed against an expanded set of pathogenic filamentous fungi and yeast isolates available at the University of Texas Health Science Centre at San Antonio (see tables 3 and 4). The Minimum Inhibitory Concentration (MIC, at 50% inhibition) of the compounds was determined using the broth microdilution method as recommended by CLSI (M27 and M38 methodologies).

### Antiparasitic Activity

To demonstrate antiparasitic activity, the compounds were evaluated against T. b. brucei Lister S427 (a drug-sensitive wild type), T. b. brucei B48 (a strain resistant to diamidines and melaminophenyl arsenicals), T. congolense IL3000 (a drug sensitive wild type), T congolense 6C₃ (a diminazene resistant strain), T. equiperdum, T. evansi and T. vaginalis.

A detailed description of the procedure for evaluating activity against trypanosomes, using an Alamar Blue (resazurin) assay, is described below.

### Parasites and Cultures

The bloodstream form (BSF) T. b. brucei s427 (see De Koning, H. P. et al., Mol. Biochem. Parasitol. 2000, 106, 181-185), T. evansi AntTat 3/3 (see Dean, S. et al., Proc. Natl. Acad. Sci. USA, 2013, 110, 14741-14746), and T. equiperdum (see Stewart, M. L. et al., Eukaryot. Cell, 2010, 9, 336-343) were cultured in HMI-9 medium (Life Technologies, Paisley, United Kingdom) supplemented with 10% heat inactivated Foetal Bovine Serum (FBS (PAA Laboratories Linz, Austria)), 14 μL/L β-mercaptoethanol (BDH, Dorset, UK), and 3.0 g/L NaHCO₃ (Sigma-Aldrich) and adjusted to pH 7.4. These cell lines were maintained at 37° C. in a humidified, 5% CO₂ environment.

The BSF T. congolense IL3000 and a clonal line, 6C₃, adapted from IL3000 in vitro with diminazene aceturate (Merck), leading to a ˜10-fold resistance, were cultured in TCBSF3 medium without red blood cells (see Zheoat, A. M. et al., 2021, Pathogens (Basel, Switzerland), 10(2), 175). Dulbecco's minimum essential medium (MEM) was supplemented with 25 mM (4-(2-hydroxyethyl)-1-piperazineethanesulfonic acid) (HEPES), 26 mM NaHCO₃, 5.6 mM Dd-glucose, 1 mM sodium pyruvate, 40 μM adenosine, 100 μM hypoxanthine, 16.5 μM thymidine, and 25 μM bathocuproinedisulfonic acid disodium salt. To this basal medium were added β-mercaptoethanol (0.0014% v/v), 1.6 mM glutamine, 100 units/mL penicillin/0.1 mg/mL streptomycin (Gibco), 20% goat serum (Gibco), and 5% Serum Plus (SAFC Bi-osciences) (see Coustou, V. et al., 2010, PLoS Negl. Trop. Dis., 4, e618 and Giordani, F. et al., J. Med. Chem., 2019, 62, 3021-3035). The BSF T. congolense IL3000 WT and its derived line, 6C3, were cultured at 34° C. in a humidified, 5% CO₂ environment.

### Resazurin-Based Drug Sensitivity Assay

A resazurin-based (Alamar blue) drug sensitivity assay was used to assess and compare the activity of standard trypanocides in the different cell lines investigated (see Gould, M. K. et al., Anal. Biochem., 2008, 382, 87-93). Briefly, 23 doubling dilutions of the test compound of the invention, starting at 100 μM, and a compound-free control were prepared in 100 μL medium in a 96-well white plate (Greiner Bio-one, Frickenhausen, Germany). The cells were adjusted to 2×10⁴ cells/mL for T. b. brucei and T. equiperdum; 4×10⁴ cells/mL for T. evansi; and 5×10⁵ cells/mL for T. congolense in the appropriate medium. 100 μL of the adjusted cells was then added to the wells containing drug dilutions and incubated for 48 h under respective culture condition. Thereafter, 20 μL of 125 mg/mL resazurin sodium dye was added, and the plate was incubated for a further 24 h. The resorufin fluorescence in each well of the plate was determined and used to calculate the Half maximal effective concentration (EC₅₀) of the drug, as described in Carruthers, L. V. et al., Mol. Microbiol., 2021, 116, 564-588.

A detailed description of the procedure for evaluating activity against Trichomonas using an resorufin assay is described in Natto M. J. et al., 2021, ACS Infect. Dis., 7:1752-1764.

### Antiviral Activity

To demonstrate antiviral activity, compounds of the invention were evaluated against Human rhinovirus (HRV-14), human respiratory syncytial virus (RSV A2), Influenza (H1N1) and SARS-CoV-2.

The procedure for evaluating activity against SARS-CoV-2, is described below.

### Cell and Virus

SARS-CoV-2 (hCoV-19/Australia/VIC01/2020) was provided by The Doherty Institute, Melbourne, Australia at P1 and passaged twice in Vero/hSLAM cells [ECACC04091501]. Whole genome sequencing was performed on the working stock at Passage 3, using both Nanopore and Illumina technologies and no significant changes in the viral sequence were observed. Virus titre was determined by a plaque assay on Vero E6 cells [ECACC 85020206]. Cell lines were obtained from the European Collection of Authenticated Cell Cultures (ECACC) PHE, Porton Down, UK. Cell cultures were maintained at 37° C. in minimal essential media (MEM) (Life Technologies, California, USA) supplemented with 10% foetal bovine serum (FBS) (Sigma, Dorset, UK) and 25 mM HEPES (Life Technologies).

### Plaque Inhibition Assay

A microplaque inhibition assay was used to assess the inhibitory effect of various compounds. Compounds were diluted 2-fold over a 12-step dilution range, in duplicate. A fixed concentration of wildtype SARS-CoV-2 was added to the diluted compounds.

Additional assay wells included virus-free and untreated virus-only controls. The plates (diluted compound & virus) were then incubated for 1 hr at 37° C. to allow the compounds to neutralise the virus. The contents of the neutralisation plates were then transferred into 96-well plates containing Vero-E6 cells and the virus was allowed to adsorb to the cells for 1 hr at 37° C. The inocula were removed from the cell plates and a viscous overlay (1% CMC) added (test compound was added to the overlay media). The plates were then incubated for 24 hours. The cells were fixed using 8% formalin for >8 hrs and an immunostaining protocol performed on the fixed cells, as described previously by Bewley K. R. et al. Nat. Protoc. 2021, 16(6):3114-3140. Stained foci were counted using an ELISpot counter (Cellular Technology Limited (CTL)). The counted foci data were then imported into RBioconductor.

A positive control, chloroquine diphosphate (50-0.02 μM), was run alongside test compounds, on each assay plate.

### Data Analysis

A mid-point probit analysis (written in R programming language for statistical computing and graphics) was used to determine the amount of compound (μM) required to inhibit SARS-CoV-2 infectious viral foci by 50% (IC₅₀) compared with the virus only control. All outliers were included in the mid-point probit analysis.

The positive control, chloroquine diphosphate (50-0.02 μM), is consistent across assays where an IC₅₀ of 0.641 μM was recorded with a 95% confidence interval range of 0.448-0.912. The dashed lines are the 95% confidence intervals.

### Activity Against H1N1, HRV-14 and RSV A2

The procedure for evaluating activity against Influenza (H1N1) Human rhinovirus (HRV-14) and human respiratory syncytial virus (RSV A2) is described below.

The percentage viral inhibition was determined by an Accelerated Viral Neuraminidase Inhibition Assay (AVINA) for influenza and Viral ToxGlo assay for RSV and HRV.

### Cell Seeding

Cells (Hep-2 for RSV A2, 15000 cells per well; Hela Ohio for HRV-14, 20000 cells per well; A549 for Influenza H1 N1, 40000 cells per well) were seeded in white (Greiner 655098, RSV A2 assay HRV-14 assay) or black (Greiner 655090, Influenza H1 N1 assay) 96 well plates in 50 μL assay media/well and incubated overnight at 37° C. (or 35° C. for Hela Ohio)/5% CO₂. Assay media was as follows: for RSV A2 assay, Eagle's Minimum Essential Medium (EMEM) [Sigma M2279] supplemented with 2% heat inactivated fetal bovine serum (HI FBS) 1% glutamine 1% Penicillin/Streptomycin; for HRV-14 assay, EMEM+2% HI FBS+1% glutamine+1% Penicillin/Streptomycin+1% Non-Essential Amino Acids; for Influenza H1N1 assay, DMEM (High Glucose) [Gibco 41966029]+1% Penicillin/Streptomycin.

### Test Article Preparation

Compounds of the invention were prepared at 10 mM in dimethylsulfoxide (DMSO) and subsequently further diluted to 2 mM stock concentration in DMSO; stocks were aliquoted and stored at 80° C. until use. 10× test solutions (200 μM) were prepared in 10% DMSO by diluting stock solutions 10-fold in sterile water in round bottom 96 well plate (Corning 3788). 10 μL 10× test article solutions were transferred to the assay plates to result in a final test concentration of 20 μM in 1% DMSO. Similarly prepared were inhibitor controls: Ribavirin (25 μM) for RSV assay, Rupintrivir (25 nM) for HRV assay, and Zanamavir (50 nM) for Influenza assay.

### Assay Set Up

For the influenza assay: 20 μL trypsin treated N-p-tosyl-phenylalanine chloromethyl ketone (TPCK) (2 μg/mL) was added to all wells of the antiviral testing plate containing the pre seeded cells, resulting in a final concentration of 0.4 μg/mL. 20 μL diluted virus stock was added to achieve a multiplicity of infection (MOI) of 0.1 (assay medium for cell control wells).

For the RSV and HRV assays: 40 μL virus stock, diluted to an MOI of 0.5, was added to all wells containing the pre seeded cells assay medium for cell control wells.

Total volume for all assays was 100 μL. All plates were incubated for 72 h at 37° C./5% CO₂ (Influenza and RSV) and 35° C./5% CO₂ (HRV). All testing was performed in triplicate on three different plates.

### Assay Endpoint and Calculation of Inhibition

For Influenza assay: Viral infection was determined by AVINA which measures neuraminidase activity by the addition of 4-methylumbelliferyl N-acetyl-α-D-neuraminic acid sodium salt (MU-NANA) substrate (Sigma M8639). Plates were read on a Biotek spectrophotometer to determine fluorescence signal (Ex: 355 nm, Em: 460 nm).

For RSV and HRV assays: RSV and HRV infected plates were evaluated for cytopathic effect by the addition of Viral ToxGlo™ (Promega G8941). After 20 minutes of incubation the luminescence signal was determined using a Biotek spectrophotometer.

Percentage viral inhibition for all assays was calculated relative to virus control (VC, 100% infection) and cell control (0% infection).

### Results

Tables 1 to 6 display the activities of the compounds against a variety of pathogenic bacteria, fungi, parasites and viruses. All of the compounds possess measurable activities against pathogens, with most compounds possessing high levels of activity across bacteria, fungi, parasites and viruses. Many compounds possess potency in the range expected of compounds that would find use in clinical applications.

TABLE 1
Antibacterial Data

MIC80 (μM)

| Compound | S. aureus | E. faecalis | E. coli |
|---|---|---|---|
| Ia1 | ** | * | — |
| Ia2 | *** | | — |
| Ia3 | *** | | — |
| Ia4 | ** | | — |
| Ia5 | *** | | — |
| Ia6 | *** | *** | — |
| Ia7 | *** | | — |
| Ia8 | ** | * | — |
| Ia9 | ** | * | — |
| Ia10 | *** | *** | — |
| Ia11 | ** | — | — |
| Ia12 | ** | *** | — |
| Ia13 | ** | *** | — |
| Ia14 | *** | *** | — |
| Ia15 | ** | ** | * |
| Ia16 | * | ** | — |
| Ia17 | * | * | — |
| Ia18 | ** | ** | * |
| Ia19 | ** | ** | * |
| Ia20 | ** | ** | * |
| Ia21 | *** | *** | — |
| Ia22 | ** | *** | — |
| Ia23 | * | — | — |
| Ia24 | *** | ***** | — |
| Ia25 | * | — | — |
| Ia26 | * | — | — |
| Ia27 | *** | *** | — |
| Ia28 | *** | *** | — |
| Ia29 | *** | ** | — |
| Ia30 | ** | ** | — |
| Ia31 | *** | ** | — |
| Ia32 | *** | — | — |
| Ia33 | ** | * | — |
| Ia34 | *** | ** | — |
| Ia35 | *** | ** | — |
| Ia36 | ** | * | — |
| Ia37 | ** | * | — |
| Ia38 | *** | * | — |
| Ia39 | *** | * | — |
| Ia40 | *** | ** | — |
| Ia41 | ** | * | — |
| Ia42 | ** | ** | — |
| Ia43 | *** | *** | — |
| Ia44 | ** | ** | — |
| Ia45 | ** | — | — |
| Ia46 | * | — | — |
| Ia47 | ** | — | — |
| Ia48 | *** | *** | — |
| Ia49 | *** | — | — |
| Ia50 | * | * | — |
| Ia51 | * | * | — |
| Ia52 | ** | — | — |
| Ia53 | *** | ** | — |
| Ia54 | ** | ** | — |
| Ia55 | ** | ** | — |
| Ia56 | ** | ** | — |
| Ia57 | *** | ** | — |
| Ia58 | *** | ** | — |
| Ia59 | *** | — | — |
| Ia60 | *** | ** | — |
| Ia61 | ** | ** | — |
| Ia62 | *** | *** | — |
| Ia63 | *** | * | — |
| Ia64 | ** | | |
| Ia65 | *** | | |
| Ib1 | *** | *** | — |
| Ib2 | *** | *** | — |
| Ib3 | *** | ** | — |
| Ib4 | *** | *** | — |
| Ib5 | *** | *** | — |
| Ib6 | ** | * | — |
| Ib7 | *** | *** | — |
| Ib8 | *** | *** | — |
| Ib9 | *** | * | — |
| Ib10 | ** | — | — |
| Ib11 | *** | ** | — |
| Ic1 | *** | *** | — |
| Ic2 | *** | *** | — |
| Ic3 | *** | *** | — |
| Ic4 | *** | *** | — |
| Ic5 | *** | ** | — |
| Ic6 | *** | ** | — |
| Ic7 | *** | — | — |
| Ic8 | *** | — | — |
| Ic9 | *** | ** | — |
| Ic10 | ** | — | — |
| Ic11 | ** | * | — |
| Ic18 | *** | *** | — |
| Ic19 | *** | ** | — |
| Ic20 | * | | |
| Ic21 | * | | |
| Ic22 | * | | |
| Ic23 | ** | | |
| Id1 | *** | — | — |
| Id2 | *** | — | — |
| II1 | * | — | — |
| II2 | ** | ** | — |
| II3 | ** | — | — |
| II4 | ** | — | — |
| IIIb1 | ** | — | — |
| IIIb2 | ** | — | — |
| IIIb3 | *** | — | — |
| IIIa1 | *** | ** | — |
| IIIa2 | *** | — | — |
| IIIa3 | ** | ** | — |
| IIIb4 | *** | — | — |
| IIIa4 | *** | *** | * |
| IIIa5 | *** | ** | — |
| IIIa6 | *** | *** | * |
| IIIb5 | *** | — | — |
| IIIa7 | *** | ** | * |
| IIIa8 | * | — | — |
| IIIa9 | ** | ** | * |
| IIIb6 | * | — | — |
| IIIb7 | ** | — | — |
| IIIb8 | *** | — | — |
| IIIb9 | ** | ** | — |
| IVa1 | *** | *** | — |
| IVa2 | *** | *** | — |
| IVa3 | * | — | — |
| IVb1 | * | — | — |
| IVa4 | *** | * | — |
| IVb2 | * | — | — |
| IVa5 | *** | *** | — |
| IVa6 | *** | *** | — |
| IVb3 | * | — | — |
| IVa7 | ** | — | — |
| IVa8 | ** | ** | — |
| IVa9 | * | — | — |
| IVa10 | ** | * | — |
| IVa11 | ** | ** | — |
| IVa12 | ** | *** | — |
| IVa13 | *** | *** | — |
| IVa14 | ** | * | — |
| IVa15 | — | * | — |
| IVa16 | *** | *** | — |
| IVa17 | *** | *** | — |
| IVa18 | *** | *** | — |

Where blank entries mean not tested, — means MIC₈₀ > 100 μM, * means 25 μM < MIC₈₀ <= 100 μM, ** means 5 μM < MIC₈₀ <= 25 μM, and *** means MIC₈₀ <= 5 μM.

TABLE 2
Antifungal Data

| | MIC₉₉ (μg/mL) | | | | | | | | | |
|---|---|---|---|---|---|---|---|---|---|---|
| | | | A. | A. | | | | | | |
| | C. | C. | fumigatus | fumigatus | A. | Rhizopus | Candida | Candida | Fusarium | Scedosporium |
| Compound | albicans | neoformans | A1160 | TR34/L98H | flavus | arrhizus | auris | glabrata | oxysporum | prolificans |
| Ia1 | | *** | ** | ** | | — | ** | *** | | |
| Ia3 | | | *** | | | | | | | |
| Ia5 | | | * | | | | | | | |
| Ia6 | *** | *** | *** | *** | *** | ** | — | *** | *** | *** |
| Ia7 | | | * | | | | | | | |
| Ia8 | * | ** | ** | ** | *** | ** | — | ** | *** | ** |
| Ia9 | ** | *** | ** | ** | ** | ** | — | ** | *** | ** |
| Ia10 | ** | *** | *** | ** | *** | ** | ** | *** | ** | *** |
| Ia11 | ** | *** | ** | ** | | ** | — | *** | | ** |
| Ia12 | | ** | — | | | | | ** | | |
| Ia13 | | ** | — | | | | | * | | |
| Ia14 | | ** | — | * | | — | — | ** | | |
| Ia15 | | *** | * | | | | | ** | | |
| Ia16 | | ** | — | | | | | * | | |
| Ia17 | | — | — | | | | | * | | |
| Ia18 | | ** | — | | | | | | | |
| Ia21 | | — | ** | ** | | ** | * | *** | | |
| Ia22 | | * | — | | | | | ** | | |
| Ia24 | | ** | * | * | | — | — | ** | | |
| Ia32 | | ** | — | — | | — | — | ** | | |
| Ia33 | | * | — | | | | | — | | |
| Ia34 | | * | — | * | | — | — | ** | | |
| Ia35 | | * | — | — | | — | — | ** | | |
| Ia36 | | — | — | | | | | * | | |
| Ia37 | | * | — | | | | | — | | |
| Ia38 | | * | — | | | | | ** | | |
| Ia39 | | * | — | | | | | * | | |
| Ia44 | | * | — | | | | | ** | | |
| Ia45 | | ** | — | | | | | * | | |
| Ib1 | | ** | ** | ** | | * | — | *** | | |
| Ib2 | | ** | ** | ** | | ** | — | *** | | |
| Ib3 | | ** | — | | | | | ** | | |
| Ib4 | | ** | - | | | | | * | | |
| Ib5 | | ** | ** | ** | | * | - | *** | | |
| Ib6 | | * | - | * | | ** | - | * | | |
| Ib7 | | ** | - | ** | | - | - | ** | | |
| Ib8 | | ** | ** | ** | | ** | - | *** | | |
| IIIb3 | — | — | * | — | — | — | — | — | — | — |
| IIIa1 | * | *** | ** | ** | ** | ** | — | *** | ** | ** |
| IIIa2 | | *** | ** | ** | | * | ** | *** | | |
| IIIa3 | | *** | ** | ** | | — | — | *** | | |
| IIIb4 | | — | — | | | | | — | | |
| IIIa4 | | *** | ** | ** | | ** | * | *** | | |
| IIIa5 | *** | | ** | ** | ** | ** | ** | ** | | |
| IIIa6 | | *** | ** | ** | | * | ** | *** | | |
| IIIb5 | | — | — | | | | | — | | |
| IIIa7 | | *** | ** | ** | | ** | ** | *** | | |
| IIIa8 | | ** | — | — | | — | — | — | | |
| IIIa9 | | *** | ** | ** | | ** | ** | *** | | |
| IVa9 | | * | — | | | | | * | | |
| IVa10 | | — | * | | | | | ** | | |
| Ic1 | | ** | — | | | | | ** | | |
| Ic2 | | ** | — | | | | | *** | | |
| Ic3 | | ** | — | | | | | ** | | |
| Ic4 | | ** | — | | | | | ** | | |
| Ic5 | | | — | | | | | ** | | |
| Ic7 | | ** | — | | | | | ** | | |
| Ic8 | | * | — | | | | | ** | | |
| Ic9 | | ** | — | — | | — | — | — | | |
| Ic10 | | — | — | | | | | * | | |
| Ic11 | | — | — | | | | | ** | | |
| Ic14 | | — | — | | | | | * | | |
| Ic15 | | * | — | | | | | — | | |
| Ic18 | | ** | — | * | | * | — | *** | | |

Where blank entries mean not tested, — means MIC₉₉ > 50 μg/mL, * means 25 μg/mL < MIC99 <= 50 μg/mL, ** means 5 μg/mL < MIC99 <= 25 μg/mL, and *** means MIC99 <= 5 μg/mL.

TABLE 3
Antifungal Data: Assessment against
an expanded set of filamentous fungi

| | | MIC₅₀ (μg/mL) | | | |
|---|---|---|---|---|---|
| Species | Isolate No. | Ia10 | Ia27 | Ia8 | Ia9 |
| Aspergillus | AF293 | *** | *** | ** | ** |
| fumigatus | DI15-106 | *** | *** | ** | ** |
| | DI15-116 | *** | *** | ** | ** |
| | DI19-187 | *** | *** | ** | ** |
| | DI20-95 | *** | *** | ** | ** |
| | DI20-103 | *** | *** | ** | ** |
| | DI19-230 | *** | *** | ** | ** |
| | DI19-231 | ** | *** | ** | ** |
| | DI19-232 | *** | *** | ** | ** |
| | DI19-233 | *** | *** | ** | ** |
| | DI19-234 | *** | *** | ** | ** |
| | DI19-236 | *** | *** | ** | ** |
| | DI19-237 | *** | *** | ** | ** |
| Aspergillus | ATCC 204304 | *** | *** | ** | ** |
| flavus | Aflav2 | *** | *** | ** | ** |
| | Aflav3 | *** | *** | ** | ** |
| Fusarium | F2 | *** | *** | *** | *** |
| oxysporum | F3 | *** | *** | *** | *** |
| Fusarium | F4 | *** | *** | *** | *** |
| solani | DI19-284 | *** | *** | *** | *** |
| Scedosporium | S1 | *** | *** | ** | ** |
| spp. | S3 | *** | *** | ** | *** |
| Lomentospora | S2 | *** | *** | ** | *** |
| prolificans | | | | | |
| Paecilomyces | MYA-3630 | ** | ** | ** | ** |
| variotii | | | | | |
| Rhizopus | 99-892 | ** | *** | ** | ** |
| arrhizus | 99-880 | ** | *** | ** | ** |
| | RA3 | ** | ** | ** | ** |
| Blastomyces | BD1 | ** | ** | ** | ** |
| dermatitidis | BD2 | ** | ** | ** | ** |
| | BD3 | * | * | ** | ** |

Where * means 8 μg/mL < MIC₅₀ <= 32 μg/mL, ** means 1 μg/mL < MIC₅₀ <= 8 μg/mL, and *** means MIC₅₀ <= 1 μg/mL.

TABLE 4
Antifungal Data: Assessment against an expanded set of yeasts

| | | MIC₅₀ (μg/mL) | | | |
|---|---|---|---|---|---|
| Species | Isolate No. | Ia10 | Ia27 | Ia8 | Ia9 |
| Candida parapsilosis | ATCC 22019 | ** | ** | ** | ** |
| C. krusei | ATCC 6258 | *** | *** | ** | ** |
| C. albicans | SC5314 | ** | ** | ** | ** |
| | CA90028 | ** | ** | ** | ** |
| | CA3 | ** | ** | ** | ** |
| C. auris | DI17-47 | ** | ** | ** | ** |
| | DI17-48 | ** | ** | ** | ** |
| | DI17-46 | ** | ** | ** | ** |
| C. glabrata | 05-62 | *** | *** | ** | ** |
| | 05-761 | *** | *** | ** | *** |
| | CG3 | *** | *** | ** | ** |
| C. guilliiermondii | Cgui1 | ** | ** | ** | ** |
| | Cgui2 | ** | ** | ** | ** |
| | Cgui3 | ** | ** | ** | ** |
| C. parapsilosis | CP1 | ** | ** | ** | ** |
| | CP2 | ** | ** | ** | ** |
| | CP3 | ** | ** | ** | ** |

Where * means 8 μg/mL < MIC₅₀ <= 32 μg/mL, ** means 1 μg/mL < MIC₅₀ <= 8 μg/mL, and *** means MIC₅₀ <= 1 μg/mL.

TABLE 5
Antiparasitic Data

| | IC₅₀ (μM) | | | | | | |
|---|---|---|---|---|---|---|---|
| | | T.b. brucei | | T. congo | | | |
| Compound | T.b. brucei | B48 | T. congo | C63 | T. equiperdum | T. evansi | T. vaginalis |
| Ia1 | * | * | ** | ** | * | ** | |
| Ia2 | — | | ** | | | | |
| Ia3 | * | | *** | | | | |
| Ia4 | — | | ** | | | | |
| Ia5 | * | | *** | | | | |
| Ia6 | ** | | *** | | | | |
| Ia7 | * | | *** | | | | |
| Ia9 | * | | * | | | | |
| Ia10 | ** | | *** | | | | |
| Ia11 | * | | ** | | | | |
| Ia12 | *** | | — | | * | * | ** |
| Ia13 | *** | | *** | | | | ** |
| Ia14 | *** | | ** | | | | *** |
| Ia15 | * | * | ** | ** | * | * | — |
| Ia16 | * | | ** | | | | * |
| Ia17 | * | | * | | | | * |
| Ia18 | * | | *** | | | | * |
| Ia19 | * | | *** | | | | * |
| Ia21 | *** | | ** | | | | *** |
| Ia22 | * | | *** | | | | ** |
| Ia23 | — | | — | | | | * |
| Ia24 | * | | ** | | | | |
| Ia26 | — | | — | | | | * |
| Ia28 | *** | | * | | | | |
| Ia32 | ** | | | | | | — |
| Ia33 | — | | — | | | | * |
| Ia34 | ** | | ** | | | | ** |
| Ia35 | ** | | ** | | | | ** |
| Ia36 | * | | * | | | | * |
| Ia37 | — | | — | | | | * |
| Ia38 | ** | | ** | | | | * |
| Ia39 | — | | — | | | | * |
| Ia44 | * | | * | | | | * |
| Ia45 | ** | | ** | | | | * |
| Ib1 | *** | | *** | | | | ** |
| Ib2 | *** | | *** | | | | *** |
| Ib3 | ** | | * | | | | * |
| 1b5 | ** | | ** | | | | ** |
| Ib6 | — | | * | | | | — |
| Ib7 | ** | | ** | | | | ** |
| Ib8 | *** | | ** | | | | ** |
| Ia46 | — | | — | | | | * |
| IIIb 1 | * | ** | — | — | ** | ** | |
| IIIa1 | **** | ** | ** | ** | ** | ** | |
| IIIa2 | ** | ** | * | * | ** | ** | |
| IIIa3 | *** | ** | * | * | ** | ** | |
| IIIb4 | ** | ** | — | — | ** | ** | |
| IIIa4 | *** | *** | ** | ** | *** | ** | |
| IIIa5 | * | * | * | X | * | | |
| IIIa6 | *** | ** | * | * | *** | ** | |
| IIIb5 | ** | ** | — | — | ** | ** | |
| IIIa7 | — | — | * | * | — | — | |
| IIIa8 | *** | ** | * | * | *** | ** | |
| IIIa9 | * | — | * | * | — | * | |
| IIIb6 | ** | — | — | — | ** | * | |
| IIIb8 | *** | *** | * | * | *** | ** | |
| IIIb9 | *** | ** | — | — | ** | * | |
| IVa1 | * | * | — | — | * | * | |
| IVa2 | * | * | — | — | * | * | |
| IVa3 | * | * | — | — | * | * | |
| IVb1 | * | | — | | | | * |
| IVa4 | * | | * | | | | * |
| IVb2 | * | | — | | | | * |
| IVa5 | * | | * | | | | ** |
| IVa6 | * | | * | | | | ** |
| IVb3 | * | | — | | | | * |
| IVa7 | — | | — | | | | * |
| IVa8 | — | | — | | | | * |
| IVa10 | * | | — | | | | * |
| Ic1 | *** | | ** | | | | *** |
| Ic2 | *** | | *** | | | | *** |
| Ic3 | ** | | *** | | | | ** |
| Ic4 | ** | | *** | | | | ** |
| Ic5 | ** | | *** | | | | ** |
| Ic7 | *** | | *** | | | | * |
| Ic8 | *** | | *** | | | | * |
| Ic9 | ** | ** | ** | ** | * | ** | * |
| Ic10 | ** | | ** | | | | * |
| Ic11 | * | | ** | | | | * |
| Ic14 | — | | — | | | | * |
| Ic15 | — | | — | | | | * |
| Ic17 | — | | — | | | | — |
| Ic18 | ** | | ** | | | | ** |

For T.b. brucei and T. congolense, blank entries mean not tested, — means IC₅₀ > 1 μM, * means 0.25 μM < IC₅₀ <= 1 μM, ** means 0.05 μM < IC₅₀ <= 0.25 μM, and *** means IC₅₀ <= 0.05 μM. For T. equiperdum and T. evansi, blank entries mean not tested, — means IC₅₀ > 1 μM, * means 0.1 μM < IC₅₀ <= 1 μM, ** means 0.01 μM < IC₅₀ <= 0.1 μM, and *** means IC₅₀ <= 0.01 μM. For T. vaginalis, blank entries mean not tested, — means IC₅₀ > 50 μM, * means 5 μM < IC₅₀ <= 50 μM, ** means 1 μM < IC₅₀ <= 5 μM, and *** means IC₅₀ <= 1 μM.

TABLE 6
Antiviral Data

IC₅₀ (μM)

| | | | | Influenza |
|---|---|---|---|---|
| Compound | SARS-CoV-2 | HRV | RSV | H1N1 |
| Ia1 | ** | | | |
| Ia9 | ** | | | |
| Ia11 | * | | | |
| Ia12 | * | * | *** | *** |
| Ia13 | * | ** | *** | *** |
| Ia14 | | | * | — |
| Ia15 | * | | * | |
| Ia16 | | | ** | ** |
| Ia17 | | | * | * |
| Ia21 | | *** | ** | ** |
| IIIa3 | | ** | *** | *** |
| IIIa5 | *** | *** | | ** |
| IIIa6 | | *** | *** | *** |
| IIIa7 | *** | | | |
| IIIa9 | *** | ** | | * |
| IIIb7 | | * | | |
| IIIb8 | | | | *** |
| IVa8 | | ** | * | * |
| IVa10 | | *** | *** | * |

For SARS-CoV-2, blank entries mean not tested, — means IC₅₀ > 32 μM, * means 8 μM < IC₅₀ <= 32 μM, ** means 2 μM < IC₅₀ <= 8 μM, and *** means IC₅₀ <= 2 μM. For HRV, RSV and Influenza H1N1 blank entries mean not tested, — means < 10% inhibition at 20 μM, * means 10% < inhibition at 20 μM <= 25%, ** means 25% < inhibition at 20 μM <= 50%, and *** means > 50% inhibition at 20 μM.

### Nucleic Acid Binding Data

The ability of compounds of the invention to bind to DNA and RNA was assessed using a fluorescence intercalator displacement assay. Genomic double stranded DNA (salmon) or PolyAU double stranded RNA is treated with probe molecule (Sybr-Safe) that increases its fluorescence upon binding to nucleic acids. When this system is treated with another compound, any reduction in fluorescence indicates that the compound has displaced the probe and bound to the nucleic acid.

### Fluorescence Intercalator Displacement (FID) Method

polyAU RNA (Polyadenylic acid—Polyuridylic acid sodium salt, double-stranded homopolymer, P1537, Merck) or genomic DNA (Deoxyribonucleic acid sodium salt from salmon testes, D1626, Merck) was dissolved in 1 mM pH 7.4 phosphate buffer (containing 0.27 mM potassium chloride, 13.7 mM sodium chloride) to a concentration of 1 mg/mL in, SybrSafe (SYBR® Safe DNA Gel Stain, ×10,000 in DMSO, S33102 Invitrogen) was used as supplied by the manufacturer in DMSO, and compounds of the invention were prepared as 10 mM stocks in DMSO. These stock solutions were diluted appropriately with each other and 1 mM phosphate buffer to give test solutions comprised of 20 μM compound of the invention, 12500-fold dilution of SybrSafe and 3.76 ng/mL RNA/DNA. Control solutions of RNA/DNA and SybrSafe, RNA/DNA, and SybrSafe at these concentrations were also prepared. Test and control solutions were heated to 30° C. and the fluorescence of each solution was measured using the SYBER filter setting of a StepOnePlus using melt analysis mode (StepOne Software v2.3). The reduction of fluorescence due to the binding of the compound of the invention to the RNA/DNA was calculated as a normalised percentage based on the fluorescence measured due to the control with SybrSafe and RNA/DNA as maximum and the control with only SybrSafe as minimum. Low normalised percentage indicates a greater ability to displace SybrSafe from the RNA/DNA, and suggests strong binding to RNA/DNA. High normalised percentages does not preclude strong RNA/DNA binding, but may indicate a compound with a high sequence specificity.

TABLE 7
Nucleic Acid Binding - Data obtained from a fluorescence
intercalator displacement assay using gDNA or polyAU
RNA and SYBR-safe as fluorescent probe.

| | % Normalised | % Normalised |
|---|---|---|
| | Fluorescence | Fluorescence with |
| Compound | with gDNA | polyAU RNA |
| Ia1 | 13 | 5 |
| Ia3 | 13 | 38 |
| Ia5 | 21 | 12 |
| Ia6 | 3 | 6 |
| Ia7 | 10 | 24 |
| Ia8 | 4 | 9 |
| Ia9 | 13 | 19 |
| Ia10 | 8 | 17 |
| Ia12 | 2 | 2 |
| Ia13 | 2 | 3 |
| Ia14 | 2 | 6 |
| Ia15 | 16 | 64 |
| Ia16 | 31 | 27 |
| Ia17 | 13 | 28 |
| Ia18 | 6 | 9 |
| Ia19 | 10 | 8 |
| Ia20 | 3 | 6 |
| Ia21 | 3 | 2 |
| Ia22 | 3 | 5 |
| Ia23 | 41 | 25 |
| Ia24 | 1 | 4 |
| Ia25 | 28 | 12 |
| Ia27 | 13 | 17 |
| Ia28 | 34 | 66 |
| Ia32 | 67 | 23 |
| Ia33 | 49 | 24 |
| Ia34 | 7 | 6 |
| Ia35 | 21 | 3 |
| Ia36 | 11 | 4 |
| Ia37 | 16 | 68 |
| Ia38 | 10 | 4 |
| Ia39 | 50 | 43 |
| Ia40 | 32 | 13 |
| Ia41 | 16 | 6 |
| Ia42 | 20 | 4 |
| Ia43 | 15 | 42 |
| Ia44 | 16 | 19 |
| Ia45 | 9 | 3 |
| Ib1 | 23 | 12 |
| Ib2 | 12 | 3 |
| Ib3 | 13 | 5 |
| Ib4 | 20 | 5 |
| Ib5 | 22 | 13 |
| Ib6 | 11 | 49 |
| Ib7 | 22 | 7 |
| Ib8 | 15 | 4 |
| Ia46 | 10 | 2 |
| IIIb1 | 47 | 30 |
| IIIb2 | 31 | 85 |
| IIIb3 | 31 | 39 |
| IIIa1 | 17 | 18 |
| IIIa2 | 17 | 21 |
| IIIa3 | 27 | 3 |
| IIIb4 | 37 | 86 |
| IIIa4 | 14 | 3 |
| IIIa5 | 11 | 3 |
| IIIa6 | 18 | 29 |
| IIIb5 | 49 | 59 |
| IIIa7 | 13 | 20 |
| IIIa8 | 31 | 10 |
| IIIa9 | 16 | 24 |
| IIIb6 | 84 | 53 |
| IIIb7 | 63 | 44 |
| IIIb8 | 37 | 26 |
| IIIb9 | 55 | 30 |
| IVa1 | 41 | 65 |
| IVa2 | 29 | 73 |
| IVa4 | 15 | 81 |
| IVa5 | 51 | 12 |
| IVa7 | 65 | 92 |
| IVa8 | 70 | 94 |
| IVa9 | 3 | 4 |
| IVa11 | 42 | 68 |
| IVa12 | 50 | 71 |
| IVa13 | 80 | 83 |
| IVa14 | 19 | 12 |
| IVa15 | 15 | 8 |
| Ic1 | 13 | 4 |
| Ic2 | 22 | 10 |
| Ic3 | 18 | 14 |
| Ic4 | 12 | 15 |
| Ic5 | 27 | 14 |
| Ic6 | 50 | 12 |
| Ic7 | 47 | 12 |
| Ic8 | 65 | 32 |
| Ic9 | 71 | 26 |
| Ic10 | 56 | 41 |
| Ic11 | 86 | 28 |
| Ic12 | 94 | 38 |
| Ic14 | 93 | 46 |
| Ic15 | 82 | 42 |
| Ic16 | 98 | 64 |
| Ic17 | 93 | 52 |
| Ic18 | 26 | 36 |
| Ic19 | 28 | 14 |

The % normalised fluorescence obtained with compounds of the invention is shown in Table 7. A reduction in normalised % is indicative of probe displacement and thus strong nucleic acid binding. Zero reduction in fluorescence (i.e. 100% fluorescence) does not preclude nucleic acid binding, but any binding would be weaker than the probe. Compounds of the invention exhibit various degrees of nucleic acid binding, with several demonstrating very strong binding affinity for both DNA and RNA.

### Selectivity Indices

Compounds of the invention were also evaluated for their cytotoxicity in a representative mammalian cell line (HEK293), and selectivity indices were then computed by dividing the cytoxicity value by the relevant measure of inhibition found in Tables 1 to 6. The resultant selectivity indices, with appropriate units, are tabulated in Tables 8 to 14. These are compared with the selectivity indices of reference compounds. Selectivity index thresholds have been used to illustrate that compounds of the invention have greater selectivity indices, and hence are more selective and safer as drugs than the reference compounds.

We note that the selected reference compounds (234 and 235 in particular) are those in the art that, to the best of the inventor's knowledge, have the greatest selectivity indices, i.e. are the most selective. Comparisons have been made with exact comparators (analogous compounds differing by the length of a specific section within the structure) wherever possible. Compounds 234 and 235 have been used as reference compounds where exact comparators are not available. Given that these compounds (to the best of the inventor's knowledge) are the most selective of the compounds known in the art, they provide a useful comparison in the absence of exact comparators.

A detailed description of the procedure for evaluating cytotoxicity against HEK293 cells, is described in Zheoat A. M. et al., 2021, Pathogens, 10:175 (in which HEK293 cells have been treated in the same way as the described procedure for HFF cells).

### Structures of Reference Compounds

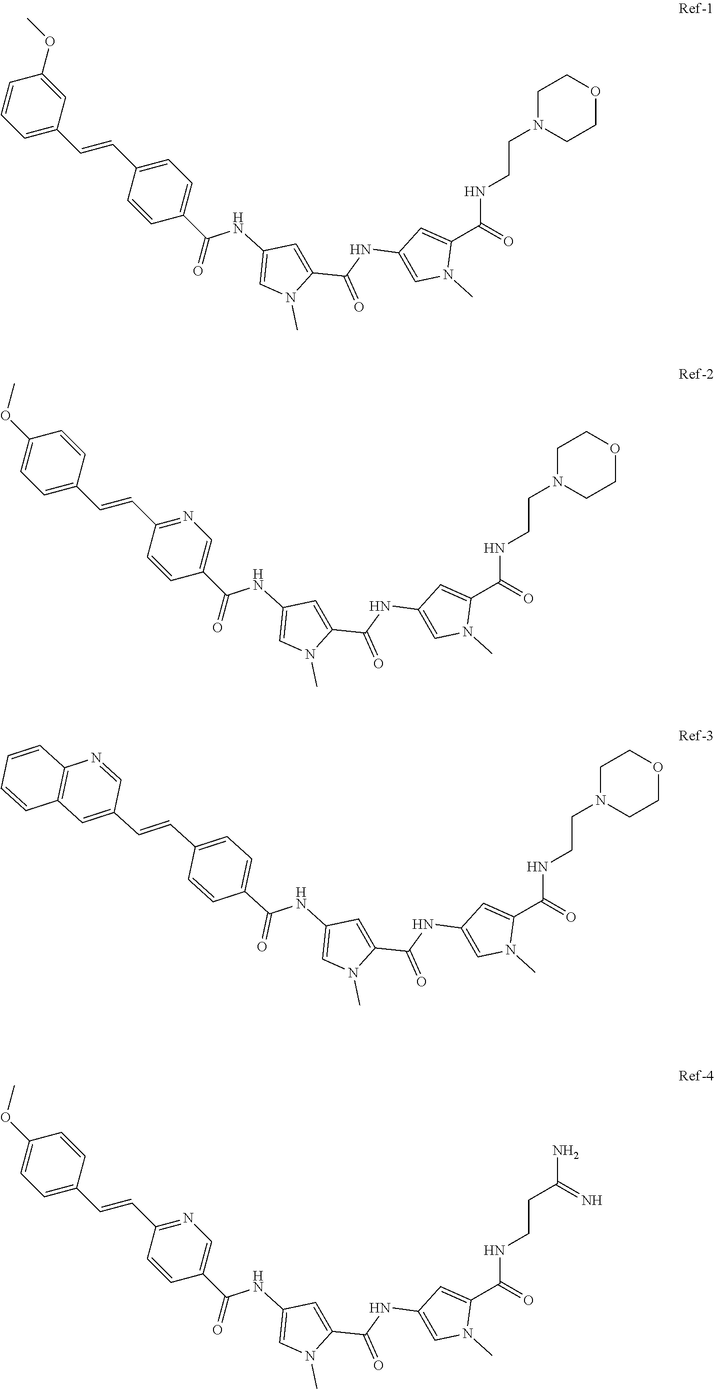 [SMILES: COc1cccc(/C=C/c2ccc(C(=O)Nc3cc(C(=O)Nc4cc(C(=O)NCCN5CCOCC5)n(C)c4)n(C)c3)cc2)c1 ; COc1ccc(/C=C/c2ccc(C(=O)Nc3cc(C(=O)Nc4cc(C(=O)NCCN5CCOCC5)n(C)c4)n(C)c3)cn2)cc1 ; Cn1cc(NC(=O)c2cc(NC(=O)c3ccc(/C=C/c4cnc5ccccc5c4)cc3)cn2C)cc1C(=O)NCCN1CCOCC1 ; COc1ccc(/C=C/c2ccc(C(=O)Nc3cc(C(=O)Nc4cc(C(=O)NCCC(=N)N)n(C)c4)n(C)c3)cn2)cc1]

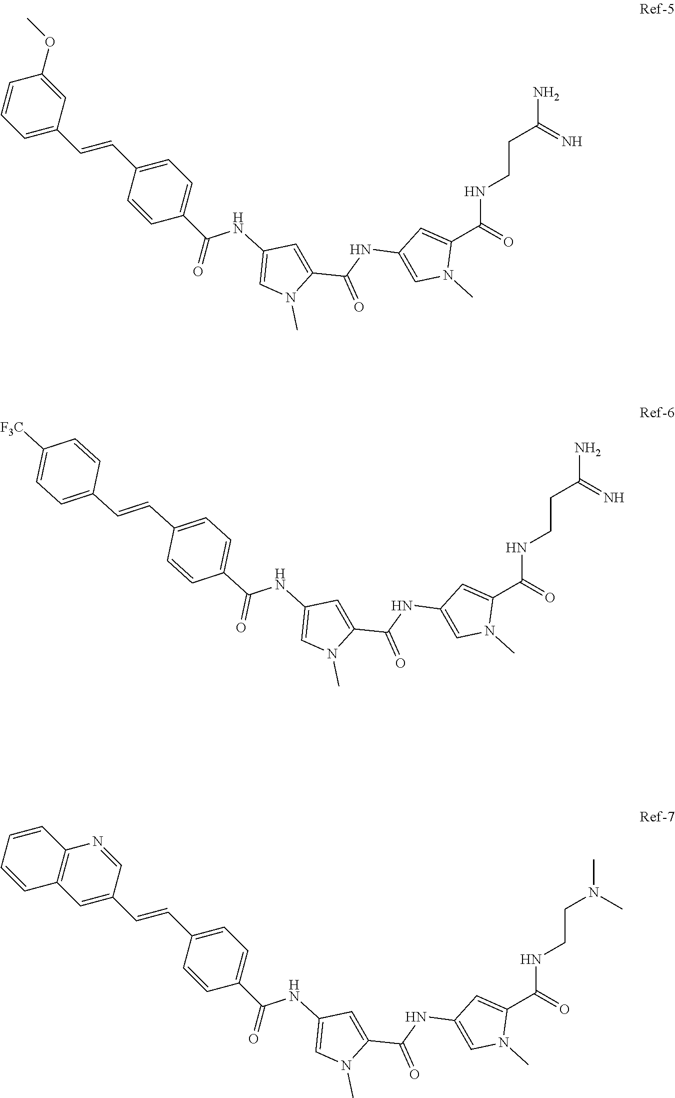 [SMILES: COc1cccc(/C=C/c2ccc(C(=O)Nc3cc(C(=O)Nc4cc(C(=O)NCCC(=N)N)n(C)c4)n(C)c3)cc2)c1 ; Cn1cc(NC(=O)c2cc(NC(=O)c3ccc(/C=C/c4ccc(C(F)(F)F)cc4)cc3)cn2C)cc1C(=O)NCCC(=N)N ; CN(C)CCNC(=O)c1cc(NC(=O)c2cc(NC(=O)c3ccc(/C=C/c4cnc5ccccc5c4)cc3)cn2C)cn1C]

TABLE 8
S. aureus Selectivity Indices
S. aureus Selectivity Indices (SI, IC₅₀ (μM)/MIC₈₀ (μM))

| | |
|---|---|
| Compound | Group |

Alkyl Amidines and NH Pyrroles
Group A: SI < 3, Group B: 3 < SI <= 5,
Group C: 5 < SI <= 10, D: SI > 10
Reference compound

| | |
|---|---|
| Ref-1 | A |
| Ref-2 | A |
| Ref-3 | A |
| Ref-4 | A |
| Ref-5 | A |
| Ref-6 | A |
| Ref-7 | A |

Compound of the invention

| | |
|---|---|
| Ia1 | B |
| Ia9 | C |
| Ia10 | B |
| Ia12 | C |
| Ia13 | C |
| Ia14 | D (SI = 44) |
| Ia15 | C |
| Ia18 | B |
| Ia19 | B |
| Ia32 | C |
| Ia34 | D (SI = 15) |
| Ia45 | B |
| Ib1 | D (SI = 11) |
| Ib3 | B |
| Ib5 | D (SI = 27) |
| Ib7 | D (SI = 26) |
| IIIa1 | C |
| IIIa2 | D (SI = 11) |
| IIIa3 | B |
| IIIa4 | D (SI = 81) |
| IIIa5 | C |
| IIIa6 | D (SI = 14) |
| IIIa7 | D (SI = 14) |
| IIIa9 | C |
| IIIb7 | B |

Amides
Group A: SI < 1.4, Group B: 1.4 < SI <= 3,
Group C: SI > 3
Reference compound

| | |
|---|---|
| Ref-3 | A |

Compound of the invention

| | |
|---|---|
| IVa1 | B |
| IVa2 | C (SI = 4) |
| IVa5 | B |

Quaternary ammoniums
Group A: SI < 2, Group B: 2 < SI <= 5,
Group C: 5 < SI <= 10, Group D: SI > 10
Reference compound

| | |
|---|---|
| Ref-7 | A |

Compound of the invention

| | |
|---|---|
| Ic4 | D (SI = 11) |
| Ic5 | C |
| Ic7 | D (SI = 13) |
| Ic8 | C (SI = 6) |
| Ic9 | B |
| Ic18 | C |

TABLE 9
E. faecalis Selectivity Indices
E. faecalis Selectivity Indices (SI, IC₅₀ (μM)/MIC₈₀ (μM))

| | |
|---|---|
| Compound | Group |

Alkyl Amidines and NH Pyrroles
Group A: SI < 3, Group B: 3 < SI <= 5,
Group C: 5 < SI <= 10, D: SI > 10
Reference compound

| | |
|---|---|
| Ref-1 | A |
| Ref-2 | A |
| Ref-3 | A |
| Ref-4 | A |
| Ref-5 | A |
| Ref-6 | A |
| Ref-7 | A |

Compound of the invention

| | |
|---|---|
| Ia12 | D (SI = 23) |
| Ia13 | D (SI = 12) |
| Ia14 | D (SI = 11) |
| Ia16 | B |
| Ia22 | C |
| Ia24 | C |
| Ib5 | C |
| Ib7 | C |
| IIIa1 | B |
| IIIa4 | D (SI = 41) |
| IIIa6 | D (SI = 14) |
| IIIa9 | C |

Amides
Group A: SI < 0.4, Group B: 0.4 < SI <= 1.5,
Group: C, 1.5 < SI <= 5, Group D: SI > 5
Reference compound

| | |
|---|---|
| Ref-3 | A |

Compound of the invention

| | |
|---|---|
| IVa1 | C (SI = 3) |
| IVa2 | D (SI = 10) |
| IVa5 | C (SI = 2) |
| IVa6 | B |

Quaternary ammoniums
Group A: SI < 0.7, Group B: 0.7 < SI <= 2,
Group C: 2 < SI <= 5, Group D: SI > 5
Reference compound

| | |
|---|---|
| Ref-7 | A |

Compound of the invention

| | |
|---|---|
| Ic3 | D (SI = 11) |
| Ic4 | C |
| Ic9 | B |
| Ic18 | D (SI = 5.1) |

TABLE 10
C. auris Selectivity Indices
C. auris Selectivity Indices (SI, IC₅₀ (μM)/MIC₉₉ (μg/mL))

| | |
|---|---|
| Compound | Group |

Alkyl Amidines and NH Pyrroles
Group A: SI < 0.4, Group B: 0.4 < SI <= 1,
Group C 1 < SI <= 3, Group D SI > 3
Reference compound

| | |
|---|---|
| Ref-1 | A |
| Ref-2 | A |
| Ref-3 | A |
| Ref-4 | A |

Compound of the invention

| | |
|---|---|
| Ia1 | B |
| Ia21 | B |
| IIIa2 | C (SI = 3) |
| IIIa4 | C |
| IIIa5 | D (SI = 5) |
| IIIa6 | D |
| IIIa7 | C |
| IIIa9 | D (SI = 6) |

TABLE 11
C. glabrata Selectivity Indices
C. glabrata Selectivity Indices (SI, IC₅₀ (μM)/MIC₉₉ (μg/mL))

| | |
|---|---|
| Compound | Group |

Alkyl Amidines and NH Pyrroles
Group A: SI < 12, Group B: 12 < SI <= 20,
Group C 20 < SI <= 50, Group D: SI > 50
Reference compound

| | |
|---|---|
| Ref-1 | A |
| Ref-2 | A |
| Ref-3 | A |
| Ref-4 | A |
| Ref-5 | A |

Compound of the invention

| | |
|---|---|
| IIIa2 | C (SI = 47) |
| IIIa3 | B |
| IIIa4 | D (SI = 652) |
| IIIa6 | D (SI = 109) |
| IIIa7 | B |
| IIIa9 | C |

TABLE 12
T.b. brucei Selectivity Indices
T.b. brucei Selectivity Indices (SI, IC₅₀ (μM)/IC₅₀ (μM))

| | |
|---|---|
| Compound | Group |

Alkyl Amidines and NH Pyrroles
Group A: SI < 70, Group B: 70 < SI <= 150,
Group C: 150 < SI <= 300, Group D:
SI > 300
Reference compound

| | |
|---|---|
| Ref-1 | A |
| Ref-2 | A |
| Ref-3 | A |
| Ref-4 | A |
| Ref-5 | A |
| Ref-6 | A |
| Ref-7 | A |

Compound of the invention

| | |
|---|---|
| Ia12 | D (SI = 730) |
| Ia13 | D (SI = 922) |
| Ia14 | D (SI = 863) |
| Ia15 | B |
| Ia16 | C (SI = 164) |
| Ia18 | B |
| Ia19 | B |
| Ia21 | D (SI = 472) |
| Ia24 | B |
| Ia32 | B (SI = 106) |
| Ia34 | B (SI = 130) |
| Ia45 | D (SI = 360) |
| Ib1 | B |
| Ib5 | D (SI = 346) |
| Ib7 | D (SI = 332) |
| IIIa1 | D (SI = 1049) |
| IIIa2 | D (SI = 600) |
| IIIa3 | D (SI = 635) |
| IIIa4 | D (SI = 3362) |
| IIIa5 | B |
| IIIa6 | D (SI = 1192) |
| IIIa8 | D (SI = 3492) |
| IIIb6 | D (SI = 366) |

Amides
Group A: SI < 0.7, Group B: 0.7 < SI <= 1.5,
Group C: 1.5 < SI <= 3, Group D: SI > 3
Reference compound

| | |
|---|---|
| Ref-3 | A |

Compound of the invention

| | |
|---|---|
| IVa1 | B |
| IVa2 | D (SI = 5) |
| IVa3 | D (SI = 23) |
| IVb1 | C |
| IVa4 | B |
| IVb2 | C |
| IVa5 | D (SI = 6) |
| IVa6 | C |
| IVb3 | D (SI = 6) |
| IVa7 | C |
| IVa8 | D |
| IVa10 | B |

Quaternary ammoniums
Group A: SI < 40, Group B: 40 < SI <= 80,
Group C: 80 < SI <= 120, Group D: SI > 120
Reference compound

| | |
|---|---|
| Ref-7 | A |

Compound of the invention

| | |
|---|---|
| Ic3 | C |
| Ic4 | C (SI = 107) |
| Ic5 | B |
| Ic7 | D (SI = 247) |
| Ic8 | D |
| Ic9 | D (SI = 131) |
| Ic10 | C |
| Ic18 | B |

TABLE 13
T. vaginalis Selectivity Indices
T. vaginalis Selectivity Indices (SI, IC₅₀ (μM)/IC₅₀ (μM))

| | |
|---|---|
| Compound | Group |

Alkyl Amidines and NH Pyrroles
Group A: SI < 8, Group B: 8 < SI <= 16,
Group C: 16 < SI <= 32, Group D: SI > 32
Reference compound

| | |
|---|---|
| Ref-1 | A |
| Ref-2 | A |
| Ref-3 | A |
| Ref-4 | A |
| Ref-5 | A |
| Ref-6 | A |
| Ref-7 | A |

Compound of the invention

| | |
|---|---|
| Ia12 | B |
| Ia13 | C (SI = 17) |
| Ia14 | D (SI = 35) |
| Ia21 | D (SI = 41) |
| Ia24 | D (SI = 69) |
| Ib5 | C |
| Ib7 | B |

Amides
Group A: SI < 0.25, Group B: 0.25 < SI <= 0.5,
Group C: SI > 0.5
Reference compound

| | |
|---|---|
| Ref-3 | A |

Compound of the invention

| | |
|---|---|
| IVa5 | C (SI = 2) |
| IVa6 | B |
| IVa8 | B |

Quaternary ammoniums
Group A: SI < 0.8, Group B: 0.8 < SI <= 1.5,
Group C: 1.5 < SI <= 3, Group D: SI > 3
Reference compound

| | |
|---|---|
| Ref-7 | A |

Compound of the invention

| | |
|---|---|
| Ic3 | D (SI = 6) |
| Ic4 | D (SI = 4) |
| Ic5 | B |
| Ic18 | C |

TABLE 14
SARS-CoV-2 Selectivity Indices
SARS-CoV-2 Selectivity Indices (SI, IC₅₀ (μM)/ IC₅₀ (μM))

| | |
|---|---|
| Compound | Group |

Alkyl Amidines and NH Pyrroles
Group A: SI < 4.1, Group B: 4.1 < SI <= 10,
Group C: 10 < SI <= 25, Group D:
SI > 25
Reference compound

| | |
|---|---|
| Ref-1 | A |
| Ref-2 | A |
| Ref-3 | A |
| Ref-4 | A |
| Ref-5 | A |

Compound of the invention

| | |
|---|---|
| Ia1 | B |
| Ia9 | B |
| IIIa5 | D (SI = 39) |
| IIIa7 | C (SI = 24) |
| IIIa9 | D (SI = 33) |

### In Vivo Data

The procedure used for in vivo pharmacokinetics experiments is described below.

### Stock Preparation

1 mL of 2 mg/mL stock solution was prepared by adding 500 μL of DMSO to 2 mg of compound and vortexed well until the compound had fully dissolved. 500 μL of Saline for Injection (SFI, warmed to 37° C.) was added dropwise, under vortexing.

Working Solutions Immediately prior to dosing, the 2 mg/mL stock solution was diluted 1:5 to produce 0.4 mg/mL dosing solution. 2 mL of 0.4 mg/mL solution was prepared by adding 400 μL of 1 mg/mL stock solution to 1600 μL diluent (10% DMSO, 90% SFI).

The dosing solution was administered to 7 male CD-1 mice, at 5 mL/kg via intravenous (IV) injection (e.g. 30 g mouse with 150 μL). 3 mice were grouped for the blood sampling study and 4 mice were grouped for the tissue sampling study.

### Blood Sampling

At each time point (0.5, 2, 4, 8 hours), 20 μL blood samples were collected from mice by tail prick into 20 μL EDTA blood collection capillary tubes on wet ice. 20 μL blood samples were added directly to the appropriate well of a 96 deep well plate and mixed with 20 μL of distilled water and the samples stored at −80° C. prior to bioanalysis.

### Tissue Sampling

At each time point (0.25 and 1 hour), the lung, both kidneys and the liver (left lateral lobe) were removed from culled mice (two per time point) and individually placed in pre-weighed bead beater tubes, which were then weighed to determine tissue weight. 4× weight/volume sterile water was added to the tissue. Tissues were homogenized in a Precellys bead beater then snap frozen at 80° C. until bioanalysis.

### Bioanalysis

### Standards and Quality Controls

Initial stock of Ia10 was made to 1 mg/mL in DMSO. Calibration and quality controls were made by spiking 1 mg/mL DMSO stock into desired matrix and serial dilution in matrix to desired concentration. Calibration range=1 ng/mL-5000 ng/mL. All samples were crashed out in 5× volume of Acetonitrile. Samples were centrifuged and supernatant diluted 1:3 in water to be analysed on LCMS.

### Lcms Information

### Sciex QTrap 5500

TABLE 15
LCMS details

| | |
|---|---|
| Mobile Phase: | A: 0.1% Formic acid in Water; |
| | B: 0.1% Formic acid in 25% |
| | Acetonitrile 75% Methanol |
| Loop Volume (uL): | 20 |
| Column Type: | Phenomenex Kinetex 2.6 μm Biphenyl |
| | 100A 50 × 2.1 mm |
| Column Position & Temp: | Left hand side: 50° C. |
| Injection Volume: | 1.000 μL |

TABLE 16
LCMS gradient

| | Time | Flow Rate | A | B |
|---|---|---|---|---|
| Step | (min) | (μl/min) | (%) | (%) |
| 0 | 0 | 800 | 98 | 2 |
| 1 | 0.1 | 800 | 98 | 2 |
| 2 | 1 | 800 | 5 | 95 |
| 3 | 1.5 | 800 | 5 | 95 |
| 4 | 1.55 | 800 | 98 | 2 |
| 5 | 1.8 | 800 | 98 | 2 |

TABLE 17
Source conditions

| | |
|---|---|
| Curtain Gas | 35 |
| IS | 5500 |
| Temp | 600 |
| GS1 | 60 |
| GS2 | 40 |
| CAD | Medium |

### Results

Compound Ia10 has been evaluated in an in vivo pharmacokinetics mouse model in order to investigate compound distribution and tolerability. In this experiment, Ia10 was found to be well tolerated at 2 mg/kg (higher doses were not investigated), and, significantly, the compound was found to have remarkable accumulation in lung tissue. This accumulation could prove to be advantageous in treating pulmonary infections.

TABLE 18
Pharmacokinetic data illustrating tolerability
and lung accumulation of Ia10.
Ia10 2 mg/kg IV dose in mice

| Blood conc @ 1 Hr | 204 nM |
|---|---|
| Lung conc @ 1 Hr | 28968 nM |
| Lung accumulation factor | ~142 |

1. A compound of any one of formulae I, II, III and IV: 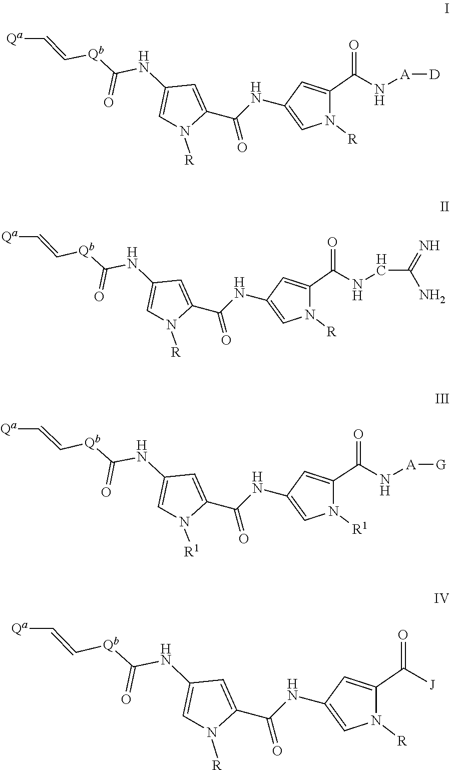
wherein:
Q^{a} is optionally substituted aryl or optionally substituted heteroaryl and Q^{b} is optionally substituted arylene or optionally substituted heteroarylene, wherein the aryl, heteroaryl, arylene and heteroarylene are optionally substituted with any one or a combination selected from the group consisting of N(C₁₋₆alkyl)₂, haloC₁₋₆alkyl, cyano, C₁₋₆alkyl, C₁₋₆alkoxy, NH(C₁₋₆alkyl), NH₂, halo and OH;
each R is independently selected from the group consisting of C₁₋₆alkyl, H and haloC₁₋₆alkyl;
each R¹ is independently selected from the group consisting of C₁₋₆alkyl, H and haloC₁₋₆alkyl, wherein at least one R¹ is H;
A is C₁₋₆alkylene or haloC₁₋₆alkylene;
E is C₄₋₆alkylene or haloC₄₋₆alkylene;
A-D is selected from the group consisting of formulae Ia to Id: 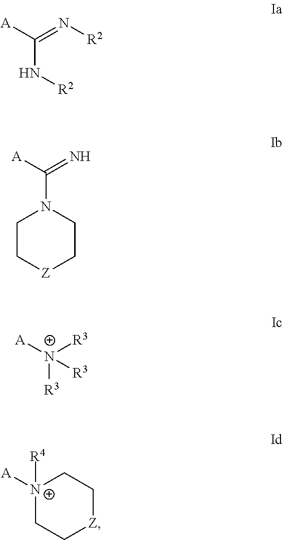
wherein:
Z is selected from the group consisting of O, CH₂, N(C₁₋₆alkyl) and S; and
each R² is independently selected from the group consisting of H, C₁₋₂₀alkyl, C₁₋₂₀alkoxy, C₂₋₂₀alkenyl, C₄₋₂₀dialkenyl and C₆₋₂₀trialkenyl, wherein at least one R² is not H, and wherein the C₁₋₂₀alkyl, C₁₋₂₀alkoxy, C₂₋₂₀alkenyl, C₄₋₂₀dialkenyl and C₆₋₂₀trialkenyl are optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo;
each R³ is independently selected from the group consisting of C₁₋₂₀alkyl, C₂₋₂₀alkenyl, C₄₋₂₀dialkenyl and C₆₋₂₀trialkenyl, optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O, and OH, wherein the aryl and heterocyclyl are optionally substituted with any one or more independently selected from the group consisting of C₁₋₄alkyl, haloC₁₋₄alkyl, C₁₋₄alkoxy and halo;
R⁴ is selected from the group consisting of C₂₋₂₀alkyl, C₂₋₂₀alkenyl, C₄₋₂₀dialkenyl and C₆₋₂₀trialkenyl, optionally substituted with any one or more independently selected from the group consisting of aryl, heterocyclyl, N(C₁₋₆alkyl)₂, methoxy(ethoxy)₁₋₃, hydroxy(ethoxy)₁₋₃, cycloC₃₋₈alkyl, halo, cyano, C₁₋₆alkoxy, NH(C₁₋₆alkyl), NH₂, ═O and OH, wherein the aryl and heterocyclyl are optionally substituted with any one or more independently selected from the group consisting of C₁₋₄alkyl, haloC₁₋₄alkyl, C₁₋₄alkoxy and halo;
A-G is selected from the group consisting of formulae IIIa and IIIb: 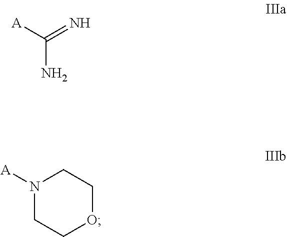
C(O)-J is any one selected from the group consisting of formulae IVa and IVb: 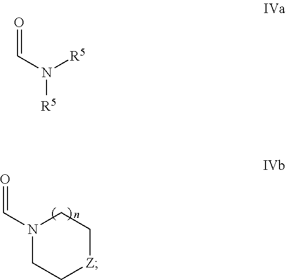
wherein:
each R⁵ is independently selected from the group consisting of H, C₁₋₂₀alkyl, C₂₋₂₀alkenyl, C₄₋₂₀dialkenyl and C₆₋₂₀trialkenyl, wherein the C₂₋₂₀alkenyl, C₄₋₂₀dialkenyl and C₆₋₂₀trialkenyl are optionally substituted with any one or more independently selected from the group consisting of methoxy(ethoxy)₁₋₃, hydroxy(ethoxy)₁₋₃, N(di(C₁₋₄alkyl)aminoC₁₋₆alkyl)₂, N((C₁₋₄alkyl)aminoC₁₋₆alkyl)₂, N(aminoC₁₋₆alkyl)₂, cycloC₃₋₈alkyl, pyrrolidinyl, piperidinyl, piperazinyl, morpholinyl, thiomorpholinyl, aryl, halo, cyano, C₁₋₆alkoxy, ═O and OH, and the C₁₋₂₀alkyl is optionally substituted with any one or more independently selected from the group consisting of methoxy(ethoxy)₁₋₃, hydroxy(ethoxy)₁₋₃, di(C₁₋₄alkyl)amino, C₁₋₄alkylamino, amino, cycloC₃₋₈alkyl, pyrrolidinyl, 1-(3-propyl)piperidine, 1-(3-propyl)piperazine, 4-(3-propyl)morpholine, 4-(3-propyl)thiomorpholine, aryl, halo, cyano, C₁₋₆alkoxy, ═O and OH, wherein the cycloC₃₋₈alkyl, pyrrolidinyl, piperidinyl, piperazinyl, morpholinyl, thiomorpholinyl, 1-(3-propyl)piperidine, 1-(3-propyl)piperazine, 4-(3-propyl)morpholine, 4-(3-propyl)thiomorpholine and aryl are optionally substituted with any one or more independently selected from the group consisting of C₁₋₄alkyl, haloC₁₋₄alkyl, C₁₋₄alkoxy and halo; and
n is 1 to 3.

2. The compound of claim 1, wherein Q^{a} and Q^{b} are optionally substituted with any one or a combination selected from the group consisting of N(C₁₋₄alkyl)₂, haloC₁₋₄alkyl, cyano, C₁₋₄alkyl, C₁₋₄alkoxy and halo.

3. The compound of claim 1, wherein Q^{a} is selected from the group consisting of optionally substituted phenyl, quinolinyl, benzoxadiazolyl, naphthalenyl, benzothiazolyl, isoquinolinyl, thiazolyl, pyridinyl, pyrimidinyl, thiophenyl, pyridazinyl, phthalazinyl, imidazolyl and pyrollyl.

4. The compound of claim 1, wherein Q^{b} is monocyclic.

5. The compound of claim 1, wherein Q^{b} is unsubstituted.

6. The compound of claim 1, wherein each R is independently selected from the group consisting of C₁₋₄alkyl and H.

7. The compound of claim 1, wherein A is C₁₋₆alkylene.

8. The compound of claim 1, wherein Z is selected from the group consisting of O, CH₂, N(CH₃) and S.

9. The compound of claim 1, wherein each R² is independently selected from the group consisting of H, C₁₋₉alkyl, C₁₋₉alkoxy, C₂₋₉alkenyl, C₄₋₉dialkenyl and C₆₋₉trialkenyl, wherein the C₁₋₉alkyl, C₁₋₉alkoxy, C₂₋₉alkenyl, C₄₋₉dialkenyl and C₆₋₉trialkenyl are optionally substituted with any one or more independently selected from the group consisting of phenyl, 1-(C₁₋₆alkyl)piperazinyl, tetrahydropyranyl, N(C₁₋₆alkyl)₂, methoxy(ethoxy)₁₋₃, hydroxy(ethoxy)₁₋₃, thiomorphinyl, C₁₋₃alkoxy and halo.

10. The compound of claim 1, wherein each R² is independently selected from the group consisting of H, C₁₋₉alkyl and C₁₋₉alkoxy, wherein the C₁₋₉alkyl and C₁₋₉alkoxy are optionally substituted with any one or more independently selected from the group consisting of phenyl, 1-(methyl)piperazinyl, tetrahydropyranyl, N(C₁₋₃alkyl)₂, methoxy(ethoxy)₁₋₂, hydroxy(ethoxy)₁₋₂, thiomorphinyl, methoxy and fluoro.

11. The compound of claim 1, wherein each R³ is independently C₁₋₂₀alkyl, optionally substituted with any one or more independently selected from the group consisting of phenyl, 1-(methyl)piperazinyl, tetrahydropyranyl, N(C₁₋₃alkyl)₂, methoxy(ethoxy)₁₋₂, and hydroxy(ethoxy)₁₋₂.

12. The compound of claim 1, wherein R⁴ is C₂₋₂₀alkyl, optionally substituted with any one or more independently selected from the group consisting of phenyl, 1-(methyl)piperazinyl, tetrahydropyranyl, N(C₁₋₃alkyl)₂, methoxy(ethoxy)₁₋₂, and hydroxy(ethoxy)₁₋₂.

13. The compound claim 1, wherein R⁴ is C₂₋₁₀alkyl.

14. The compound of claim 1, wherein A-D is selected from the group consisting of formulae Ia and Ib.

15. The compound of claim 1, wherein E is C₄₋₆alkylene.

16. The compound of claim 1, wherein each R¹ is independently selected from the group consisting of C₁₋₄alkyl and H.

17. The compound of claim 1, wherein A-G is of formulae IIIa.

18. The compound of claim 1, wherein each R⁵ is independently selected from the group consisting of H, C₁₋₁₂alkyl, C₂₋₁₂alkenyl, C₄₋₁₂dialkenyl and C₆₋₁₂trialkenyl, wherein the C₁₋₁₂alkyl is optionally substituted with any one or more independently selected from the group consisting of methoxy(ethoxy)₁₋₂, hydroxy(ethoxy)₁₋₂, dimethylamino, methylamino, amino, cycloC₅₋₇alkyl, pyrrolidinyl, 1-(3-propyl)piperidine, 1-(3-propyl)piperazine, 4-(3-propyl)morpholine, 4-(3-propyl)thiomorpholine, aryl and C₁₋₃alkoxy.

19. The compound of claim 1, wherein each R⁵ is independently selected from the group consisting of H, C₁₋₁₂alkyl and C₄₋₁₂dialkenyl, wherein the C₁₋₁₂alkyl is optionally substituted with any one or more independently selected from the group consisting of methoxy(ethoxy)₁₋₂, hydroxy(ethoxy)₁₋₂, dimethylamino, methylamino, amino, cycloC₅₋₇alkyl, pyrrolidinyl, 1-(3-propyl)piperidine and methoxy.

20. The compound of claim 1, wherein n is 1 or 2.

21. The compound of claim 1, which is any one of the following formulae:

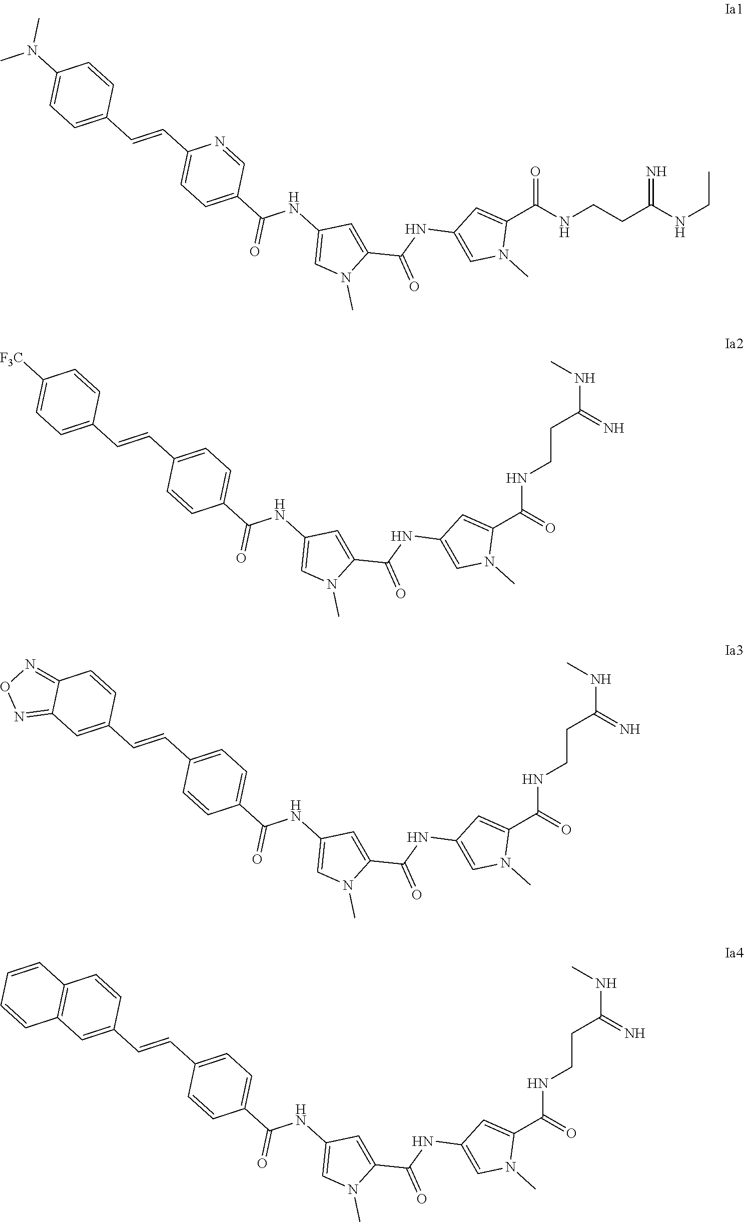 [SMILES: CCNC(=N)CCNC(=O)c1cc(NC(=O)c2cc(NC(=O)c3ccc(/C=C/c4ccc(N(C)C)cc4)nc3)cn2C)cn1C ; CNC(=N)CCNC(=O)c1cc(NC(=O)c2cc(NC(=O)c3ccc(/C=C/c4ccc(C(F)(F)F)cc4)cc3)cn2C)cn1C ; CNC(=N)CCNC(=O)c1cc(NC(=O)c2cc(NC(=O)c3ccc(/C=C/c4ccc5nonc5c4)cc3)cn2C)cn1C ; CNC(=N)CCNC(=O)c1cc(NC(=O)c2cc(NC(=O)c3ccc(/C=C/c4ccc5ccccc5c4)cc3)cn2C)cn1C]

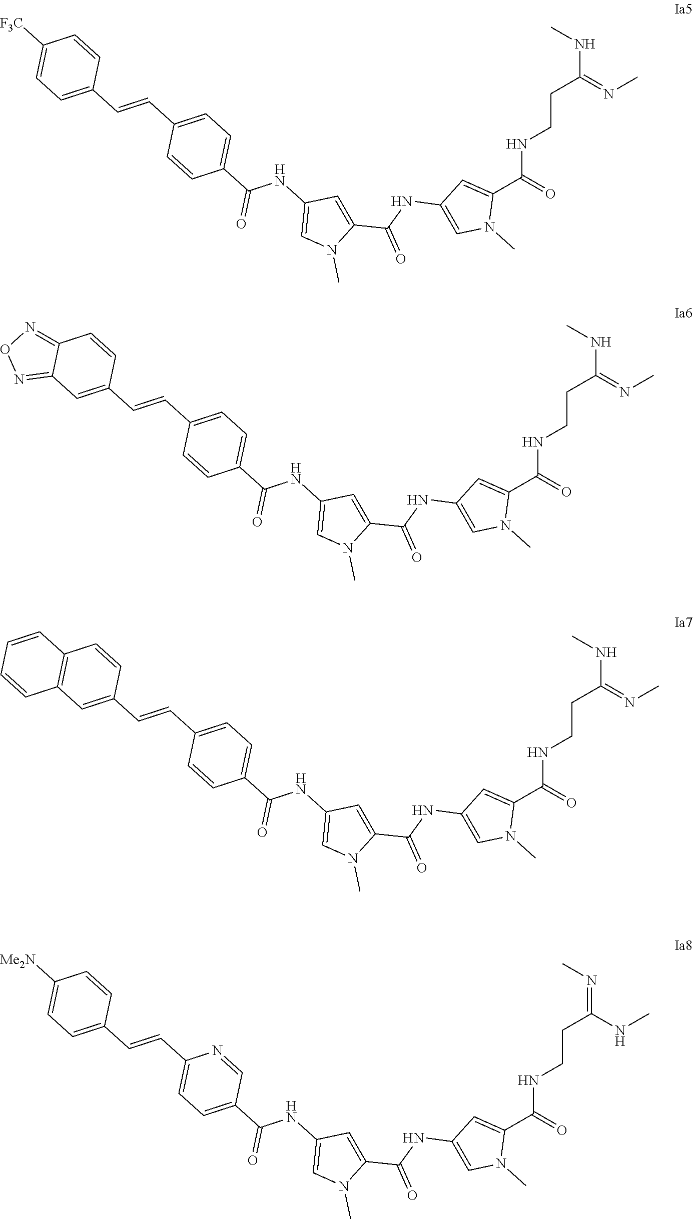 [SMILES: C/N=C(/CCNC(=O)c1cc(NC(=O)c2cc(NC(=O)c3ccc(/C=C/c4ccc(C(F)(F)F)cc4)cc3)cn2C)cn1C)NC ; C/N=C(/CCNC(=O)c1cc(NC(=O)c2cc(NC(=O)c3ccc(/C=C/c4ccc5nonc5c4)cc3)cn2C)cn1C)NC ; C/N=C(/CCNC(=O)c1cc(NC(=O)c2cc(NC(=O)c3ccc(/C=C/c4ccc5ccccc5c4)cc3)cn2C)cn1C)NC ; C/N=C(\CCNC(=O)c1cc(NC(=O)c2cc(NC(=O)c3ccc(/C=C/c4ccc(N(C)C)cc4)nc3)cn2C)cn1C)NC]

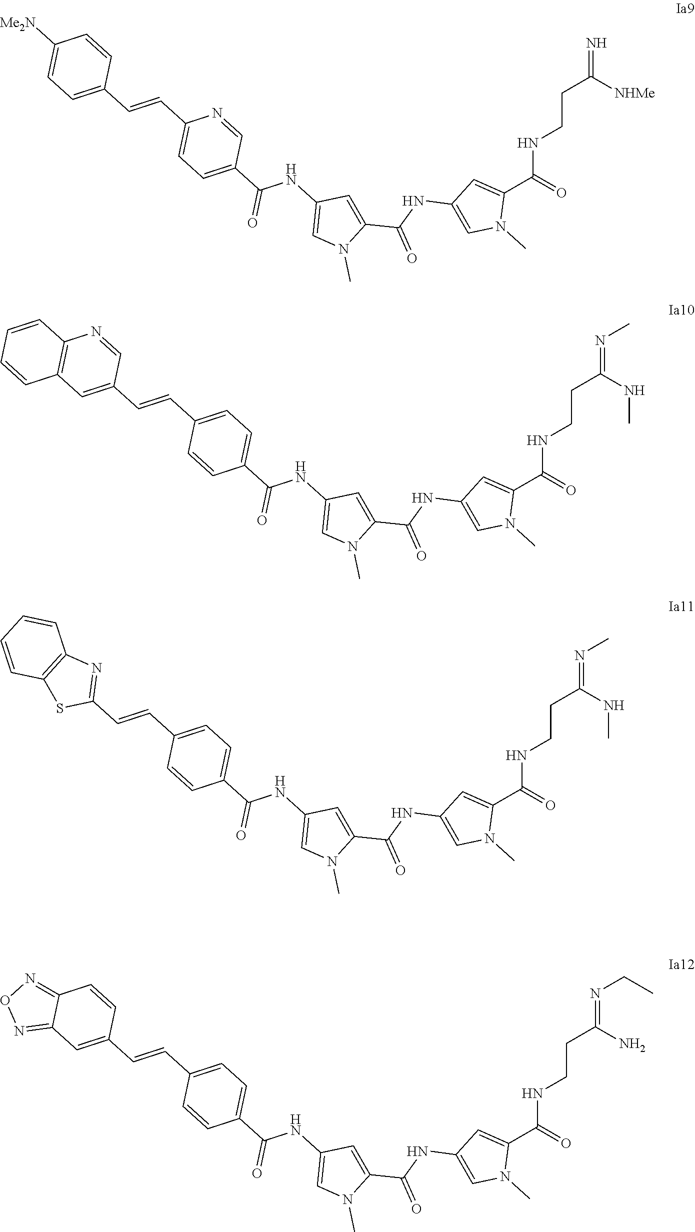 [SMILES: CNC(=N)CCNC(=O)c1cc(NC(=O)c2cc(NC(=O)c3ccc(/C=C/c4ccc(N(C)C)cc4)nc3)cn2C)cn1C ; C/N=C(/CCNC(=O)c1cc(NC(=O)c2cc(NC(=O)c3ccc(/C=C/c4cnc5ccccc5c4)cc3)cn2C)cn1C)NC ; C/N=C(/CCNC(=O)c1cc(NC(=O)c2cc(NC(=O)c3ccc(/C=C/c4nc5ccccc5s4)cc3)cn2C)cn1C)NC ; CC/N=C(\N)CCNC(=O)c1cc(NC(=O)c2cc(NC(=O)c3ccc(/C=C/c4ccc5nonc5c4)cc3)cn2C)cn1C]

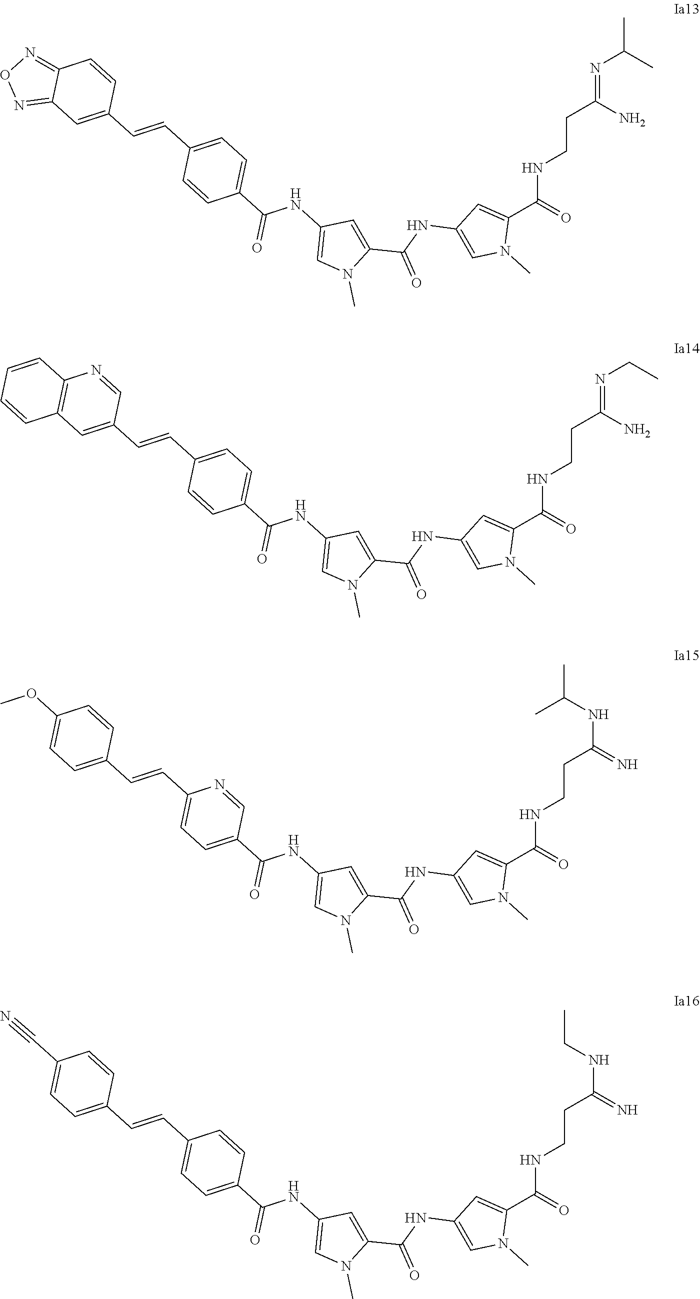

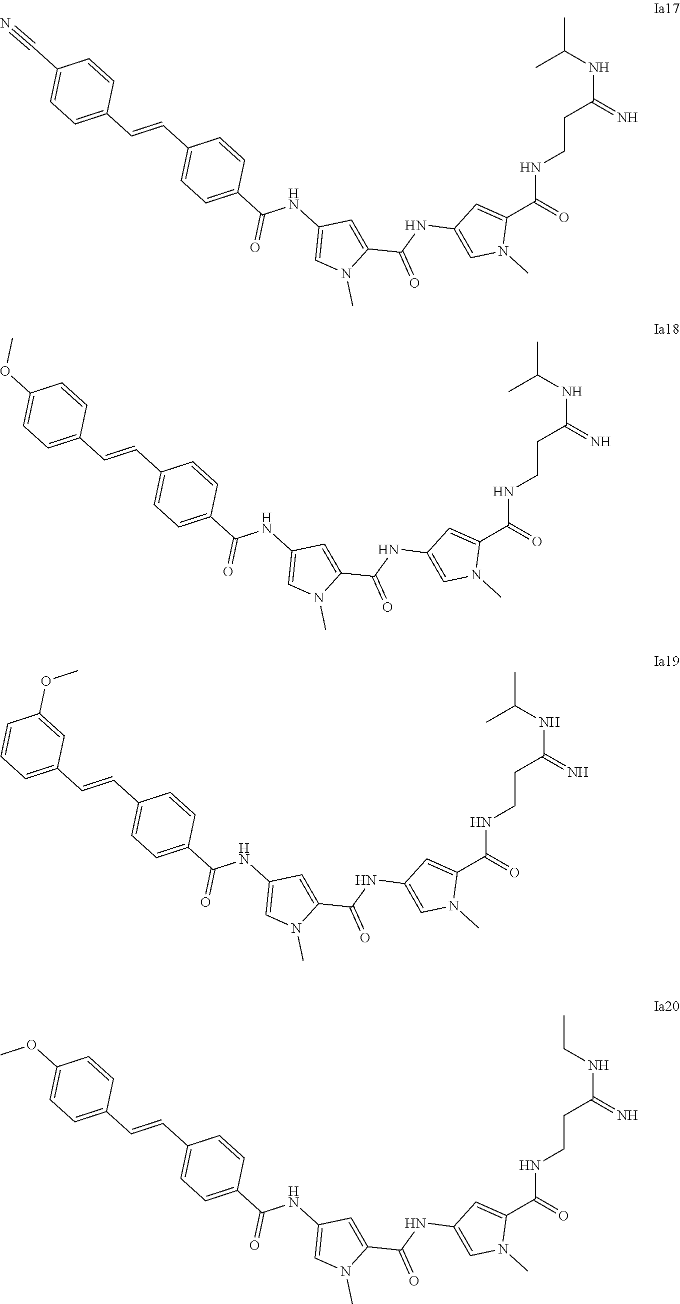 [SMILES: CC(C)NC(=N)CCNC(=O)c1cc(NC(=O)c2cc(NC(=O)c3ccc(/C=C/c4ccc(C#N)cc4)cc3)cn2C)cn1C ; COc1ccc(/C=C/c2ccc(C(=O)Nc3cc(C(=O)Nc4cc(C(=O)NCCC(=N)NC(C)C)n(C)c4)n(C)c3)cc2)cc1 ; COc1cccc(/C=C/c2ccc(C(=O)Nc3cc(C(=O)Nc4cc(C(=O)NCCC(=N)NC(C)C)n(C)c4)n(C)c3)cc2)c1 ; CCNC(=N)CCNC(=O)c1cc(NC(=O)c2cc(NC(=O)c3ccc(/C=C/c4ccc(OC)cc4)cc3)cn2C)cn1C]

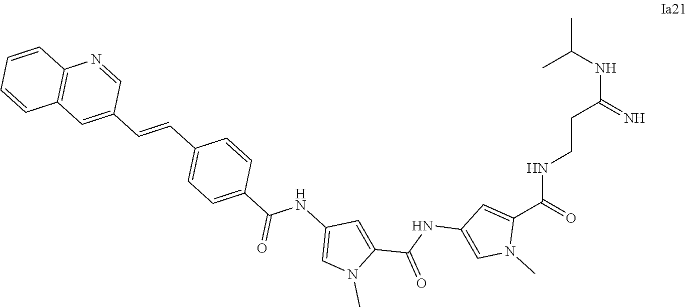 [SMILES: CC(C)NC(=N)CCNC(=O)c1cc(NC(=O)c2cc(NC(=O)c3ccc(/C=C/c4cnc5ccccc5c4)cc3)cn2C)cn1C]

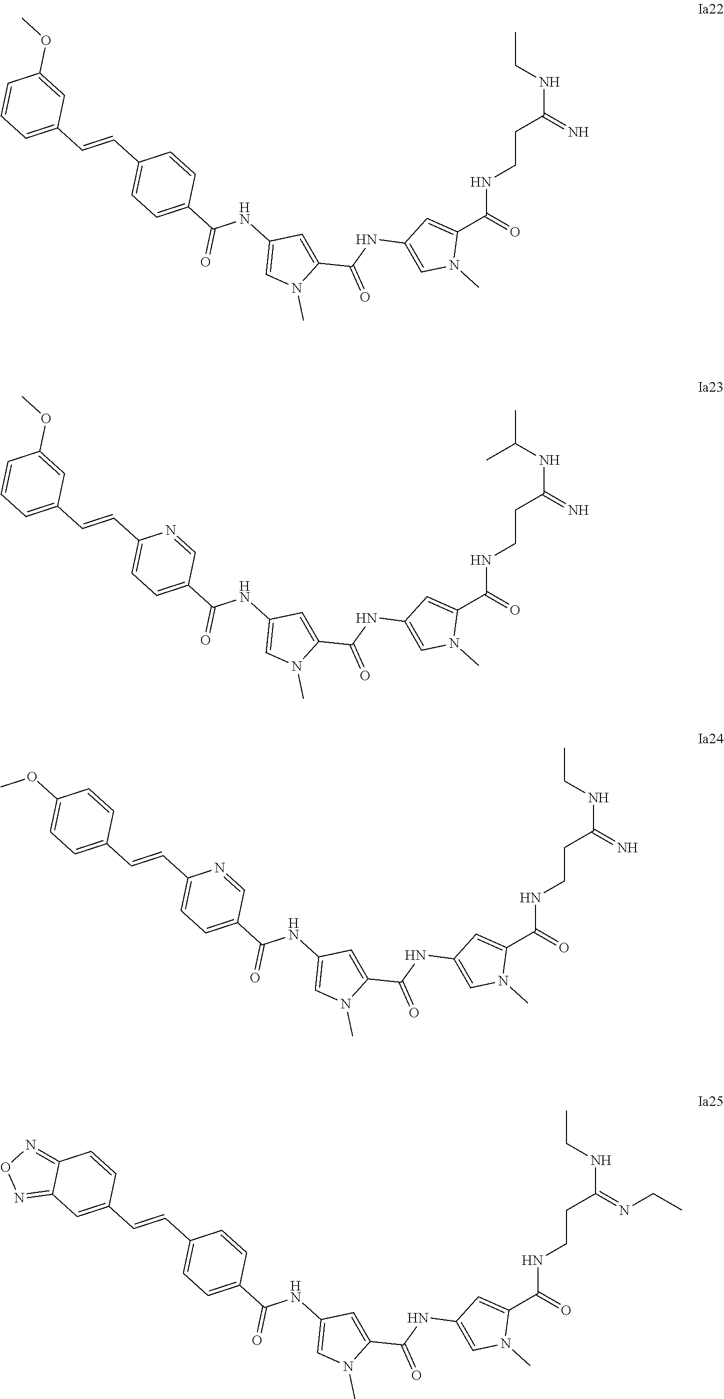 [SMILES: CCNC(=N)CCNC(=O)c1cc(NC(=O)c2cc(NC(=O)c3ccc(/C=C/c4cccc(OC)c4)cc3)cn2C)cn1C ; COc1cccc(/C=C/c2ccc(C(=O)Nc3cc(C(=O)Nc4cc(C(=O)NCCC(=N)NC(C)C)n(C)c4)n(C)c3)cn2)c1 ; CCNC(=N)CCNC(=O)c1cc(NC(=O)c2cc(NC(=O)c3ccc(/C=C/c4ccc(OC)cc4)nc3)cn2C)cn1C ; CC/N=C(/CCNC(=O)c1cc(NC(=O)c2cc(NC(=O)c3ccc(/C=C/c4ccc5nonc5c4)cc3)cn2C)cn1C)NCC]

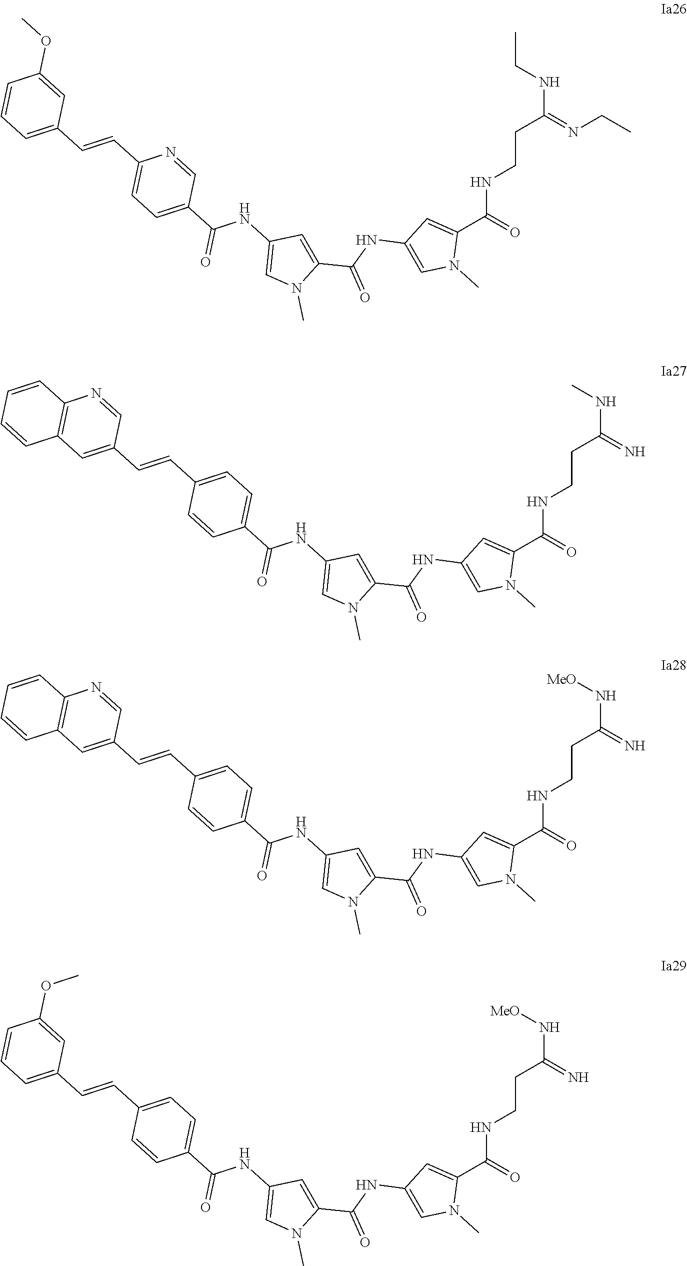 [SMILES: CC/N=C(/CCNC(=O)c1cc(NC(=O)c2cc(NC(=O)c3ccc(/C=C/c4cccc(OC)c4)nc3)cn2C)cn1C)NCC ; CNC(=N)CCNC(=O)c1cc(NC(=O)c2cc(NC(=O)c3ccc(/C=C/c4cnc5ccccc5c4)cc3)cn2C)cn1C ; CONC(=N)CCNC(=O)c1cc(NC(=O)c2cc(NC(=O)c3ccc(/C=C/c4cnc5ccccc5c4)cc3)cn2C)cn1C ; CONC(=N)CCNC(=O)c1cc(NC(=O)c2cc(NC(=O)c3ccc(/C=C/c4cccc(OC)c4)cc3)cn2C)cn1C]

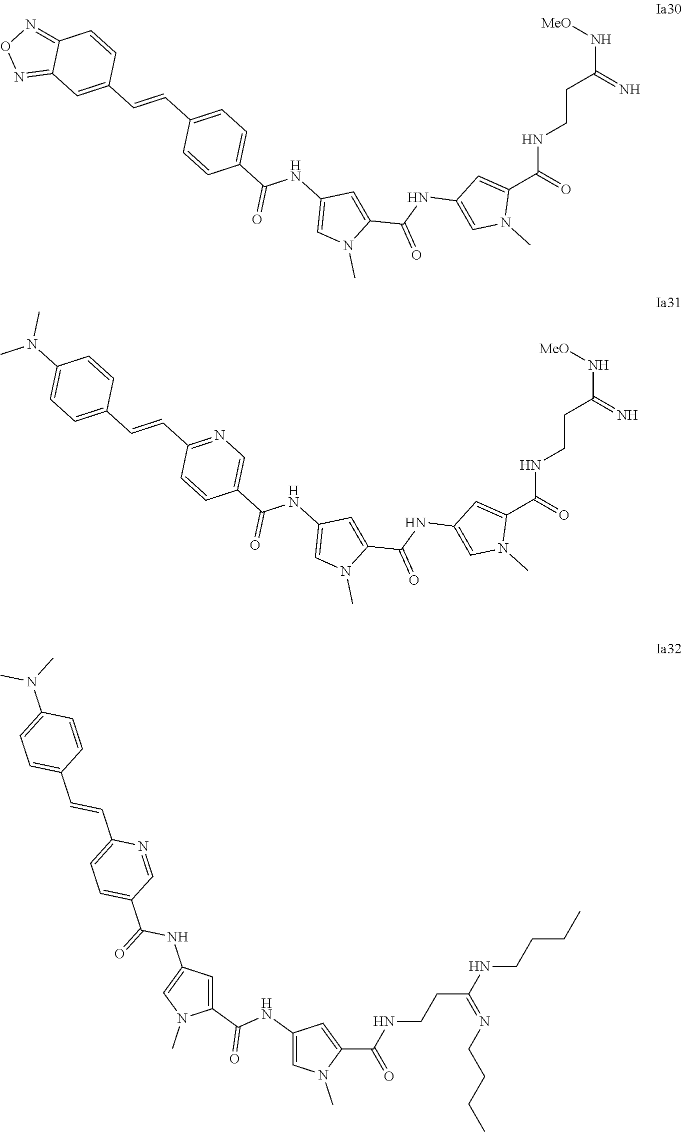 [SMILES: CONC(=N)CCNC(=O)c1cc(NC(=O)c2cc(NC(=O)c3ccc(/C=C/c4ccc5nonc5c4)cc3)cn2C)cn1C ; CONC(=N)CCNC(=O)c1cc(NC(=O)c2cc(NC(=O)c3ccc(/C=C/c4ccc(N(C)C)cc4)nc3)cn2C)cn1C ; CCCC/N=C(\CCNC(=O)c1cc(NC(=O)c2cc(NC(=O)c3ccc(/C=C/c4ccc(N(C)C)cc4)nc3)cn2C)cn1C)NCCCC]

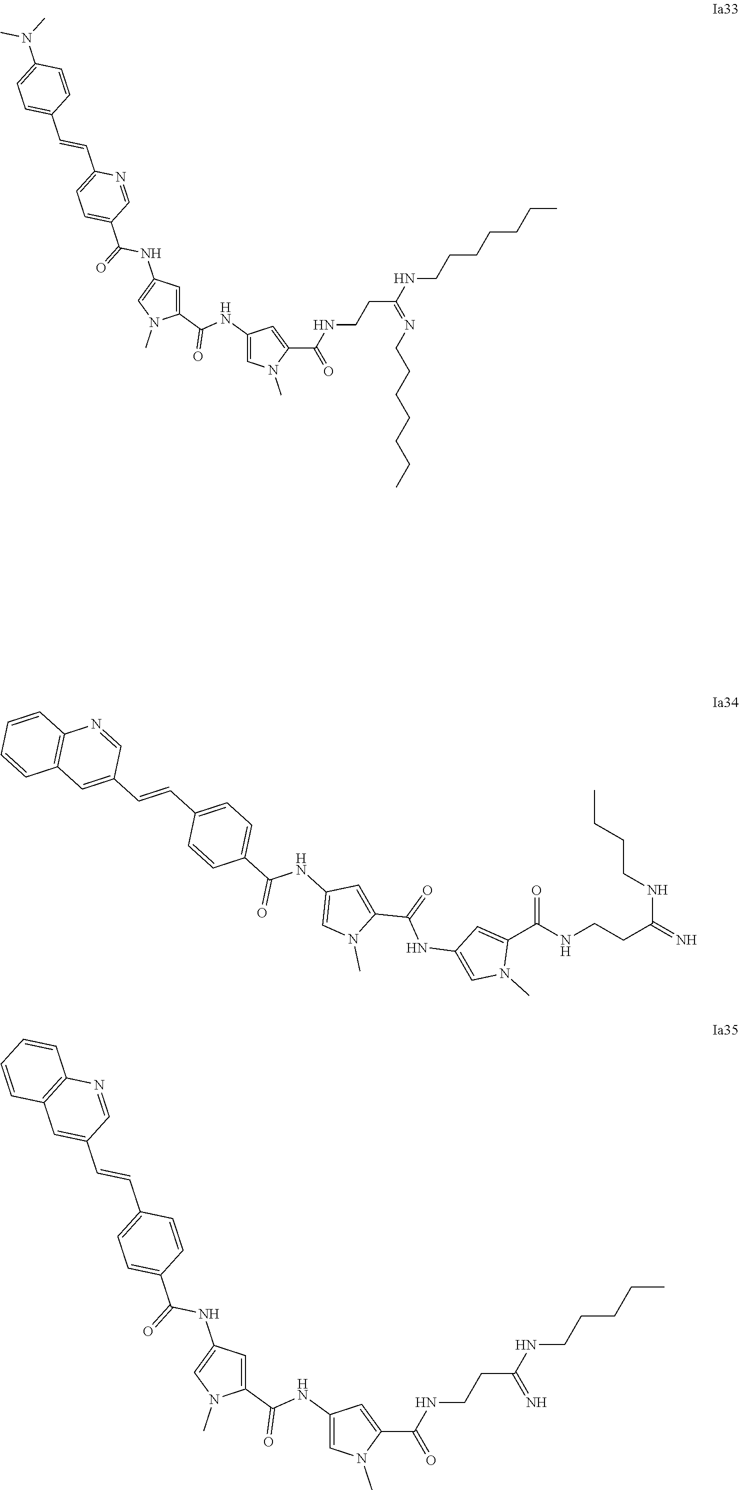 [SMILES: CCCCCCC/N=C(\CCNC(=O)c1cc(NC(=O)c2cc(NC(=O)c3ccc(/C=C/c4ccc(N(C)C)cc4)nc3)cn2C)cn1C)NCCCCCCC ; CCCCNC(=N)CCNC(=O)c1cc(NC(=O)c2cc(NC(=O)c3ccc(/C=C/c4cnc5ccccc5c4)cc3)cn2C)cn1C ; CCCCCNC(=N)CCNC(=O)c1cc(NC(=O)c2cc(NC(=O)c3ccc(/C=C/c4cnc5ccccc5c4)cc3)cn2C)cn1C]

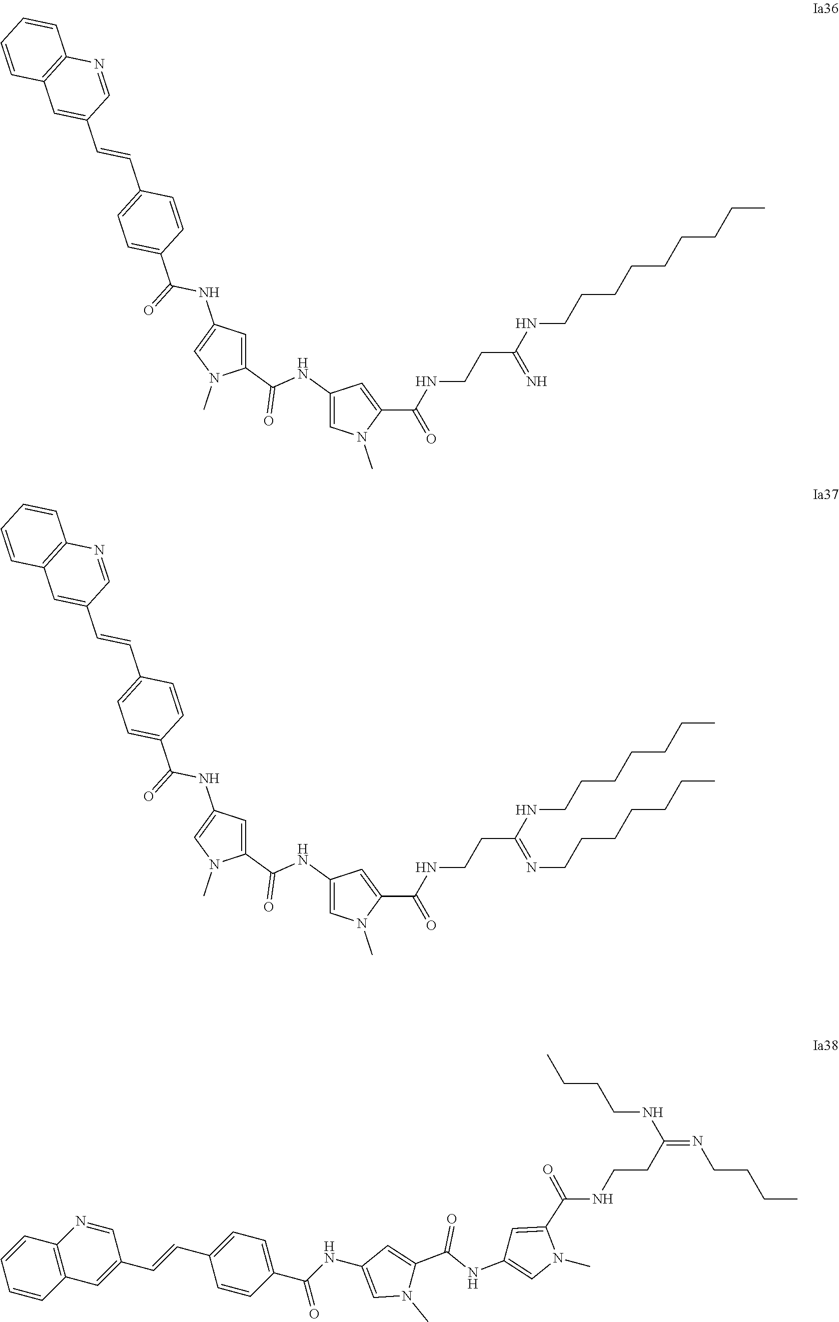 [SMILES: CCCCCCCCCNC(=N)CCNC(=O)c1cc(NC(=O)c2cc(NC(=O)c3ccc(/C=C/c4cnc5ccccc5c4)cc3)cn2C)cn1C ; CCCCCCC/N=C(/CCNC(=O)c1cc(NC(=O)c2cc(NC(=O)c3ccc(/C=C/c4cnc5ccccc5c4)cc3)cn2C)cn1C)NCCCCCCC ; CCCC/N=C(\CCNC(=O)c1cc(NC(=O)c2cc(NC(=O)c3ccc(/C=C/c4cnc5ccccc5c4)cc3)cn2C)cn1C)NCCCC]

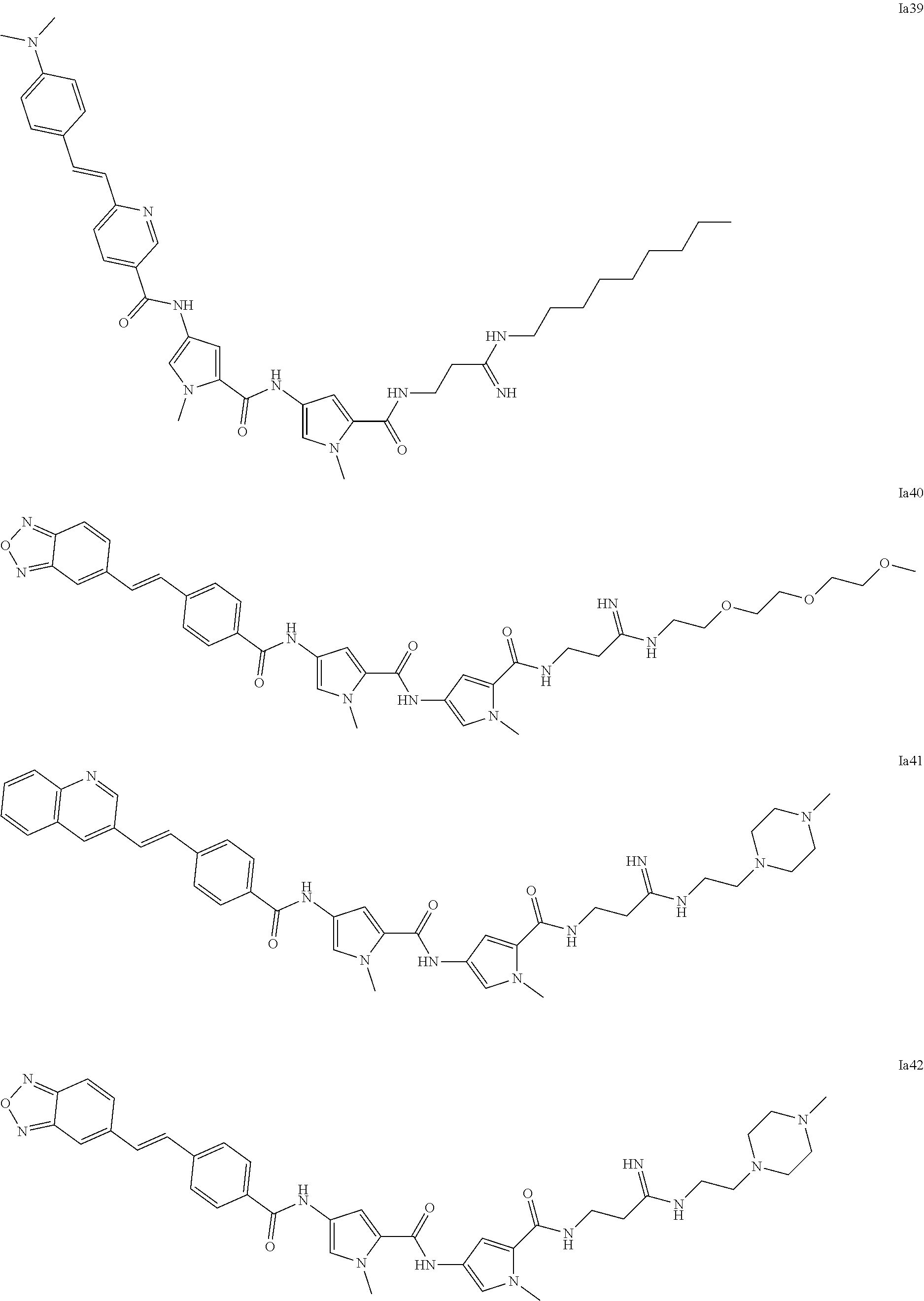 [SMILES: CCCCCCCCCNC(=N)CCNC(=O)c1cc(NC(=O)c2cc(NC(=O)c3ccc(/C=C/c4ccc(N(C)C)cc4)nc3)cn2C)cn1C ; COCCOCCOCCNC(=N)CCNC(=O)c1cc(NC(=O)c2cc(NC(=O)c3ccc(/C=C/c4ccc5nonc5c4)cc3)cn2C)cn1C ; CN1CCN(CCNC(=N)CCNC(=O)c2cc(NC(=O)c3cc(NC(=O)c4ccc(/C=C/c5cnc6ccccc6c5)cc4)cn3C)cn2C)CC1 ; CN1CCN(CCNC(=N)CCNC(=O)c2cc(NC(=O)c3cc(NC(=O)c4ccc(/C=C/c5ccc6nonc6c5)cc4)cn3C)cn2C)CC1]

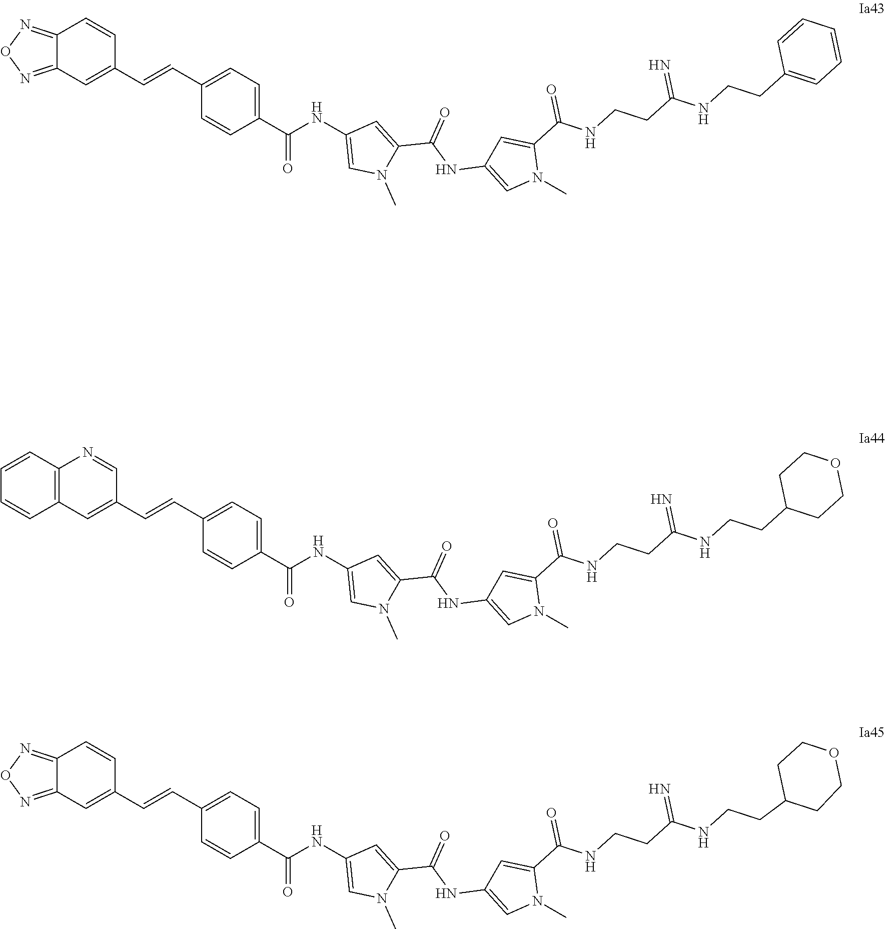 [SMILES: Cn1cc(NC(=O)c2cc(NC(=O)c3ccc(/C=C/c4ccc5nonc5c4)cc3)cn2C)cc1C(=O)NCCC(=N)NCCc1ccccc1 ; Cn1cc(NC(=O)c2cc(NC(=O)c3ccc(/C=C/c4cnc5ccccc5c4)cc3)cn2C)cc1C(=O)NCCC(=N)NCCC1CCOCC1 ; Cn1cc(NC(=O)c2cc(NC(=O)c3ccc(/C=C/c4ccc5nonc5c4)cc3)cn2C)cc1C(=O)NCCC(=N)NCCC1CCOCC1]

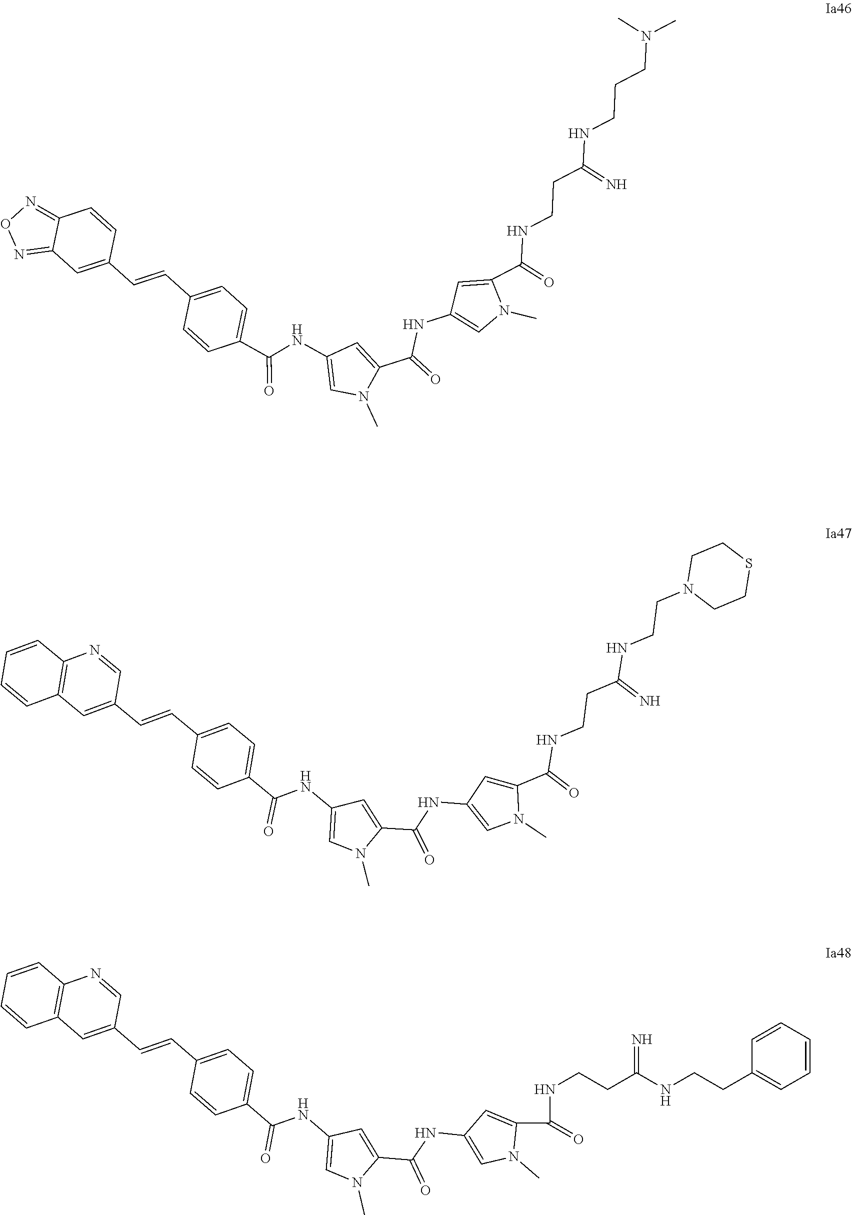 [SMILES: CN(C)CCCNC(=N)CCNC(=O)c1cc(NC(=O)c2cc(NC(=O)c3ccc(/C=C/c4ccc5nonc5c4)cc3)cn2C)cn1C ; Cn1cc(NC(=O)c2cc(NC(=O)c3ccc(/C=C/c4cnc5ccccc5c4)cc3)cn2C)cc1C(=O)NCCC(=N)NCCN1CCSCC1 ; Cn1cc(NC(=O)c2cc(NC(=O)c3ccc(/C=C/c4cnc5ccccc5c4)cc3)cn2C)cc1C(=O)NCCC(=N)NCCc1ccccc1]

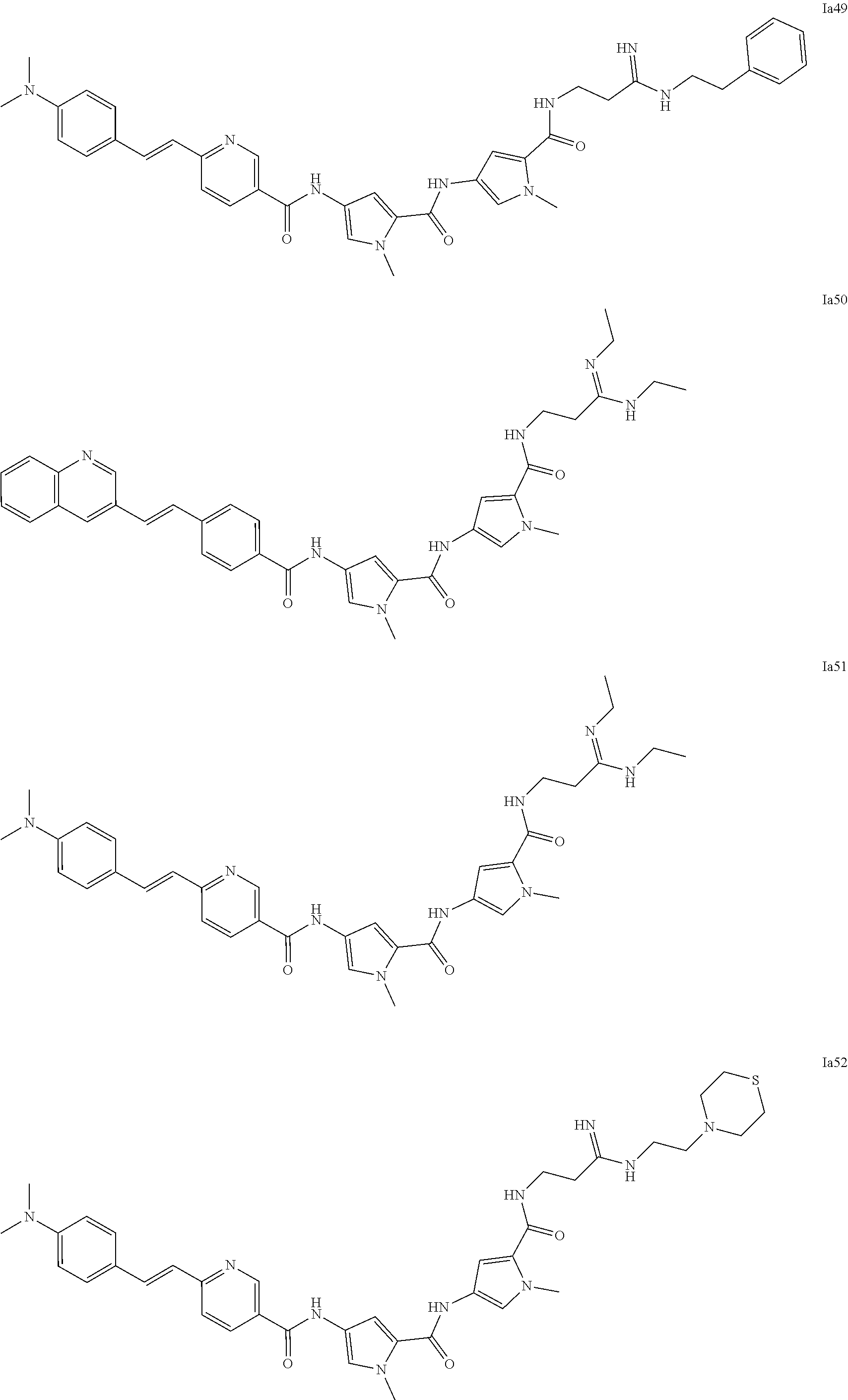

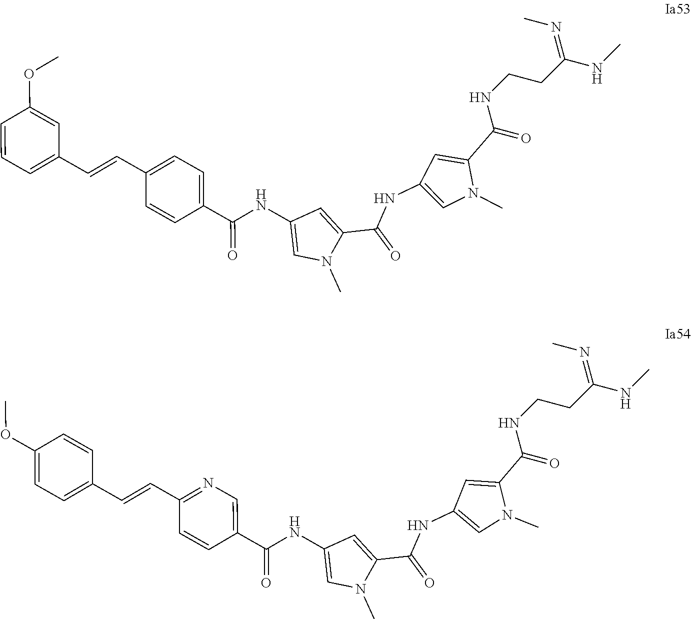

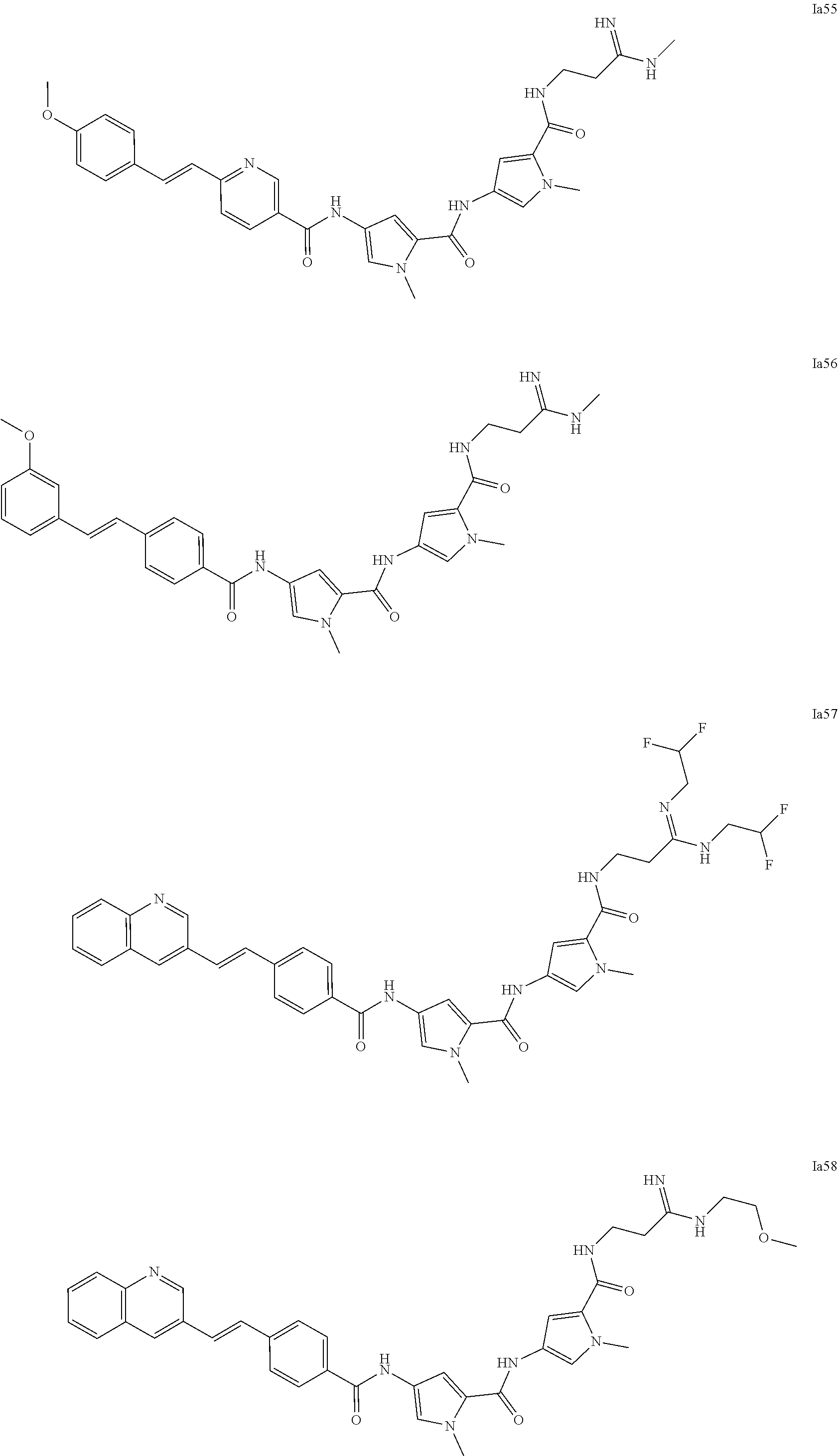

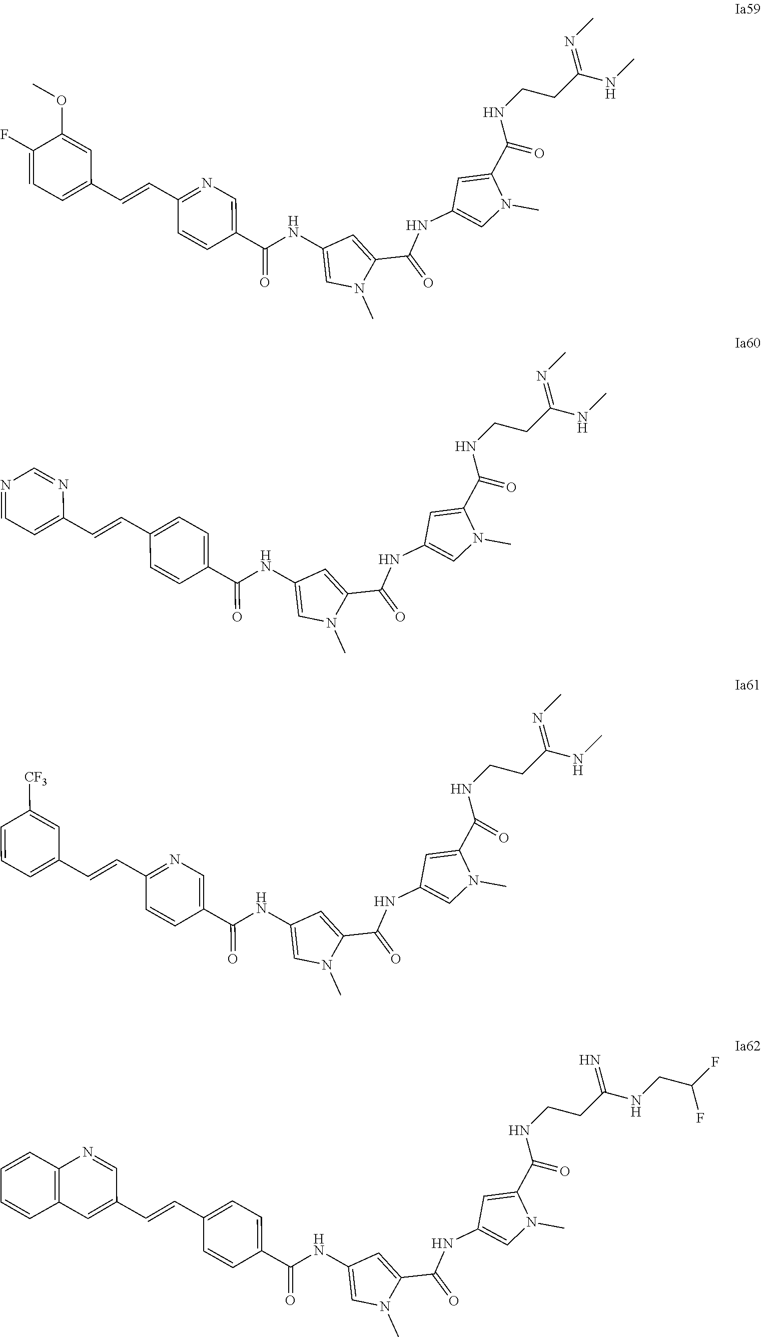 [SMILES: C/N=C(/CCNC(=O)c1cc(NC(=O)c2cc(NC(=O)c3ccc(/C=C/c4ccc(F)c(OC)c4)nc3)cn2C)cn1C)NC ; C/N=C(/CCNC(=O)c1cc(NC(=O)c2cc(NC(=O)c3ccc(/C=C/c4ccncn4)cc3)cn2C)cn1C)NC ; C/N=C(/CCNC(=O)c1cc(NC(=O)c2cc(NC(=O)c3ccc(/C=C/c4cccc(C(F)(F)F)c4)nc3)cn2C)cn1C)NC ; Cn1cc(NC(=O)c2cc(NC(=O)c3ccc(/C=C/c4cnc5ccccc5c4)cc3)cn2C)cc1C(=O)NCCC(=N)NCC(F)F]

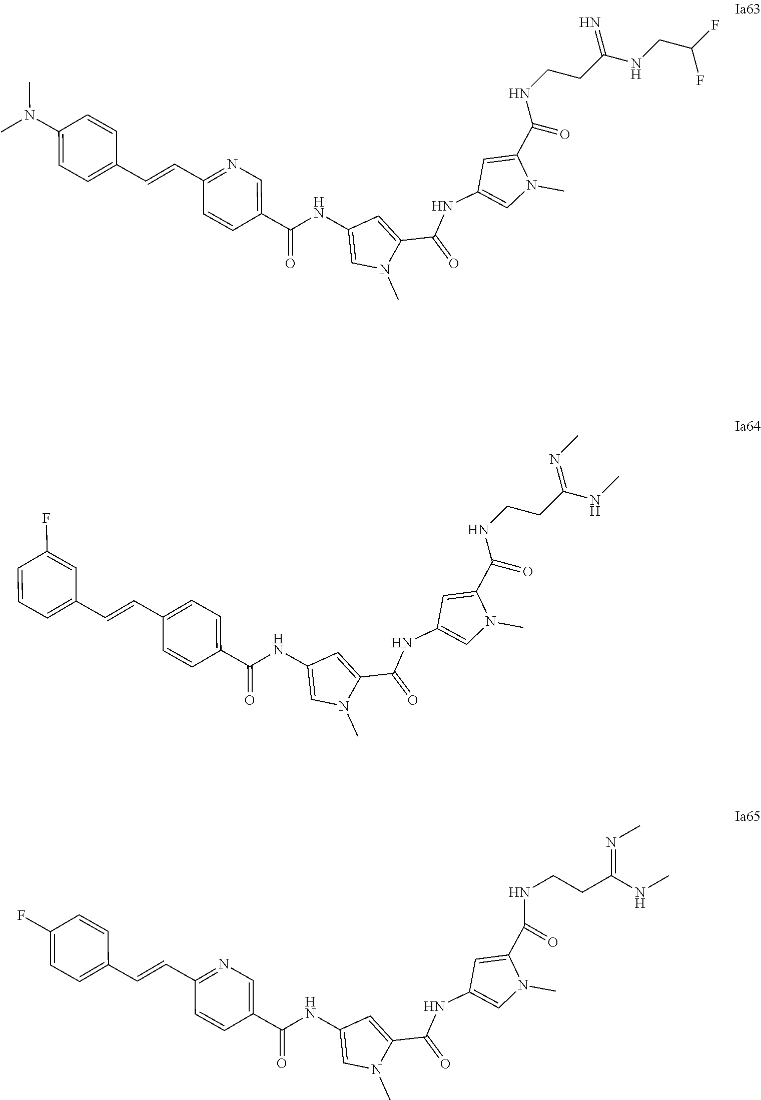 [SMILES: CN(C)c1ccc(/C=C/c2ccc(C(=O)Nc3cc(C(=O)Nc4cc(C(=O)NCCC(=N)NCC(F)F)n(C)c4)n(C)c3)cn2)cc1 ; C/N=C(/CCNC(=O)c1cc(NC(=O)c2cc(NC(=O)c3ccc(/C=C/c4cccc(F)c4)cc3)cn2C)cn1C)NC ; C/N=C(/CCNC(=O)c1cc(NC(=O)c2cc(NC(=O)c3ccc(/C=C/c4ccc(F)cc4)nc3)cn2C)cn1C)NC]

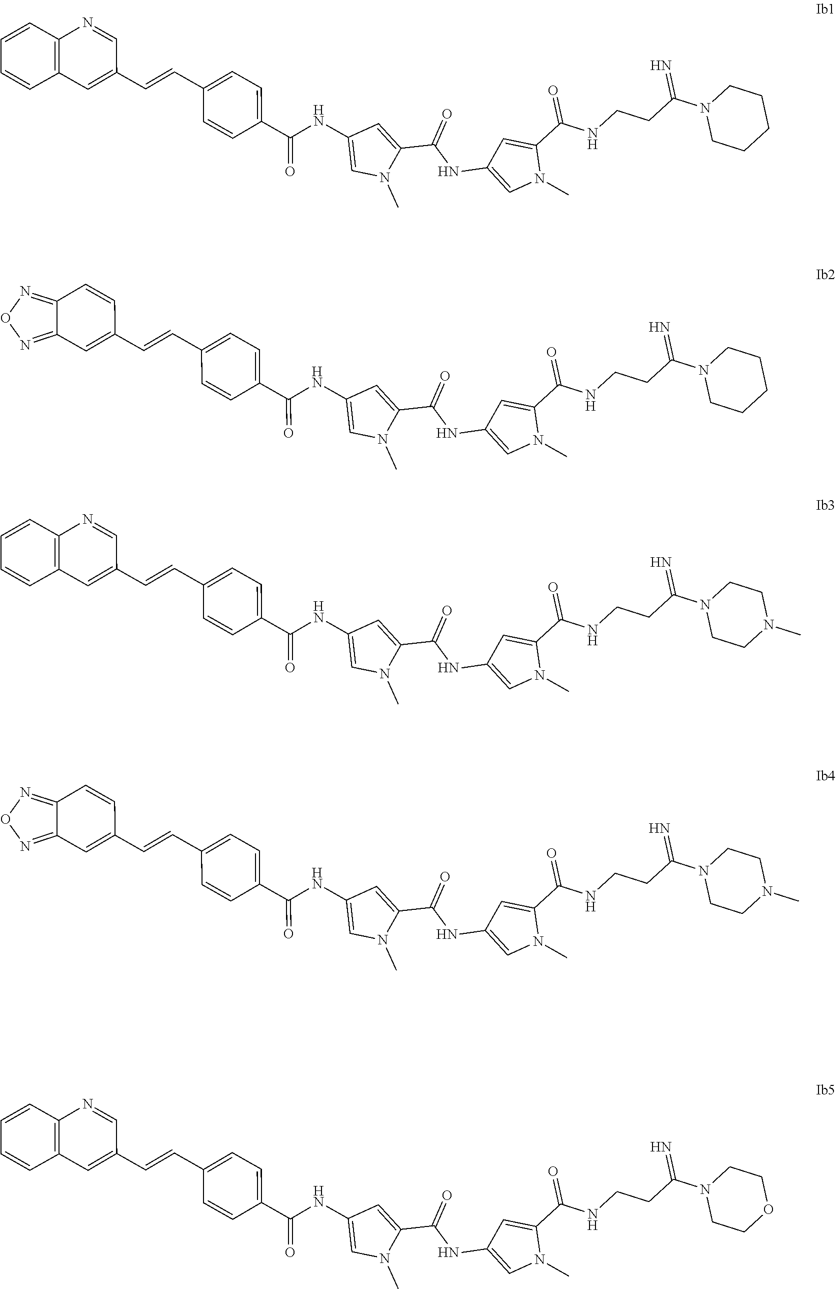 [SMILES: Cn1cc(NC(=O)c2cc(NC(=O)c3ccc(/C=C/c4cnc5ccccc5c4)cc3)cn2C)cc1C(=O)NCCC(=N)N1CCCCC1 ; Cn1cc(NC(=O)c2cc(NC(=O)c3ccc(/C=C/c4ccc5nonc5c4)cc3)cn2C)cc1C(=O)NCCC(=N)N1CCCCC1 ; CN1CCN(C(=N)CCNC(=O)c2cc(NC(=O)c3cc(NC(=O)c4ccc(/C=C/c5cnc6ccccc6c5)cc4)cn3C)cn2C)CC1 ; CN1CCN(C(=N)CCNC(=O)c2cc(NC(=O)c3cc(NC(=O)c4ccc(/C=C/c5ccc6nonc6c5)cc4)cn3C)cn2C)CC1 ; Cn1cc(NC(=O)c2cc(NC(=O)c3ccc(/C=C/c4cnc5ccccc5c4)cc3)cn2C)cc1C(=O)NCCC(=N)N1CCOCC1]

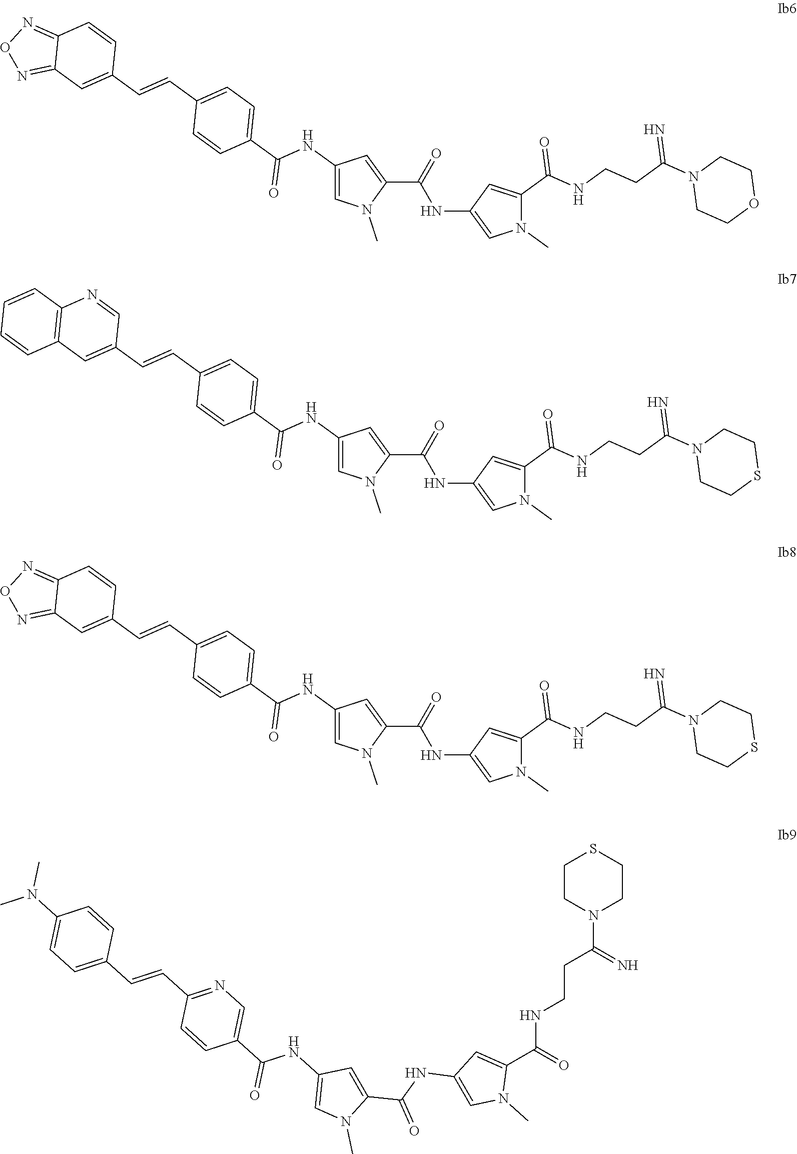 [SMILES: Cn1cc(NC(=O)c2cc(NC(=O)c3ccc(/C=C/c4ccc5nonc5c4)cc3)cn2C)cc1C(=O)NCCC(=N)N1CCOCC1 ; Cn1cc(NC(=O)c2cc(NC(=O)c3ccc(/C=C/c4cnc5ccccc5c4)cc3)cn2C)cc1C(=O)NCCC(=N)N1CCSCC1 ; Cn1cc(NC(=O)c2cc(NC(=O)c3ccc(/C=C/c4ccc5nonc5c4)cc3)cn2C)cc1C(=O)NCCC(=N)N1CCSCC1 ; CN(C)c1ccc(/C=C/c2ccc(C(=O)Nc3cc(C(=O)Nc4cc(C(=O)NCCC(=N)N5CCSCC5)n(C)c4)n(C)c3)cn2)cc1]

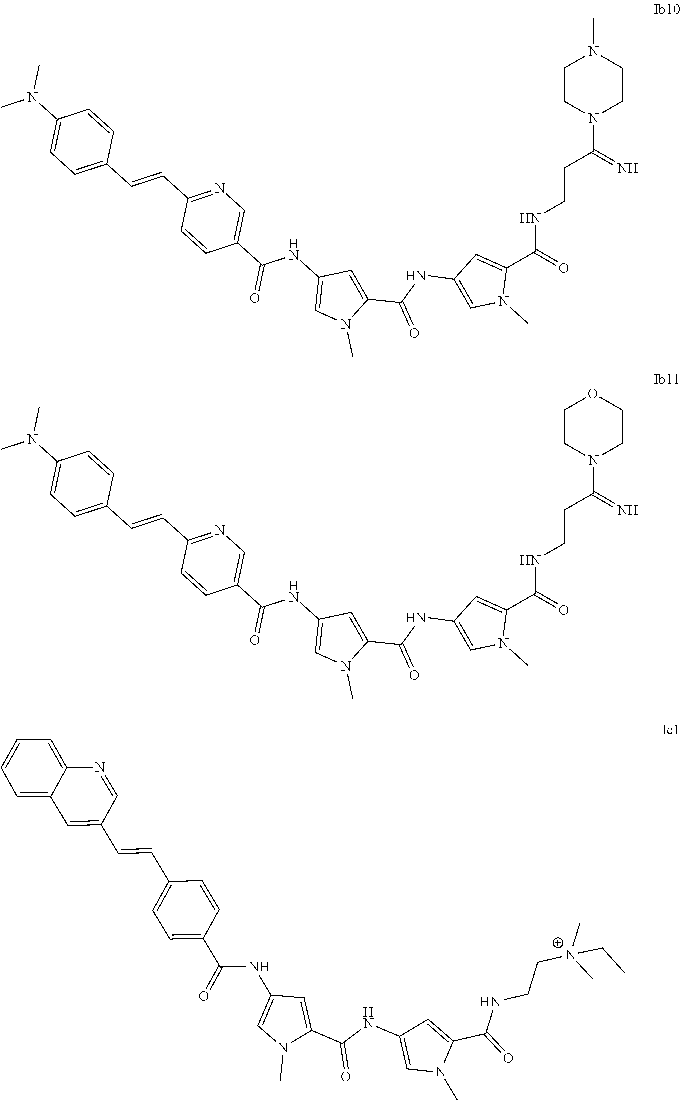 [SMILES: CN1CCN(C(=N)CCNC(=O)c2cc(NC(=O)c3cc(NC(=O)c4ccc(/C=C/c5ccc(N(C)C)cc5)nc4)cn3C)cn2C)CC1 ; CN(C)c1ccc(/C=C/c2ccc(C(=O)Nc3cc(C(=O)Nc4cc(C(=O)NCCC(=N)N5CCOCC5)n(C)c4)n(C)c3)cn2)cc1 ; CC[N+](C)(C)CCNC(=O)c1cc(NC(=O)c2cc(NC(=O)c3ccc(/C=C/c4cnc5ccccc5c4)cc3)cn2C)cn1C]

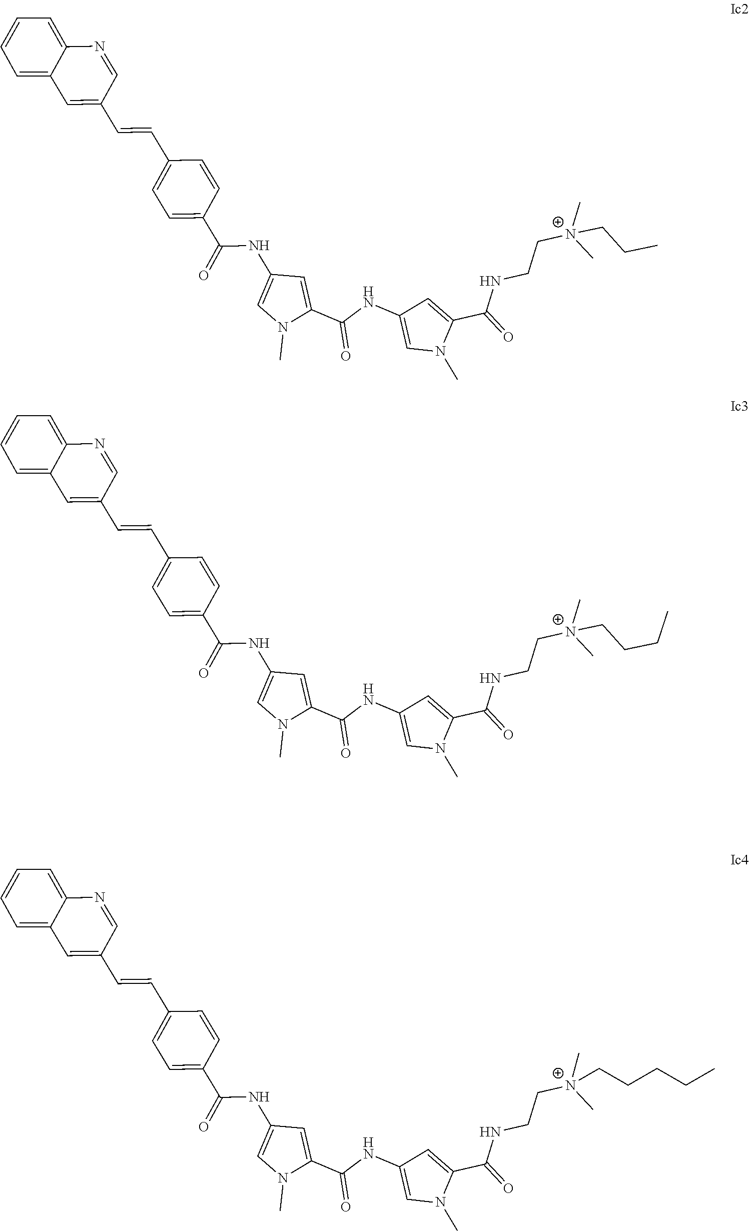 [SMILES: CCC[N+](C)(C)CCNC(=O)c1cc(NC(=O)c2cc(NC(=O)c3ccc(/C=C/c4cnc5ccccc5c4)cc3)cn2C)cn1C ; CCCC[N+](C)(C)CCNC(=O)c1cc(NC(=O)c2cc(NC(=O)c3ccc(/C=C/c4cnc5ccccc5c4)cc3)cn2C)cn1C ; CCCCC[N+](C)(C)CCNC(=O)c1cc(NC(=O)c2cc(NC(=O)c3ccc(/C=C/c4cnc5ccccc5c4)cc3)cn2C)cn1C]

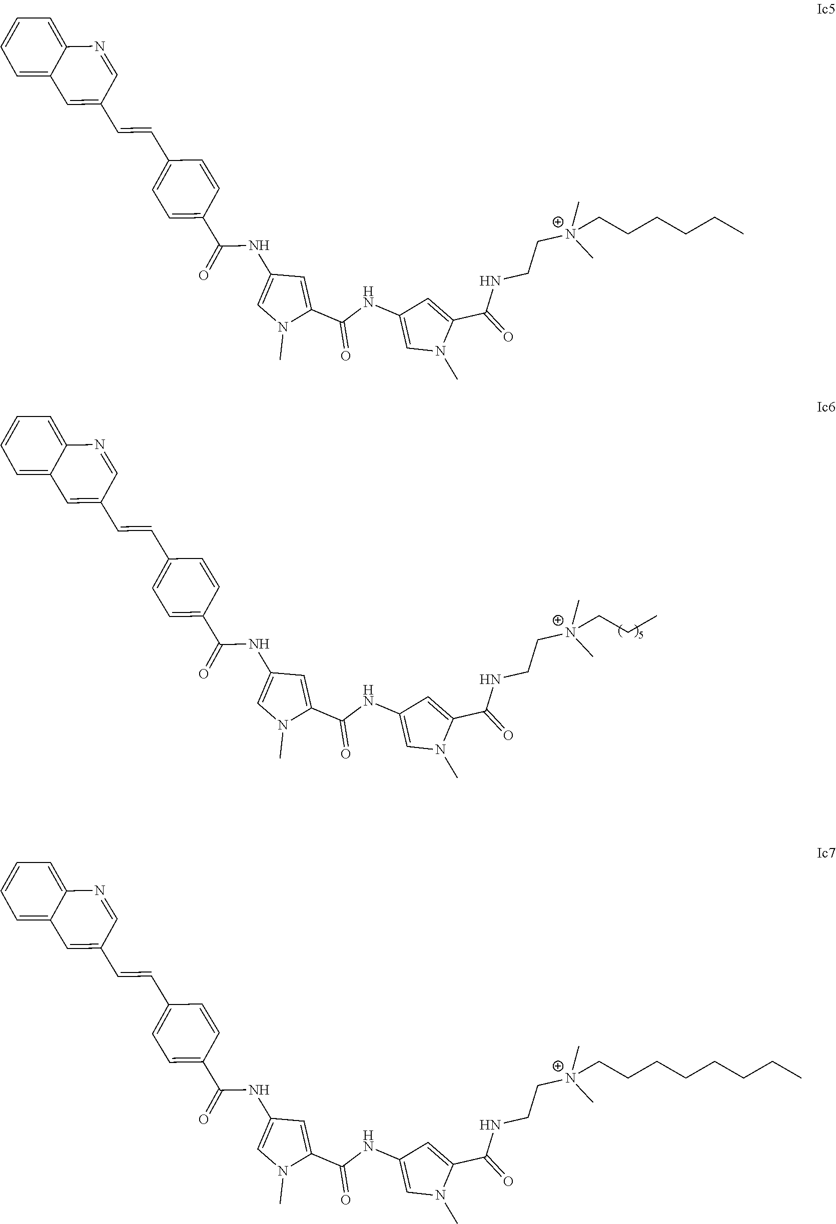

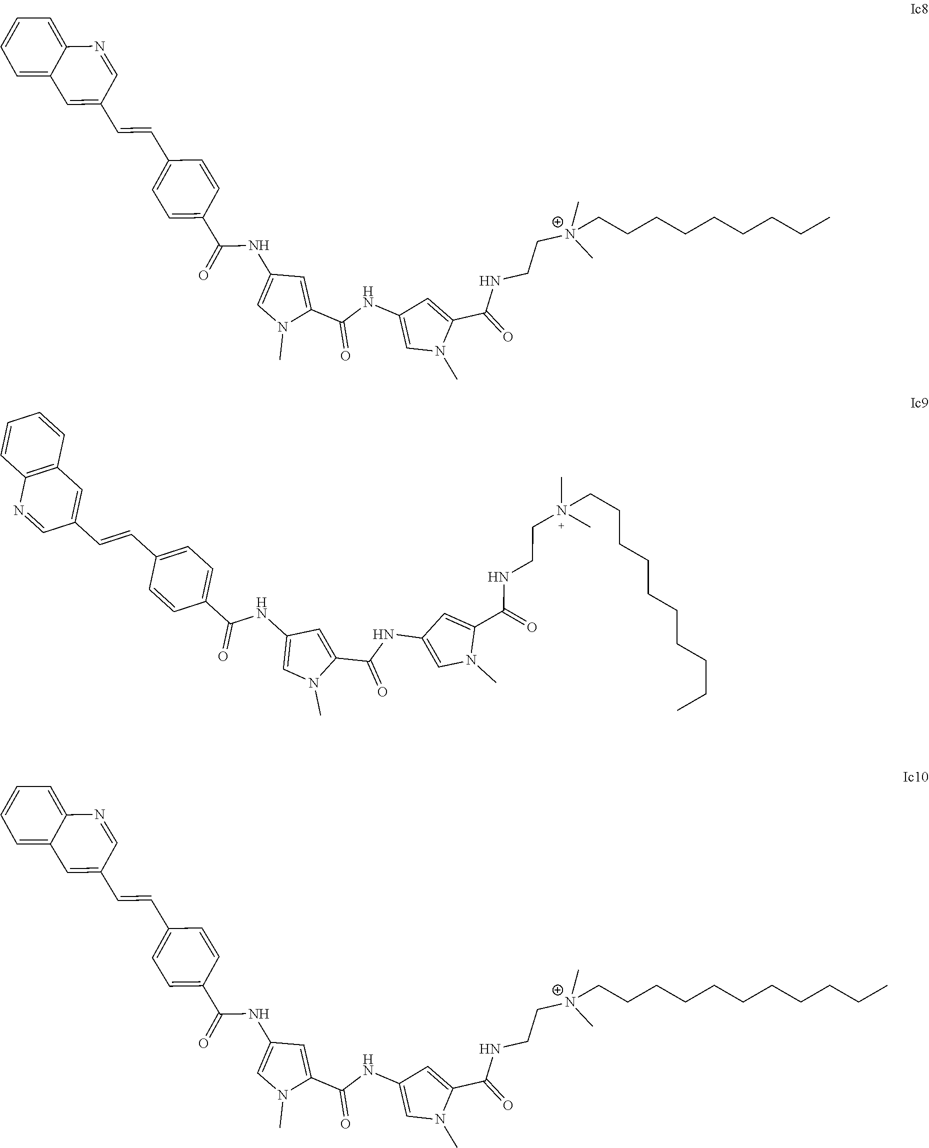 [SMILES: CCCCCCCCC[N+](C)(C)CCNC(=O)c1cc(NC(=O)c2cc(NC(=O)c3ccc(/C=C/c4cnc5ccccc5c4)cc3)cn2C)cn1C ; CCCCCCCCCC[N+](C)(C)CCNC(=O)c1cc(NC(=O)c2cc(NC(=O)c3ccc(/C=C/c4cnc5ccccc5c4)cc3)cn2C)cn1C ; CCCCCCCCCCC[N+](C)(C)CCNC(=O)c1cc(NC(=O)c2cc(NC(=O)c3ccc(/C=C/c4cnc5ccccc5c4)cc3)cn2C)cn1C]

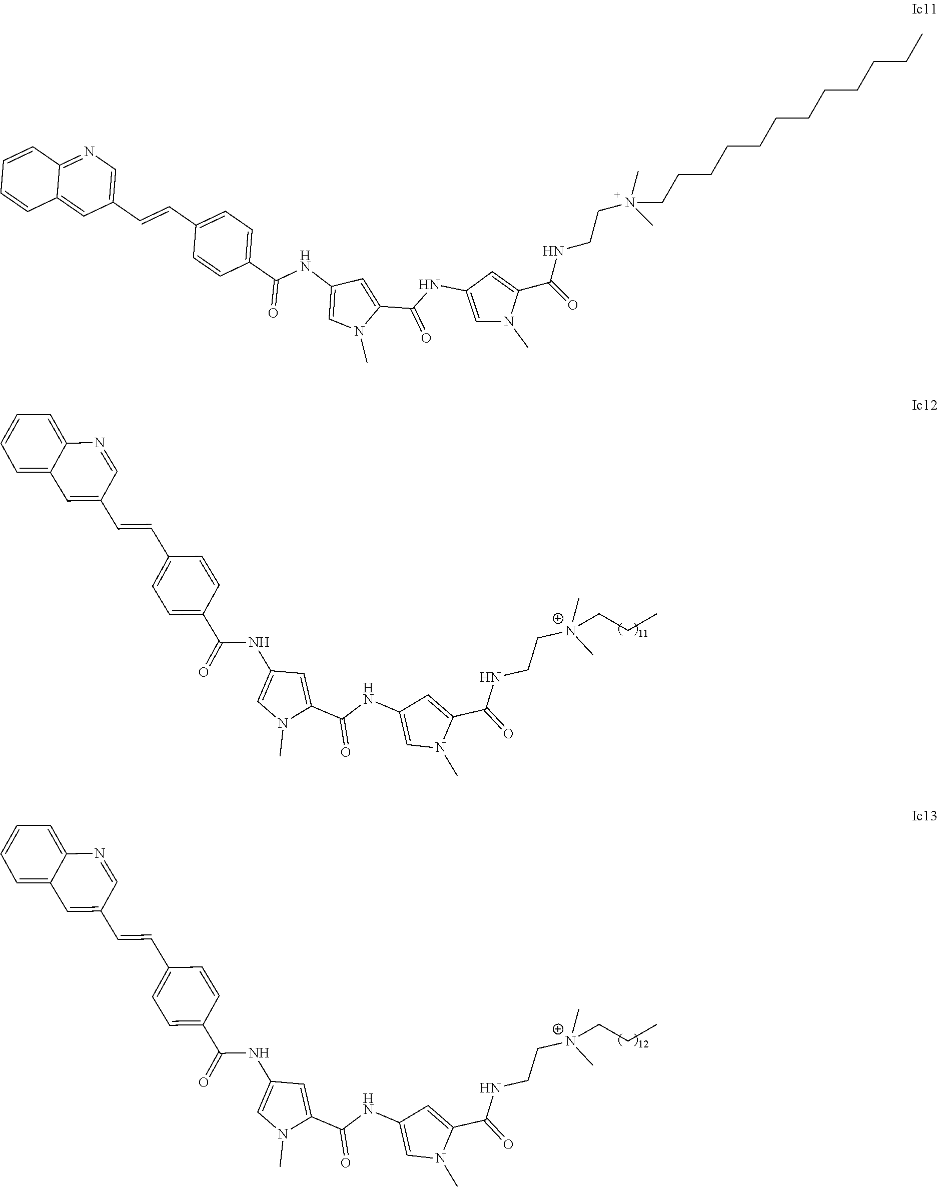

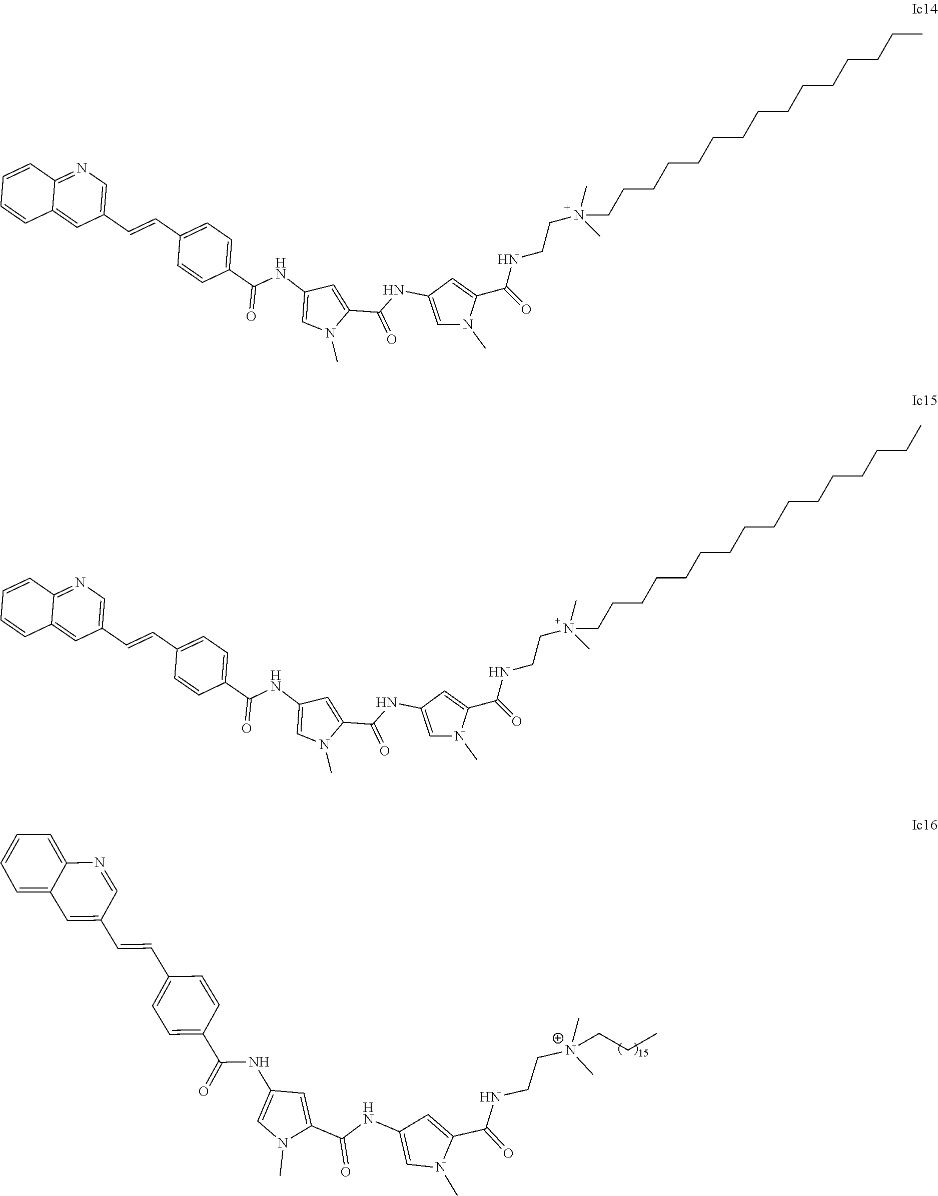

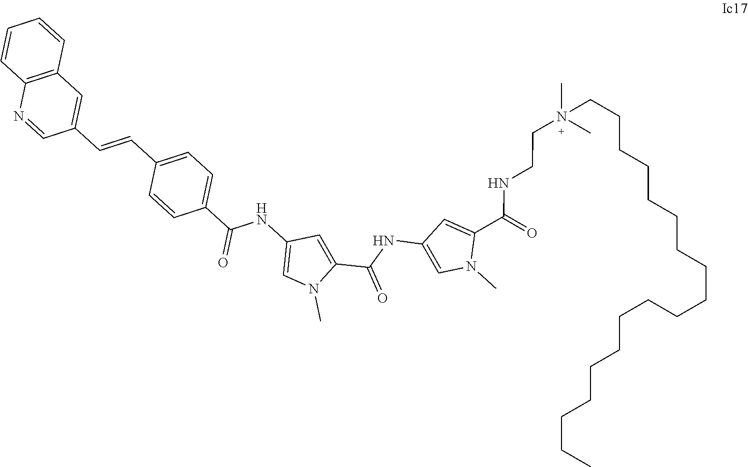

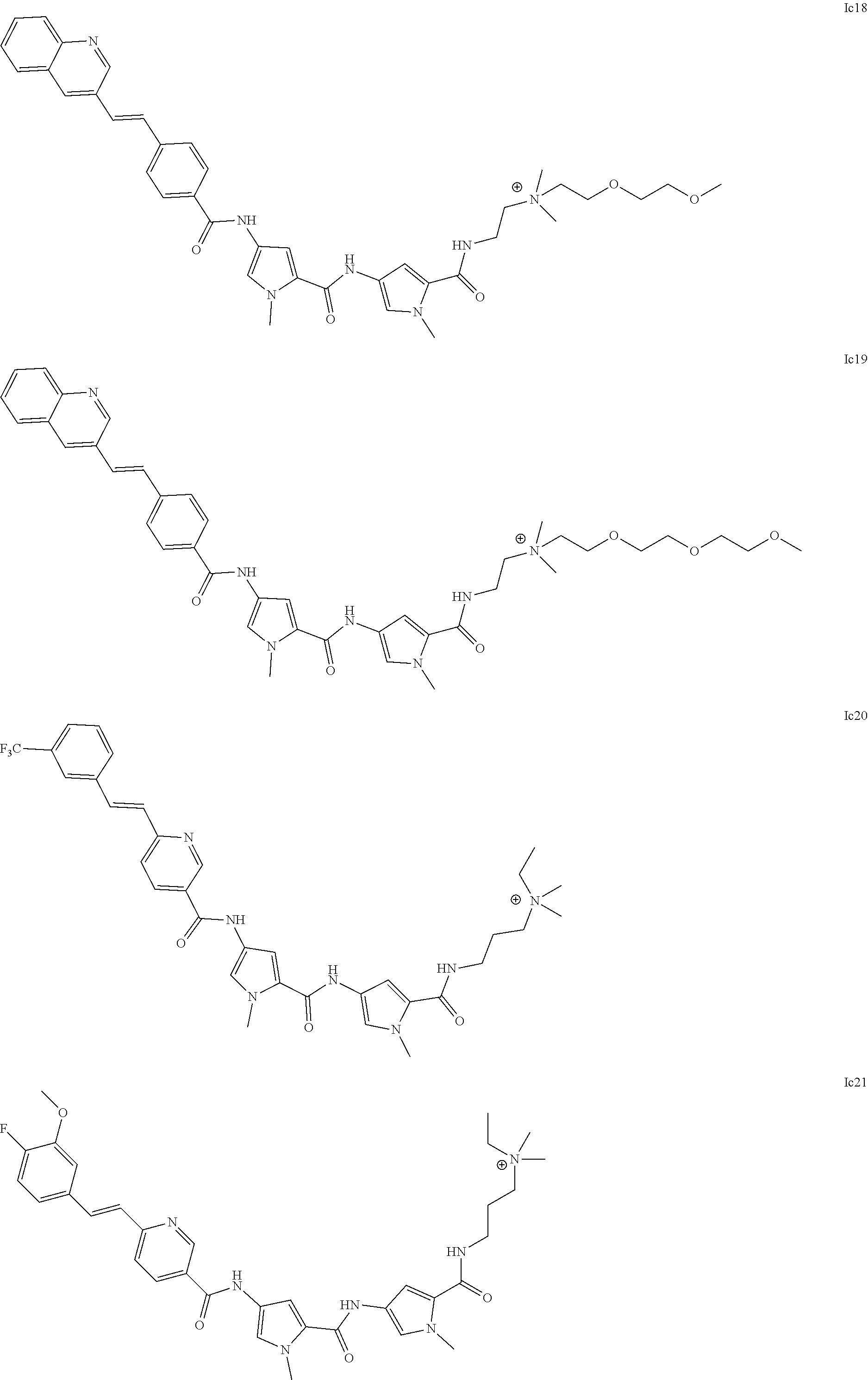 [SMILES: COCCOCC[N+](C)(C)CCNC(=O)c1cc(NC(=O)c2cc(NC(=O)c3ccc(/C=C/c4cnc5ccccc5c4)cc3)cn2C)cn1C ; COCCOCCOCC[N+](C)(C)CCNC(=O)c1cc(NC(=O)c2cc(NC(=O)c3ccc(/C=C/c4cnc5ccccc5c4)cc3)cn2C)cn1C ; CC[N+](C)(C)CCCNC(=O)c1cc(NC(=O)c2cc(NC(=O)c3ccc(/C=C/c4cccc(C(F)(F)F)c4)nc3)cn2C)cn1C ; CC[N+](C)(C)CCCNC(=O)c1cc(NC(=O)c2cc(NC(=O)c3ccc(/C=C/c4ccc(F)c(OC)c4)nc3)cn2C)cn1C]

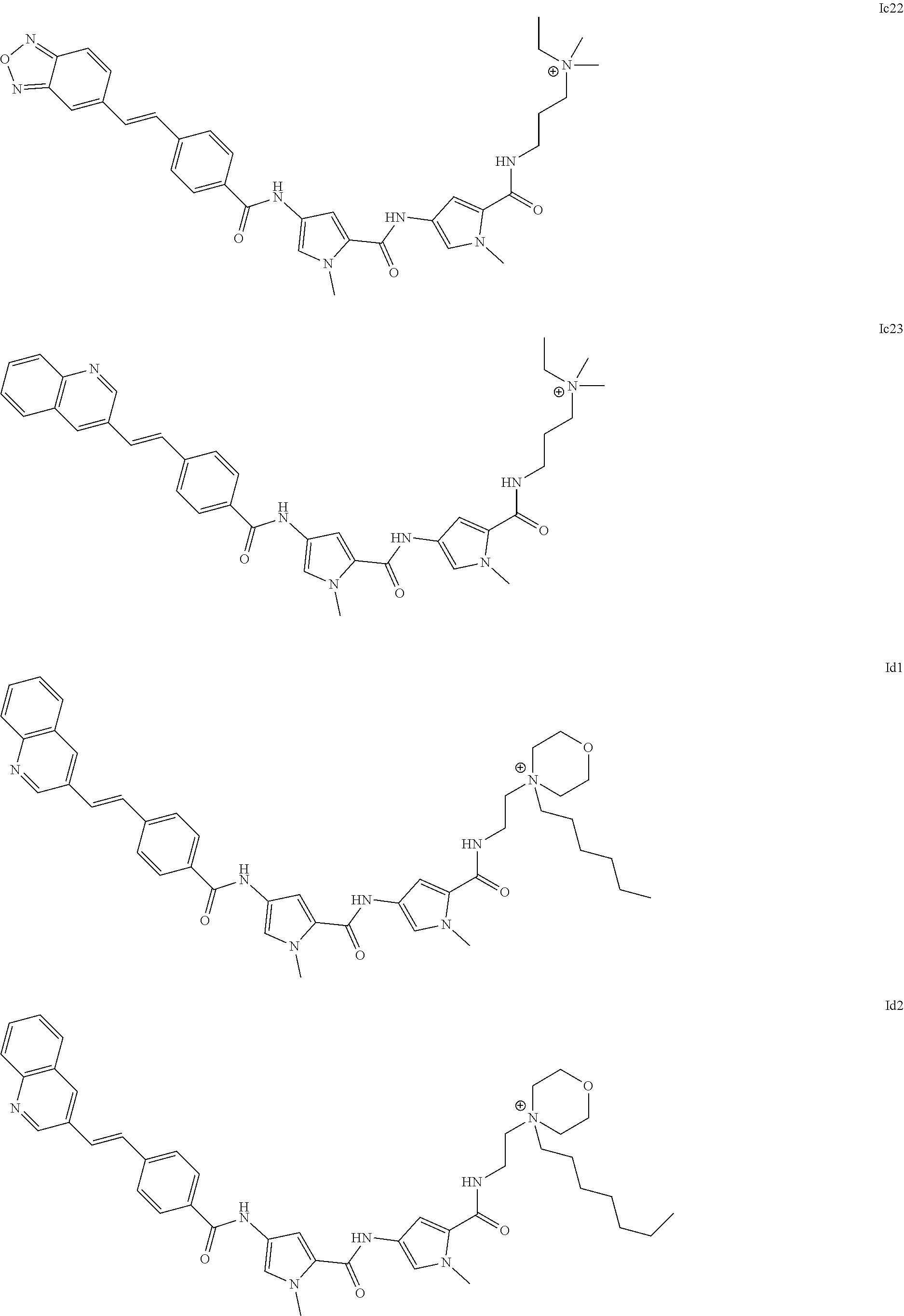 [SMILES: CC[N+](C)(C)CCCNC(=O)c1cc(NC(=O)c2cc(NC(=O)c3ccc(/C=C/c4ccc5nonc5c4)cc3)cn2C)cn1C ; CC[N+](C)(C)CCCNC(=O)c1cc(NC(=O)c2cc(NC(=O)c3ccc(/C=C/c4cnc5ccccc5c4)cc3)cn2C)cn1C ; CCCCCC[N+]1(CCNC(=O)c2cc(NC(=O)c3cc(NC(=O)c4ccc(/C=C/c5cnc6ccccc6c5)cc4)cn3C)cn2C)CCOCC1 ; CCCCCCC[N+]1(CCNC(=O)c2cc(NC(=O)c3cc(NC(=O)c4ccc(/C=C/c5cnc6ccccc6c5)cc4)cn3C)cn2C)CCOCC1]

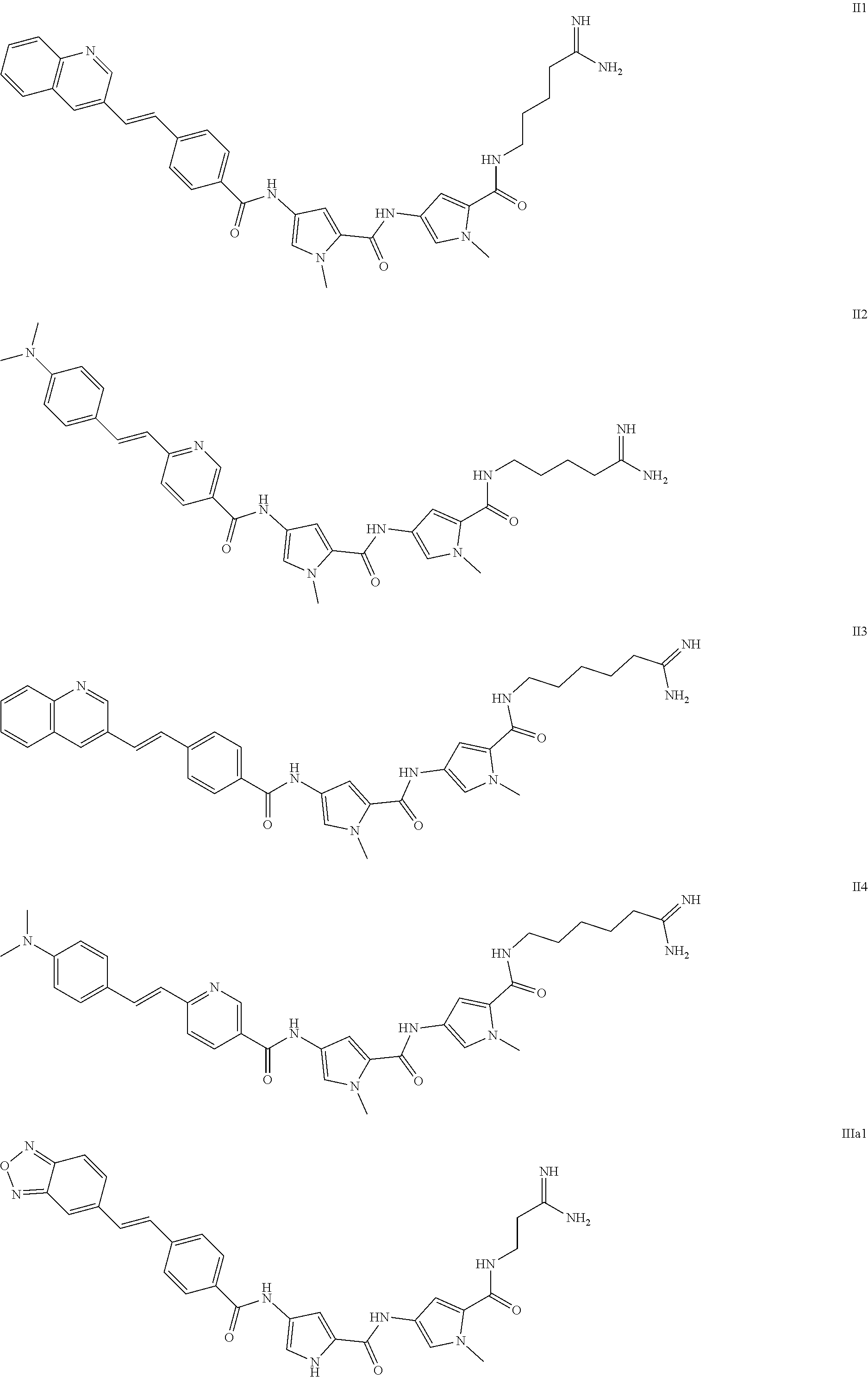 [SMILES: Cn1cc(NC(=O)c2cc(NC(=O)c3ccc(/C=C/c4cnc5ccccc5c4)cc3)cn2C)cc1C(=O)NCCCCC(=N)N ; CN(C)c1ccc(/C=C/c2ccc(C(=O)Nc3cc(C(=O)Nc4cc(C(=O)NCCCCC(=N)N)n(C)c4)n(C)c3)cn2)cc1 ; Cn1cc(NC(=O)c2cc(NC(=O)c3ccc(/C=C/c4cnc5ccccc5c4)cc3)cn2C)cc1C(=O)NCCCCCC(=N)N ; CN(C)c1ccc(/C=C/c2ccc(C(=O)Nc3cc(C(=O)Nc4cc(C(=O)NCCCCCC(=N)N)n(C)c4)n(C)c3)cn2)cc1 ; Cn1cc(NC(=O)c2cc(NC(=O)c3ccc(/C=C/c4ccc5nonc5c4)cc3)c[nH]2)cc1C(=O)NCCC(=N)N]

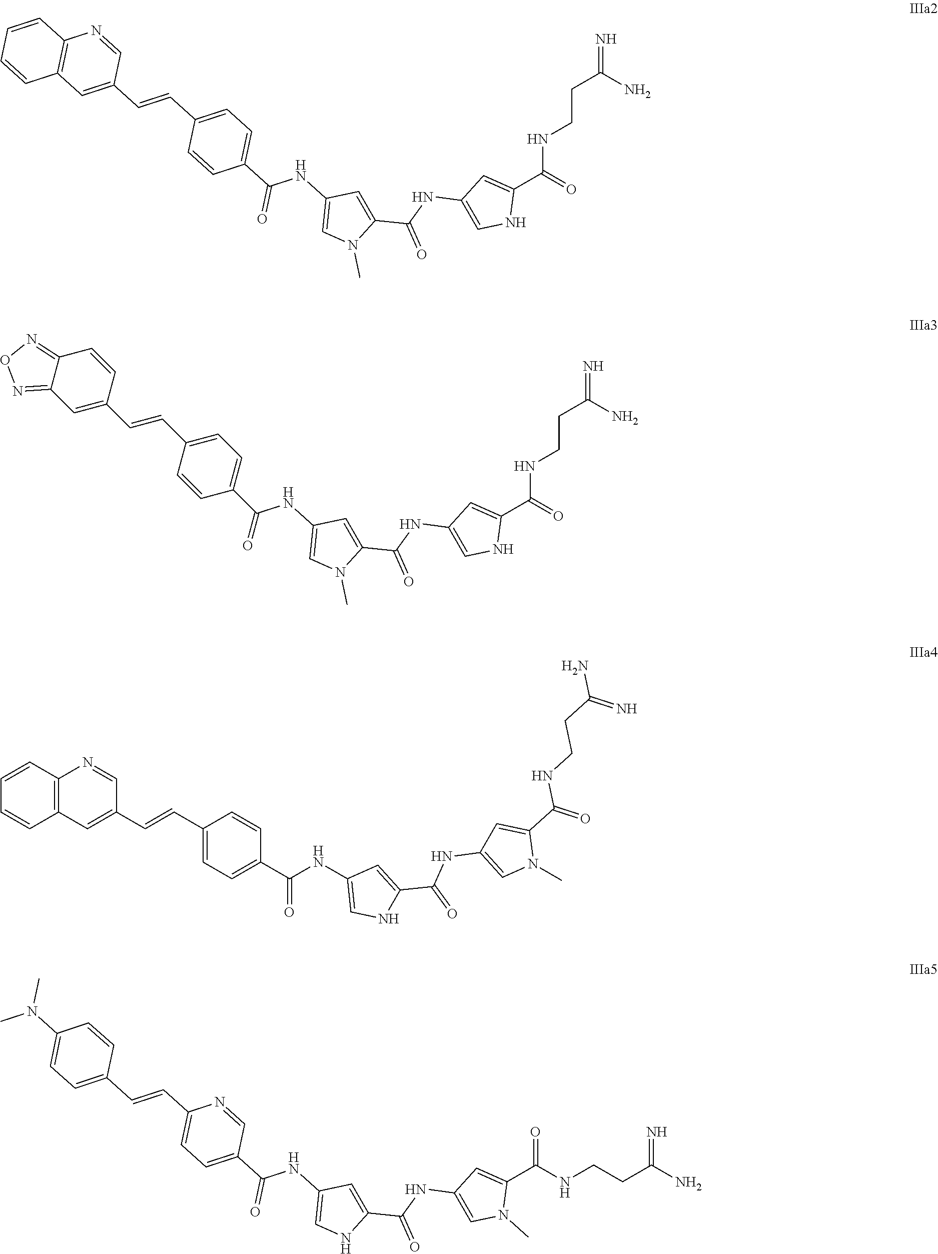 [SMILES: Cn1cc(NC(=O)c2ccc(/C=C/c3cnc4ccccc4c3)cc2)cc1C(=O)Nc1c[nH]c(C(=O)NCCC(=N)N)c1 ; Cn1cc(NC(=O)c2ccc(/C=C/c3ccc4nonc4c3)cc2)cc1C(=O)Nc1c[nH]c(C(=O)NCCC(=N)N)c1 ; Cn1cc(NC(=O)c2cc(NC(=O)c3ccc(/C=C/c4cnc5ccccc5c4)cc3)c[nH]2)cc1C(=O)NCCC(=N)N ; CN(C)c1ccc(/C=C/c2ccc(C(=O)Nc3c[nH]c(C(=O)Nc4cc(C(=O)NCCC(=N)N)n(C)c4)c3)cn2)cc1]

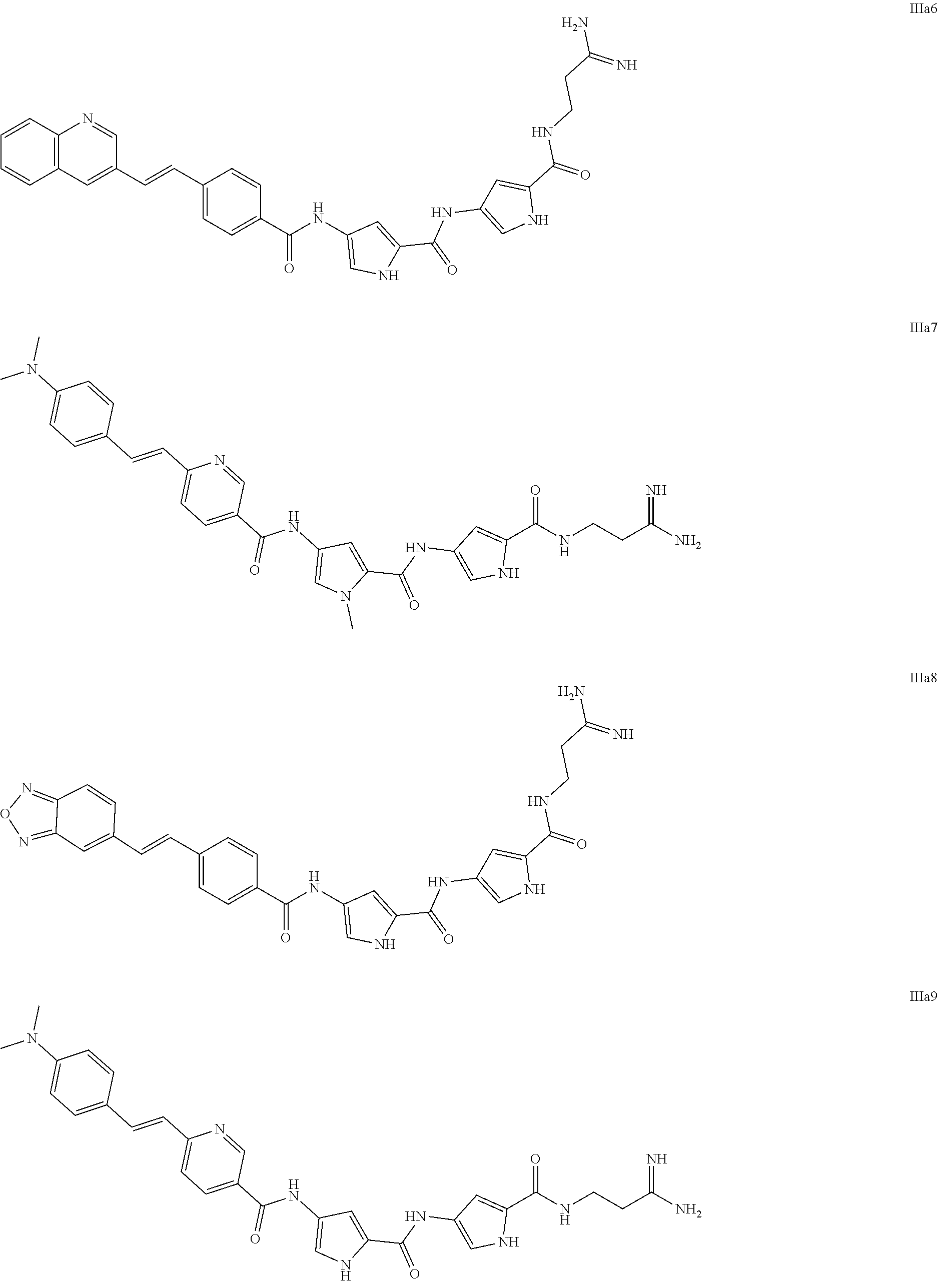 [SMILES: N=C(N)CCNC(=O)c1cc(NC(=O)c2cc(NC(=O)c3ccc(/C=C/c4cnc5ccccc5c4)cc3)c[nH]2)c[nH]1 ; CN(C)c1ccc(/C=C/c2ccc(C(=O)Nc3cc(C(=O)Nc4c[nH]c(C(=O)NCCC(=N)N)c4)n(C)c3)cn2)cc1 ; N=C(N)CCNC(=O)c1cc(NC(=O)c2cc(NC(=O)c3ccc(/C=C/c4ccc5nonc5c4)cc3)c[nH]2)c[nH]1 ; CN(C)c1ccc(/C=C/c2ccc(C(=O)Nc3c[nH]c(C(=O)Nc4c[nH]c(C(=O)NCCC(=N)N)c4)c3)cn2)cc1]

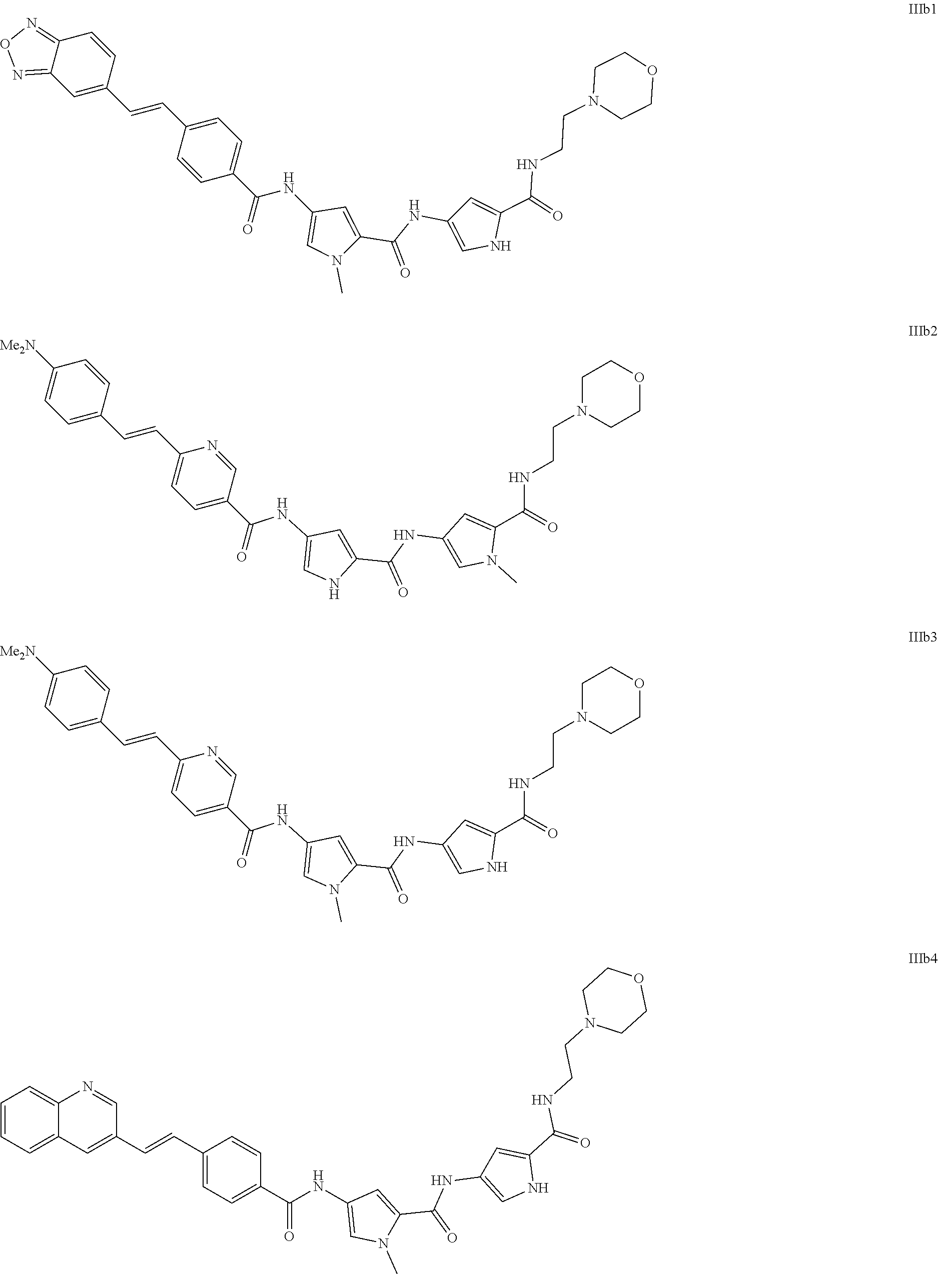 [SMILES: Cn1cc(NC(=O)c2ccc(/C=C/c3ccc4nonc4c3)cc2)cc1C(=O)Nc1c[nH]c(C(=O)NCCN2CCOCC2)c1 ; CN(C)c1ccc(/C=C/c2ccc(C(=O)Nc3c[nH]c(C(=O)Nc4cc(C(=O)NCCN5CCOCC5)n(C)c4)c3)cn2)cc1 ; CN(C)c1ccc(/C=C/c2ccc(C(=O)Nc3cc(C(=O)Nc4c[nH]c(C(=O)NCCN5CCOCC5)c4)n(C)c3)cn2)cc1 ; Cn1cc(NC(=O)c2ccc(/C=C/c3cnc4ccccc4c3)cc2)cc1C(=O)Nc1c[nH]c(C(=O)NCCN2CCOCC2)c1]

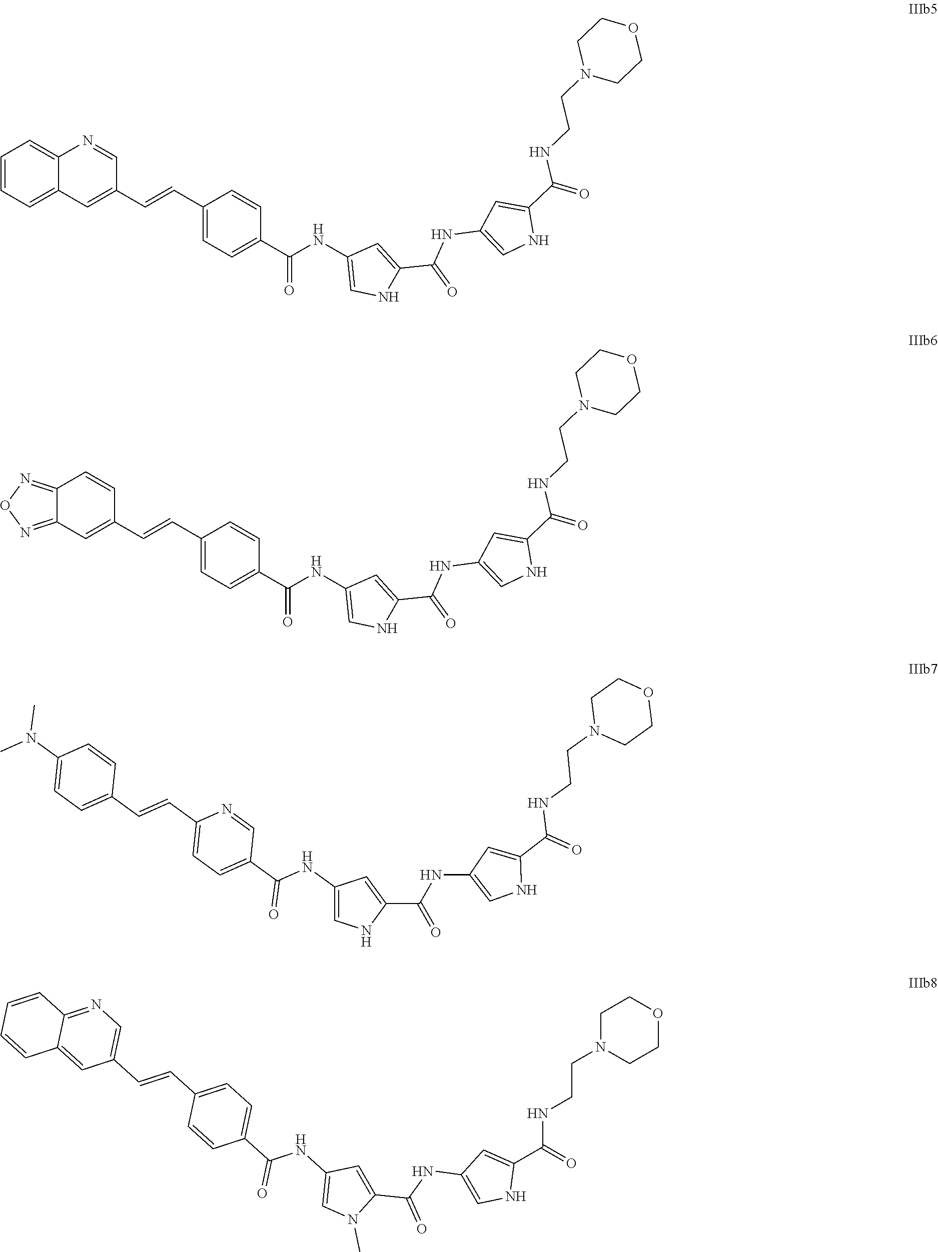 [SMILES: O=C(Nc1c[nH]c(C(=O)Nc2c[nH]c(C(=O)NCCN3CCOCC3)c2)c1)c1ccc(/C=C/c2cnc3ccccc3c2)cc1 ; O=C(Nc1c[nH]c(C(=O)Nc2c[nH]c(C(=O)NCCN3CCOCC3)c2)c1)c1ccc(/C=C/c2ccc3nonc3c2)cc1 ; CN(C)c1ccc(/C=C/c2ccc(C(=O)Nc3c[nH]c(C(=O)Nc4c[nH]c(C(=O)NCCN5CCOCC5)c4)c3)cn2)cc1 ; Cn1cc(NC(=O)c2ccc(/C=C/c3cnc4ccccc4c3)cc2)cc1C(=O)Nc1c[nH]c(C(=O)NCCN2CCOCC2)c1]

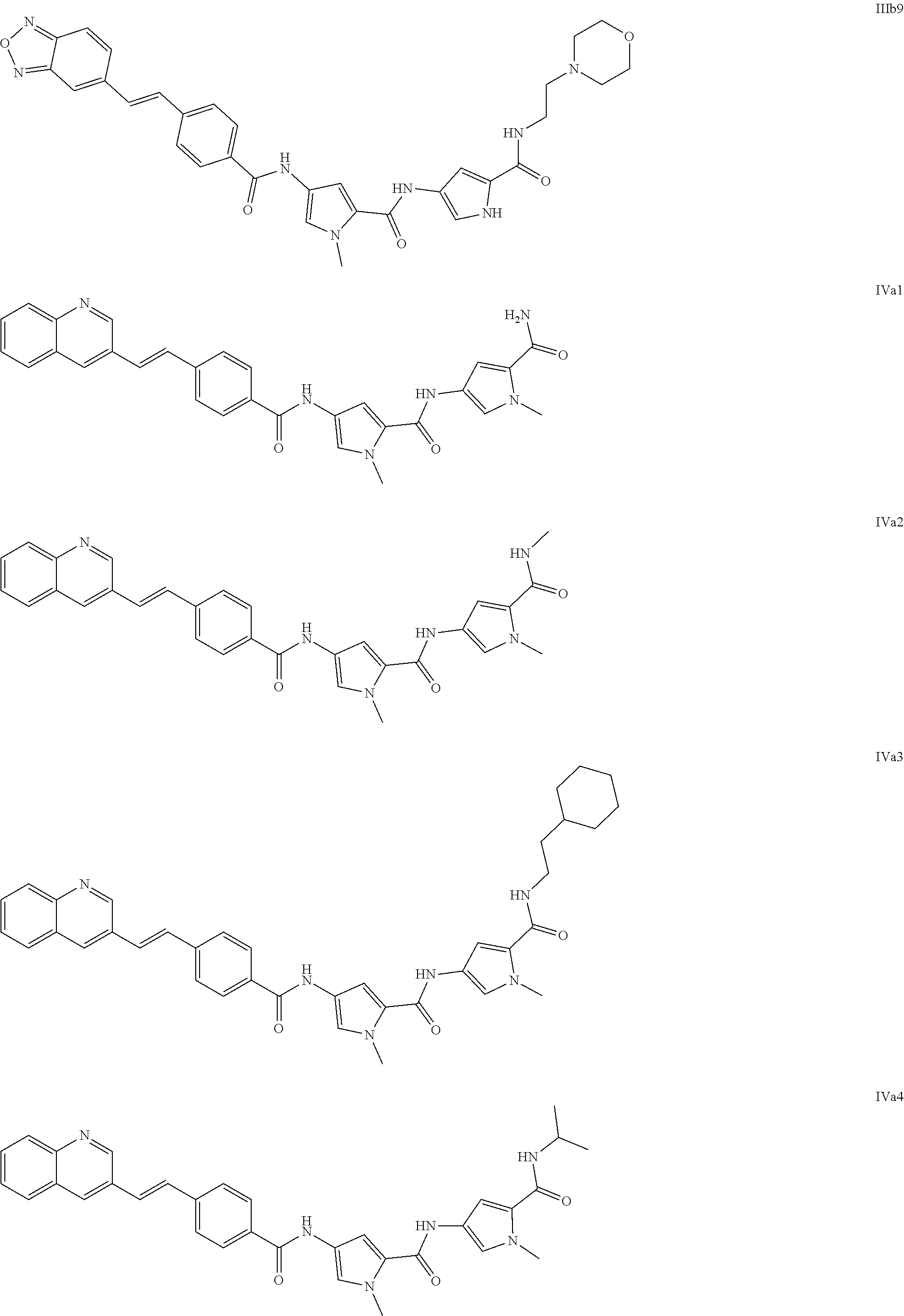 [SMILES: Cn1cc(NC(=O)c2ccc(/C=C/c3ccc4nonc4c3)cc2)cc1C(=O)Nc1c[nH]c(C(=O)NCCN2CCOCC2)c1 ; Cn1cc(NC(=O)c2cc(NC(=O)c3ccc(/C=C/c4cnc5ccccc5c4)cc3)cn2C)cc1C(N)=O ; CNC(=O)c1cc(NC(=O)c2cc(NC(=O)c3ccc(/C=C/c4cnc5ccccc5c4)cc3)cn2C)cn1C ; Cn1cc(NC(=O)c2cc(NC(=O)c3ccc(/C=C/c4cnc5ccccc5c4)cc3)cn2C)cc1C(=O)NCCC1CCCCC1 ; CC(C)NC(=O)c1cc(NC(=O)c2cc(NC(=O)c3ccc(/C=C/c4cnc5ccccc5c4)cc3)cn2C)cn1C]

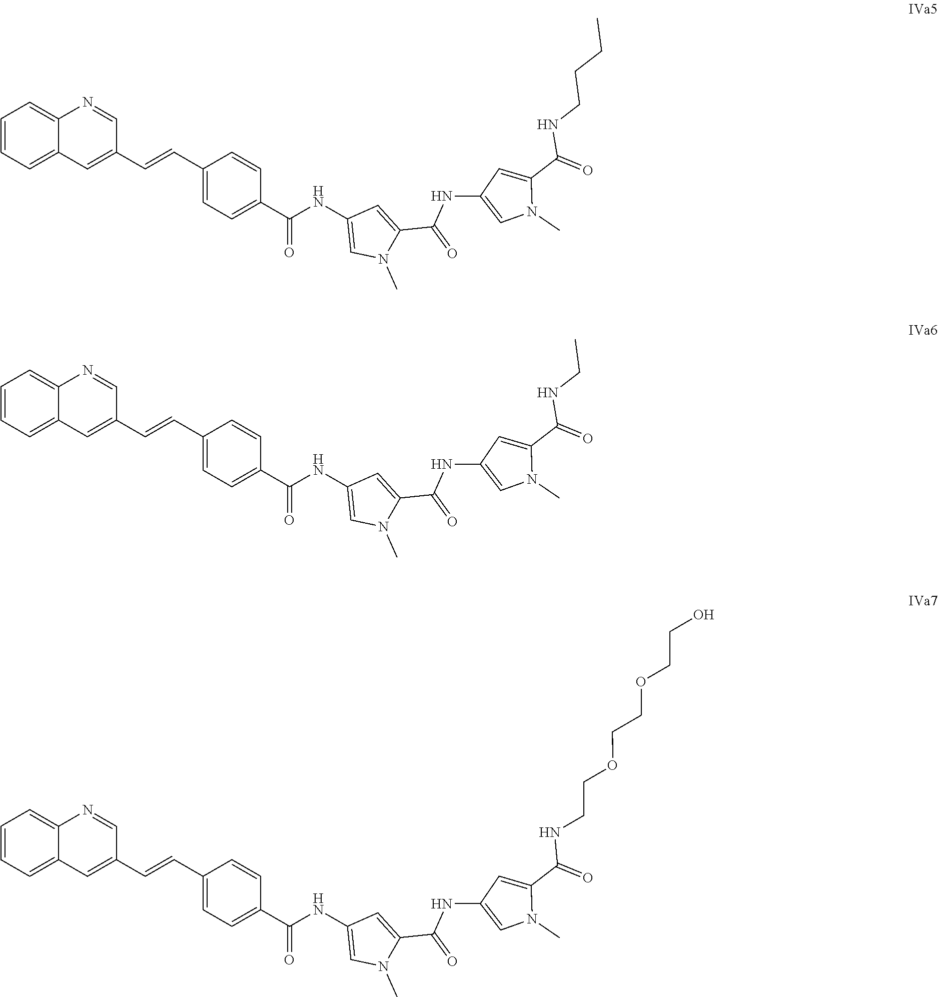 [SMILES: CCCCNC(=O)c1cc(NC(=O)c2cc(NC(=O)c3ccc(/C=C/c4cnc5ccccc5c4)cc3)cn2C)cn1C ; CCNC(=O)c1cc(NC(=O)c2cc(NC(=O)c3ccc(/C=C/c4cnc5ccccc5c4)cc3)cn2C)cn1C ; Cn1cc(NC(=O)c2cc(NC(=O)c3ccc(/C=C/c4cnc5ccccc5c4)cc3)cn2C)cc1C(=O)NCCOCCOCCO]

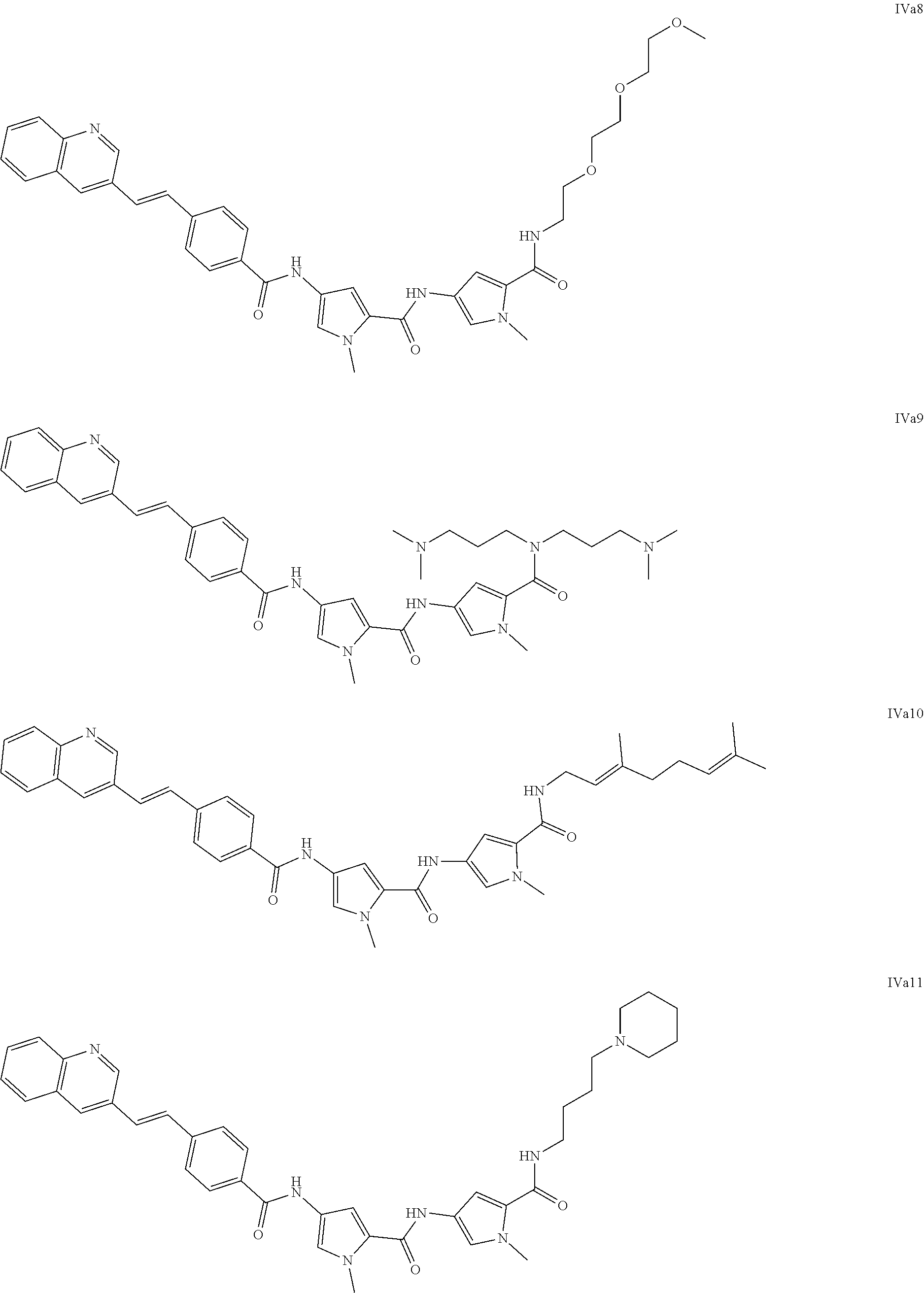 [SMILES: COCCOCCOCCNC(=O)c1cc(NC(=O)c2cc(NC(=O)c3ccc(/C=C/c4cnc5ccccc5c4)cc3)cn2C)cn1C ; CN(C)CCCN(CCCN(C)C)C(=O)c1cc(NC(=O)c2cc(NC(=O)c3ccc(/C=C/c4cnc5ccccc5c4)cc3)cn2C)cn1C ; CC(C)=CCC/C(C)=C/CNC(=O)c1cc(NC(=O)c2cc(NC(=O)c3ccc(/C=C/c4cnc5ccccc5c4)cc3)cn2C)cn1C ; Cn1cc(NC(=O)c2cc(NC(=O)c3ccc(/C=C/c4cnc5ccccc5c4)cc3)cn2C)cc1C(=O)NCCCCN1CCCCC1]

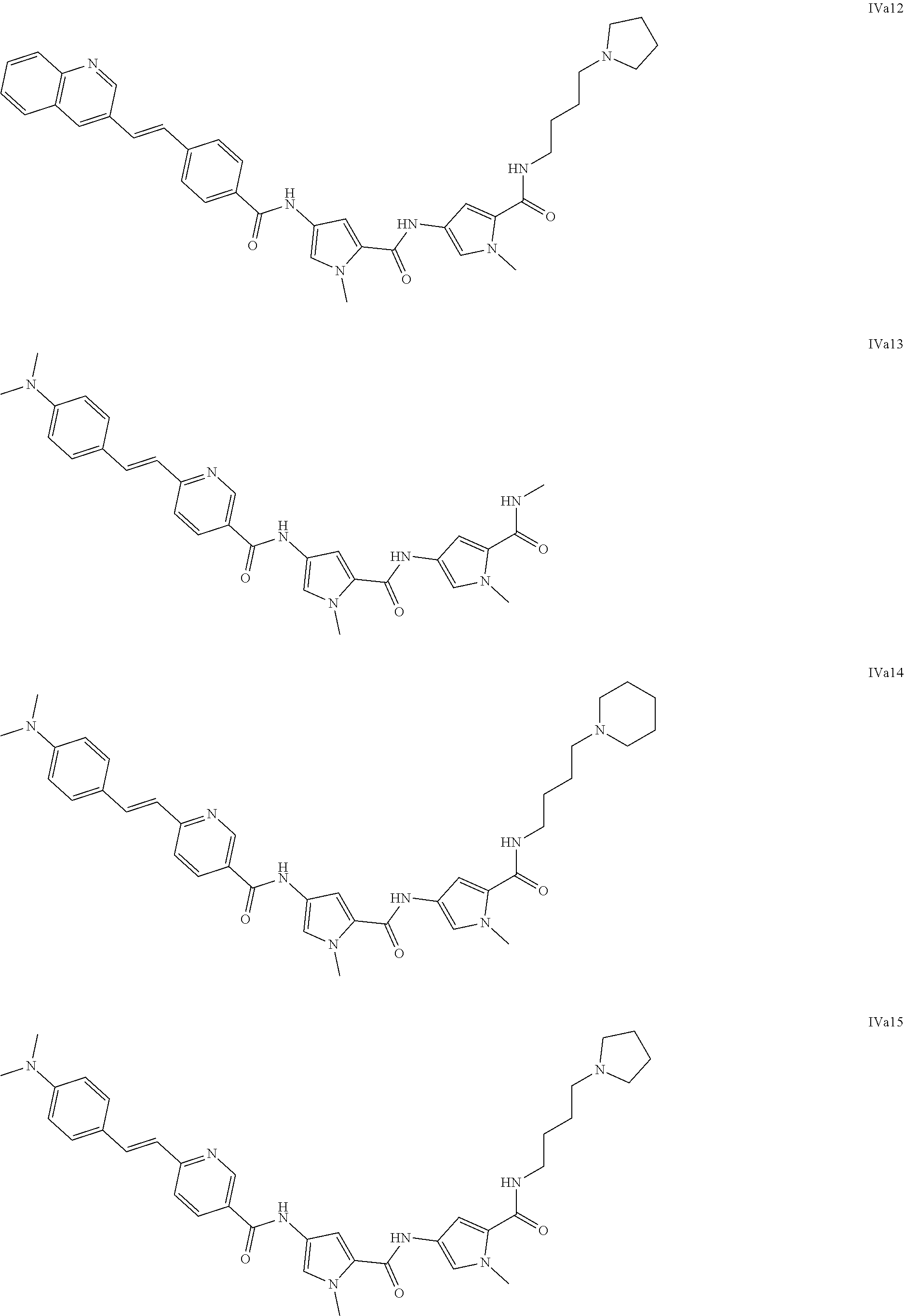

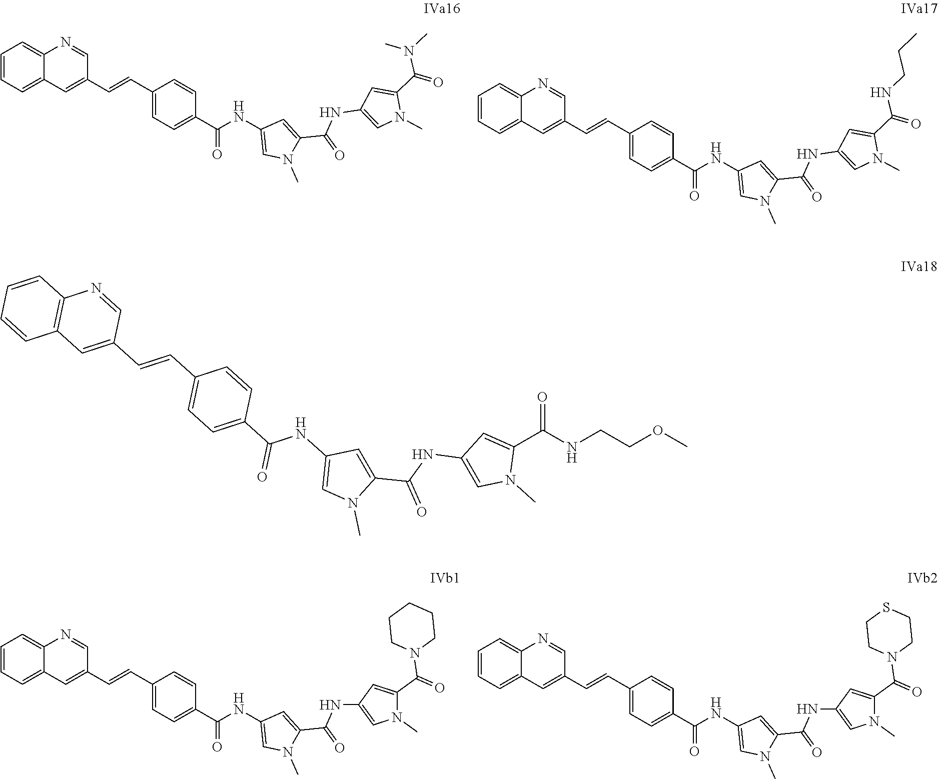 [SMILES: CN(C)C(=O)c1cc(NC(=O)c2cc(NC(=O)c3ccc(/C=C/c4cnc5ccccc5c4)cc3)cn2C)cn1C ; CCCNC(=O)c1cc(NC(=O)c2cc(NC(=O)c3ccc(/C=C/c4cnc5ccccc5c4)cc3)cn2C)cn1C ; COCCNC(=O)c1cc(NC(=O)c2cc(NC(=O)c3ccc(/C=C/c4cnc5ccccc5c4)cc3)cn2C)cn1C ; Cn1cc(NC(=O)c2ccc(/C=C/c3cnc4ccccc4c3)cc2)cc1C(=O)Nc1cc(C(=O)N2CCCCC2)n(C)c1 ; Cn1cc(NC(=O)c2ccc(/C=C/c3cnc4ccccc4c3)cc2)cc1C(=O)Nc1cc(C(=O)N2CCSCC2)n(C)c1]

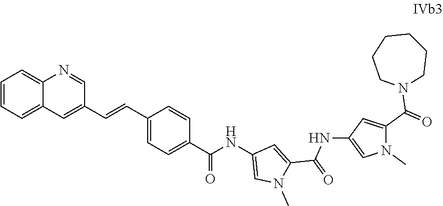 [SMILES: Cn1cc(NC(=O)c2ccc(/C=C/c3cnc4ccccc4c3)cc2)cc1C(=O)Nc1cc(C(=O)N2CCCCCC2)n(C)c1]

22. A composition comprising one or more compounds according to claim 1 and a pharmaceutically acceptable excipient.

23. (canceled)

24. (canceled)

25. A method of treatment of any one or more conditions selected from the group consisting of a viral, bacterial, fungal and parasitic infection, and cancer, said method comprising administering an effective amount of a compound according to claim 1 to a patient in need thereof.

26. The method of claim 25, wherein:
(i) the viral infection is caused by any one of the group consisting of Respiratory Syncytial Virus, Human Rhino Virus, Human Influenza Virus, Influenza virus such as Influenza viruses A and B, Norovirus, Dengue virus, Yellow fever virus, West Nile virus, Zika Virus, Rift Valley fever virus, African swine fever virus, Japanese encephalitis virus, Nipah virus, coronavirus such as SARS-CoV-2, adenoviruses such as Titi monkey adenovirus, herpes viruses such as Macacine herpes virus, and polyomaviruses;
(ii) the bacterial infection is caused by any one of the group consisting of Staphylococcus aureus, Enterococci, Streptococci, Clostridia, Corynebacteria, tubercular and non-tubercular mycobacteria, Enterobacteriaceae, Acinetobacter baumannii, Pseudomonas aeruginosa, Helicobacter pylori, Campylobacter spp., Salmonellae, Neisseria gonorrhoeae, Haemophilus influenzae, and Shigella spp;
(iii) the fungal infection is caused by any one of the group consisting of Candida albicans, Candida auris, Candida glabrata, Candida parapsilosis, Candida krusei, Candida guilliiermondii, Cryptococccus neoformans, Cryptococcus gattii, Aspergillus fumigatus, Aspergillus flavus, Rhizopus arrhizus, Fusarium oxysporum, Fusarium solani, Scedosporium prolificans, Lomentospora prolificans, Blastomyces dermatitidis, Paecilomyces variotii, Mucor spp., Pneumocystis jirovecii, Histoplasma capsulatum, Coccidioides immitis and Coccidioides posadasii; and
(iv) the parasitic infection is caused by any one of the group consisting of Trypanosoma brucei brucei, Trypanosoma brucei gambiense, Trypanosoma brucei rhodesiense, Trypanosoma congolense, Trypanosoma equiperdum, Trypanosoma evansi, Trypanosoma cruzi, Trichomonas vaginalis, Toxoplasma gondii, Plasmodia spp., Leishmania spp., and Acanthamoeba spp.

27. (canceled)

28. A method of treatment of any one or more conditions selected from the group consisting of a viral, bacterial, fungal and parasitic infection, and cancer, said method comprising administering an effective amount of a composition according to claim 22 to a patient in need thereof.

29. The method of claim 28, wherein:
(i) the viral infection is caused by any one of the group consisting of Respiratory Syncytial Virus, Human Rhino Virus, Human Influenza Virus, Influenza virus such as Influenza viruses A and B, Norovirus, Dengue virus, Yellow fever virus, West Nile virus, Zika Virus, Rift Valley fever virus, African swine fever virus, Japanese encephalitis virus, Nipah virus, coronavirus such as SARS-CoV-2, adenoviruses such as Titi monkey adenovirus, herpes viruses such as Macacine herpes virus, and polyomaviruses;
(ii) the bacterial infection is caused by any one of the group consisting of Staphylococcus aureus, Enterococci, Streptococci, Clostridia, Corynebacteria, tubercular and non-tubercular mycobacteria, Enterobacteriaceae, Acinetobacter baumannii, Pseudomonas aeruginosa, Helicobacter pylori, Campylobacter spp., Salmonellae, Neisseria gonorrhoeae, Haemophilus influenzae, and Shigella spp;
(iii) the fungal infection is caused by any one of the group consisting of Candida albicans, Candida auris, Candida glabrata, Candida parapsilosis, Candida krusei, Candida guilliiermondii, Cryptococccus neoformans, Cryptococcus gattii, Aspergillus fumigatus, Aspergillus flavus, Rhizopus arrhizus, Fusarium oxysporum, Fusarium solani, Scedosporium prolificans, Lomentospora prolificans, Blastomyces dermatitidis, Paecilomyces variotii, Mucor spp., Pneumocystis jirovecii, Histoplasma capsulatum, Coccidioides immitis and Coccidioides posadasii; and
(iv) the parasitic infection is caused by any one of the group consisting of Trypanosoma brucei brucei, Trypanosoma brucei gambiense, Trypanosoma brucei rhodesiense, Trypanosoma congolense, Trypanosoma equiperdum, Trypanosoma evansi, Trypanosoma cruzi, Trichomonas vaginalis, Toxoplasma gondii, Plasmodia spp., Leishmania spp., and Acanthamoeba spp.

30. A method of inhibiting the binding to DNA and/or RNA of one or more enzymes or regulatory proteins comprising contacting the DNA and/or RNA with a compound according to claim 1, wherein the binding is inhibited ex vivo or in vitro.
